(19) United States
(12) Patent Application Publication
(10) Pub. No.: US 2009/0186034 A1
(43) Pub. Date: Jul. 23, 2009

(54) Gene expression markers for inflammatory bowel disease

(75) Inventors: Alexander Abbas, San Carlos, CA (US); Hilary Clark, San Francisco, CA (US); Lauri Diehl, Los Altos, CA (US)

Correspondence Address: Goodwin Procter LLP;Attn: Patent Administrator, 135 Commonwealth Drive, Menlo Park, CA, 94025-1105, US

(73) Assignee: Genetech, Inc., South San Francisco, CA (US)

(21) Appl. No.: 12/004,525

(22) Filed: Dec. 19, 2007

Related U.S. Application Data
(60) Provisional application No. 60/870,811, filed on Dec. 19, 2006.

(51) Int. Cl. C12Q 1/68; A61K 31/4402; A61K 31/195; A61K 31/196; A61K 31/58; A61K 31/573; A61K 31/52; A61K 31/519; A61K 38/13; A61K 39/395

(52) U.S. Cl. 424/141.1; 435/6; 514/352; 514/565; 514/567; 514/563; 514/174; 514/177; 514/263.3; 514/263.2; 514/249; 514/11

(57) ABSTRACT

The present invention relates to methods of gene expression profiling for inflammatory bowel disease pathogenesis, in which the differential expression in a test sample from a mammalian subject of one or more IBD markers relative to a control is determined, wherein the differential expression in the test sample is indicative of an IBD in the mammalian subject from which the test sample was obtained.

### CROSS-REFERENCE TO RELATED APPLICATIONS

This application claims priority under Section 119(e) and the benefit of U.S. Provisional Application Ser. No. 60/870,811 filed Dec. 19, 2006 and United States Provisional Application having attorney docket number 39766-0288 PR-1 filed Nov. 29, 2007, the entire disclosures of which are incorporated herein in their entirety.

### BACKGROUND OF THE INVENTION

1. Field of the Invention

The present invention relates to gene expression profiles in inflammatory bowel disease pathogenesis, including use in the detection and diagnosis of inflammatory bowel disease.

2. Description of Related Art

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

Many immune related diseases are known and have been extensively studied. Such diseases include immune-mediated inflammatory diseases, non-immune-mediated inflammatory diseases, infectious diseases, immunodeficiency diseases, neoplasia, etc.

The term inflammatory bowel disorder (“IBD”) describes a group of chronic inflammatory disorders of unknown causes in which the intestine (bowel) becomes inflamed, often causing recurring cramps or diarrhea. The prevalence of IBD in the US is estimated to be about 200 per 100,000 population. Patients with IBD can be divided into two major groups, those with ulcerative colitis (“UC”) and those with Crohn's disease (“CD”). Both UC and CD are chronic relapsing diseases and are complex clinical entities that occur in genetically susceptible individuals who are exposed to as yet poorly defined environmental stimuli. (Bonen and Cho, Gastroenterology. 2003; 124:521-536; Gaya et al. Lancet. 2006; 367:1271-1284)

Although the cause of IBD remains unknown, several factors such as genetic, infectious and immunologic susceptibility have been implicated. IBD is much more common in Caucasians, especially those of Jewish descent. The chronic inflammatory nature of the condition has prompted an intense search for a possible infectious cause. Although agents have been found which stimulate acute inflammation, none has been found to cause the chronic inflammation associated with IBD. The hypothesis that IBD is an autoimmune disease is supported by the previously mentioned extraintestinal manifestation of IBD as joint arthritis, and the known positive response to IBD by treatment with therapeutic agents such as adrenal glucocorticoids, cyclosporine and azathioprine, which are known to suppress immune response. In addition, the GI tract, more than any other organ of the body, is continuously exposed to potential antigenic substances such as proteins from food, bacterial byproducts (LPS), etc.

There is sufficient overlap in the diagnostic criteria for UC and CD that it is sometimes impossible to say which a given patient has; however, the type of lesion typically seen is different, as is the localization. UC mostly appears in the colon, proximal to the rectum, and the characteristic lesion is a superficial ulcer of the mucosa; CD can appear anywhere in the bowel, with occasional involvement of stomach, esophagus and duodenum, and the lesions are usually described as extensive linear fissures.

The current therapy of IBD usually involves the administration of antiinflammatory or immunosuppressive agents, such as sulfasalazine, corticosteroids, 6-mercaptopurine/azathioprine, or cyclosporine, which usually bring only partial results. If anti-inflammatory/immunosuppressive therapies fail, colectomies are the last line of defense. The typical operation for CD not involving the rectum is resection (removal of a diseased segment of bowel) and anastomosis (reconnection) without an ostomy. Sections of the small or large intestine may be removed. About 30% of CD patients will need surgery within the first year after diagnosis. In the subsequent years, the rate is about 5% per year. Unfortunately, CD is characterized by a high rate of recurrence; about 5% of patients need a second surgery each year after initial surgery.

Refining a diagnosis of inflammatory bowel disease involves evaluating the progression status of the diseases using standard classification criteria. The classification systems used in IBD include the Truelove and Witts Index (Truelove S. C. and Witts, L. J. Br Med J. 1955; 2:1041-1048), which classifies colitis as mild, moderate, or severe, as well as Lennard-Jones. (Lennard-Jones J E. Scand J Gastroenterol Suppl 1989; 170:2-6) and the simple clinical colitis activity index (SCCAI). (Walmsley et. al. Gut. 1998; 43:29-32) These systems track such variables as daily bowel movements, rectal bleeding, temperature, heart rate, hemoglobin levels, erythrocyte sedimentation rate, weight, hematocrit score, and the level of serum albumin.

In approximately 10-15% of cases, a definitive diagnosis of ulcerative colitis or Crohn's disease cannot be made and such cases are often referred to as “indeterminate colitis.” Two antibody detection tests are available that can help the diagnosis, each of which assays for antibodies in the blood. The antibodies are “perinuclear anti-neutrophil antibody” (pANCA) and “anti-Saccharomyces cervisiae antibody” (ASCA). Most patients with ulcerative colitis have the pANCA antibody but not the ASCA antibody, while most patients with Crohn's disease have the ASCA antibody but not the pANCA antibody. However, these two tests have shortcomings as some patients have neither antibody and some Crohn's disease patients may have only the pANCA antibody.

For clinical practice, a reliable test that would indicate the presence and/or progression of an IBD based on molecular markers rather than the measurement of a multitude of variables would be useful for identifying and/or treating individuals with an IBD.

Hypothesis free, linkage and association studies have identified genetic loci that have been associated with UC, notably the MHC region on chromosome 6, (Rioux et al. Am J Hum Genet. 2000; 66:1863-1870; Stokkers et al. Gut. 1999; 45:395-401; Van Heel et al. Hum Mol. Genet. 2004; 13:763-770) the IBD2 locus on chromosome 12 (Parkes et al. Am J Hum Genet. 2000; 67:1605-1610; Satsangi et al. Nat. Genet. 1996; 14:199-202) and the IBD5 locus on chromosome 5. (Giallourakis et. al. Am J. Hum Genet. 2003; 73:205-211; Palmieri et. al Aliment Pharmacol Ther. 2006; 23:497-506; Russell et. al. Gut. 2006; 55:1114-1123; Waller et. al. Gut. 2006; 55:809-814) Following a UK wide linkage scan identifying a putative loci of association for UC on chromosome 7q, further studies have implicated variants in the ABCB1 (MDR1) gene which is involved in cellular detoxification with UC. (Satsangi et. al. Nat. Genet. 1996; 14:199-202; Brant et. al. Am J Hum Genet. 2003; 73:1282-1292; Ho et. al. Gastroenterology. 2005; 128:288-296).

A complementary approach towards the identification and understanding of the complex gene-gene and gene-environment relationships that result in the chronic intestinal inflammation observed in inflammatory bowel disease (IBD) is microarray gene expression analysis. Microarrays allow a comprehensive picture of gene expression at the tissue and cellular level, thus helping understand the underlying patho-physiological processes. (Stoughton et. al. Annu Rev Biochem. 2005; 74:53-82) Microarray analysis was first applied to patients with IBD in 1997, comparing expression of 96 genes in surgical resections of patients with CD to synovial tissue of patients with rheumatoid arthritis. (Heller et. al. Proc Natl Acad Sci USA. 1997; 94:2150-2155) Further studies using microarray platforms to interrogate surgical specimens from patients with IBD identified an number of novel genes that were differentially regulated when diseased samples were compared to controls. (Dieckgraefe et. al. Physiol Genomics. 2000; 4:1-11; Lawrance et. al. Hum Mol. Genet. 2001; 10:445-456).

Endoscopic pinch mucosal biopsies have allowed investigators to microarray tissue from a larger range of patients encompassing those with less severe disease. Langmann et. al. used microarray technology to analyze 22,283 genes in biopsy specimens from macroscopically non affected areas of the colon and terminal ileum. (Langmann et. al. Gastroenterology. 2004; 127:26-40) Genes which were involved in cellular detoxification and biotransformation (Pregnane X receptor and MDR1) were significantly downregulated in the colon of patients with UC, however, there was no change in the expression of these genes in the biopsies from patients with CD. Costello and colleagues (Costello et. al. PLoS Med. 2005; 2:e199) looked at the expression of 33792 sequences in endoscopic sigmoid colon biopsies obtained from healthy controls, patients with CD and UC. A number of sequences representing novel proteins were differentially regulated and in silico analysis suggested that these proteins had putative functions related to disease pathogenesis-transcription factors, signaling molecules and cell adhesion.

In a study of patients with UC, Okahara et al. (Aliment Pharmacol Ther. 2005; 21:1091-1097) observed that (migration inhibitory factor-related protein 14 (MRP14), growth-related oncogene gamma (GROγ) and serum amyloid A1 (SAA1) were upregulated where as TIMP1 and elfin were down regulated in the inflamed biopsies when compared to the non-inflamed biopsies. When observing 41 chemokines and 21 chemokine receptors, Puleston et al demonstrated that chemokines CXCLs 1-3 and 8 and CCL20 were upregulated in active colonic CD and UC. (Aliment Pharmacol Ther. 2005; 21:109-120) Overall these studies illustrate the heterogeneity of early microarray platforms and tissue collection. However, despite these problems differential expression of a number of genes was consistently observed.

Despite the above identified advances in IBD research, there is a great need for additional diagnostic and therapeutic agents capable of detecting IBD in a mammal and for effectively treating this disorder. Accordingly, the present invention provides polynucleotides and polypeptides that are overexpressed in IBD as compared to normal tissue, and methods of using those polypeptides, and their encoding nucleic acids, for to detect or diagnose the presence of an IBD in mammalian subjects and subsequently to treat those subjects in which an IBD is detected with suitable IBD therapeutic agents.

The present invention provides methods for detecting the presence of and determining the progression of inflammatory bowel disease (IBD), including Crohn's disease (CD).

The invention disclosed herein provides methods and assays examining expression of one or more gene expression markers in a mammalian tissue or cell sample, wherein the expression of one or more such biomarkers is predictive of whether the mammalian subject from which the tissue or cell sample was taken is more likely to have an IBD. In various embodiments of the invention, the methods and assays examine the expression of gene expression markers such as those provided herein as SEQ ID NOS: 1-426 and determine whether expression is higher or lower than a control sample.

These and further embodiments of the present invention will be apparent to those of ordinary skill in the art.

### SUMMARY OF THE INVENTION

In one aspect, the invention concerns a method of detecting or diagnosing an inflammatory bowel disease (IBD) in a mammalian subject comprising determining, in a biological sample obtained from the subject, that expression level of one or more nucleic acids encoding a polypeptide shown as any one of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 10, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, and 426 is different relative to the expression level in a control, wherein the difference in expression indicates the subject is more likely to have an IBD. In all embodiments, the expression level of RNA transcripts or their expression products of one or more genes selected from SEQ ID NOS:1-426 may be determined.

In one embodiment, the methods of diagnosing or detecting the presence of an IBD in a mammalian subject comprise determining that the expression level of one or more nucleic acids encoding a polypeptide shown as any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, and 316 in a test sample obtained from the subject is higher relative to the level of expression in a control, wherein the higher level of expression is indicative of the presence of an IBD in the subject from which the test sample was obtained. In all embodiments, the expression level of RNA transcripts or their expression products of one or more genes selected from SEQ ID NOS:1-316 may be determined.

In another embodiment, the methods of diagnosing or detecting the presence of an IBD in a mammalian subject comprise determining that the expression level of one or more nucleic acid(s) encoding a polypeptide shown as any one of SEQ ID NOS: 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, and 426 in a test sample obtained from the subject is lower relative to the level of expression in a control, wherein the lower level of expression is indicative of the presence of an IBD in the subject from which the test sample was obtained. In all embodiments, the expression level of RNA transcripts or their expression products of one or more genes selected from SEQ ID NOS:317-426 may be determined.

In one aspect, the methods are directed to diagnosing or detecting a recurrence of an IBD in mammalian subject that was previously diagnosed with an IBD and is currently in remission. The subject may have completed treatment for the IBD or is currently undergoing treatment for the IBD. In one embodiment, the methods comprise determining, in a biological sample obtained from the mammalian subject, that one or more nucleic acids encoding a polypeptide shown as SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, and 316 is different relative to the expression level in a control, wherein the difference in expression indicates the presence of the recurrence of an IBD. In a preferred embodiment, the recurrence of the IBD is detected in a mammalian subject with an inactive IBD. Alternatively, the test sample may be compared to a prior test sample of the mammalian subject, if available, obtained before, after, or at the time of the initial IBD diagnosis. In all embodiments, the expression level of RNA transcripts or their expression products of one or more genes selected from SEQ ID NOS:1-426 may determined.

In all aspects, the recurrence of the IBD is a flare-up of an inactive IBD.

In all aspects, the mammalian subject preferably is a human patient, such as a human patient diagnosed with or at risk of developing an IBD. The subject may also be an IBD patient who has received prior treatment for an IBD but is at risk of a recurrence of the IBD.

For all aspects of the method of the invention, determining the expression level of one or more nucleic acids encoding a polypeptide described herein (or the expression level of RNA transcripts or their expression products of one or more genes selected from SEQ ID NOS:1-426) may be obtained, for example, by a method of gene expression profiling. The method of gene expression profiling may be, for example, a PCR-based method.

In various embodiments, the diagnosis includes quantification of the expression level of one or more nucleic acids encoding a polypeptide described herein (or the expression level of RNA transcripts or their expression products of one or more genes selected from SEQ ID NOS: 1-426) such as by immunohistochemistry (IHC) and/or fluorescence in situ hybridization (FISH).

For all aspects of the invention, the expression levels of the nucleic acids (or RNA transcripts or their expression products of one or more genes selected from SEQ ID NOS: 1-426) may be normalized relative to the expression levels of one or more reference genes, or their expression products.

For all aspects of the invention, the method may further comprise determining evidence of the expression levels of at least two of said nucleic acids (or genes), or their expression products. It is further contemplated that the method of the invention may further comprise determining evidence of the expression levels of at least three of said nucleic acids (or genes), or their expression products. It is also contemplated that the method of the invention may further comprise determining evidence of the expression levels of at least four of said nucleic acids (or genes), or their expression products. It is also contemplated that the method of the invention may further comprise determining evidence of the expression levels of at least five of said nucleic acids (or genes), or their expression products. It is also contemplated that the method of the invention may further comprise determining evidence of the expression levels of at least six of said nucleic acids (or genes), or their expression products. It is also contemplated that the method of the invention may further comprise determining evidence of the expression levels of at least seven of said nucleic acids (or genes), or their expression products. It is also contemplated that the method of the invention may further comprise determining evidence of the expression levels of at least eight of said nucleic acids (or genes), or their expression products. It is also contemplated that the method of the invention may further comprise determining evidence of the expression levels of at least nine of said nucleic acids (or genes), or their expression products. It is also contemplated that the method of the invention may further comprise determining evidence of the expression levels of at least ten of said nucleic acids (or genes), or their expression products. It is also contemplated that the method of the invention may further comprise determining evidence of the expression levels of at least eleven of said nucleic acids (or genes), or their expression products. It is also contemplated that the method of the invention may further comprise determining evidence of the expression levels of at least twelve of said nucleic acids (or genes), or their expression products. It is also contemplated that the method of the invention may further comprise determining evidence of the expression levels of at least thirteen of said nucleic acids (or genes), or their expression products. It is also contemplated that the method of the invention may further comprise determining evidence of the expression levels of at least fourteen of said nucleic acids (or genes), or their expression products. It is also contemplated that the method of the invention may further comprise determining evidence of the expression levels of at least fifteen of said nucleic acids (or genes), or their expression products. It is also contemplated that the method of the invention may further comprise determining evidence of the expression levels of at least sixteen of said nucleic acids (or genes), or their expression products. It is also contemplated that the method of the invention may further comprise determining evidence of the expression levels of at least seventeen of said nucleic acids (or genes), or their expression products. It is also contemplated that the method of the invention may further comprise determining evidence of the expression levels of at least eighteen of said nucleic acids (or genes), or their expression products. It is also contemplated that the method of the invention may further comprise determining evidence of the expression levels of at least nineteen of said nucleic acids (or genes), or their expression products. It is also contemplated that the method of the invention may further comprise determining evidence of the expression levels of at least twenty of said nucleic acids (or genes), or their expression products.

In another aspect, the methods of present invention also contemplate the use of a “panel” of such nucleic acids or genes (i.e. IBD markers as disclosed herein) based on evidence of their level of expression. Such panels may be used to screen a mammalian subject in order to make a determination on whether an IBD is present in the subject. In some embodiments, the panel of IBD markers will include at least 1 IBD marker, at least 2 IBD markers, at least 3 IBD markers, at least 4 IBD markers, at least 5 IBD markers, at least 6 IBD markers, at least 7 IBD marker, at least 8 IBD markers, at least 9 IBD markers, at least 10 IBD markers, at least 11 IBD markers, at least 12 IBD markers, at least 13 IBD markers, at least 14 IBD markers, at least 15 IBD markers, at least 16 IBD markers, at least 17 IBD markers, at least 18 IBD markers, at least 19 IBD markers, or at least 20 IBD markers. The panel may include an IBD marker that is overexpressed in IBD relative to a control, an IBD marker that is underexpressed in IBD relative to a control, or IBD markers that are both overexpressed and underexpressed in IBD relative to a control. In one embodiment, the IBD markers that make up the panel are selected from SEQ ID NOS: 1-426. Such panels may be used to screen a mammalian subject for the differential expression of one or more IBD markers in order to make a determination on whether an IBD is present in the subject, or whether a recurrence of an inactive IBD is present in the subject.

In one embodiment, the IBD markers that make up the panel are selected from SEQ ID NOS:1-426. In a preferred embodiment, the methods of diagnosing or detecting the presence of an IBD in a mammalian subject comprise determining a differential expression level of RNA transcripts or expression products thereof from a panel of IBD markers in a test sample obtained from the subject relative to the level of expression in a control, wherein the differential level of expression is indicative of the presence of an IBD in the subject from which the test sample was obtained. The differential expression in the test sample may be higher and/or lower relative to a control as discussed herein.

In one embodiment, the panel includes only those IBD markers that are up-regulated in Crohn's disease. In a preferred embodiment, the panel of up-regulated markers includes one or more of the markers shown as SEQ ID NOS:1-316. In another embodiment, the panel includes only those IBD markers that are down-regulated in Crohn's disease. In a preferred embodiment, the panel of down-regulated markers include one or more of the markers shown as SEQ ID NOS:317-426.

For all aspects of the invention, the method may further comprise the step of creating a report summarizing said prediction.

For all aspects, the IBD diagnosed or detected according to the methods of the present invention is Crohn's disease (CD).

For all aspects of the invention, the test sample obtained from a mammalian subject may be derived from a colonic tissue biopsy. In a preferred embodiment, the biopsy is a tissue selected from the group consisting of terminal ileum, the ascending colon, the descending colon, and the sigmoid colon. In other preferred embodiments, the biopsy is from an inflamed colonic area or from a non-inflamed colonic area. The inflamed colonic area may be acutely inflamed or chronically inflamed. In a preferred embodiment, the biopsy is tissue from the terminal ileum.

For all aspects, determination of expression levels may occur at more than one time. For all aspects of the invention, the determination of expression levels may occur before the patient is subjected to any therapy before and/or after any surgery. In some embodiments, the determining step is indicative of a recurrence of an IBD in the mammalian subject following surgery or indicative of a recurrence of said IBD in said mammalian subject. In a preferred embodiment, the IBD is Crohn's disease. In all aspects, the recurrence of an IBD may be a flare-up of an inactive IBD. In another preferred embodiment, the recurrence of an IBD is a flare-up of Crohn's disease that had previously been inactive or in remission.

In another aspect, the present invention concerns methods of treating a mammalian subject in which the presence of an IBD has been detected by the methods described herein. For example, following a determination that a test sample obtained from the mammalian subject exhibits differential expression relative to a control of one or more nucleic acids encoding a polypeptide described herein (or of the RNA transcripts or the corresponding gene products) corresponding to an IBD marker described herein, the mammalian subject may be administered an IBD therapeutic agent.

In one embodiment, the methods of treating an IBD in a mammalian subject in need thereof, comprise (a) determining a differential level of expression of one or more nucleic acids encoding a polypeptide shown as any one of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, and 426 in a test sample obtained from said subject relative to the level of expression in a control, wherein said differential level of expression is indicative of the presence of an IBD in the subject from which the test sample was obtained; and (b) administering to said subject an effective amount of an IBD therapeutic agent. In all embodiments, the expression level of RNA transcripts or their expression products of one or more genes selected from SEQ ID NOS:1-426 may be determined.

In a preferred embodiment, the methods of treating an IBD comprise (a) determining that the expression level of one or more nucleic acids encoding a polypeptide shown as any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, and 316 in a test sample obtained from the subject is higher relative to the level of expression in a control, wherein the higher level of expression is indicative of the presence of an IBD in the subject from which the test sample was obtained; and (b) administering to said subject an effective amount of an IBD therapeutic agent. In another preferred embodiment, the methods of treating an IBD comprise (a) determining that the expression level of one or more nucleic acid(s) encoding a polypeptide shown as any one of SEQ ID NOS: 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, and 426 in a test sample obtained from the subject is lower relative to the level of expression in a control, wherein the lower level of expression is indicative of the presence of an IBD in the subject from which the test sample was obtained. In some preferred embodiments, the IBD therapeutic agent is one or more of an aminosalicylate, a corticosteroid, and an immunosuppressive agent.

In one aspect, the panel of IBD markers discussed above is useful in methods of treating an IBD in a mammalian subject. In one embodiment, the mammalian subject is screened against the panel of markers and if the presence of an IBD is determined, IBD therapeutic agent(s) may be administered as discussed herein.

In a different aspect the invention concerns a kit comprising one or more of (1) extraction buffer/reagents and protocol; (2) reverse transcription buffer/reagents and protocol; and (3) qPCR buffer/reagents and protocol suitable for performing the methods of this invention. The kit may comprise data retrieval and analysis software.

All patent, patent application, and literature references cited in the present specification are hereby incorporated by reference in their entirety.

### BRIEF DESCRIPTION OF DRAWINGS

FIG. 1 shows a nucleotide sequence (SEQ ID NO:1).

FIG. 2 shows the amino acid sequence (SEQ ID NO:2) derived from the coding sequence of SEQ ID NO:1 shown in FIG. 1.

FIG. 3 shows a nucleotide sequence (SEQ ID NO:3).

FIG. 4 shows the amino acid sequence (SEQ ID NO:4) derived from the coding sequence of SEQ ID NO:3 shown in FIG. 3.

FIG. 5 shows a nucleotide sequence (SEQ ID NO:5).

FIG. 6 shows the amino acid sequence (SEQ ID NO:6) derived from the coding sequence of SEQ ID NO:5 shown in FIG. 5.

FIG. 7 shows a nucleotide sequence (SEQ ID NO:7).

FIG. 8 shows the amino acid sequence (SEQ ID NO:8) derived from the coding sequence of SEQ ID NO:7 shown in FIG. 7.

FIG. 9 shows a nucleotide sequence (SEQ ID NO:9).

FIG. 10 shows the amino acid sequence (SEQ ID NO:10) derived from the coding sequence of SEQ ID NO:9 shown in FIG. 9.

FIG. 11 shows a nucleotide sequence (SEQ ID NO:11).

FIG. 12 shows the amino acid sequence (SEQ ID NO:12) derived from the coding sequence of SEQ ID NO:11 shown in FIG. 11.

FIG. 13 shows a nucleotide sequence (SEQ ID NO:13).

FIG. 14 shows the amino acid sequence (SEQ ID NO:14) derived from the coding sequence of SEQ ID NO:13 shown in FIG. 13.

FIG. 15 shows a nucleotide sequence (SEQ ID NO:15).

FIG. 16 shows the amino acid sequence (SEQ ID NO:16) derived from the coding sequence of SEQ ID NO:15 shown in FIG. 15.

FIG. 17 shows a nucleotide sequence (SEQ ID NO:17).

FIG. 18 shows the amino acid sequence (SEQ ID NO:18) derived from the coding sequence of SEQ ID NO:17 shown in FIG. 17.

FIG. 19 shows a nucleotide sequence (SEQ ID NO:19).

FIG. 20 shows the amino acid sequence (SEQ ID NO:20) derived from the coding sequence of SEQ ID NO:19 shown in FIG. 19.

FIG. 21 shows a nucleotide sequence (SEQ ID NO:21).

FIG. 22 shows the amino acid sequence (SEQ ID NO:22) derived from the coding sequence of SEQ ID NO:21 shown in FIG. 21.

FIG. 23 shows a nucleotide sequence (SEQ ID NO:23).

FIG. 24 shows the amino acid sequence (SEQ ID NO:24) derived from the coding sequence of SEQ ID NO:23 shown in FIG. 23.

FIG. 25 shows a nucleotide sequence (SEQ ID NO:25).

FIG. 26 shows the amino acid sequence (SEQ ID NO:26) derived from the coding sequence of SEQ ID NO:25 shown in FIG. 25.

FIG. 27 shows a nucleotide sequence (SEQ ID NO:27).

FIG. 28 shows the amino acid sequence (SEQ ID NO:28) derived from the coding sequence of SEQ ID NO:27 shown in FIG. 27.

FIG. 29 shows a nucleotide sequence (SEQ ID NO:29).

FIG. 30 shows the amino acid sequence (SEQ ID NO:30) derived from the coding sequence of SEQ ID NO:29 shown in FIG. 29.

FIG. 31 shows a nucleotide sequence (SEQ ID NO:31).

FIG. 32 shows the amino acid sequence (SEQ ID NO:32) derived from the coding sequence of SEQ ID NO:31 shown in FIG. 31.

FIG. 33 shows a nucleotide sequence (SEQ ID NO:33).

FIG. 34 shows the amino acid sequence (SEQ ID NO:34) derived from the coding sequence of SEQ ID NO:33 shown in FIG. 33.

FIG. 35 shows a nucleotide sequence (SEQ ID NO:35).

FIG. 36 shows the amino acid sequence (SEQ ID NO:36) derived from the coding sequence of SEQ ID NO:35 shown in FIG. 35.

FIG. 37 shows a nucleotide sequence (SEQ ID NO:37).

FIG. 38 shows the amino acid sequence (SEQ ID NO:38) derived from the coding sequence of SEQ ID NO:37 shown in FIG. 37.

FIG. 39 shows a nucleotide sequence (SEQ ID NO:39).

FIG. 40 shows the amino acid sequence (SEQ ID NO:40) derived from the coding sequence of SEQ ID NO:39 shown in FIG. 39.

FIG. 41 shows a nucleotide sequence (SEQ ID NO:41).

FIG. 42 shows the amino acid sequence (SEQ ID NO:42) derived from the coding sequence of SEQ ID NO:41 shown in FIG. 41.

FIG. 43 shows a nucleotide sequence (SEQ ID NO:43).

FIG. 44 shows the amino acid sequence (SEQ ID NO:44) derived from the coding sequence of SEQ ID NO:43 shown in FIG. 43.

FIG. 45 shows a nucleotide sequence (SEQ ID NO:45).

FIG. 46 shows the amino acid sequence (SEQ ID NO:46) derived from the coding sequence of SEQ ID NO:45 shown in FIG. 45.

FIG. 47 shows a nucleotide sequence (SEQ ID NO:47).

FIG. 48 shows the amino acid sequence (SEQ ID NO:48) derived from the coding sequence of SEQ ID NO:47 shown in FIG. 47.

FIG. 49 shows a nucleotide sequence (SEQ ID NO:49).

FIG. 50 shows the amino acid sequence (SEQ ID NO:50) derived from the coding sequence of SEQ ID NO:49 shown in FIG. 49.

FIG. 51 shows a nucleotide sequence (SEQ ID NO:51).

FIG. 52 shows the amino acid sequence (SEQ ID NO:52) derived from the coding sequence of SEQ ID NO:51 shown in FIG. 51.

FIG. 53 shows a nucleotide sequence (SEQ ID NO:53).

FIG. 54 shows the amino acid sequence (SEQ ID NO:54) derived from the coding sequence of SEQ ID NO:53 shown in FIG. 53.

FIG. 55 shows a nucleotide sequence (SEQ ID NO:55).

FIG. 56 shows the amino acid sequence (SEQ ID NO:56) derived from the coding sequence of SEQ ID NO:55 shown in FIG. 55.

FIG. 57 shows a nucleotide sequence (SEQ ID NO:57).

FIG. 58 shows the amino acid sequence (SEQ ID NO:58) derived from the coding sequence of SEQ ID NO:57 shown in FIG. 57.

FIG. 59 shows a nucleotide sequence (SEQ ID NO:59).

FIG. 60 shows the amino acid sequence (SEQ ID NO:60) derived from the coding sequence of SEQ ID NO:59 shown in FIG. 59.

FIG. 61 shows a nucleotide sequence (SEQ ID NO:61).

FIG. 62 shows the amino acid sequence (SEQ ID NO:62) derived from the coding sequence of SEQ ID NO:61 shown in FIG. 61.

FIG. 63 shows a nucleotide sequence (SEQ ID NO:63).

FIG. 64 shows the amino acid sequence (SEQ ID NO:64) derived from the coding sequence of SEQ ID NO:63 shown in FIG. 63.

FIG. 65 shows a nucleotide sequence (SEQ ID NO:65).

FIG. 66 shows the amino acid sequence (SEQ ID NO:66) derived from the coding sequence of SEQ ID NO:65 shown in FIG. 65.

FIG. 67 shows a nucleotide sequence (SEQ ID NO:67).

FIG. 68 shows the amino acid sequence (SEQ ID NO:68) derived from the coding sequence of SEQ ID NO:67 shown in FIG. 67.

FIG. 69 shows a nucleotide sequence (SEQ ID NO:69).

FIG. 70 shows the amino acid sequence (SEQ ID NO:70) derived from the coding sequence of SEQ ID NO:69 shown in FIG. 69.

FIG. 71 shows a nucleotide sequence (SEQ ID NO:71).

FIG. 72 shows the amino acid sequence (SEQ ID NO:72) derived from the coding sequence of SEQ ID NO:71 shown in FIG. 71.

FIG. 73 shows a nucleotide sequence (SEQ ID NO:73).

FIG. 74 shows the amino acid sequence (SEQ ID NO:74) derived from the coding sequence of SEQ ID NO:73 shown in FIG. 73.

FIG. 75 shows a nucleotide sequence (SEQ ID NO:75).

FIG. 76 shows the amino acid sequence (SEQ ID NO:76) derived from the coding sequence of SEQ ID NO:75 shown in FIG. 75.

FIG. 77 shows a nucleotide sequence (SEQ ID NO:77).

FIG. 78 shows the amino acid sequence (SEQ ID NO:78) derived from the coding sequence of SEQ ID NO:77 shown in FIG. 77.

FIG. 79 shows a nucleotide sequence (SEQ ID NO:79).

FIG. 80 shows the amino acid sequence (SEQ ID NO:80) derived from the coding sequence of SEQ ID NO:79 shown in FIG. 79.

FIG. 81 shows a nucleotide sequence (SEQ ID NO:81).

FIG. 82 shows the amino acid sequence (SEQ ID NO:82) derived from the coding sequence of SEQ ID NO:81 shown in FIG. 81.

FIG. 83 shows a nucleotide sequence (SEQ ID NO:83).

FIG. 84 shows the amino acid sequence (SEQ ID NO:84) derived from the coding sequence of SEQ ID NO:83 shown in FIG. 83.

FIG. 85 shows a nucleotide sequence (SEQ ID NO:85).

FIG. 86 shows the amino acid sequence (SEQ ID NO:86) derived from the coding sequence of SEQ ID NO:85 shown in FIG. 85.

FIG. 87 shows a nucleotide sequence (SEQ ID NO:87).

FIG. 88 shows the amino acid sequence (SEQ ID NO:88) derived from the coding sequence of SEQ ID NO:87 shown in FIG. 87.

FIG. 89 shows a nucleotide sequence (SEQ ID NO:89).

FIG. 90 shows the amino acid sequence (SEQ ID NO:90) derived from the coding sequence of SEQ ID NO:89 shown in FIG. 89.

FIG. 91 shows a nucleotide sequence (SEQ ID NO:91).

FIG. 92 shows the amino acid sequence (SEQ ID NO:92) derived from the coding sequence of SEQ ID NO:91 shown in FIG. 91.

FIG. 93 shows a nucleotide sequence (SEQ ID NO:93).

FIG. 94 shows the amino acid sequence (SEQ ID NO:94) derived from the coding sequence of SEQ ID NO:93 shown in FIG. 93.

FIG. 95 shows a nucleotide sequence (SEQ ID NO:95).

FIG. 96 shows the amino acid sequence (SEQ ID NO:96) derived from the coding sequence of SEQ ID NO:95 shown in FIG. 95.

FIG. 97 shows a nucleotide sequence (SEQ ID NO:97).

FIG. 98 shows the amino acid sequence (SEQ ID NO:98) derived from the coding sequence of SEQ ID NO:97 shown in FIG. 97.

FIG. 99 shows a nucleotide sequence (SEQ ID NO:99).

FIG. 100 shows the amino acid sequence (SEQ ID NO:100) derived from the coding sequence of SEQ ID NO:99 shown in FIG. 99.

FIG. 101 shows a nucleotide sequence (SEQ ID NO:101).

FIG. 102 shows the amino acid sequence (SEQ ID NO:102) derived from the coding sequence of SEQ ID NO:101 shown in FIG. 101.

FIG. 103 shows a nucleotide sequence (SEQ ID NO:103).

FIG. 104 shows the amino acid sequence (SEQ ID NO:104) derived from the coding sequence of SEQ ID NO:103 shown in FIG. 103.

FIG. 105 shows a nucleotide sequence (SEQ ID NO:105).

FIG. 106 shows the amino acid sequence (SEQ ID NO:106) derived from the coding sequence of SEQ ID NO:105 shown in FIG. 105.

FIG. 107 shows a nucleotide sequence (SEQ ID NO:107).

FIG. 108 shows the amino acid sequence (SEQ ID NO:108) derived from the coding sequence of SEQ ID NO:107 shown in FIG. 107.

FIG. 109 shows a nucleotide sequence (SEQ ID NO:109).

FIG. 110 shows the amino acid sequence (SEQ ID NO:110) derived from the coding sequence of SEQ ID NO:109 shown in FIG. 109.

FIG. 111 shows a nucleotide sequence (SEQ ID NO:111).

FIG. 112 shows the amino acid sequence (SEQ ID NO:112) derived from the coding sequence of SEQ ID NO:111 shown in FIG. 111.

FIG. 113 shows a nucleotide sequence (SEQ ID NO:113).

FIG. 114 shows the amino acid sequence (SEQ ID NO:114) derived from the coding sequence of SEQ ID NO:113 shown in FIG. 113.

FIG. 115 shows a nucleotide sequence (SEQ ID NO:115).

FIG. 116 shows the amino acid sequence (SEQ ID NO:116) derived from the coding sequence of SEQ ID NO:115 shown in FIG. 115.

FIG. 117 shows a nucleotide sequence (SEQ ID NO:117):

FIG. 118 shows the amino acid sequence (SEQ ID NO:118) derived from the coding sequence of SEQ ID NO:117 shown in FIG. 117.

FIG. 119 shows a nucleotide sequence (SEQ ID NO:119).

FIG. 120 shows the amino acid sequence (SEQ ID NO:120) derived from the coding sequence of SEQ ID NO:119 shown in FIG. 119.

FIG. 121 shows a nucleotide sequence (SEQ ID NO:121).

FIG. 122 shows the amino acid sequence (SEQ ID NO:122) derived from the coding sequence of SEQ ID NO:121 shown in FIG. 121.

FIG. 123 shows a nucleotide sequence (SEQ ID NO:123).

FIG. 124 shows the amino acid sequence (SEQ ID NO:124) derived from the coding sequence of SEQ ID NO:123 shown in FIG. 123.

FIG. 125 shows a nucleotide sequence (SEQ ID NO:125).

FIG. 126 shows the amino acid sequence (SEQ ID NO:126) derived from the coding sequence of SEQ ID NO:125 shown in FIG. 125.

FIG. 127 shows a nucleotide sequence (SEQ ID NO:127).

FIG. 128 shows the amino acid sequence (SEQ ID NO:128) derived from the coding sequence of SEQ ID NO:127 shown in FIG. 127.

FIG. 129 shows a nucleotide sequence (SEQ ID NO:129).

FIG. 130 shows the amino acid sequence (SEQ ID NO:130) derived from the coding sequence of SEQ ID NO:129 shown in FIG. 129.

FIG. 131 shows a nucleotide sequence (SEQ ID NO:131).

FIG. 132 shows the amino acid sequence (SEQ ID NO:132) derived from the coding sequence of SEQ ID NO:131 shown in FIG. 131.

FIG. 133 shows a nucleotide sequence (SEQ ID NO:133).

FIG. 134 shows the amino acid sequence (SEQ ID NO:134) derived from the coding sequence of SEQ ID NO:133 shown in FIG. 133.

FIG. 135 shows a nucleotide sequence (SEQ ID NO:135).

FIG. 136 shows the amino acid sequence (SEQ ID NO:136) derived from the coding sequence of SEQ ID NO:135 shown in FIG. 135.

FIG. 137 shows a nucleotide sequence (SEQ ID NO:137).

FIG. 138 shows the amino acid sequence (SEQ ID NO:138) derived from the coding sequence of SEQ ID NO:137 shown in FIG. 137.

FIG. 139 shows a nucleotide sequence (SEQ ID NO:139).

FIG. 140 shows the amino acid sequence (SEQ ID NO:140) derived from the coding sequence of SEQ ID NO:139 shown in FIG. 139.

FIG. 141 shows a nucleotide sequence (SEQ ID NO:141).

FIG. 142 shows the amino acid sequence (SEQ ID NO:142) derived from the coding sequence of SEQ ID NO:141 shown in FIG. 141.

FIG. 143 shows a nucleotide sequence (SEQ ID NO:143).

FIG. 144 shows the amino acid sequence (SEQ ID NO:144) derived from the coding sequence of SEQ ID NO:143 shown in FIG. 143.

FIG. 145 shows a nucleotide sequence (SEQ ID NO:145).

FIG. 146 shows the amino acid sequence (SEQ ID NO:146) derived from the coding sequence of SEQ ID NO:145 shown in FIG. 145.

FIG. 147 shows a nucleotide sequence (SEQ ID NO:147).

FIG. 148 shows the amino acid sequence (SEQ ID NO:148) derived from the coding sequence of SEQ ID NO:147 shown in FIG. 147.

FIG. 149 shows a nucleotide sequence (SEQ ID NO:149).

FIG. 150 shows the amino acid sequence (SEQ ID NO:150) derived from the coding sequence of SEQ ID NO:149 shown in FIG. 149.

FIG. 151 shows a nucleotide sequence (SEQ ID NO:151).

FIG. 152 shows the amino acid sequence (SEQ ID NO:152) derived from the coding sequence of SEQ ID NO:151 shown in FIG. 151.

FIG. 153 shows a nucleotide sequence (SEQ ID NO:153).

FIG. 154 shows the amino acid sequence (SEQ ID NO: 154) derived from the coding sequence of SEQ ID NO:153 shown in FIG. 153.

FIG. 155 shows a nucleotide sequence (SEQ ID NO:155).

FIG. 156 shows the amino acid sequence (SEQ ID NO:156) derived from the coding sequence of SEQ ID NO:155 shown in FIG. 155.

FIG. 157 shows a nucleotide sequence (SEQ ID NO:157).

FIG. 158 shows the amino acid sequence (SEQ ID NO:158) derived from the coding sequence of SEQ ID NO:157 shown in FIG. 157.

FIG. 159 shows a nucleotide sequence (SEQ ID NO:159).

FIG. 160 shows the amino acid sequence (SEQ ID NO:160) derived from the coding sequence of SEQ ID NO:159 shown in FIG. 159.

FIG. 161 shows a nucleotide sequence (SEQ ID NO:161).

FIG. 162 shows the amino acid sequence (SEQ ID NO:162) derived from the coding sequence of SEQ ID NO:161 shown in FIG. 161.

FIG. 163 shows a nucleotide sequence (SEQ ID NO:163).

FIG. 164 shows the amino acid sequence (SEQ ID NO:164) derived from the coding sequence of SEQ ID NO:163 shown in FIG. 163.

FIG. 165 shows a nucleotide sequence (SEQ ID NO:165).

FIG. 166 shows the amino acid sequence (SEQ ID NO:166) derived from the coding sequence of SEQ ID NO:165 shown in FIG. 165.

FIG. 167 shows a nucleotide sequence (SEQ ID NO:167).

FIG. 168 shows the amino acid sequence (SEQ ID NO:168) derived from the coding sequence of SEQ ID NO:167 shown in FIG. 167.

FIG. 169 shows a nucleotide sequence (SEQ ID NO:169).

FIG. 170 shows the amino acid sequence (SEQ ID NO:170) derived from the coding sequence of SEQ ID NO:169 shown in FIG. 169.

FIG. 171 shows a nucleotide sequence (SEQ ID NO:171).

FIG. 172 shows the amino acid sequence (SEQ ID NO:172) derived from the coding sequence of SEQ ID NO:171 shown in FIG. 171.

FIG. 173 shows a nucleotide sequence (SEQ ID NO:173).

FIG. 174 shows the amino acid sequence (SEQ ID NO:174) derived from the coding sequence of SEQ ID NO:173 shown in FIG. 173.

FIG. 175 shows a nucleotide sequence (SEQ ID NO:175).

FIG. 176 shows the amino acid sequence (SEQ ID NO:176) derived from the coding sequence of SEQ ID NO:175 shown in FIG. 175.

FIG. 177 shows a nucleotide sequence (SEQ ID NO:177).

FIG. 178 shows the amino acid sequence (SEQ ID NO:178) derived from the coding sequence of SEQ ID NO:177 shown in FIG. 177.

FIG. 179 shows a nucleotide sequence (SEQ ID NO:179).

FIG. 180 shows the amino acid sequence (SEQ ID NO:180) derived from the coding sequence of SEQ ID NO:179 shown in FIG. 179.

FIG. 181 shows a nucleotide sequence (SEQ ID NO:181).

FIG. 182 shows the amino acid sequence (SEQ ID NO:182) derived from the coding sequence of SEQ ID NO:181 shown in FIG. 181.

FIG. 183 shows a nucleotide sequence (SEQ ID NO:183).

FIG. 184 shows the amino acid sequence (SEQ ID NO:184) derived from the coding sequence of SEQ ID NO:183 shown in FIG. 183.

FIG. 185 shows a nucleotide sequence (SEQ ID NO:185).

FIG. 186 shows the amino acid sequence (SEQ ID NO:186) derived from the coding sequence of SEQ ID NO:185 shown in FIG. 185.

FIG. 187 shows a nucleotide sequence (SEQ ID NO:187).

FIG. 188 shows the amino acid sequence (SEQ ID NO:188) derived from the coding sequence of SEQ ID NO:187 shown in FIG. 187.

FIG. 189 shows a nucleotide sequence (SEQ ID NO:189).

FIG. 190 shows the amino acid sequence (SEQ ID NO:190) derived from the coding sequence of SEQ ID NO:189 shown in FIG. 189.

FIG. 191 shows a nucleotide sequence (SEQ ID NO:191).

FIG. 192 shows the amino acid sequence (SEQ ID NO:192) derived from the coding sequence of SEQ ID NO:191 shown in FIG. 191.

FIG. 193 shows a nucleotide sequence (SEQ ID NO:193).

FIG. 194 shows the amino acid sequence (SEQ ID NO:194) derived from the coding sequence of SEQ ID NO:193 shown in FIG. 193.

FIG. 195 shows a nucleotide sequence (SEQ ID NO:195).

FIG. 196 shows the amino acid sequence (SEQ ID NO:196) derived from the coding sequence of SEQ ID NO:195 shown in FIG. 195.

FIG. 197 shows a nucleotide sequence (SEQ ID NO:197).

FIG. 198 shows the amino acid sequence (SEQ ID NO:198) derived from the coding sequence of SEQ ID NO:197 shown in FIG. 197.

FIG. 199 shows a nucleotide sequence (SEQ ID NO:199).

FIG. 200 shows the amino acid sequence (SEQ ID NO:200) derived from the coding sequence of SEQ ID NO:199 shown in FIG. 199.

FIG. 201 shows a nucleotide sequence (SEQ ID NO:201).

FIG. 202 shows the amino acid sequence (SEQ ID NO:202) derived from the coding sequence of SEQ ID NO:201 shown in FIG. 201.

FIG. 203 shows a nucleotide sequence (SEQ ID NO:203).

FIG. 204 shows the amino acid sequence (SEQ ID NO:204) derived from the coding sequence of SEQ ID NO:203 shown in FIG. 203.

FIG. 205 shows a nucleotide sequence (SEQ ID NO:205).

FIG. 206 shows the amino acid sequence (SEQ ID NO:206) derived from the coding sequence of SEQ ID NO:205 shown in FIG. 205.

FIG. 207 shows a nucleotide sequence (SEQ ID NO:207).

FIG. 208 shows the amino acid sequence (SEQ ID NO:208) derived from the coding sequence of SEQ ID NO:207 shown in FIG. 207.

FIG. 209 shows a nucleotide sequence (SEQ ID NO:209).

FIG. 210 shows the amino acid sequence (SEQ ID NO:210) derived from the coding sequence of SEQ ID NO:209 shown in FIG. 209.

FIG. 211 shows a nucleotide sequence (SEQ ID NO:211).

FIG. 212 shows the amino acid sequence (SEQ ID NO:212) derived from the coding sequence of SEQ ID NO:211 shown in FIG. 211.

FIG. 213 shows a nucleotide sequence (SEQ ID NO:213).

FIG. 214 shows the amino acid sequence (SEQ ID NO:214) derived from the coding sequence of SEQ ID NO:213 shown in FIG. 213.

FIG. 215 shows a nucleotide sequence (SEQ ID NO:215).

FIG. 216 shows the amino acid sequence (SEQ ID NO:216) derived from the coding sequence of SEQ ID NO:215 shown in FIG. 215.

FIG. 217 shows a nucleotide sequence (SEQ ID NO:217).

FIG. 218 shows the amino acid sequence (SEQ ID NO:218) derived from the coding sequence of SEQ ID NO:217 shown in FIG. 217.

FIG. 219 shows a nucleotide sequence (SEQ ID NO:219).

FIG. 220 shows the amino acid sequence (SEQ ID NO:220) derived from the coding sequence of SEQ ID NO:219 shown in FIG. 219.

FIG. 221 shows a nucleotide sequence (SEQ ID NO:221).

FIG. 222 shows the amino acid sequence (SEQ ID NO:222) derived from the coding sequence of SEQ ID NO:221 shown in FIG. 221.

FIG. 223 shows a nucleotide sequence (SEQ ID NO:223).

FIG. 224 shows the amino acid sequence (SEQ ID NO:224) derived from the coding sequence of SEQ ID NO:223 shown in FIG. 223.

FIG. 225 shows a nucleotide sequence (SEQ ID NO:225).

FIG. 226 shows the amino acid sequence (SEQ ID NO:226) derived from the coding sequence of SEQ ID NO:225 shown in FIG. 225.

FIG. 227 shows a nucleotide sequence (SEQ ID NO:227).

FIG. 228 shows the amino acid sequence (SEQ ID NO:228) derived from the coding sequence of SEQ ID NO:227 shown in FIG. 227.

FIG. 229 shows a nucleotide sequence (SEQ ID NO:229).

FIG. 230 shows the amino acid sequence (SEQ ID NO:230) derived from the coding sequence of SEQ ID NO:229 shown in FIG. 229.

FIG. 231 shows a nucleotide sequence (SEQ ID NO:231).

FIG. 232 shows the amino acid sequence (SEQ ID NO:232) derived from the coding sequence of SEQ ID NO:231 shown in FIG. 231.

FIG. 233 shows a nucleotide sequence (SEQ ID NO:233).

FIG. 234 shows the amino acid sequence (SEQ ID NO:234) derived from the coding sequence of SEQ ID NO:233 shown in FIG. 233.

FIG. 235 shows a nucleotide sequence (SEQ ID NO:235).

FIG. 236 shows the amino acid sequence (SEQ ID NO:236) derived from the coding sequence of SEQ ID NO:235 shown in FIG. 235.

FIG. 237 shows a nucleotide sequence (SEQ ID NO:237).

FIG. 238 shows the amino acid sequence (SEQ ID NO:238) derived from the coding sequence of SEQ ID NO:237 shown in FIG. 237.

FIG. 239 shows a nucleotide sequence (SEQ ID NO:239).

FIG. 240 shows the amino acid sequence (SEQ ID NO:240) derived from the coding sequence of SEQ ID NO:239 shown in FIG. 239.

FIG. 241 shows a nucleotide sequence (SEQ ID NO:241).

FIG. 242 shows the amino acid sequence (SEQ ID NO:242) derived from the coding sequence of SEQ ID NO:241 shown in FIG. 241.

FIG. 243 shows a nucleotide sequence (SEQ ID NO:243).

FIG. 244 shows the amino acid sequence (SEQ ID NO:244) derived from the coding sequence of SEQ ID NO:243 shown in FIG. 243.

FIG. 245 shows a nucleotide sequence (SEQ ID NO:245).

FIG. 246 shows the amino acid sequence (SEQ ID NO:246) derived from the coding sequence of SEQ ID NO:245 shown in FIG. 245.

FIG. 247 shows a nucleotide sequence (SEQ ID NO:247).

FIG. 248 shows the amino acid sequence (SEQ ID NO:248) derived from the coding sequence of SEQ ID NO:247 shown in FIG. 247.

FIG. 249 shows a nucleotide sequence (SEQ ID NO:249).

FIG. 250 shows the amino acid sequence (SEQ ID NO:250) derived from the coding sequence of SEQ ID NO:249 shown in FIG. 249.

FIG. 251 shows a nucleotide sequence (SEQ ID NO:251).

FIG. 252 shows the amino acid sequence (SEQ ID NO:252) derived from the coding sequence of SEQ ID NO:251 shown in FIG. 251.

FIG. 253 shows a nucleotide sequence (SEQ ID NO:253).

FIG. 254 shows the amino acid sequence (SEQ ID NO:254) derived from the coding sequence of SEQ ID NO. 253 shown in FIG. 253.

FIG. 255 shows a nucleotide sequence (SEQ ID NO:255).

FIG. 256 shows the amino acid sequence (SEQ ID NO:256) derived from the coding sequence of SEQ ID NO:255 shown in FIG. 255.

FIG. 257 shows a nucleotide sequence (SEQ ID NO:257).

FIG. 258 shows the amino acid sequence (SEQ ID NO:258) derived from the coding sequence of SEQ ID NO:257 shown in FIG. 257.

FIG. 259 shows a nucleotide sequence (SEQ ID NO:259).

FIG. 260 shows the amino acid sequence (SEQ ID NO:260) derived from the coding sequence of SEQ ID NO:259 shown in FIG. 259.

FIG. 261 shows a nucleotide sequence (SEQ ID NO:261).

FIG. 262 shows the amino acid sequence (SEQ ID NO:262) derived from the coding sequence of SEQ ID NO:261 shown in FIG. 261.

FIG. 263 shows a nucleotide sequence (SEQ ID NO:263).

FIG. 264 shows the amino acid sequence (SEQ ID NO:264) derived from the coding sequence of SEQ ID NO:263 shown in FIG. 263.

FIG. 265 shows a nucleotide sequence (SEQ ID NO:265).

FIG. 266 shows the amino acid sequence (SEQ ID NO:266) derived from the coding sequence of SEQ ID NO: 265 shown in FIG. 265.

FIG. 267 shows a nucleotide sequence (SEQ ID NO:267).

FIG. 268 shows the amino acid sequence (SEQ ID NO:268) derived from the coding sequence of SEQ ID NO:267 shown in FIG. 267.

FIG. 269 shows a nucleotide sequence (SEQ ID NO:269).

FIG. 270 shows the amino acid sequence (SEQ ID NO:270) derived from the coding sequence of SEQ ID NO:269 shown in FIG. 269.

FIG. 271 shows a nucleotide sequence (SEQ ID NO:271).

FIG. 272 shows the amino acid sequence (SEQ ID NO:272) derived from the coding sequence of SEQ ID NO:271 shown in FIG. 271.

FIG. 273 shows a nucleotide sequence (SEQ ID NO:273).

FIG. 274 shows the amino acid sequence (SEQ ID NO:274) derived from the coding sequence of SEQ ID NO:273 shown in FIG. 273.

FIG. 275 shows a nucleotide sequence (SEQ ID NO:275).

FIG. 276 shows the amino acid sequence (SEQ ID NO:276) derived from the coding sequence of SEQ ID NO:275 shown in FIG. 275.

FIG. 277 shows a nucleotide sequence (SEQ ID NO:277).

FIG. 278 shows the amino acid sequence (SEQ ID NO:278) derived from the coding sequence of SEQ ID NO:277 shown in FIG. 277.

FIG. 279 shows a nucleotide sequence (SEQ ID NO:279).

FIG. 280 shows the amino acid sequence (SEQ ID NO:280) derived from the coding sequence of SEQ ID NO:279 shown in FIG. 279.

FIG. 281 shows a nucleotide sequence (SEQ ID NO:281).

FIG. 282 shows the amino acid sequence (SEQ ID NO:282) derived from the coding sequence of SEQ ID NO:281 shown in FIG. 281.

FIG. 283 shows a nucleotide sequence (SEQ ID NO:283).

FIG. 284 shows the amino acid sequence (SEQ ID NO:284) derived from the coding sequence of SEQ ID NO:283 shown in FIG. 283.

FIG. 285 shows a nucleotide sequence (SEQ ID NO:285).

FIG. 286 shows the amino acid sequence (SEQ ID NO:286) derived from the coding sequence of SEQ ID NO:285 shown in FIG. 285.

FIG. 287 shows a nucleotide sequence (SEQ ID NO:287).

FIG. 288 shows the amino acid sequence (SEQ ID NO:288) derived from the coding sequence of SEQ ID NO:287 shown in FIG. 287.

FIG. 289 shows a nucleotide sequence (SEQ ID NO:289).

FIG. 290 shows the amino acid sequence (SEQ ID NO:290) derived from the coding sequence of SEQ ID NO:289 shown in FIG. 289.

FIG. 291 shows a nucleotide sequence (SEQ ID NO:291).

FIG. 292 shows the amino acid sequence (SEQ ID NO:292) derived from the coding sequence of SEQ ID NO:291 shown in FIG. 291.

FIG. 293 shows a nucleotide sequence (SEQ ID NO:293).

FIG. 294 shows the amino acid sequence (SEQ ID NO:294) derived from the coding sequence of SEQ ID NO:293 shown in FIG. 293.

FIG. 295 shows a nucleotide sequence (SEQ ID NO:295).

FIG. 296 shows the amino acid sequence (SEQ ID NO:296) derived from the coding sequence of SEQ ID NO:295 shown in FIG. 295.

FIG. 297 shows a nucleotide sequence (SEQ ID NO:297).

FIG. 298 shows the amino acid sequence (SEQ ID NO:298) derived from the coding sequence of SEQ ID NO:297 shown in FIG. 297.

FIG. 299 shows a nucleotide sequence (SEQ ID NO:299).

FIG. 300 shows the amino acid sequence (SEQ ID NO:300) derived from the coding sequence of SEQ ID NO:299 shown in FIG. 299.

FIG. 301 shows a nucleotide sequence (SEQ ID NO:301).

FIG. 302 shows the amino acid sequence (SEQ ID NO:302) derived from the coding sequence of SEQ ID NO:301 shown in FIG. 301.

FIG. 303 shows a nucleotide sequence (SEQ ID NO:303).

FIG. 304 shows the amino acid sequence (SEQ ID NO:304) derived from the coding sequence of SEQ ID NO:303 shown in FIG. 303.

FIG. 305 shows a nucleotide sequence (SEQ ID NO:305).

FIG. 306 shows the amino acid sequence (SEQ ID NO:306) derived from the coding sequence of SEQ ID NO:305 shown in FIG. 305.

FIG. 307 shows a nucleotide sequence (SEQ ID NO:307).

FIG. 308 shows the amino acid sequence (SEQ ID NO:308) derived from the coding sequence of SEQ ID NO:307 shown in FIG. 307.

FIG. 309 shows a nucleotide sequence (SEQ ID NO:309).

FIG. 310 shows the amino acid sequence (SEQ ID NO:310) derived from the coding sequence of SEQ ID NO:309 shown in FIG. 309.

FIG. 311 shows a nucleotide sequence (SEQ ID NO:311).

FIG. 312 shows the amino acid sequence (SEQ ID NO:312) derived from the coding sequence of SEQ ID NO:311 shown in FIG. 311.

FIG. 313 shows a nucleotide sequence (SEQ ID NO:313).

FIG. 314 shows the amino acid sequence (SEQ ID NO:314) derived from the coding sequence of SEQ ID NO:313 shown in FIG. 313.

FIG. 315 shows a nucleotide sequence (SEQ ID NO:315).

FIG. 316 shows the amino acid sequence (SEQ ID NO:316) derived from the coding sequence of SEQ ID NO:315 shown in FIG. 315.

FIG. 317 shows a nucleotide sequence (SEQ ID NO:317).

FIG. 318 shows the amino acid sequence (SEQ ID NO:318) derived from the coding sequence of SEQ ID NO:317 shown in FIG. 317.

FIG. 319 shows a nucleotide sequence (SEQ ID NO:319).

FIG. 320 shows the amino acid sequence (SEQ ID NO:320) derived from the coding sequence of SEQ ID NO:319 shown in FIG. 319.

FIG. 321 shows a nucleotide sequence (SEQ ID NO:321).

FIG. 322 shows the amino acid sequence (SEQ ID NO:322) derived from the coding sequence of SEQ ID NO:321 shown in FIG. 321.

FIG. 323 shows a nucleotide sequence (SEQ ID NO:323).

FIG. 324 shows the amino acid sequence (SEQ ID NO:324) derived from the coding sequence of SEQ ID NO:323 shown in FIG. 323.

FIG. 325 shows a nucleotide sequence (SEQ ID NO:325).

FIG. 326 shows the amino acid sequence (SEQ ID NO:326) derived from the coding sequence of SEQ ID NO:325 shown in FIG. 325.

FIG. 327 shows a nucleotide sequence (SEQ ID NO:327).

FIG. 328 shows the amino acid sequence (SEQ ID NO:328) derived from the coding sequence of SEQ ID NO:327 shown in FIG. 327.

FIG. 329 shows a nucleotide sequence (SEQ ID NO:329).

FIG. 330 shows the amino acid sequence (SEQ ID NO:330) derived from the coding sequence of SEQ ID NO:329 shown in FIG. 329.

FIG. 331 shows a nucleotide sequence (SEQ ID NO:331).

FIG. 332 shows the amino acid sequence (SEQ ID NO:332) derived from the coding sequence of SEQ ID NO:331 shown in FIG. 331.

FIG. 333 shows a nucleotide sequence (SEQ ID NO:333).

FIG. 334 shows the amino acid sequence (SEQ ID NO:334) derived from the coding sequence of SEQ ID NO:333 shown in FIG. 333.

FIG. 335 shows a nucleotide sequence (SEQ ID NO:335).

FIG. 336 shows the amino acid sequence (SEQ ID NO:336) derived from the coding sequence of SEQ ID NO:335 shown in FIG. 335.

FIG. 337 shows a nucleotide sequence (SEQ ID NO:337).

FIG. 338 shows the amino acid sequence (SEQ ID NO:338) derived from the coding sequence of SEQ ID NO:337 shown in FIG. 337.

FIG. 339 shows a nucleotide sequence (SEQ ID NO:339).

FIG. 340 shows the amino acid sequence (SEQ ID NO:340) derived from the coding sequence of SEQ ID NO:339 shown in FIG. 339.

FIG. 341 shows a nucleotide sequence (SEQ ID NO:341).

FIG. 342 shows the amino acid sequence (SEQ ID NO:342) derived from the coding sequence of SEQ ID NO:341 shown in FIG. 341.

FIG. 343 shows a nucleotide sequence (SEQ ID NO:343).

FIG. 344 shows the amino acid sequence (SEQ ID NO:344) derived from the coding sequence of SEQ ID NO:343 shown in FIG. 343.

FIG. 345 shows a nucleotide sequence (SEQ ID NO:345).

FIG. 346 shows the amino acid sequence (SEQ ID NO:346) derived from the coding sequence of SEQ ID NO:345 shown in FIG. 345.

FIG. 347 shows a nucleotide sequence (SEQ ID NO:347).

FIG. 348 shows the amino acid sequence (SEQ ID NO:348) derived from the coding sequence of SEQ ID NO:347 shown in FIG. 347.

FIG. 349 shows a nucleotide sequence (SEQ ID NO:349).

FIG. 350 shows the amino acid sequence (SEQ ID NO:350) derived from the coding sequence of SEQ ID NO:349 shown in FIG. 349.

FIG. 351 shows a nucleotide sequence (SEQ ID NO:351).

FIG. 352 shows the amino acid sequence (SEQ ID NO:352) derived from the coding sequence of SEQ ID NO:351 shown in FIG. 351.

FIG. 353 shows a nucleotide sequence (SEQ ID NO:353).

FIG. 354 shows the amino acid sequence (SEQ ID NO:354) derived from the coding sequence of SEQ ID NO:353 shown in FIG. 353.

FIG. 355 shows a nucleotide sequence (SEQ ID NO:355).

FIG. 356 shows the amino acid sequence (SEQ ID NO:356) derived from the coding sequence of SEQ ID NO:355 shown in FIG. 355.

FIG. 357 shows a nucleotide sequence (SEQ ID NO:357).

FIG. 358 shows the amino acid sequence (SEQ ID NO:358) derived from the coding sequence of SEQ ID NO:357 shown in FIG. 357.

FIG. 359 shows a nucleotide sequence (SEQ ID NO:359).

FIG. 360 shows the amino acid sequence (SEQ ID NO:360) derived from the coding sequence of SEQ ID NO:359 shown in FIG. 359.

FIG. 361 shows a nucleotide sequence (SEQ ID NO:361).

FIG. 362 shows the amino acid sequence (SEQ ID NO:362) derived from the coding sequence of SEQ ID NO:361 shown in FIG. 361.

FIG. 363 shows a nucleotide sequence (SEQ ID NO:363).

FIG. 364 shows the amino acid sequence (SEQ ID NO:364) derived from the coding sequence of SEQ ID NO:363 shown in FIG. 363.

FIG. 365 shows a nucleotide sequence (SEQ ID NO:365).

FIG. 366 shows the amino acid sequence (SEQ ID NO:366) derived from the coding sequence of SEQ ID NO:365 shown in FIG. 365.

FIG. 367 shows a nucleotide sequence (SEQ ID NO:367).

FIG. 368 shows the amino acid sequence (SEQ ID NO:368) derived from the coding sequence of SEQ ID NO:367 shown in FIG. 367.

FIG. 369 shows a nucleotide sequence (SEQ ID NO:369).

FIG. 370 shows the amino acid sequence (SEQ ID NO:370) derived from the coding sequence of SEQ ID NO:369 shown in FIG. 369.

FIG. 371 shows a nucleotide sequence (SEQ ID NO:371).

FIG. 372 shows the amino acid sequence (SEQ ID NO:372) derived from the coding sequence of SEQ ID NO:371 shown in FIG. 371.

FIG. 373 shows a nucleotide sequence (SEQ ID NO:373).

FIG. 374 shows the amino acid sequence (SEQ ID NO:374) derived from the coding sequence of SEQ ID NO:373 shown in FIG. 373.

FIG. 375 shows a nucleotide sequence (SEQ ID NO:375).

FIG. 376 shows the amino acid sequence (SEQ ID NO:376) derived from the coding sequence of SEQ ID NO:375 shown in FIG. 375.

FIG. 377 shows a nucleotide sequence (SEQ ID NO:377).

FIG. 378 shows the amino acid sequence (SEQ ID NO:378) derived from the coding sequence of SEQ ID NO:377 shown in FIG. 377.

FIG. 379 shows a nucleotide sequence (SEQ ID NO:379).

FIG. 380 shows the amino acid sequence (SEQ ID NO:380) derived from the coding sequence of SEQ ID NO:379 shown in FIG. 379.

FIG. 381 shows a nucleotide sequence (SEQ ID NO:381).

FIG. 382 shows the amino acid sequence (SEQ ID NO:382) derived from the coding sequence of SEQ ID NO:381 shown in FIG. 381.

FIG. 383 shows a nucleotide sequence (SEQ ID NO:383).

FIG. 384 shows the amino acid sequence (SEQ ID NO:384) derived from the coding sequence of SEQ ID NO:383 shown in FIG. 383.

FIG. 385 shows a nucleotide sequence (SEQ ID NO:385).

FIG. 386 shows the amino acid sequence (SEQ ID NO:386) derived from the coding sequence of SEQ ID NO:385 shown in FIG. 385.

FIG. 387 shows a nucleotide sequence (SEQ ID NO:387).

FIG. 388 shows the amino acid sequence (SEQ ID NO:388) derived from the coding sequence of SEQ ID NO:387 shown in FIG. 387.

FIG. 389 shows a nucleotide sequence (SEQ ID NO:389).

FIG. 390 shows the amino acid sequence (SEQ ID NO:390) derived from the coding sequence of SEQ ID NO:389 shown in FIG. 389.

FIG. 391 shows a nucleotide sequence (SEQ ID NO:391).

FIG. 392 shows the amino acid sequence (SEQ ID NO:392) derived from the coding sequence of SEQ ID NO:391 shown in FIG. 391.

FIG. 393 shows a nucleotide sequence (SEQ ID NO:393).

FIG. 394 shows the amino acid sequence (SEQ ID NO:394) derived from the coding sequence of SEQ ID NO:393 shown in FIG. 393.

FIG. 395 shows a nucleotide sequence (SEQ ID NO:395).

FIG. 396 shows the amino acid sequence (SEQ ID NO:396) derived from the coding sequence of SEQ ID NO:395 shown in FIG. 395.

FIG. 397 shows a nucleotide sequence (SEQ ID NO:397).

FIG. 398 shows the amino acid sequence (SEQ ID NO:398) derived from the coding sequence of SEQ ID NO:397 shown in FIG. 397.

FIG. 399 shows a nucleotide sequence (SEQ ID NO:399).

FIG. 400 shows the amino acid sequence (SEQ ID NO:400) derived from the coding sequence of SEQ ID NO:399 shown in FIG. 399.

FIG. 401 shows a nucleotide sequence (SEQ ID NO:401).

FIG. 402 shows the amino acid sequence (SEQ ID NO:402) derived from the coding sequence of SEQ ID NO:401 shown in FIG. 401.

FIG. 403 shows a nucleotide sequence (SEQ ID NO:403).

FIG. 404 shows the amino acid sequence (SEQ ID NO:404) derived from the coding sequence of SEQ ID NO:403 shown in FIG. 403.

FIG. 405 shows a nucleotide sequence (SEQ ID NO:405).

FIG. 406 shows the amino acid sequence (SEQ ID NO:406) derived from the coding sequence of SEQ ID NO:405 shown in FIG. 405.

FIG. 407 shows a nucleotide sequence (SEQ ID NO:407).

FIG. 408 shows the amino acid sequence (SEQ ID NO:408) derived from the coding sequence of SEQ ID NO:407 shown in FIG. 407.

FIG. 409 shows a nucleotide sequence (SEQ ID NO:409).

FIG. 410 shows the amino acid sequence (SEQ ID NO:410) derived from the coding sequence of SEQ ID NO:409 shown in FIG. 409.

FIG. 411 shows a nucleotide sequence (SEQ ID NO:411).

FIG. 412 shows the amino acid sequence (SEQ ID NO:412) derived from the coding sequence of SEQ ID NO:411 shown in FIG. 411.

FIG. 413 shows a nucleotide sequence (SEQ ID NO:413).

FIG. 414 shows the amino acid sequence (SEQ ID NO:414) derived from the coding sequence of SEQ ID NO:413 shown in FIG. 413.

FIG. 415 shows a nucleotide sequence (SEQ ID NO:415).

FIG. 416 shows the amino acid sequence (SEQ ID NO:416) derived from the coding sequence of SEQ ID NO:415 shown in FIG. 415.

FIG. 417 shows a nucleotide sequence (SEQ ID NO:417).

FIG. 418 shows the amino acid sequence (SEQ ID NO:418) derived from the coding sequence of SEQ ID NO:417 shown in FIG. 417.

FIG. 419 shows a nucleotide sequence (SEQ ID NO:419).

FIG. 420 shows the amino acid sequence (SEQ ID NO:420) derived from the coding sequence of SEQ ID NO:419 shown in FIG. 419.

FIG. 421 shows a nucleotide sequence (SEQ ID NO:421).

FIG. 422 shows the amino acid sequence (SEQ ID NO:422) derived from the coding sequence of SEQ ID NO:421 shown in FIG. 421.

FIG. 423 shows a nucleotide sequence (SEQ ID NO:423).

FIG. 424 shows the amino acid sequence (SEQ ID NO:424) derived from the coding sequence of SEQ ID NO:423 shown in FIG. 423.

FIG. 425 shows a nucleotide sequence (SEQ ID NO:425).

FIG. 426 shows the amino acid sequence (SEQ ID NO:426) derived from the coding sequence of SEQ ID NO:425 shown in FIG. 425.

### DETAILED DESCRIPTION OF THE PREFERRED EMBODIMENT

### A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “inflammatory bowel disease” or “IBD” is used as a collective term for ulcerative colitis and Crohn's disease. Although the two diseases are generally considered as two different entities, their common characteristics, such as patchy necrosis of the surface epithelium, focal accumulations of leukocytes adjacent to glandular crypts, and an increased number of intraepithelial lymphocytes (IEL) and certain macrophage subsets, justify their treatment as a single disease group.

The term “Crohn's disease” or “CD” is used herein to refer to a condition involving chronic inflammation of the gastrointestinal tract. Crohn's-related inflammation usually affects the intestines, but may occur anywhere from the mouth to the anus. CD differs from UC in that the inflammation extends through all layers of the intestinal wall and involves mesentery as well as lymph nodes. The disease is often discontinuous, i.e., severely diseased segments of bowel are separated from apparently disease-free areas. In CD, the bowel wall also thickens which can lead to obstructions, and the development of fistulas and fissures are not uncommon. As used herein, CD may be one or more of several types of CD, including without limitation, ileocolitis (affects the ileum and the large intestine); ileitis (affects the ileum); gastroduodenal CD (inflammation in the stomach and the duodenum); jejunoileitis (spotty patches of inflammation in the jejunum); and Crohn's (granulomatous) colitis (only affects the large intestine).

The term “ulcerative colitis” or “UC” is used herein to refer to a condition involving inflammation of the large intestine and rectum. In patients with UC, there is an inflammatory reaction primarily involving the colonic mucosa. The inflammation is typically uniform and continuous with no intervening areas of normal mucosa. Surface mucosal cells as well as crypt epithelium and submucosa are involved in an inflammatory reaction with neutrophil infiltration. Ultimately, this reaction typically progresses to epithelial damage and loss of epithelial cells resulting in multiple ulcerations, fibrosis, dysplasia and longitudinal retraction of the colon.

The term “inactive” IBD is used herein to mean an IBD that was previously diagnosed in an individual but is currently in remission. This is in contrast to an “active” IBD in which an individual has been diagnosed with and IBD but has not undergone treatment. In addition, the active IBD may be a recurrence of a previously diagnosed and treated IBD that had gone into remission (i.e. become an inactive IBD). Such recurrences may also be referred to herein as “flare-ups” of an IBD. Mammalian subjects having an active autoimmune disease, such as an IBD, may be subject to a flare-up, which is a period of heightened disease activity or a return of corresponding symptoms. Flare-ups may occur in response to severe infection, allergic reactions, physical stress, emotional trauma, surgery, or environmental factors.

The term “modulate” is used herein to mean that the expression of the gene, or level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator.

The terms “inhibit”, “down-regulate”, “underexpress” and “reduce” are used interchangeably and mean that the expression of a gene, or level of RNA molecules or equivalent RNA molecules encoding one or more proteins or protein subunits, or activity of one or more proteins or protein subunits, is reduced relative to one or more controls, such as, for example, one or more positive and/or negative controls.

The term “up-regulate” or “overexpress” is used to mean that the expression of a gene, or level of RNA molecules or equivalent RNA molecules encoding one or more proteins or protein subunits, or activity of one or more proteins or protein subunits, is elevated relative to one or more controls, such as, for example, one or more positive and/or negative controls.

The term “diagnosis” is used herein to refer to the identification of a molecular or pathological state, disease or condition, such as the identification of IBD.

The term “prognosis” is used herein to refer to the prediction of the likelihood of IBD development or progression, including autoimmune flare-ups and recurrences following surgery. Prognostic factors are those variables related to the natural history of IBD, which influence the recurrence rates and outcome of patients once they have developed IBD. Clinical parameters that may be associated with a worse prognosis include, for example, an abdominal mass or tenderness, skin rash, swollen joints, mouth ulcers, and borborygmus (gurgling or splashing sound over the intestine). Prognostic factors may be used to categorize patients into subgroups with different baseline recurrence risks.

The “pathology” of an IBD includes all phenomena that compromise the well-being of the patient. IBD pathology is primarily attributed to abnormal activation of the immune system in the intestines that can lead to chronic or acute inflammation in the absence of any known foreign antigen, and subsequent ulceration. Clinically, IBD is characterized by diverse manifestations often resulting in a chronic, unpredictable course. Bloody diarrhea and abdominal pain are often accompanied by fever and weight loss. Anemia is not uncommon, as is severe fatigue. Joint manifestations ranging from arthralgia to acute arthritis as well as abnormalities in liver function are commonly associated with IBD. During acute “attacks” of IBD, work and other normal activity are usually impossible, and often a patient is hospitalized.

The aetiology of these diseases is unknown and the initial lesion has not been clearly defined; however, patchy necrosis of the surface epithelium, focal accumulations of leukocytes adjacent to glandular crypts, and an increased number of intraepithelial lymphocytes and certain macrophage subsets have been described as putative early changes, especially in Crohn's disease.

The term “treatment” refers to both therapeutic treatment and prophylactic or preventative measures for IBD, wherein the object is to prevent or slow down (lessen) the targeted pathologic condition or disorder. Those in need of treatment include those already with an IBD as well as those prone to have an IBD or those in whom the IBD is to be prevented. Once the diagnosis of an IBD has been made by the methods disclosed herein, the goals of therapy are to induce and maintain a remission.

Various agents that are suitable for use as an “IBD therapeutic agent” are known to those of ordinary skill in the art. As described herein, such agents include without limitation, aminosalicylates, corticosteroids, and immunosuppressive agents.

The term “test sample” refers to a sample from a mammalian subject suspected of having an IBD, known to have an IBD, or known to be in remission from an IBD. The test sample may originate from various sources in the mammalian subject including, without limitation, blood, semen, serum, urine, bone marrow, mucosa, tissue, etc. The test sample may also be derived from a tissue biopsy.

The term “control” or “control sample” refers a negative control in which a negative result is expected to help correlate a positive result in the test sample. Controls that are suitable for the present invention include, without limitation, a sample known- to have normal levels of gene expression, a sample obtained from a mammalian subject known not to have an IBD, and a sample obtained from a mammalian subject known to be normal. A control may also be a sample obtained from a subject previously diagnosed and treated for an IBD who is currently in remission; and such a control is useful in determining any recurrence of an IBD in a subject who is in remission. In addition, the control may be a sample containing normal cells that have the same origin as cells contained in the test sample. Those of skill in the art will appreciate other controls suitable for use in the present invention.

The term “microarray” refers to an ordered arrangement of hybridizable array elements, preferably polynucleotide probes, on a substrate.

The term “polynucleotide,” when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The terms “differentially expressed gene,” “differential gene expression” and their synonyms, which are used interchangeably, refer to a gene whose expression is activated to a higher or lower level in a subject suffering from a disease, specifically an IBD, such as UC or CD, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, specifically an IBD, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is at least an about two-fold, preferably at least about four-fold, more preferably at least about six-fold, most preferably at least about ten-fold difference between the expression of a given gene in normal and diseased subjects, or in various stages of disease development in a diseased subject.

The term “over-expression” with regard to an RNA transcript is used to refer to the level of the transcript determined by normalization to the level of reference mRNAs, which might be all transcripts detected in the specimen or a particular reference set of mRNAs.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

In general, the term “marker” or “biomarker” or refers to an identifiable physical location on a chromosome, such as a restriction endonuclease recognition site or a gene, whose inheritance can be monitored. The marker may be an expressed region of a gene referred to as a “gene expression marker”, or some segment of DNA with no known coding function. An “IBD marker” as used herein refers to one or more of the molecules shown as SEQ ID NOS:1-426.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2 ×SSC (sodium chloride/sodium citrate) and 50% formamide, followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

In the context of the present invention, reference to “at least one,” “at least two,” “at least five,” etc. of the nucleic acids encoding a polypeptide described herein (or genes) listed in any particular gene set means any one or any and all combinations of the genes listed.

The terms “splicing” and “RNA splicing” are used interchangeably and refer to RNA processing that removes introns and joins exons to produce mature mRNA with continuous coding sequence that moves into the cytoplasm of an eukaryotic cell.

In theory, the term “exon” refers to any segment of an interrupted gene that is represented in the mature RNA product (B. Lewin. Genes IV Cell Press, Cambridge Mass. 1990). In theory the term “intron” refers to any segment of DNA that is transcribed but removed from within the transcript by splicing together the exons on either side of it. Operationally, exon sequences occur in the mRNA sequence of a gene as defined by Ref. SEQ ID numbers. Operationally, intron sequences are the intervening sequences within the genomic DNA of a gene, bracketed by exon sequences and having GT and AG splice consensus sequences at their 5′ and 3′ boundaries.

An “interfering RNA” or “small interfering RNA (siRNA)” is a double stranded RNA molecule usually less than about 30 nucleotides in length that reduces expression of a target gene. Interfering RNAs may be identified and synthesized using known methods (Shi Y., Trends in Genetics 19(1):9-12 (2003), WO/2003056012 and WO2003064621), and siRNA libraries are commercially available, for example from Dharmacon, Lafayette, Colo.

A “native sequence” polypeptide is one which has the same amino acid sequence as a polypeptide derived from nature, including naturally occurring or allelic variants. Such native sequence polypeptides can be isolated from nature or can be produced by recombinant or synthetic means. Thus, a native sequence polypeptide can have the amino acid sequence of naturally occurring human polypeptide, murine polypeptide, or polypeptide from any other mammalian species.

The term “antibody” herein is used in the broadest sense and specifically covers monoclonal antibodies, polyclonal antibodies, multispecific antibodies (e.g. bispecific antibodies), and antibody fragments, so long as they exhibit the desired biological activity. The present invention particularly contemplates antibodies against one or more of the IBD markers disclosed herein. Such antibodies may be referred to as “anti-IBD marker antibodies”.

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc) and human constant region sequences, as well as “humanized” antibodies.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity.

An “intact antibody” herein is one which comprises two antigen binding regions, and an Fc region. Preferably, the intact antibody has a functional Fc region.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragment(s).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)).

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab=fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Unless indicated otherwise, herein the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a variable heavy domain (V_{H}) connected to a variable light domain (V_{L}) in the same polypeptide chain (V_{H} -V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

A “naked antibody” is an antibody that is not conjugated to a heterologous molecule, such as a small molecule or radiolabel.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An “affinity matured” antibody is one with one or more alterations in one or more hypervariable regions thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

An “amino acid sequence variant” antibody herein is an antibody with an amino acid sequence which differs from a main species antibody. Ordinarily, amino acid sequence variants will possess at least about 70% homology with the main species antibody, and preferably, they will be at least about 80%, more preferably at least about 90% homologous with the main species antibody. The amino acid sequence variants possess substitutions, deletions, and/or additions at certain positions within or adjacent to the amino acid sequence of the main species antibody. Examples of amino acid sequence variants herein include an acidic variant (e.g. deamidated antibody variant), a basic variant, an antibody with an amino-terminal leader extension (e.g. VHS-) on one or two light chains thereof, an antibody with a C-terminal lysine residue on one or two heavy chains thereof, etc., and includes combinations of variations to the amino acid sequences of heavy and/or light chains. The antibody variant of particular interest herein is the antibody comprising an amino-terminal leader extension on one or two light chains thereof, optionally further comprising other amino acid sequence and/or glycosylation differences relative to the main species antibody.

A “glycosylation variant” antibody herein is an antibody with one or more carbohydrate moieties attached thereto which differ from one or more carbohydrate moieties attached to a main species antibody. Examples of glycosylation variants herein include antibody with a G1 or G2 oligosaccharide structure, instead a G0 oligosaccharide structure, attached to an Fc region thereof, antibody with one or two carbohydrate moieties attached to one or two light chains thereof, antibody with no carbohydrate attached to one or two heavy chains of the antibody, etc., and combinations of glycosylation alterations.

Where the antibody has an Fc region, an oligosaccharide structure may be attached to one or two heavy chains of the antibody, e.g. at residue 299 (298, Eu numbering of residues). For pertuzumab, G0 was the predominant oligosaccharide structure, with other oligosaccharide structures such as G0-F, G-1, Man5, Man6, G1-1, G1(1-6), G1(1-3) and G2 being found in lesser amounts in the pertuzumab composition.

Unless indicated otherwise, a “G1 oligosaccharide structure” herein includes G-1, G1-1, G1(1-6) and G1(1-3) structures.

An “amino-terminal leader extension” herein refers to one or more amino acid residues of the amino-terminal leader sequence that are present at the amino-terminus of any one or more heavy or light chains of an antibody. An exemplary amino-terminal leader extension comprises or consists of three amino acid residues, VHS, present on one or both light chains of an antibody variant.

A “deamidated” antibody is one in which one or more asparagine residues thereof has been derivatized, e.g. to an aspartic acid, a succinimide, or an iso-aspartic acid.

### B.1 General Description of the Invention

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

As discussed above, the detection or diagnosis of IBD is currently obtained by various classification systems that rely on a number of variables observed in a patient. The present invention is based on the identification of genes that are associated with IBD. Accordingly, the expression levels of such genes can serve as diagnostic markers to identify patients with IBD. As described in the Examples, the differential expression of a number of genes in IBD patients has been observed. Thus, according to the present invention, the molecules listed in SEQ ID NOS:1-426 have been identified as differentially expressed in IBD. SEQ ID NOS:1-316 are upregulated in IBD, while SEQ ID NOS: 317-426 are downregulated in IBD.

a. Biomarkers of the Invention

The present invention provides numerous gene expression markers or biomarkers for IBD shown as SEQ ID NOS:1-426. Table 1 above provides a list of polypeptides and their encoding nucleic acids that are up- or down-regulated in Crohn's disease. Each nucleotide sequence is indicated by an odd numbered SEQ ID NO and corresponding Figure, which is immediately followed by an even numbered SEQ ID NO and corresponding Figure which shows the polypeptide encoded by the previous SEQ ID NO. For example, the nucleotide sequence of SEQ ID NO:1 encodes the polypeptide shown as SEQ ID NO:2.

TABLE 1

| Gene name | SEQ ID NO | FIG. |
|---|---|---|
| C6orf32 | 1 | 1 |
| C6orf32 | 2 | 2 |
| ECGF1 | 3 | 3 |
| ECGF1 | 4 | 4 |
| JAK2 | 5 | 5 |
| JAK2 | 6 | 6 |
| S100A8 | 7 | 7 |
| S100A8 | 8 | 8 |
| CXCL2 | 9 | 9 |
| CXCL2 | 10 | 10 |
| BIRC3 | 11 | 11 |
| BIRC3 | 12 | 12 |
| CASP5 | 13 | 13 |
| CASP5 | 14 | 14 |
| GBP1 | 15 | 15 |
| GBP1 | 16 | 16 |
| STAT1 | 17 | 17 |
| STAT1 | 18 | 18 |
| PIM3 | 19 | 19 |
| PIM3 | 20 | 20 |
| GBP2 | 21 | 21 |
| GBP2 | 22 | 22 |
| S100A9 | 23 | 23 |
| S100A9 | 24 | 24 |
| TRIM40 | 25 | 25 |
| TRIM40 | 26 | 26 |
| PAPLN | 27 | 27 |
| PAPLN | 28 | 28 |
| TAP1 | 29 | 29 |
| TAP1 | 30 | 30 |
| SAMD9 | 31 | 31 |
| SAMD9 | 32 | 32 |
| PARP14 | 33 | 33 |
| PARP14 | 34 | 34 |
| KRT81 | 35 | 35 |
| KRT81 | 36 | 36 |
| IFIT2 | 37 | 37 |
| IFIT2 | 38 | 38 |
| UBD | 39 | 39 |
| UBD | 40 | 40 |
| MNDA | 41 | 41 |
| MNDA | 42 | 42 |
| CASP1 | 43 | 43 |
| CASP1 | 44 | 44 |
| C10orf63 | 45 | 45 |
| C10orf63 | 46 | 46 |
| DHX57 | 47 | 47 |
| DHX57 | 48 | 48 |
| BHLHB5 | 49 | 49 |
| BHLHB5 | 50 | 50 |
| SAA1 | 51 | 51 |
| SAA1 | 52 | 52 |
| ACP5 | 53 | 53 |
| ACP5 | 54 | 54 |
| CCDC88 | 55 | 55 |
| CCDC88 | 56 | 56 |
| PARP8 | 57 | 57 |
| PARP8 | 58 | 58 |
| ZBP1 | 59 | 59 |
| ZBP1 | 60 | 60 |
| ZBTB32 | 61 | 61 |
| ZBTB32 | 62 | 62 |
| TNIP3 | 63 | 63 |
| TNIP3 | 64 | 64 |
| ZC3H12A | 65 | 65 |
| ZC3H12A | 66 | 66 |
| XAF1 | 67 | 67 |
| XAF1 | 68 | 68 |
| SAMD9L | 69 | 69 |
| SAMD9L | 70 | 70 |
| PARP9 | 71 | 71 |
| PARP9 | 72 | 72 |
| PSCD4 | 73 | 73 |
| PSCD4 | 74 | 74 |
| TNFAIP2 | 75 | 75 |
| TNFAIP2 | 76 | 76 |
| S100P | 77 | 77 |
| S100P | 78 | 78 |
| p67-phox | 79 | 79 |
| p67-phox | 80 | 80 |
| AIM2 | 81 | 81 |
| AIM2 | 82 | 82 |
| SP140 | 83 | 83 |
| SP140 | 84 | 84 |
| P47 | 85 | 85 |
| P47 | 86 | 86 |
| INDO | 87 | 87 |
| INDO | 88 | 88 |
| Staf50 | 89 | 89 |
| Staf50 | 90 | 90 |
| GBP4 | 91 | 91 |
| GBP4 | 92 | 92 |
| EPSTI1 | 93 | 93 |
| EPSTI1 | 94 | 94 |
| PRODH2 | 95 | 95 |
| PRODH2 | 96 | 96 |
| cathepsin O | 97 | 97 |
| cathepsin O | 98 | 98 |
| HOXB4 | 99 | 99 |
| HOXB4 | 100 | 100 |
| MAP3K8 | 101 | 101 |
| MAP3K8 | 102 | 102 |
| TLK1 | 103 | 103 |
| TLK1 | 104 | 104 |
| PLA2G2A | 105 | 105 |
| PLA2G2A | 106 | 106 |
| HLA-DMA | 107 | 107 |
| HLA-DMA | 108 | 108 |
| HLA-F | 109 | 109 |
| HLA-F | 110 | 110 |
| HLA-E | 111 | 111 |
| HLA-E | 112 | 112 |
| LOC732037 | 113 | 113 |
| LOC732037 | 114 | 114 |
| HLA-DRB1 | 115 | 115 |
| HLA-DRB1 | 116 | 116 |
| HLA-DPA1 | 117 | 117 |
| HLA-DPA1 | 118 | 118 |
| HLA-H | 119 | 119 |
| HLA-H | 120 | 120 |
| CD74 | 121 | 121 |
| CD74 | 122 | 122 |
| TNFRSF6B | 123 | 123 |
| TNFRSF6B | 124 | 124 |
| LTA | 125 | 125 |
| LTA | 126 | 126 |
| TNFRSF4 | 127 | 127 |
| TNFRSF4 | 128 | 128 |
| TLR2 | 129 | 129 |
| TLR2 | 130 | 130 |
| LYPD5 | 131 | 131 |
| LYPD5 | 132 | 132 |
| IGHG1 | 133 | 133 |
| IGHG1 | 134 | 134 |
| FCRL5 | 135 | 135 |
| FCRL5 | 136 | 136 |
| IGHM | 137 | 137 |
| IGHM | 138 | 138 |
| FAM26F | 139 | 139 |
| FAM26F | 140 | 140 |
| IGSF6 | 141 | 141 |
| IGSF6 | 142 | 142 |
| IFI44 | 143 | 143 |
| IFI44 | 144 | 144 |
| LILRB1 | 145 | 145 |
| LILRB1 | 146 | 146 |
| FCGR3B | 147 | 147 |
| FCGR3B | 148 | 148 |
| HM74 | 149 | 149 |
| HM74 | 150 | 150 |
| CLEC4E | 151 | 151 |
| CLEC4E | 152 | 152 |
| AQP9 | 153 | 153 |
| AQP9 | 154 | 154 |
| IFITM1 | 155 | 155 |
| IFITM1 | 156 | 156 |
| SLAMF7 | 157 | 157 |
| SLAMF7 | 158 | 158 |
| ITGB2 | 159 | 159 |
| ITGB2 | 160 | 160 |
| SELL | 161 | 161 |
| SELL | 162 | 162 |
| SELE | 163 | 163 |
| SELE | 164 | 164 |
| PCDH17 | 165 | 165 |
| PCDH17 | 166 | 166 |
| DUOX2 | 167 | 167 |
| DUOX2 | 168 | 168 |
| ADCY4 | 169 | 169 |
| ADCY4 | 170 | 170 |
| SLC8A1 | 171 | 171 |
| SLC8A1 | 172 | 172 |
| CD55 | 173 | 173 |
| CD55 | 174 | 174 |
| MUC1 | 175 | 175 |
| MUC1 | 176 | 176 |
| GPR110 | 177 | 177 |
| GPR110 | 178 | 178 |
| MALT1 | 179 | 179 |
| MALT1 | 180 | 180 |
| SLC40A1 | 181 | 181 |
| SLC40A1 | 182 | 182 |
| PDZK1IP1 | 183 | 183 |
| PDZK1IP1 | 184 | 184 |
| IFITM3 | 185 | 185 |
| IFITM3 | 186 | 186 |
| FGF5 | 187 | 187 |
| FGF5 | 188 | 188 |
| TMC5 | 189 | 189 |
| TMC5 | 190 | 190 |
| KCND3 | 191 | 191 |
| KCND3 | 192 | 192 |
| EBI2 | 193 | 193 |
| EBI2 | 194 | 194 |
| SLC6A14 | 195 | 195 |
| SLC6A14 | 196 | 196 |
| CMKLR1 | 197 | 197 |
| CMKLR1 | 198 | 198 |
| IL23A | 199 | 199 |
| IL23A | 200 | 200 |
| BLR1 | 201 | 201 |
| BLR1 | 202 | 202 |
| IL2RA | 203 | 203 |
| IL2RA | 204 | 204 |
| IL3RA | 205 | 205 |
| IL3RA | 206 | 206 |
| CXCL1 | 207 | 207 |
| CXCL1 | 208 | 208 |
| CXCL6 | 209 | 209 |
| CXCL6 | 210 | 210 |
| IFNG | 211 | 211 |
| IFNG | 212 | 212 |
| CXCL13 | 213 | 213 |
| CXCL13 | 214 | 214 |
| CXCL11 | 215 | 215 |
| CXCL11 | 216 | 216 |
| CXCL3 | 217 | 217 |
| CXCL3 | 218 | 218 |
| IL1B | 219 | 219 |
| IL1B | 220 | 220 |
| CXCL11 | 221 | 221 |
| CXCL11 | 222 | 222 |
| CXCL12 | 223 | 223 |
| CXCL12 | 224 | 224 |
| IL8 | 225 | 225 |
| IL8 | 226 | 226 |
| CXCL10 | 227 | 227 |
| CXCL10 | 228 | 228 |
| CXCL9 | 229 | 229 |
| CXCL9 | 230 | 230 |
| CCL20 | 231 | 231 |
| CCL20 | 232 | 232 |
| MDK | 233 | 233 |
| MDK | 234 | 234 |
| CCL11 | 235 | 235 |
| CCL11 | 236 | 236 |
| MMP3 | 237 | 237 |
| MMP3 | 238 | 238 |
| MMP7 | 239 | 239 |
| MMP7 | 240 | 240 |
| MMP9 | 241 | 241 |
| MMP9 | 242 | 242 |
| MMP12 | 243 | 243 |
| MMP12 | 244 | 244 |
| DEFA5 | 245 | 245 |
| DEFA5 | 246 | 246 |
| DEFA6 | 247 | 247 |
| DEFA6 | 248 | 248 |
| REG1A | 249 | 249 |
| REG1A | 250 | 250 |
| REG4 | 251 | 251 |
| REG4 | 252 | 252 |
| REG3G | 253 | 253 |
| REG3G | 254 | 254 |
| REG1B | 255 | 255 |
| REG1B | 256 | 256 |
| REG3A | 257 | 257 |
| REG3A | 258 | 258 |
| SAA2 | 259 | 259 |
| SAA2 | 260 | 260 |
| PROK2 | 261 | 261 |
| PROK2 | 262 | 262 |
| B2M | 263 | 263 |
| B2M | 264 | 264 |
| CHI3L1 | 265 | 265 |
| CHI3L1 | 266 | 266 |
| NLRC5 | 267 | 267 |
| NLRC5 | 268 | 268 |
| ANGPTL4 | 269 | 269 |
| ANGPTL4 | 270 | 270 |
| NOD27 | 271 | 271 |
| NOD27 | 272 | 272 |
| LCN2 | 273 | 273 |
| LCN2 | 274 | 274 |
| PLA2G7 | 275 | 275 |
| PLA2G7 | 276 | 276 |
| SEPP1 | 277 | 277 |
| SEPP1 | 278 | 278 |
| UBR1 | 279 | 279 |
| UBR1 | 280 | 280 |
| ANGPT2 | 281 | 281 |
| ANGPT2 | 282 | 282 |
| UNC5CL | 283 | 283 |
| UNC5CL | 284 | 284 |
| OLFM4 | 285 | 285 |
| OLFM4 | 286 | 286 |
| SPON2 | 287 | 287 |
| SPON2 | 288 | 288 |
| LST1 | 289 | 289 |
| LST1 | 290 | 290 |
| DMBT1 | 291 | 291 |
| DMBT1 | 292 | 292 |
| CHRDL2 | 293 | 293 |
| CHRDL2 | 294 | 294 |
| ADM | 295 | 295 |
| ADM | 296 | 296 |
| C4BPA | 297 | 297 |
| C4BPA | 298 | 298 |
| C3 | 299 | 299 |
| C3 | 300 | 300 |
| C1QB | 301 | 301 |
| C1QB | 302 | 302 |
| C1S | 303 | 303 |
| C1S | 304 | 304 |
| CFB | 305 | 305 |
| CFB | 306 | 306 |
| IGJ | 307 | 307 |
| IGJ | 308 | 308 |
| PCK1 | 309 | 309 |
| PCK1 | 310 | 310 |
| CHP2 | 311 | 311 |
| CHP2 | 312 | 312 |
| TNNC2 | 313 | 313 |
| TNNC2 | 314 | 314 |
| FRMD1 | 315 | 315 |
| FRMD1 | 316 | 316 |
| ADH1C | 317 | 317 |
| ADH1C | 318 | 318 |
| HMGCS2 | 319 | 319 |
| HMGCS2 | 320 | 320 |
| CA2 | 321 | 321 |
| CA2 | 322 | 322 |
| AKR1B10 | 323 | 323 |
| AKR1B10 | 324 | 324 |
| ADH1A | 325 | 325 |
| ADH1A | 326 | 326 |
| CDKN2B | 327 | 327 |
| CDKN2B | 328 | 328 |
| LOC340888 | 329 | 329 |
| LOC340888 | 330 | 330 |
| PHLPPL | 331 | 331 |
| PHLPPL | 332 | 332 |
| CES2 | 333 | 333 |
| CES2 | 334 | 334 |
| TCEA3 | 335 | 335 |
| TCEA3 | 336 | 336 |
| FABP5 | 337 | 337 |
| FABP5 | 338 | 338 |
| ARRDC4 | 339 | 339 |
| ARRDC4 | 340 | 340 |
| HIGD1A | 341 | 341 |
| HIG1 | 342 | 342 |
| SAMD13 | 343 | 343 |
| SAMD13 | 344 | 344 |
| PPARG | 345 | 345 |
| PPARG | 346 | 346 |
| ZNF575 | 347 | 347 |
| ZNF575 | 348 | 348 |
| ETFDH | 349 | 349 |
| ETFDH | 350 | 350 |
| TLN2 | 351 | 351 |
| TLN2 | 352 | 352 |
| NFKBIL2 | 353 | 353 |
| NFKBIL2 | 354 | 354 |
| KLF5 | 355 | 355 |
| KLF5 | 356 | 356 |
| PLCE1 | 357 | 357 |
| PLCE1 | 358 | 358 |
| ANKRD15 | 359 | 359 |
| ANKRD15 | 360 | 360 |
| CYP2J2 | 361 | 361 |
| CYP2J2 | 362 | 362 |
| MAOA | 363 | 363 |
| MAOA | 364 | 364 |
| UGT2A3 | 365 | 365 |
| UGT2A3 | 366 | 366 |
| PDE6A | 367 | 367 |
| PDE6A | 368 | 368 |
| HSD17B2 | 369 | 369 |
| HSD17B2 | 370 | 370 |
| NEU4 | 371 | 371 |
| NEU4 | 372 | 372 |
| UGT1A6 | 373 | 373 |
| UGT1A6 | 374 | 374 |
| GBA3 | 375 | 375 |
| GBA3 | 376 | 376 |
| CA12 | 377 | 377 |
| CA12 | 378 | 378 |
| RDH5 | 379 | 379 |
| RDH5 | 380 | 380 |
| IL1R2 | 381 | 381 |
| IL1R2 | 382 | 382 |
| SLC38A4 | 383 | 383 |
| SLC38A4 | 384 | 384 |
| SLC16A9 | 385 | 385 |
| SLC16A9 | 386 | 386 |
| AQP8 | 387 | 387 |
| AQP8 | 388 | 388 |
| ABCG2 | 389 | 389 |
| ABCG2 | 390 | 390 |
| SLC4A4 | 391 | 391 |
| SLC4A4 | 392 | 392 |
| SLITRK6 | 393 | 393 |
| SLITRK6 | 394 | 394 |
| SLC22A5 | 395 | 395 |
| SLC22A5 | 396 | 396 |
| SLC16A5 | 397 | 397 |
| SLC16A5 | 398 | 398 |
| SLC39A5 | 399 | 399 |
| SLC39A5 | 400 | 400 |
| GJB1 | 401 | 401 |
| GJB1 | 402 | 402 |
| DISP2 | 403 | 403 |
| DISP2 | 404 | 404 |
| TRPM4 | 405 | 405 |
| TRPM4 | 406 | 406 |
| MCOLN2 | 407 | 407 |
| MCOLN2 | 408 | 408 |
| SLC26A2 | 409 | 409 |
| SLC26A2 | 410 | 410 |
| HHLA2 | 411 | 411 |
| HHLA2 | 412 | 412 |
| CNTN4 | 413 | 413 |
| CNTN4 | 414 | 414 |
| ANPEP | 415 | 415 |
| ANPEP | 416 | 416 |
| GUCA2A | 417 | 417 |
| GUCA2A | 418 | 418 |
| SPON1 | 419 | 419 |
| SPON1 | 420 | 420 |
| RHOU | 421 | 421 |
| RHOU | 422 | 422 |
| ANKRD9 | 423 | 423 |
| ANKRD9 | 424 | 424 |
| AATK | 425 | 425 |
| AATK | 426 | 426 |

As described in Example 1, the molecules shown in SEQ ID NOS:1-316 were found to be up-regulated in Crohn's disease and the molecules shown in SEQ ID NOS:317-426 were found to be down-regulated in Crohn's disease. In one embodiment of the present invention, the biomarkers are suitable for use in a panel of markers (as described herein). Such panels may include one or more markers from SEQ ID NOS:1-426; one or more markers from SEQ ID NOS:1-316; and one or more markers from SEQ ID NOS:317-426. Those of ordinary skill in the art will appreciate the various combinations of biomarkers from SEQ ID NOS:1-426 that are suitable for use in the panels described herein.

In one embodiment of the present invention, a preferred set of IBD markers identified by microarray analysis, includes markers that are upregulated in an IBD. Preferably, the set of upregulated markers includes S100 calcium binding protein A (calgranulin A) (SEQ ID NO:8); S100 calcium binding protein A9 (calgranulin B) (SEQ ID NO:24); CD74 molecule, major histocompatibility complex, class II invariant chain (SEQ ID NO:122); tumor necrosis factor receptor superfamily, member 4 (SEQ ID NO:128); toll-like receptor 2 (SEQ ID NO:130); Fc receptor-like 5 (SEQ ID NO:136); mitogen-activated protein kinase kinase kinase 8 (SEQ ID NO:102); C-type lectin domain family 4, member E (SEQ ID NO:152); lipocalin 2 (oncogene 24p3) (SEQ ID NO:274); complement component 4 binding protein, alpha (SEQ ID NO:298); complement component 3 (SEQ ID NO:300); complement component 1, q subcomponent, B chain (SEQ ID NO:302); complement component 1, s subcomponent (SEQ ID NO:304); and complement factor B (SEQ ID NO:306). A panel of biomarkers as described herein may include one of, more than one of, or all of these markers.

The nucleotide and/or amino acid sequences provided herein, as single markers or in any combination, are preferred for use in prognostic and diagnostic assays of the present invention. The IBD markers of the present invention are differentially expressed nucleic acids encoding a polypeptide shown as any one of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, and 426 (or genes or regions of genes). A differential level of expression of one or more markers in a test sample from a mammalian subject relative to a control can determined from the level of expression of such nucleic acids (or RNA transcripts or expression products) detected by one or more of the methods described in further detail below.

Based on evidence of such differential expression in cells from a mammalian subject having IBD relative to normal cells, the present invention provides gene markers for IBD. The IBD markers and associated information provided by the present invention allow physicians to make more intelligent treatment decisions, and to customize the treatment of IBD to the needs of individual patients, thereby maximizing the benefit of treatment and minimizing the exposure of patients to unnecessary treatments, which do not provide any significant benefits and often carry serious risks due to toxic side-effects.

Multi-analyte gene expression tests can measure the expression level of one or more genes involved in each of several relevant physiologic processes or component cellular characteristics. In some instances the predictive power of the test, and therefore its utility, can be improved by using the expression values obtained for individual genes to calculate a score which is more highly correlated with outcome than is the expression value of the individual genes. For example, the calculation of a quantitative score (recurrence score) that predicts the likelihood of recurrence in estrogen receptor-positive, node-negative breast cancer is describe in U.S. Patent application (Publication Number 20050048542). The equation used to calculate such a recurrence score may group genes in order to maximize the predictive value of the recurrence score. The grouping of genes may be performed at least in part based on knowledge of their contribution to physiologic functions or component cellular characteristics such as discussed above. The formation of groups, in addition, can facilitate the mathematical weighting of the contribution of various expression values to the recurrence score. The weighting of a gene group representing a physiological process or component cellular characteristic can reflect the contribution of that process or characteristic to the pathology of the IBD and clinical outcome. Accordingly, in an important aspect, the present invention also provides specific groups of the genes identified herein, that together are more reliable and powerful predictors of outcome than the individual genes or random combinations of the genes identified.

In addition, based on the determination of a recurrence score, one can choose to partition patients into subgroups at any particular value(s) of the recurrence score, where all patients with values in a given range can be classified as belonging to a particular risk group. Thus, the values chosen will define subgroups of patients with respectively greater or lesser risk.

The utility of a gene marker in predicting the development or progression of an IBD may not be unique to that marker. An alternative marker having a expression pattern that is closely similar to a particular test marker may be substituted for or used in addition to a test marker and have little impact on the overall predictive utility of the test. The closely similar expression patterns of two genes may result from involvement of both genes in a particular process and/or being under common regulatory control. The present invention specifically includes and contemplates the use of such substitute genes or gene sets in the methods of the present invention.

The markers and associated information provided by the present invention predicting the development and/or progression of an IBD also have utility in screening patients for inclusion in clinical trials that test the efficacy of drug compounds for the treatment of patients with IBD.

The markers and associated information provided by the present invention predicting the presence, development and/or progression of an IBD are useful as criterion for determining whether IBD treatment is appropriate. For example, IBD treatment may be appropriate where the results of the test indicate that an IBD marker is differentially expressed in a test sample from an individual relative to a control sample. The individual may be an individual not known to have an IBD, an individual known to have an IBD, an individual previously diagnosed with an IBD undergoing treatment for the IBD, or an individual previously diagnosed with an IBD and having had surgery to address the IBD. In addition, the present invention contemplates methods of treating an IBD. As described below, the diagnostic methods of the present invention may further comprise the step of administering an IBD therapeutic agent to the mammalian subject that provided the test sample in which the differential expression of one or more IBD markers was observed relative to a control. Such methods of treatment would therefore comprise (a) determining the presence of an IBD in a mammalian subject, and (b) administering an IBD therapeutic agent to the mammalian subject.

In another embodiment, the IBD markers and associated information are used to design or produce a reagent that modulates the level or activity of the gene's transcript or its expression product. Said reagents may include but are not limited to an antisense RNA, a small inhibitory RNA (siRNA), a ribozyme, a monoclonal or polyclonal antibody. In a further embodiment, said gene or its transcript, or more particularly, an expression product of said transcript is used in an (screening) assay to identify a drug compound, wherein said drug compounds is used in the development of a drug to treat an IBD.

In various embodiments of the inventions, various technological approaches described below are available for determination of expression levels of the disclosed genes. In particular embodiments, the expression level of each gene may be determined in relation to various features of the expression products of the gene including exons, introns, protein epitopes and protein activity. In other embodiments, the expression level of a gene may be inferred from analysis of the structure of the gene, for example from the analysis of the methylation pattern of gene's promoter(s).

b. Diagnostic Methods of the Invention

The present invention provides methods of detecting or diagnosing an IBD in a mammalian subject based on differential expression of an IBD marker. In one embodiment, the methods comprise the use of a panel of IBD markers as discussed above. The panels may include one or more IBD markers selected from SEQ ID NOS:1-426. In some embodiments, the panel of IBD markers will include at least 1 IBD marker, at least two IBD markers, at least three IBD markers, at least 4 IBD markers, at least five IBD markers, at least 6 IBD markers, at least 7 IBD marker, at least 8 IBD markers, at least 9 IBD markers, at least 10 IBD markers, at least 11 IBD markers, at least 12 IBD markers, at least 13 IBD markers, at least 14 IBD markers, at least 15 IBD markers, at least 16 IBD markers, at least 17 IBD markers, at least 18 IBD markers, at least 19 IBD markers, or at least 20 IBD markers. In one embodiment, the panel includes markers in increments of five. In another embodiment, the panel includes markers in increments of ten. The panel may include an IBD marker that is overexpressed in IBD relative to a control, an IBD marker that is underexpressed in IBD relative to a control, or IBD markers that are both overexpressed and underexpressed in IBD relative to a control. In a preferred embodiment, the panel includes one or more markers that are upregulated in CD and one or more markers that are downregulated in CD.

In another embodiment, the panels of the present invention may include an IBD marker that is overexpressed in an active IBD relative to a control, underexpressed in an active IBD relative to a control, or IBD markers that are both overexpressed and underexpressed in an active IBD relative to a control. In another embodiment, the panels of the present invention may include an IBD marker that is overexpressed in an inactive IBD relative to a control, underexpressed in an inactive IBD relative to a control, or IBD markers that are both overexpressed and underexpressed in an inactive IBD relative to a control. In a preferred embodiment, the active IBD is CD. In another preferred embodiment, the inactive IBD is CD.

In a preferred embodiment, the methods of diagnosing or detecting the presence of an IBD in a mammalian subject comprise determining a differential expression level of RNA transcripts or expression products thereof from a panel of IBD markers in a test sample obtained from the subject relative to the level of expression in a control, wherein the differential level of expression is indicative of the presence of an IBD in the subject from which the test sample was obtained. The differential expression in the test sample may be higher and/or lower relative to a control as discussed herein.

Differential expression or activity of one or more of the nucleic acids encoding a polypeptide described herein (or genes or the corresponding RNA molecules) in a biological sample obtained from the patient, relative to control, indicates the presence of an IBD in the patient. The control can, for example, be a gene, present in the same cell, which is known to be up-regulated (or down-regulated) in an IBD patient (positive control). Alternatively, or in addition, the control can be the expression level of the same gene in a normal cell of the same cell type (negative control). Expression levels can also be normalized, for example, to the expression levels of housekeeping genes, such as glyceraldehyde-3-phosphate-dehydrogenase (GAPDH) and/or β-actin, or to the expression levels of all genes in the sample tested. In one embodiment, expression of one or more of the above noted genes is deemed positive expression if it is at the median or above, e.g. compared to other samples of the same type. The median expression level can be determined essentially contemporaneously with measuring gene expression, or may have been determined previously. These and other methods are well known in the art, and are apparent to those skilled in the art.

Methods for identifying IBD patients are provided herein. Of this patient population, patients with an IBD can be identified by determining the expression level of one or more of the genes, the corresponding RNA molecules or encoded proteins in a biological sample comprising cells obtained from the patient. The biological sample can, for example, be a tissue biopsy as described herein.

The methods of the present invention concern IBD diagnostic assays, and imaging methodologies. In one embodiment, the assays are performed using antibodies as described herein. The invention also provides various immunological assays useful for the detection and quantification of proteins. These assays are performed within various immunological assay formats well known in the art, including but not limited to various types of radioimmunoassays, enzyme-linked immunosorbent assays (ELISA), enzyme-linked immunofluorescent assays (ELIFA), and the like. In addition, immunological imaging methods capable of detecting an IBD characterized by expression of a molecule described herein are also provided by the invention, including but not limited to radioscintigraphic imaging methods using labeled antibodies. Such assays are clinically useful in the detection, monitoring, diagnosis and prognosis of IBD characterized by expression of one or more molecules described herein.

Another aspect of the present invention relates to methods for identifying a cell that expresses a molecule described herein. The expression profile of a molecule(s) described herein make it a diagnostic marker for IBD. Accordingly, the status of the expression of the molecule(s) provides information useful for predicting a variety of factors including susceptibility to advanced stages of disease, rate of progression, and/or sudden and severe onset of symptoms in an active inactive IBD, i.e. flare-ups.

In one embodiment, the present invention provides methods of detecting an IBD. A test sample from a mammalian subject and a control sample from a known normal mammal are each contacted with an anti-IBD marker antibody or a fragment thereof. The level of IBD marker expression is measured and a differential level of expression in the test sample relative to the control sample is indicative of an IBD in the mammalian subject from which the test sample was obtained. In some embodiments, the level of IBD marker expression in the test sample is determined to be higher than the level of expression in the control, wherein the higher level of expression indicates the presence of an IBD in the subject from which the test sample was obtained. In another embodiments, the level of IBD marker expression in the test sample is determined to be lower than the level of expression in the control, wherein the lower level of expression indicates the presence of an IBD in the subject from which the test sample was obtained.

In another embodiment, the IBD detected by the methods of the present invention is the recurrence or flareup of an IBD in the mammalian subject.

In preferred embodiments, the methods are employed to detect the flare-up of an IBD or a recurrence of an IBD in a mammalian subject previously determined to have an IBD who underwent treatment for the IBD, such as drug therapy or a surgical procedure. Following initial detection of an IBD, additional test samples may be obtained from the mammalian subject found to have an IBD. The additional sample may be obtained hours, days, weeks, or months after the initial sample was taken. Those of skill in the art will appreciate the appropriate schedule for obtaining such additional samples, which may include second, third, fourth, fifth, sixth, etc. test samples. The initial test sample and the additional sample (and alternately a control sample as described herein) are contacted with an anti-IBD marker antibody. The level of IBD marker expression is measured and a differential level of expression in the additional test sample as compared to the initial test sample is indicative of a flare-up in or a recurrence of an IBD in the mammalian subject from which the test sample was obtained.

In one aspect, the methods of the present invention are directed to a determining step. In one embodiment, the determining step comprises measuring the level of expression of one or more IBD markers in a test sample relative to a control. Typically, measuring the level of IBD marker expression, as described herein, involves analyzing a test sample for differential expression of an IBD marker relative to a control by performing one or more of the techniques described herein. The expression level data obtained from a test sample and a control are compared for differential levels of expression. In another embodiment, the determining step further comprises an examination of the test sample and control expression data to assess whether an IBD is present in the subject from which the test sample was obtained.

In some embodiments, the determining step comprises the use of a software program executed by a suitable processor for the purpose of (i) measuring the differential level of IBD marker expression in a test sample and a control; and/or (ii) analyzing the data obtained from measuring differential level of IBD marker expression in a test sample and a control. Suitable software and processors are well known in the art and are commercially available. The program may be embodied in software stored on a tangible medium such as CD-ROM, a floppy disk, a hard drive, a DVD, or a memory associated with the processor, but persons of ordinary skill in the art will readily appreciate that the entire program or parts thereof could alternatively be executed by a device other than a processor, and/or embodied in firmware and/or dedicated hardware in a well known manner.

Following the determining step, the measurement results, findings, diagnoses, predictions and/or treatment recommendations are typically recorded and communicated to technicians, physicians and/or patients, for example. In certain embodiments, computers will be used to communicate such information to interested parties, such as, patients and/or the attending physicians. In some embodiments, the assays will be performed or the assay results analyzed in a country or jurisdiction which differs from the country or jurisdiction to which the results or diagnoses are communicated.

In a preferred embodiment, a diagnosis, prediction and/or treatment recommendation based on the level of expression of one or more IBD markers disclosed herein measured in a test subject of having one or more of the IBD markers herein is communicated to the subject as soon as possible after the assay is completed and the diagnosis and/or prediction is generated. The results and/or related information may be communicated to the subject by the subject's treating physician. Alternatively, the results may be communicated directly to a test subject by any means of communication, including writing, electronic forms of communication, such as email, or telephone. Communication may be facilitated by use of a computer, such as in case of email communications. In certain embodiments, the communication containing results of a diagnostic test and/or conclusions drawn from and/or treatment recommendations based on the test, may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

The invention provides assays for detecting the differential expression of an IBD marker in tissues associated with the gastrointestinal tract including, without limitation, ascending colon tissue, descending colon tissue, sigmoid colon tissue, and terminal ileum tissue; as well expression in other biological samples such as serum, semen, bone, prostate, urine, cell preparations, and the like. Methods for detecting differential expression of an IBD marker are also well known and include, for example, immunoprecipitation, immunohistochemical analysis, Western blot analysis, molecular binding assays, ELISA, ELIFA and the like. For example, a method of detecting the differential expression of an IBD marker in a biological sample comprises first contacting the sample with an anti-IBD marker antibody, an IBD marker-reactive fragment thereof, or a recombinant protein containing an antigen-binding region of an anti-IBD marker antibody; and then detecting the binding of an IBD marker protein in the sample.

In various embodiments of the inventions, various technological approaches are available for determination of expression levels of the disclosed genes, including, without limitation, RT-PCR, microarrays, serial analysis of gene expression (SAGE) and Gene Expression Analysis by Massively Parallel Signature Sequencing (MPSS), which will be discussed in detail below. In particular embodiments, the expression level of each gene may be determined in relation to various features of the expression products of the gene including exons, introns, protein epitopes and protein activity. In other embodiments, the expression level of a gene may be inferred from analysis of the structure of the gene, for example from the analysis of the methylation pattern of gene's promoter(s).

c. Therapeutic Methods of the Invention

The present invention provides therapeutic methods of treating an IBD in a subject in need that comprise detecting the presence of an IBD in a mammalian subject by the diagnostic methods described herein and then administering to the mammalian subject an IBD therapeutic agent. Those of ordinary skill in the art will appreciate the various IBD therapeutic agents that may be suitable for use in the present invention (see St Clair Jones, Hospital Pharmacist, May 2006, Vol. 13; pages 161-166, hereby incorporated by reference in its entirety). The present invention contemplates methods of IBD treatment in which one or more IBD therapeutic agents are administered to a subject in need. In one embodiment, the IBD therapeutic agent is one or more of an aminosalicylate, a corticosteroid, and an immunosuppressive agent. In a preferred embodiment, the aminosalicylate is one of sulfasalazine, olsalazine, mesalamine, balsalazide, and asacol. In another preferred embodiment, multiple aminosalicylates are co-administered, such as a combination of sulfasalazine and olsalazine. In other preferred embodiments, the corticosteroid may be budesonide, prednisone, prednisolone, methylprednisolone, 6-mercaptopurine (6-MP), azathioprine, methotrexate, and cyclosporin. In other preferred embodiments, the IBD therapeutic agent may an antibiotic, such as ciprofloxacin and/or metronidazole; or an antibody-based agent such as infliximab (Remicade®).

The least toxic IBD therapeutic agents which patients are typically treated with are the aminosalicylates. Sulfasalazine (Azulfidine), typically administered four times a day, consists of an active molecule of aminosalicylate (5-ASA) which is linked by an azo bond to a sulfapyridine. Anaerobic bacteria in the colon split the azo bond to release active 5-ASA. However, at least 20% of patients cannot tolerate sulfapyridine because it is associated with significant side-effects such as reversible sperm abnormalities, dyspepsia or allergic reactions to the sulpha component. These side effects are reduced in patients taking olsalazine. However, neither sulfasalazine nor olsalazine are effective for the treatment of small bowel inflammation. Other formulations of 5-ASA have been developed which are released in the small intestine (e.g. mesalamine and asacol). Normally it takes 6-8 weeks for 5-ASA therapy to show full efficacy. Patients who do not respond to 5-ASA therapy, or who have a more severe disease, are prescribed corticosteroids. Clinical remission is achieved with corticosteroids within 2-4 weeks. however the side effects include Cushing goldface, facial hair, severe mood swings and sleeplessness. The response to sulfasalazine and 5-aminosalicylate preparations may be poor in CD, fair to mild in early ulcerative colitis and poor in severe UC. If these agents fail, powerful immunosuppressive agents such as cyclosporine, prednisone, 6-mercaptopurine or azathioprine (converted in the liver to 6-mercaptopurine) are typically tried. For CD patients, the use of corticosteroids and other immunosuppressives must be carefully monitored because of the high risk of intra-abdominal sepsis originating in the fistulas and abscesses common in this disease. Approximately 25% of IBD patients will require surgery (colectomy) during the course of the disease.

Treatment of an IBD may include a surgical procedure, including without limitation, a bowel resection, anastomosis, a colectomy, a proctocolectomy, and an ostomy, or any combination thereof.

In addition to pharmaceutical medicine and surgery, nonconventional treatments for IBD such as nutritional therapy have also been attempted. For example, Flexical®, a semi-elemental formula, has been shown to be as effective as the steroid prednisolone. Sanderson et al., Arch. Dis. Child. 51:123-7 (1987). However, semi-elemental formulas are relatively expensive and are typically unpalatable—thus their use has been restricted. Nutritional therapy incorporating whole proteins has also been attempted to alleviate the symptoms of IBD. Giafer et al., Lancet 335: 816-9 (1990). U.S. Pat. No. 5,461,033 describes the use of acidic casein isolated from bovine milk and TGF-2. Beattie et al., Aliment. Pharmacol. Ther. 8: 1-6 (1994) describes the use of casein in infant formula in children with IBD. U.S. Pat. No. 5,952,295 describes the use of casein in an enteric formulation for the treatment of IBD. However, while nutritional therapy is non-toxic, it is a palliative treatment and does not treat the underlying cause of the disease.

The present invention contemplates methods of IBD treatment, including for example, in vitro, ex vivo and in vivo therapeutic methods. The invention provides methods useful for treating an IBD in a subject in need upon the detection of an IBD disease state in the subject associated with the expression of one or more IBD markers disclosed herein, such as increased and/or decreased IBD marker expression. In one preferred embodiment, the method comprises (a) determining that a differential level of expression of one or more nucleic acids encoding a polypeptide shown as any one of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, and 426 in a test sample obtained from said subject relative to the level of expression in a control, wherein said differential level of expression is indicative of the presence of an IBD in the subject from which the test sample was obtained; and (b) administering to said subject an effective amount of an IBD therapeutic agent. The determining step (a) may comprise the measurement of the expression of multiple IBD marker. Where the differential level of expression is higher relative to a control, the expression level of one or more nucleic acids encoding a polypeptide shown as any one of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316 is determined. Where the differential level of expression is lower relative to a control, the expression level of one or more nucleic acids encoding a polypeptide shown as any one of SEQ ID NOS:318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, and 426 is determined.

The method of treatment comprises detecting the IBD and administering an effective amount of an IBD therapeutic agent to a subject in need of such treatment. In some embodiments, the IBD disease state is associated with an increased and/or decrease in expression of one or more IBD markers.

In one aspect, the invention provides methods for treating or preventing an IBD; the methods comprising detecting the presence of an IBD in a subject and administering an effective amount of an IBD therapeutic agent to the subject. It is understood that any suitable IBD therapeutic agent may be used in the methods of treatment, including aminosalicylates, corticosteroids, and immunosuppressive agents as discussed herein.

In any of the methods herein, one may administer to the subject or patient along with a single IBD therapeutic agent discussed herein an effective amount of a second medicament (where the single IBD therapeutic agent herein is a first medicament), which is another active agent that can treat the condition in the subject that requires treatment. For instance, an aminosalicylate may be co-administered with a corticosteroid, an immunsuppressive agent, or another aminosalicylate. The type of such second medicament depends on various factors, including the type of IBD, its severity, the condition and age of the patient, the type and dose of first medicament employed, etc.

Such treatments using first and second medicaments include combined administration (where the two or more agents are included in the same or separate formulations), and separate administration, in which case, administration of the first medicament can occur prior to, and/or following, administration of the second medicament. In general, such second medicaments may be administered within 48 hours after the first medicaments are administered, or within 24 hours, or within 12 hours, or within 3-12 hours after the first medicament, or may be administered over a pre-selected period of time, which is preferably about 1 to 2 days, about 2 to 3 days, about 3 to 4 days, about 4 to 5 days, about 5 to 6 days, or about 6 to 7 days.

The first and second medicaments can be administered concurrently, sequentially, or alternating with the first and second medicament or upon non-responsiveness with other therapy. Thus, the combined administration of a second medicament includes co-administration (concurrent administration), using separate formulations or a single pharmaceutical formulation, and consecutive administration in either order, wherein preferably there is a time period while both (or all) medicaments simultaneously exert their biological activities. All these second medicaments may be used in combination with each other or by themselves with the first medicament, so that the express “second medicament” as used herein does not mean it is the only medicament besides the first medicament, respectively. Thus, the second medicament need not be one medicament, but may constitute or comprise more than one such drug. These second medicaments as set forth herein are generally used in the same dosages and with administration routes as the first medicaments, or about from 1 to 99% of the dosages of the first medicaments. If such second medicaments are used at all, preferably, they are used in lower amounts than if the first medicament were not present, especially in subsequent dosings beyond the initial dosing with the first medicament, so as to eliminate or reduce side effects caused thereby.

Where the methods of the present invention comprise administering one or more IBD therapeutic agent to treat or prevent an IBD, it may be particularly desirable to combine the administering step with a surgical procedure that is also performed to treat or prevent the IBD. The IBD surgical procedures contemplated by the present invention include, without limitation, a bowel resection, anastomosis, a colectomy, a proctocolectomy, and an ostomy, or any combination thereof. For instance, an IBD therapeutic agent described herein may be combined with a colectomy in a treatment scheme, e.g. in treating an IBD. Such combined therapies include and separate administration, in which case, administration of the IBD therapeutic agent can occur prior to, and/or following, the surgical procedure.

Treatment with a combination of one or more IBD therapeutic agents; or a combination of one or more IBD therapeutic agents and a surgical procedure described herein preferably results in an improvement in the signs or symptoms of an IBD. For instance, such therapy may result in an improvement in the subject receiving the IBD therapeutic agent treatment regimen and a surgical procedure, as evidenced by a reduction in the severity of the pathology of the IBD.

The IBD therapeutic agent(s) is/are administered by any suitable means, including topical, parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic.

The IBD therapeutic agent(s) compositions administered according to the methods of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The first medicament(s) need not be, but is optionally formulated with one or more additional medicament(s) (e.g. second, third, fourth, etc. medicaments) described herein. The effective amount of such additional medicaments depends on the amount of the first medicament present in the formulation, the type of disorder or treatment, and other factors discussed above. These are generally used in the same dosages and with administration routes as used hereinbefore or about from 1 to 99% of the heretofore employed dosages.

For the prevention or treatment of an IBD, the appropriate dosage of an IBD therapeutic agent (when used alone or in combination with other agents) will depend on the type of disease to be treated, the type of IBD therapeutic agent(s), the severity and course of the disease, whether the IBD therapeutic agent is administered for preventive or therapeutic purposes, previous therapy, the patient's clinical history and response to the IBD therapeutic agent, and the discretion of the attending physician. The IBD therapeutic agent is suitably administered to the patient at one time or over a series of treatments. Depending on the type and severity of the disease, about 1 ug/kg to 15 mg/kg (e.g. 0.1 mg/kg-10 mg/kg) of IBD therapeutic agent is an initial candidate dosage for administration to the patient, whether, for example, by one or more separate administrations, or by continuous infusion. One typical daily dosage might range from about 1 ug/kg to 100 mg/kg or more, depending on the factors mentioned above. For repeated administrations over several days or longer, depending on the condition, the treatment is sustained until a desired suppression of disease symptoms occurs. One exemplary dosage of the IBD therapeutic agent would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, e.g. about six doses of the IBD therapeutic agent). An initial higher loading dose, followed by one or more lower doses may be administered. An exemplary dosing regimen comprises administering an initial loading dose of about 4 mg/kg, followed by a weekly maintenance dose of about 2 mg/kg of the IBD therapeutic agent. However, other dosage regimens may be useful. The progress of this therapy is easily monitored by conventional techniques and assays.

### B.2. Gene Expression Profiling

In general, methods of gene expression profiling can be divided into two large groups: methods based on hybridization analysis of polynucleotides, and other methods based on biochemical detection or sequencing of polynucleotides. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and reverse transcription polymerase chain reaction (RT-PCR) (Weis et al., Trends in Genetics 8:263-264 (1992)). Alternatively, antibodies may be employed that can recognize specific duplexes, including DNA duplexes RNA duplexes, and DNA-RNA hybrid duplexes or DNA-protein duplexes. Various methods for determining expression of mRNA or protein include, but are not limited to, gene expression profiling, polymerase chain reaction (PCR) including quantitative real time PCR (qRT-PCR), microarray analysis that can be performed by commercially available equipment, following manufacturer's protocols, such as by using the Affymetrix GenChip technology, serial analysis of gene expression (SAGE) (Velculescu et al., Science 270:484-487 (1995); and Velculescu et al., Cell 88:243-51 (1997)), MassARRAY, Gene Expression Analysis by Massively Parallel Signature Sequencing (MPSS) (Brenner et al., Nature Biotechnology 18:630-634 (2000)), proteomics, immunohistochemistry (IHC), etc. Preferably mRNA is quantified. Such mRNA analysis is preferably performed using the technique of polymerase chain reaction (PCR), or by microarray analysis. Where PCR is employed, a preferred form of PCR is quantitative real time PCR (qRT-PCR).

a. Reverse Transcriptase PCR (RT-PCR)

Of the techniques listed above, the most sensitive and most flexible quantitative method is RT-PCR, which can be used to compare mRNA levels in different sample populations, in normal and test sample tissues, to characterize patterns of gene expression, to discriminate between closely related mRNAs, and to analyze RNA structure.

The first step is the isolation of mRNA from a target sample. The starting material is typically total RNA isolated from colonic tissue biopsies. Thus, RNA can be isolated from a variety of tissues, including without limitation, the terminal ileum, the ascending colon, the descending colon, and the sigmoid colon. In addition, the colonic tissue from which a biopsy is obtained may be from an inflamed and/or a non-inflamed colonic area.

In one embodiment, the mRNA is obtained from a biopsy as defined above wherein the biopsy is obtained from the left colon or from the right colon. As used herein, the “left colon” refers to the sigmoideum and rectosigmoideum and the “right colon” refers to the cecum.

General methods for mRNA extraction are well known in the art and are disclosed in standard textbooks of molecular biology, including Ausubel et al., Current Protocols of Molecular Biology, John Wiley and Sons (1997). In particular, RNA isolation can be performed using purification kit, buffer set and protease from commercial manufacturers, such as Qiagen, according to the manufacturer's instructions. Total RNA from tissue samples can be isolated using RNA Stat-60 (Tel-Test). RNA prepared from a biopsy can be isolated, for example, by cesium chloride density gradient centrifugation.

As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, CA, USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700TM Sequence Detection SystemTM (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700TM Sequence Detection SystemTM. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Cₜ).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

According to one aspect of the present invention, PCR primers and probes are designed based upon intron sequences present in the gene to be amplified. In this embodiment, the first step in the primer/probe design is the delineation of intron sequences within the genes. This can be done by publicly available software, such as the DNA BLAT software developed by Kent, W. J., Genome Res. 12(4):656-64 (2002), or by the BLAST software including its variations. Subsequent steps follow well established methods of PCR primer and probe design.

In order to avoid non-specific signals, it is important to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. In: Krawetz S, Misener S (eds) Bioinformatics Methods and Protocols: Methods in Molecular Biology. Humana Press, Totowa, N. J., pp 365-386).

The most important factors considered in PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases. Tm's between 50 and 80° C., e.g. about 50 to 70° C. are typically preferred.

For further guidelines for PCR primer and probe design see, e.g. Dieffenbach, C. W. et al., “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods Mol. Biol. 70:520-527 (1997), the entire disclosures of which are hereby expressly incorporated by reference.

Further PCR-based techniques include, for example, differential display (Liang and Pardee, Science 257:967-971 (1992)); amplified fragment length polymorphism (iAFLP) (Kawamoto et al., Genome Res. 12:1305-1312 (1999)); BeadArray™ technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex 100 LabMAP system and multiple color-coded microspheres (Luminex Corp., Austin, Tex.) in a rapid assay for gene expression (Yang et al., Genome Res. 11:1888-1898 (2001)); and high coverage expression profiling (HiCEP) analysis (Fukumura et al., Nucl. Acids. Res. 31(16) e94 (2003)).

b. Microarrays

Differential gene expression can also be identified, or confirmed using the microarray technique. Thus, the expression profile of IBD-associated genes can be measured in either fresh or paraffin-embedded tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from biopsy tissue or cell lines derived from cells obtained from a subject having an IBD, and corresponding normal tissues or cell lines. Thus RNA can be isolated from a variety of colonic tissues or colonic tissue-based cell lines.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106-149 (1996)). Microarray analysis can be performed by commercially available equipment, following manufacturer's protocols, such as by using the Affymetrix GenChip technology, or Incyte's microarray technology, or Agilent's Whole Human Genome microarray technology.

c. Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

d. MassARRAY Technology

In the MassARRAY-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059-3064 (2003).

e. Gene Expression Analysis by Massively Parallel Signature Sequencing (MPSS)

This method, described by Brenner et al., Nature Biotechnology 18:630-634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

The steps of a representative protocol for profiling gene expression using fixed, paraffin-embedded tissues as the RNA source, including mRNA isolation, purification, primer extension and amplification are given in various published journal articles (for example: Godfrey et al. J. Molec. Diagnostics 2: 84-91 (2000); Specht et al., Am. J. Pathol. 158: 419-29 (2001)). Briefly, a representative process starts with cutting about 10 microgram thick sections of paraffin-embedded tissue samples. The mRNA is then extracted, and protein and DNA are removed. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrés et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tissues can be isolated, for example, by cesium chloride density gradient centrifugation. After analysis of the RNA concentration, RNA repair and/or amplification steps may be included, if necessary, and RNA is reverse transcribed using gene specific promoters followed by PCR. Preferably, real time PCR is used, which is compatible both with quantitative competitive PCR, where internal competitor for each target sequence is used for normalization, and with quantitative comparative PCR using a normalization gene contained within the sample, or a housekeeping gene for RT-PCR. For further details see, e.g. “PCR: The Polymerase Chain Reaction”, Mullis et al., eds., 1994; and Held et al., Genome Research 6:986-994 (1996). Finally, the data are analyzed to identify the best treatment option(s) available to the patient on the basis of the characteristic gene expression pattern identified in the sample examined.

f. Immunohistochemistry

Immunohistochemistry methods are also suitable for detecting the expression levels of the IBD markers of the present invention. Thus, antibodies or antisera, preferably polyclonal antisera, and most preferably monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Expression levels can also be determined at the protein level, for example, using various types of immunoassays or proteomics techniques.

In immunoassays, the target diagnostic protein marker is detected by using an antibody specifically binding to the markes. The antibody typically will be labeled with a detectable moiety. Numerous labels are available which can be generally grouped into the following categories:

Radioisotopes, such as 35%, ¹⁴C, ¹²⁵I, ³H, and ¹³¹I. The antibody can be labeled with the radioisotope using the techniques described in Current Protocols in Immunology, Volumes 1 and 2, Coligen et al. (1991) Ed. Wiley-Interscience, New York, N. Y., Pubs. for example and radioactivity can be measured using scintillation counting.

Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra, for example. Fluorescence can be quantified using a fluorimeter.

Various enzyme-substrate labels are available and U.S. Pat. No. 4,275,149 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al. (1981) Methods for the Preparation of Enzyme-Antibody Conjugates for use in Enzyme Immunoassay, in Methods in Enzym. (ed J. Langone & H. Van Vunakis), Academic press, New York 73:147-166.

Examples of enzyme-substrate combinations include, for example: horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB)); alkaline phosphatase (AP) with para-Nitrophenyl phosphate as chromogenic substrate; and β-D-galactosidase (β-D-Gal) with a chromogenic substrate (e.g., p-nitrophenyl-β-D-galactosidase) or fluorogenic substrate 4-methylumbelliferyl-β-D-galactosidase.

Numerous other enzyme-substrate combinations are available to those skilled in the art. For a general review of these, see U.S. Pat. Nos. 4,275,149 and 4,318,980.

Sometimes, the label is indirectly conjugated with the antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). Thus, indirect conjugation of the label with the antibody can be achieved.

In other versions of immunoassay techniques, the antibody need not be labeled, and the presence thereof can be detected using a labeled antibody which binds to the antibody.

Thus, the diagnostic immunoassays herein may be in any assay format, including, for example, competitive binding assays, direct and indirect sandwich assays, and immunoprecipitation assays. Zola, Monoclonal Antibodies: A Manual of Techniques, pp. 147-158 (CRC Press, Inc. 1987).

Competitive binding assays rely on the ability of a labeled standard to compete with the test sample analyze for binding with a limited amount of antibody. The amount of antigen in the test sample is inversely proportional to the amount of standard that becomes bound to the antibodies. To facilitate determining the amount of standard that becomes bound, the antibodies generally are insolubilized before or after the competition, so that the standard and analyze that are bound to the antibodies may conveniently be separated from the standard and analyze which remain unbound.

Sandwich assays involve the use of two antibodies, each capable of binding to a different immunogenic portion, or epitope, of the protein to be detected. In a sandwich assay, the test sample analyze is bound by a first antibody which is immobilized on a solid support, and thereafter a second antibody binds to the analyze, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

g. Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the markers of the present invention.

h. 5′-Multiplexed Gene Specific Priming of Reverse Transcription

RT-PCR requires reverse transcription of the test RNA population as a first step. The most commonly used primer for reverse transcription is oligo-dT, which works well when RNA is intact. However, this primer will not be effective when RNA is highly fragmented.

The present invention includes the use of gene specific primers, which are roughly 20 bases in length with a Tm optimum between about 58° C. and 60° C. These primers will also serve as the reverse primers that drive PCR DNA amplification.

An alternative approach is based on the use of random hexamers as primers for cDNA synthesis. However, we have experimentally demonstrated that the method of using a multiplicity of gene-specific primers is superior over the known approach using random hexamers.

i. Promoter Methylation Analysis

A number of methods for quantization of RNA transcripts (gene expression analysis) or their protein translation products are discussed herein. The expression level of genes may also be inferred from information regarding chromatin structure, such as for example the methylation status of gene promoters and other regulatory elements and the acetylation status of histones.

In particular, the methylation status of a promoter influences the level of expression of the gene regulated by that promoter. Aberrant methylation of particular gene promoters has been implicated in expression regulation, such as for example silencing of tumor suppressor genes. Thus, examination of the methylation status of a gene's promoter can be utilized as a surrogate for direct quantization of RNA levels.

Several approaches for measuring the methylation status of particular DNA elements have been devised, including methylation-specific PCR (Herman J. G. et al. (1996) Methylation-specific PCR: a novel PCR assay for methylation status of CpG islands. Proc. Natl. Acad. Sci. USA. 93, 9821-9826.) and bisulfite DNA sequencing (Frommer M. et al. (1992) A genomic sequencing protocol that yields a positive display of 5-methylcytosine residues in individual DNA strands. Proc. Natl. Acad. Sci. USA. 89, 1827-1831.). More recently, microarray-based technologies have been used to characterize promoter methylation status (Chen C. M. (2003) Methylation target array for rapid analysis of CpG island hypermethylation in multiple tissue genomes. Am. J. Pathol. 163, 3745.).

j. Coexpression of Genes

A further aspect of the invention is the identification of gene expression clusters. Gene expression clusters can be identified by analysis of expression data using statistical analyses known in the art, including pairwise analysis of correlation based on Pearson correlation coefficients (Pearson K. and Lee A. (1902) Biometrika 2, 357).

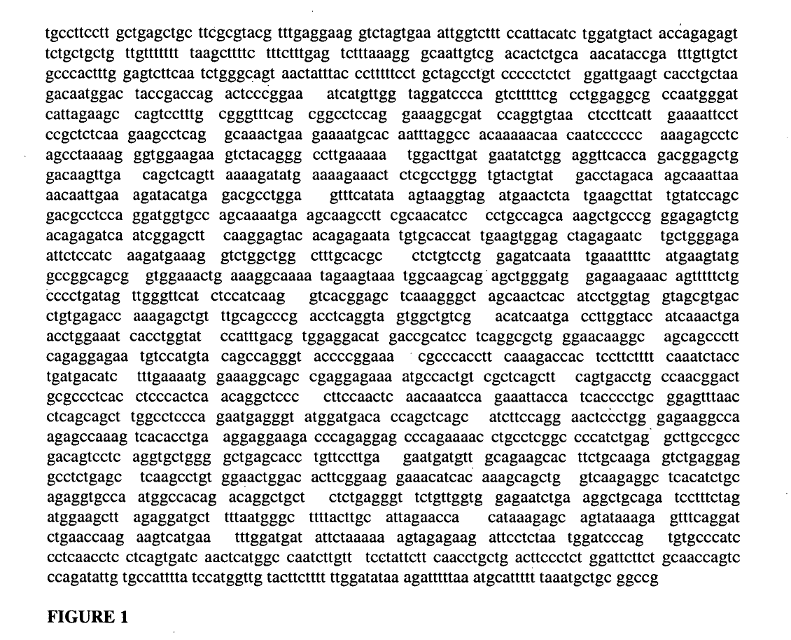

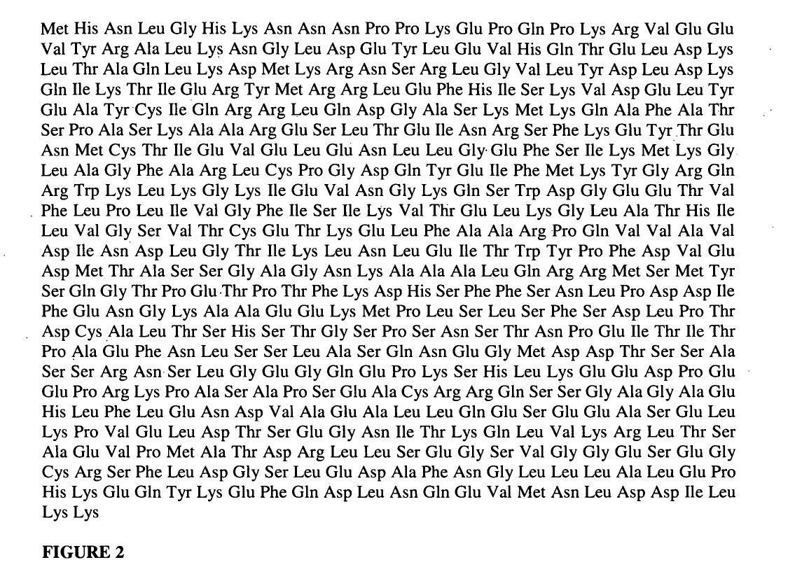

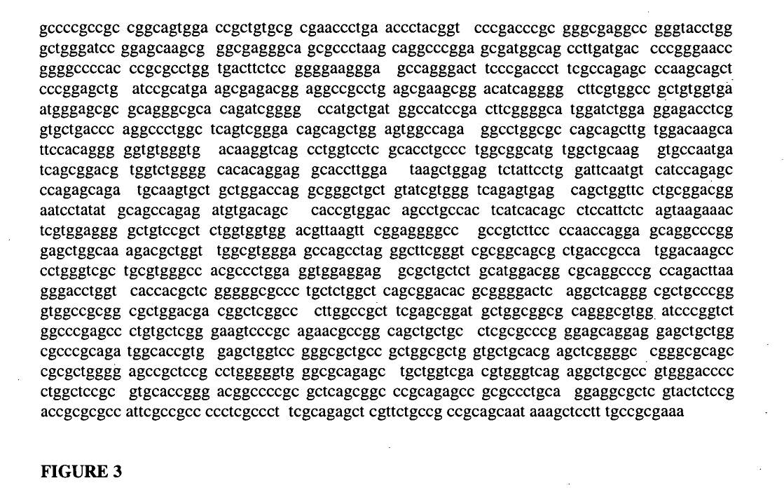

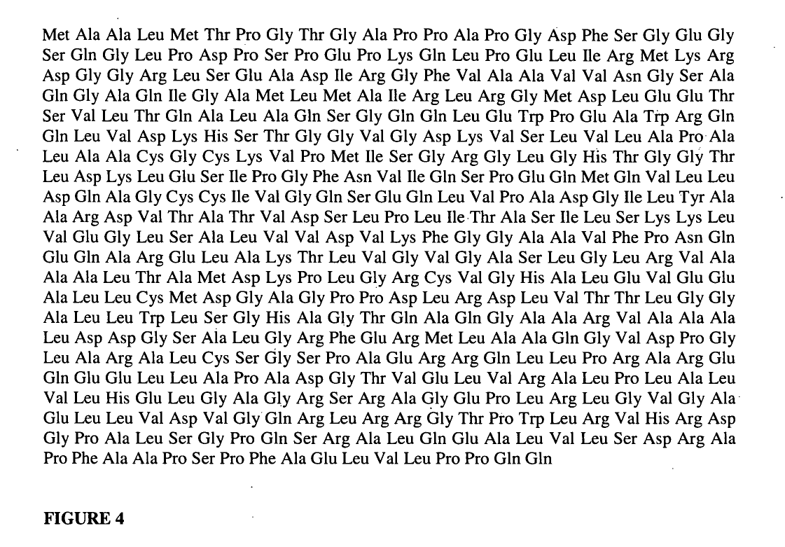

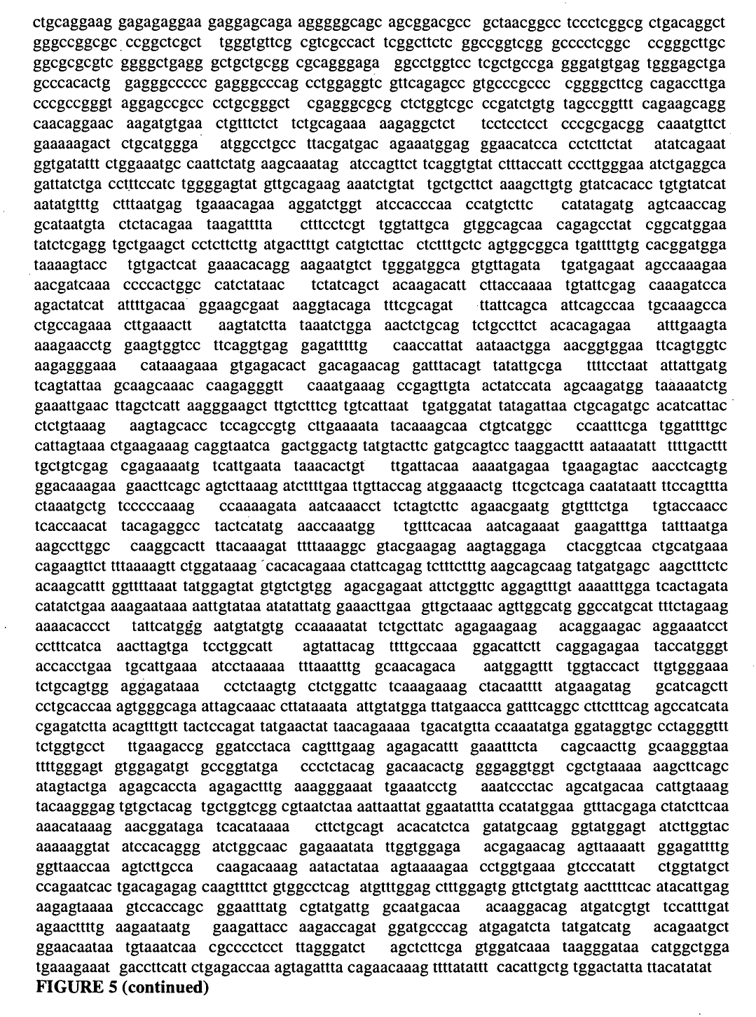

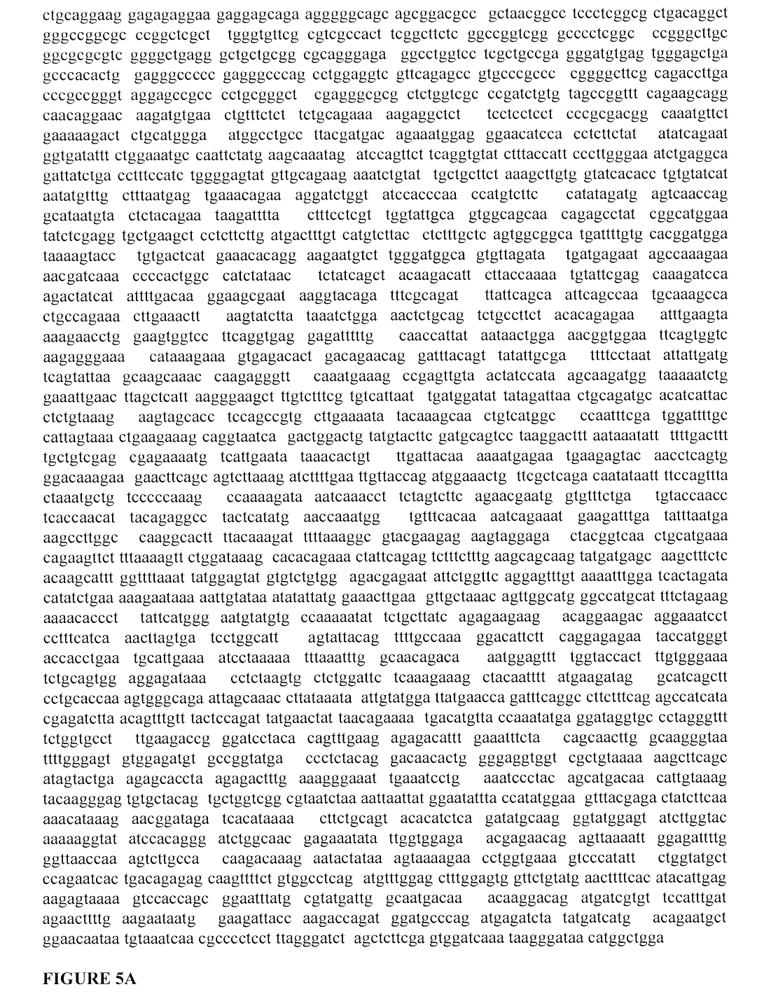

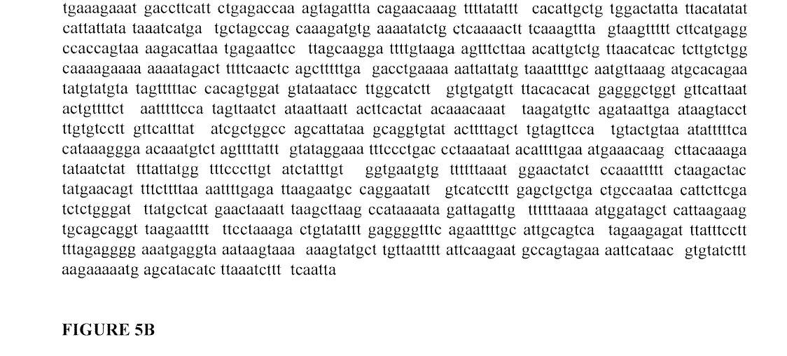

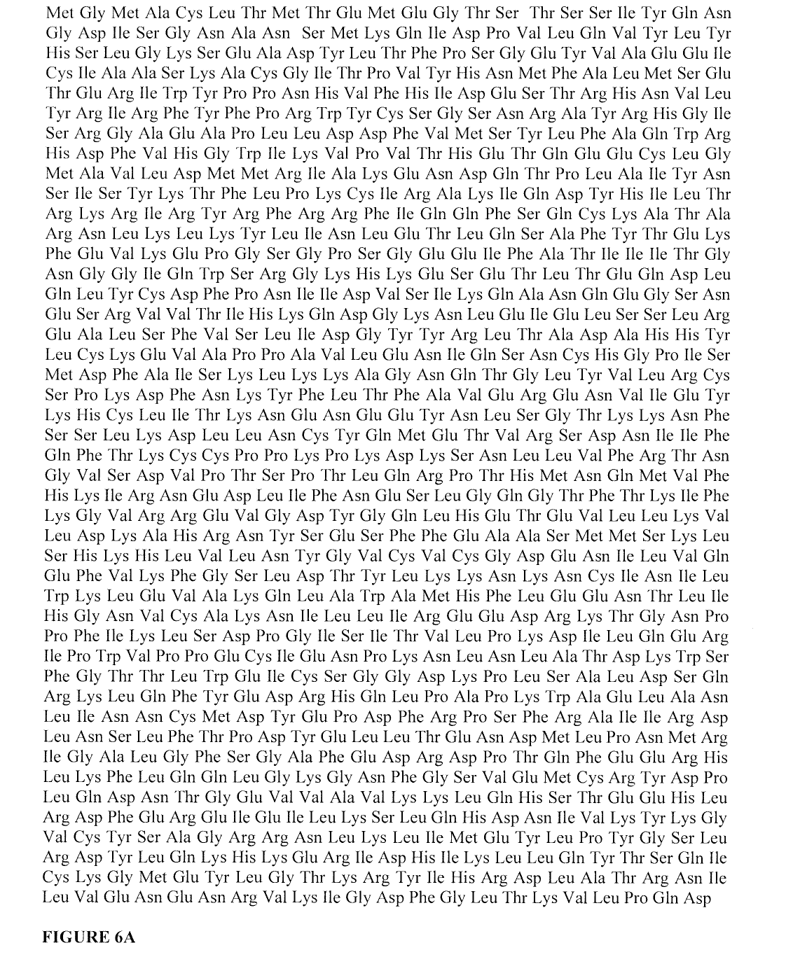

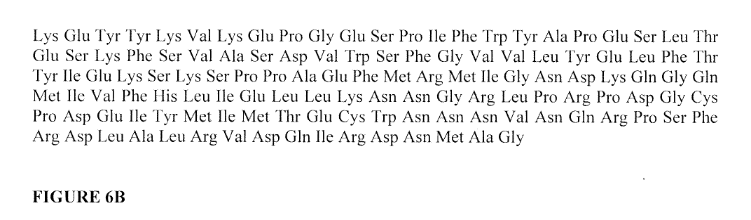

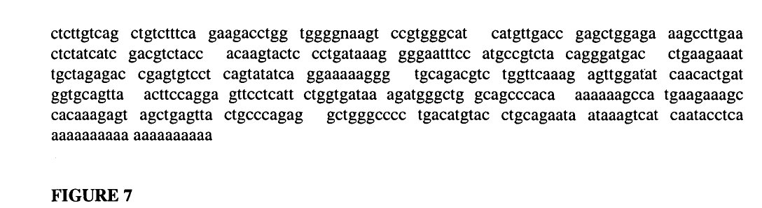

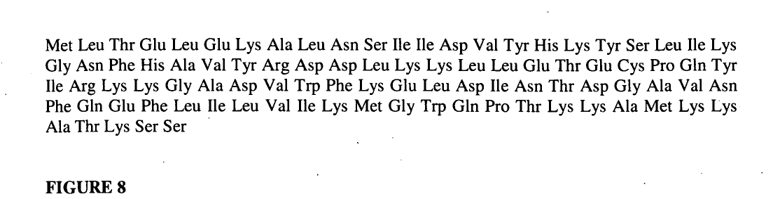

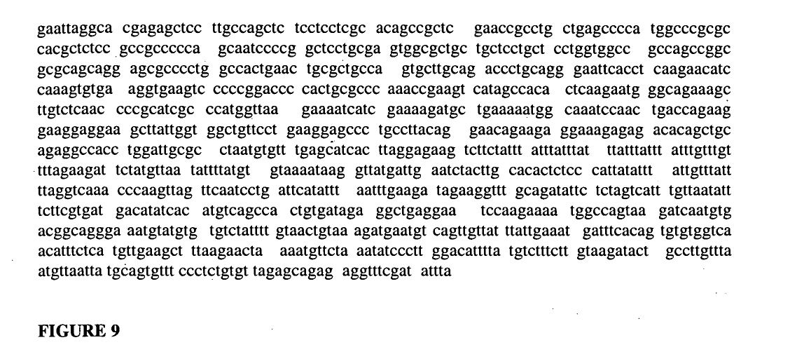

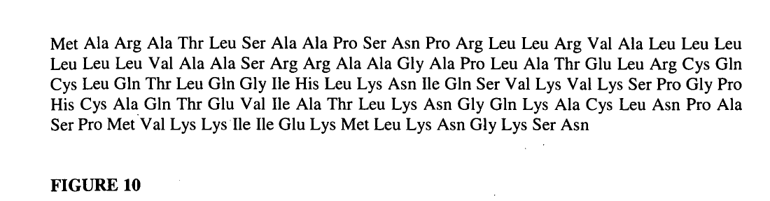

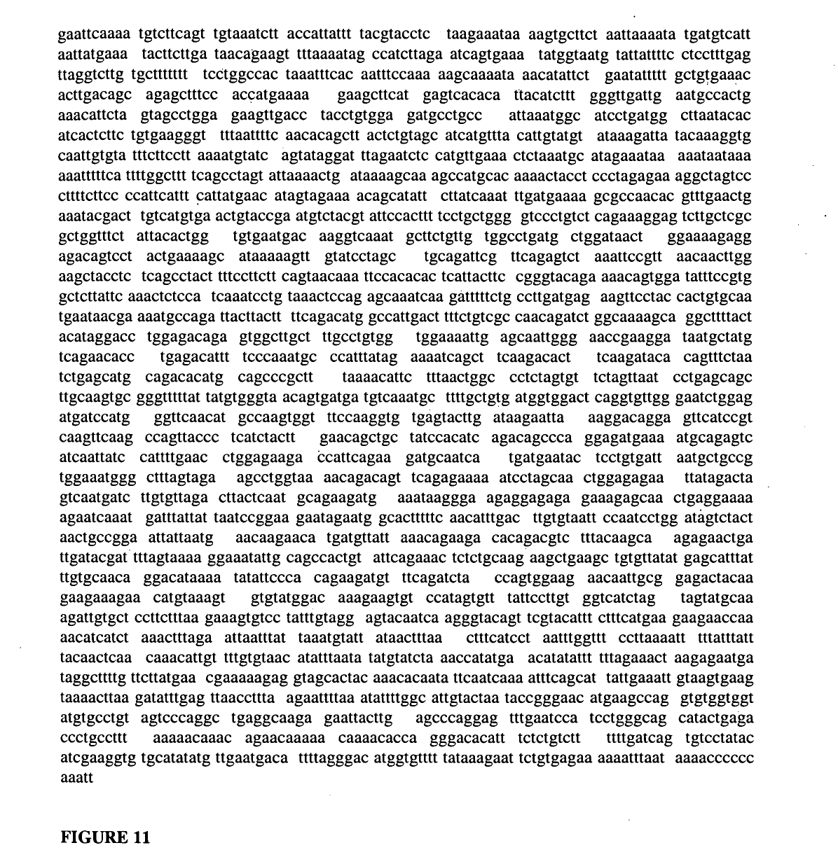

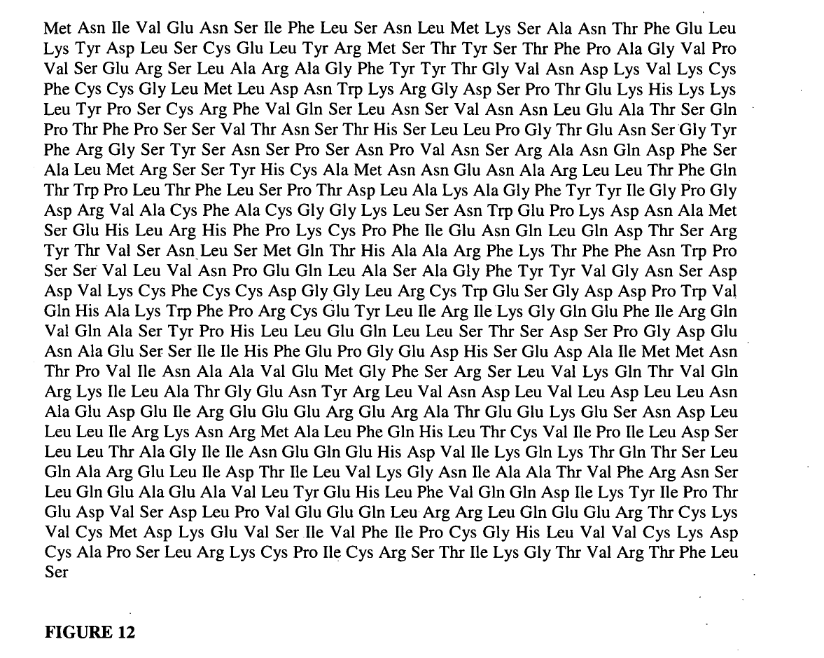

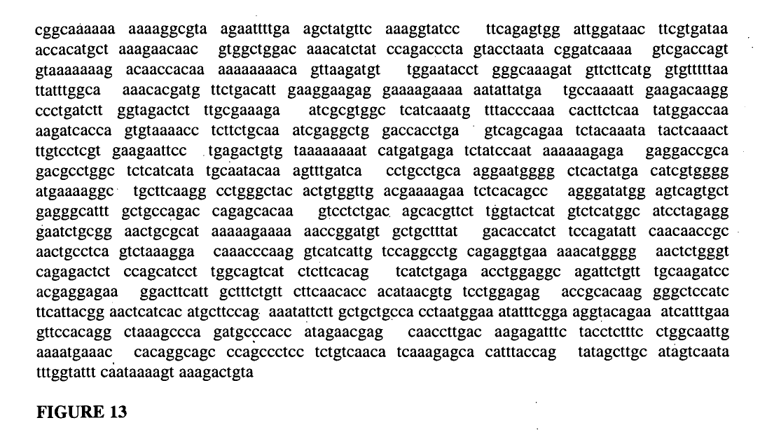

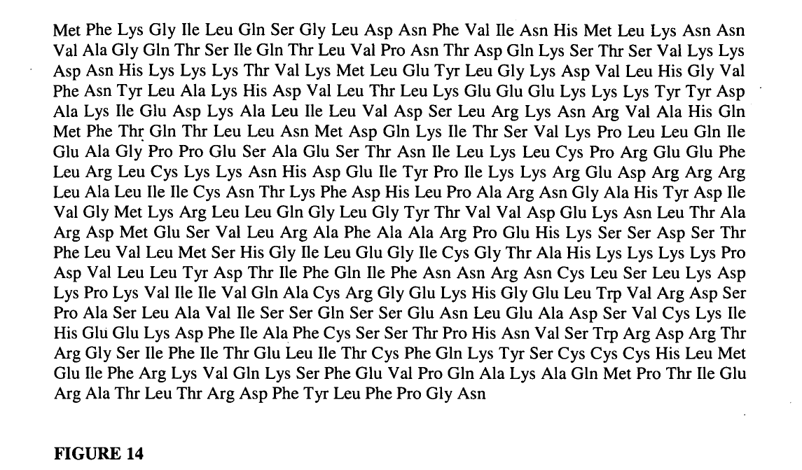

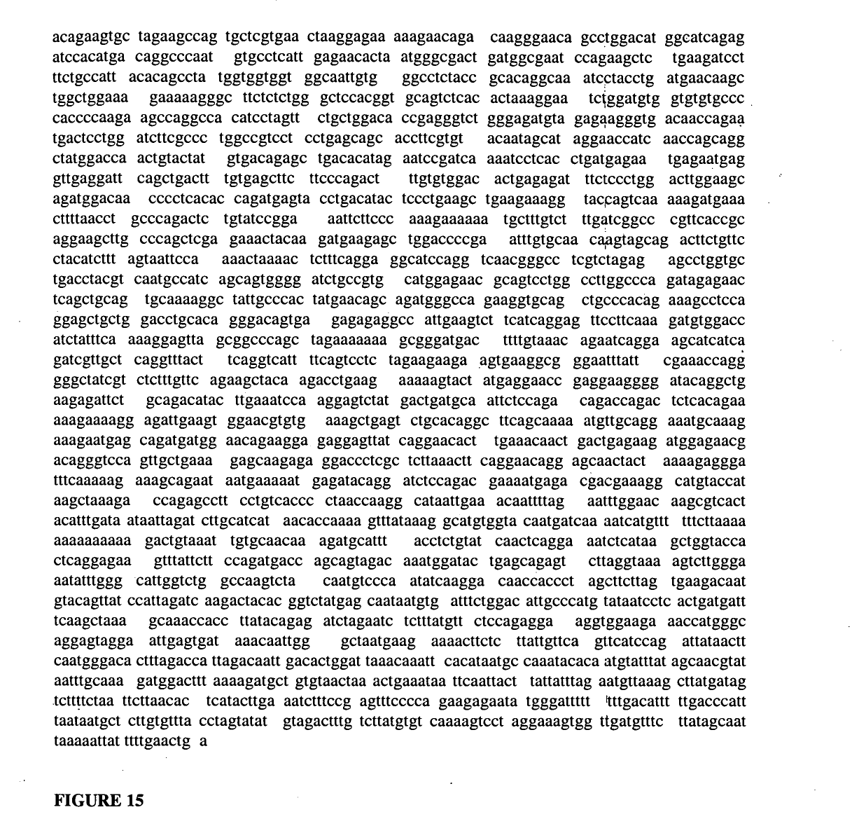

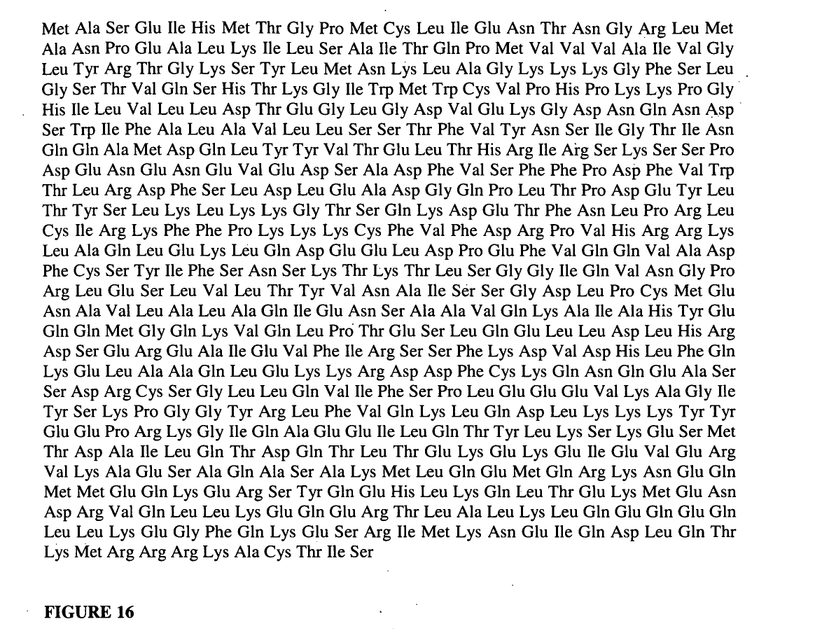

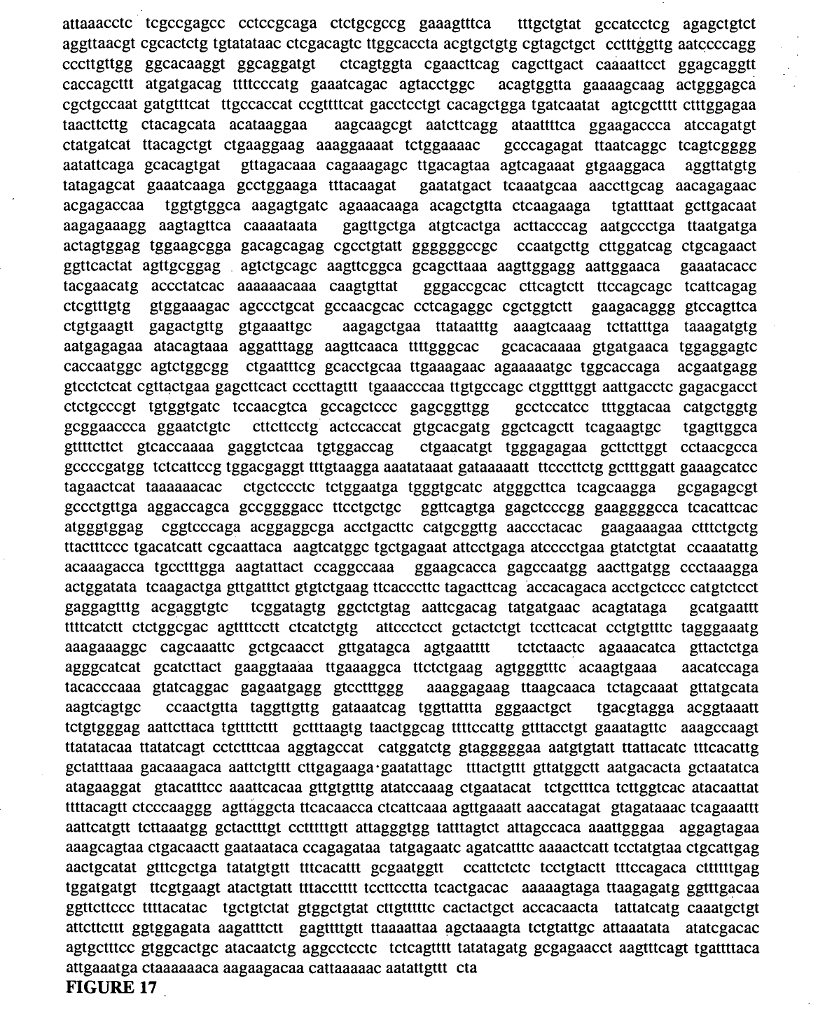

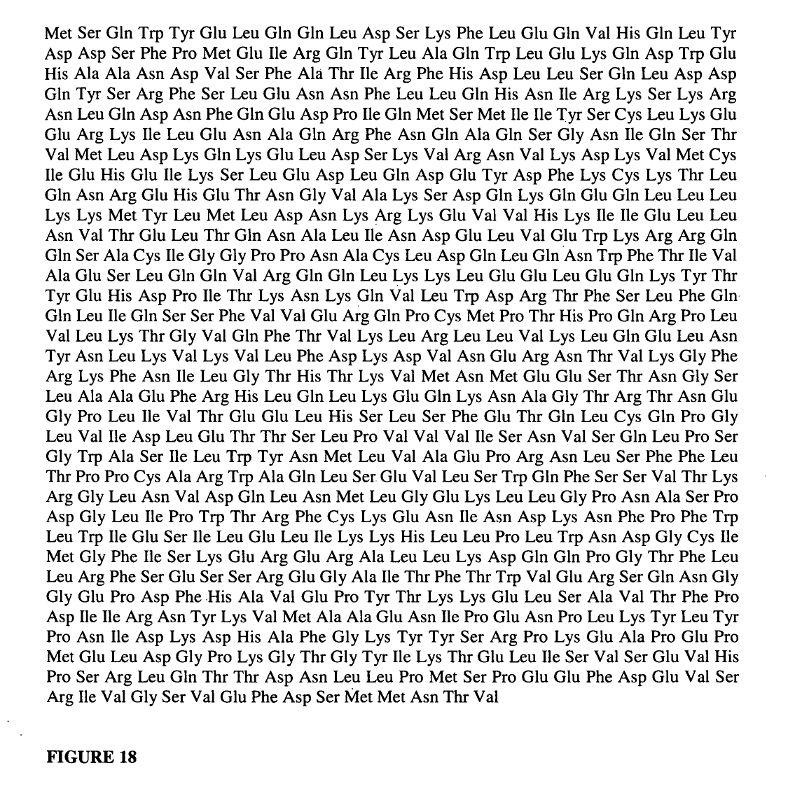

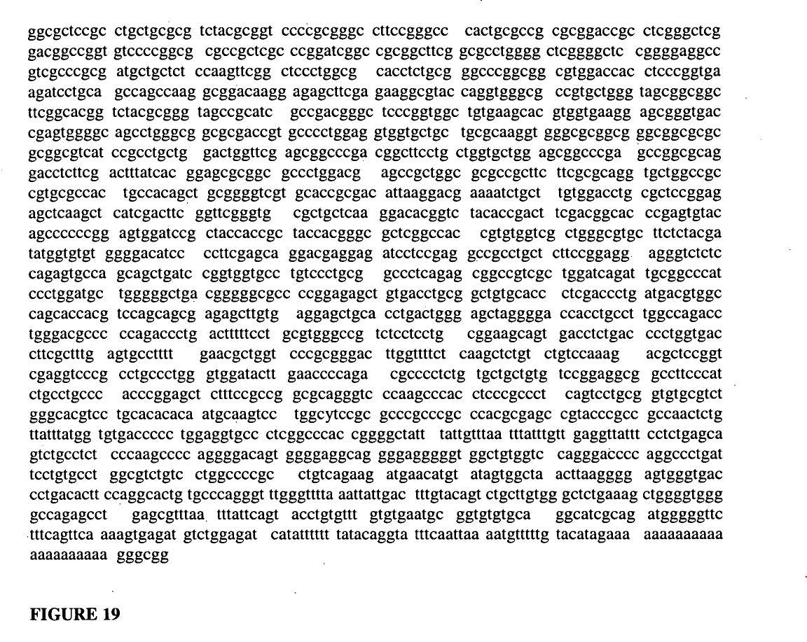

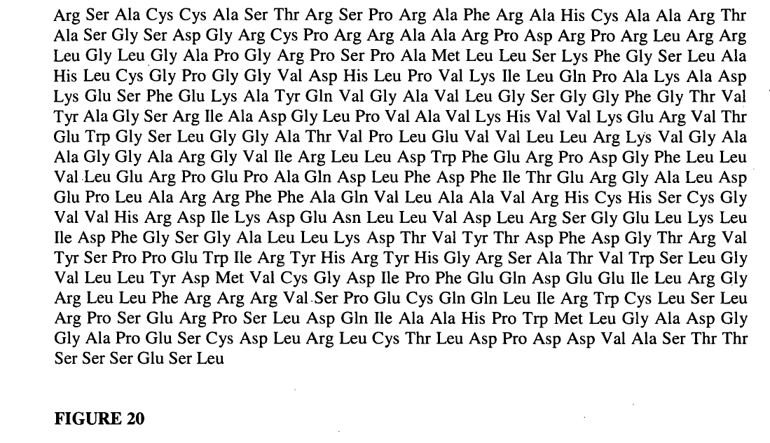

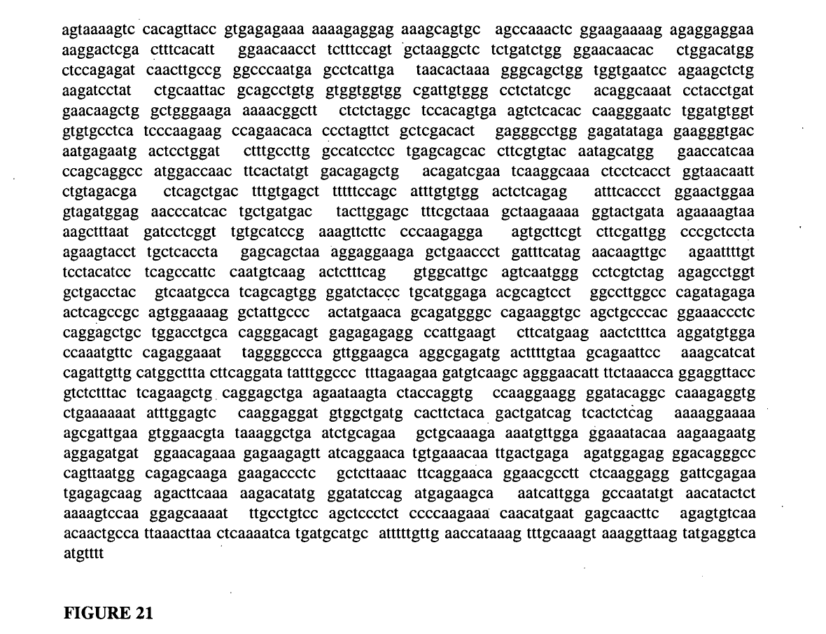

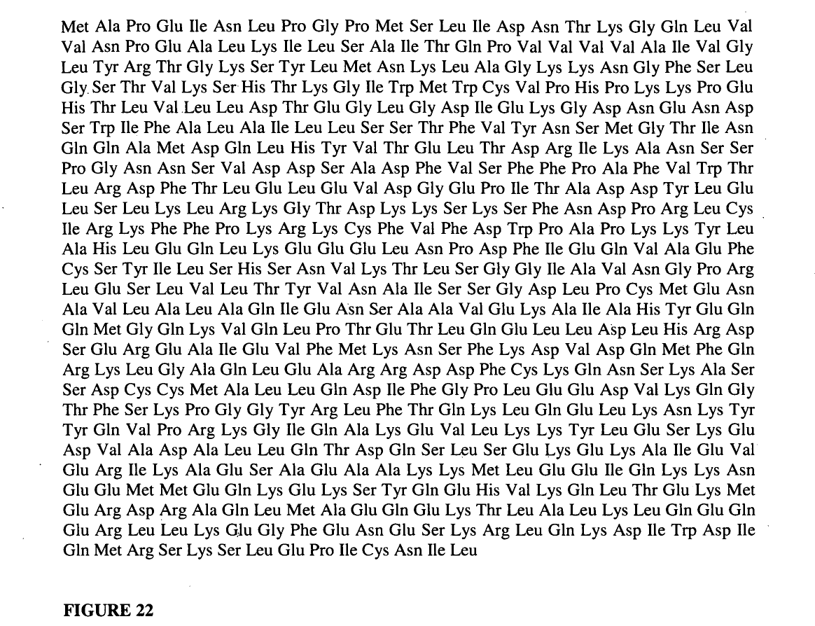

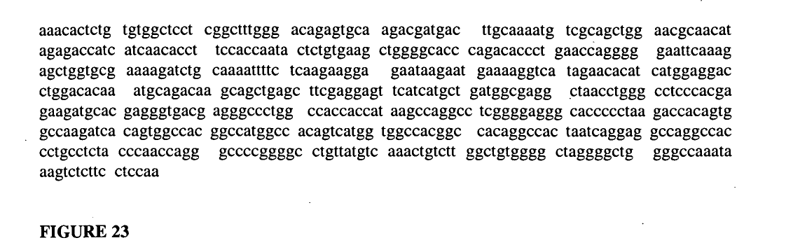

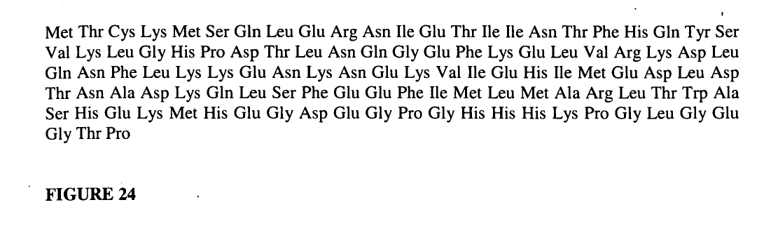

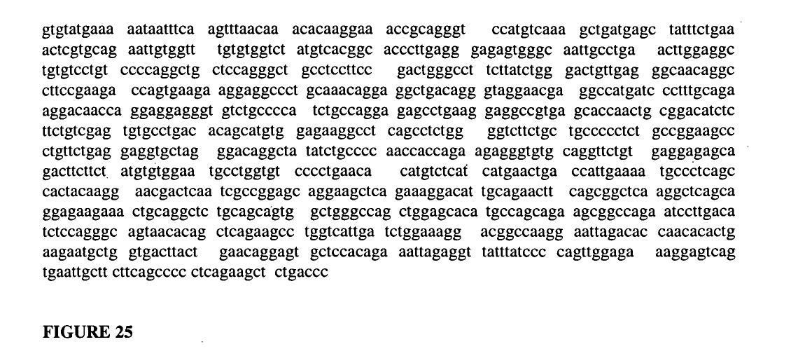

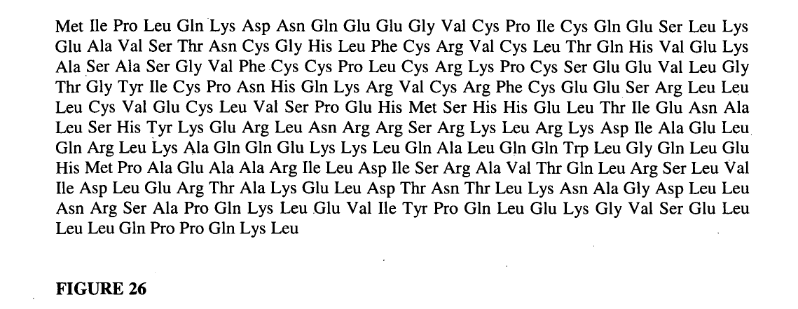

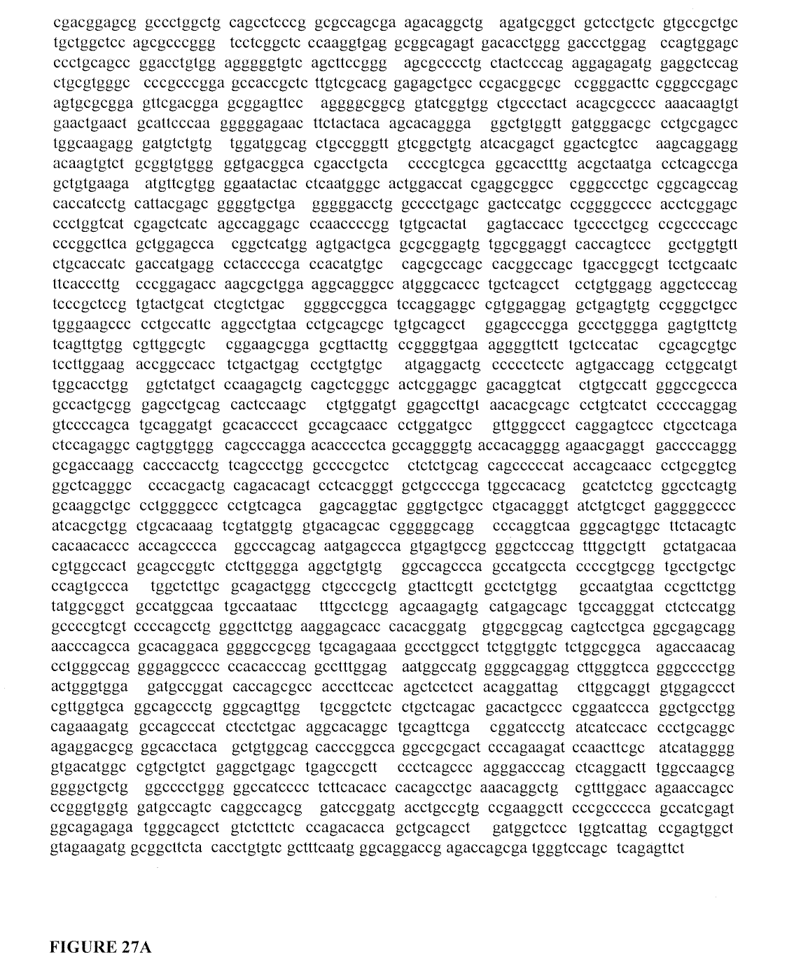

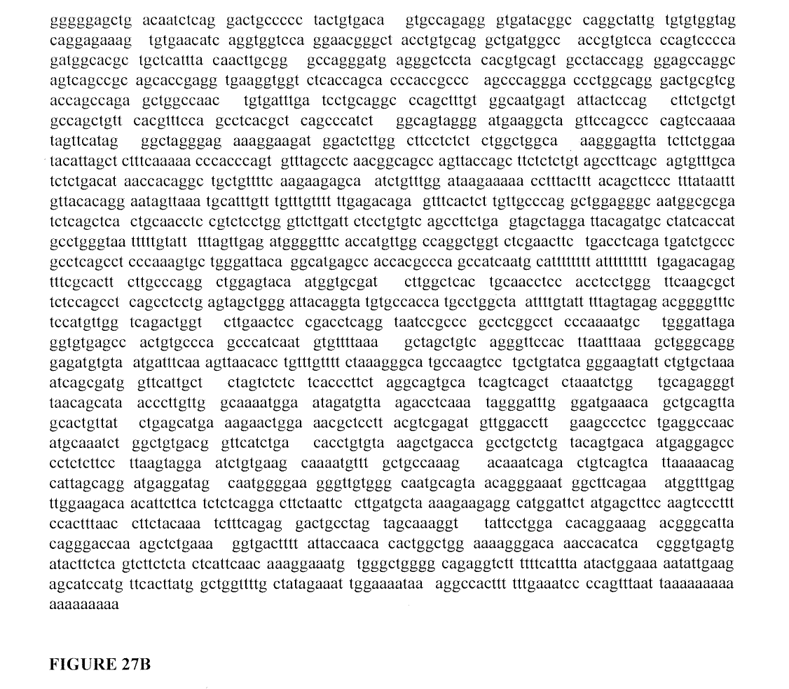

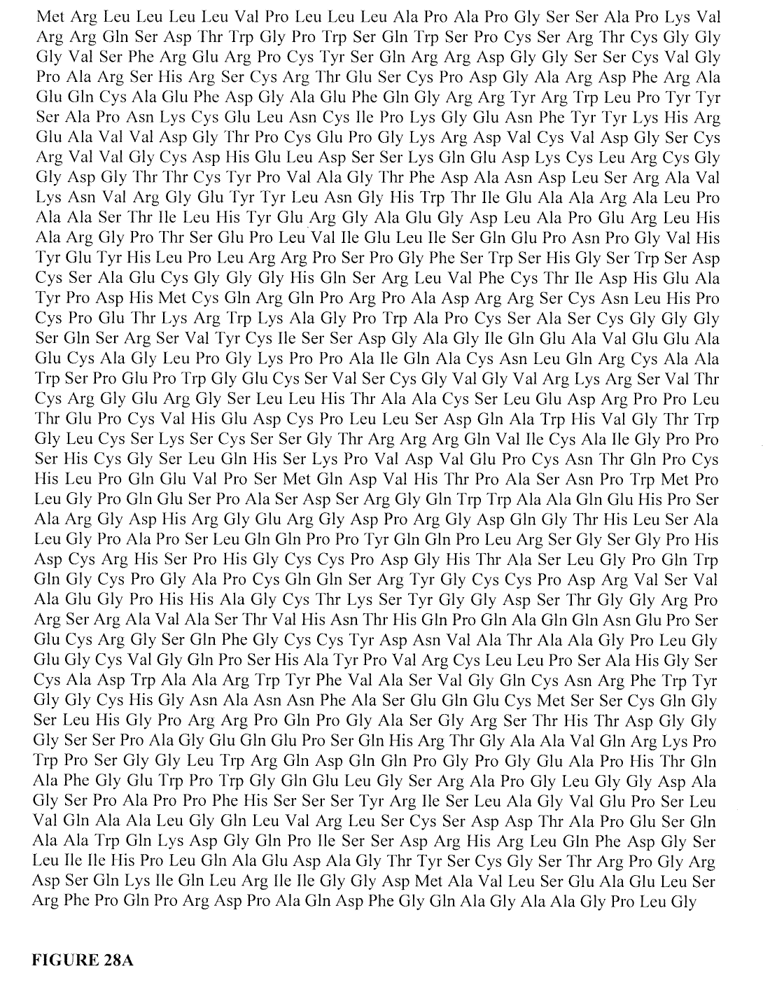

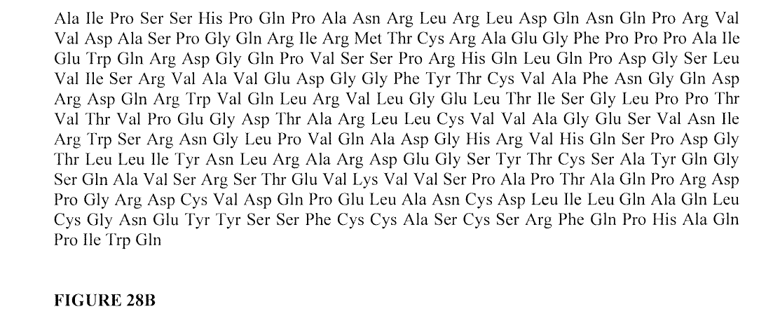

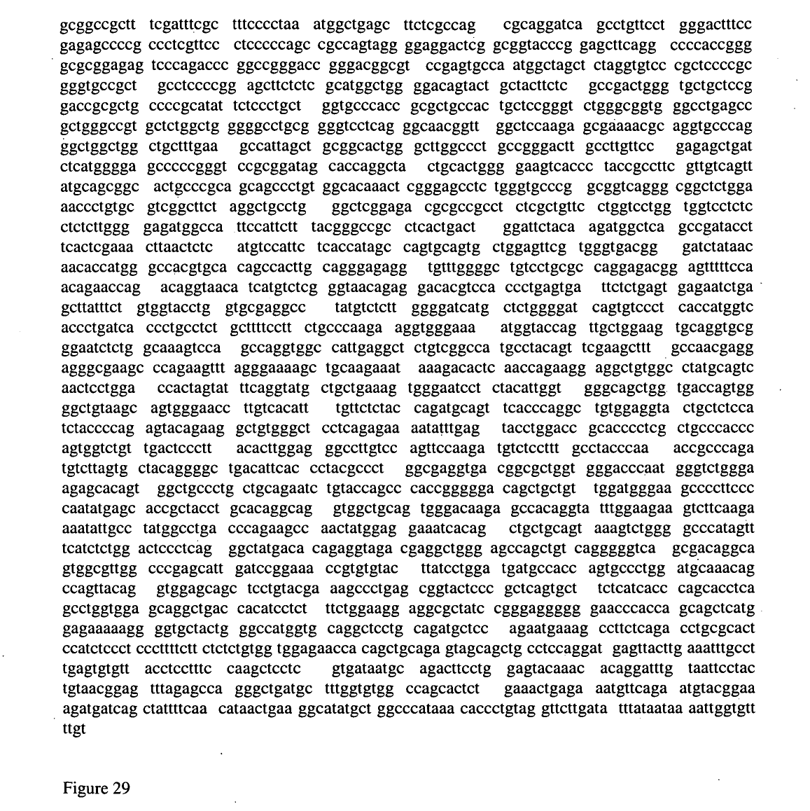

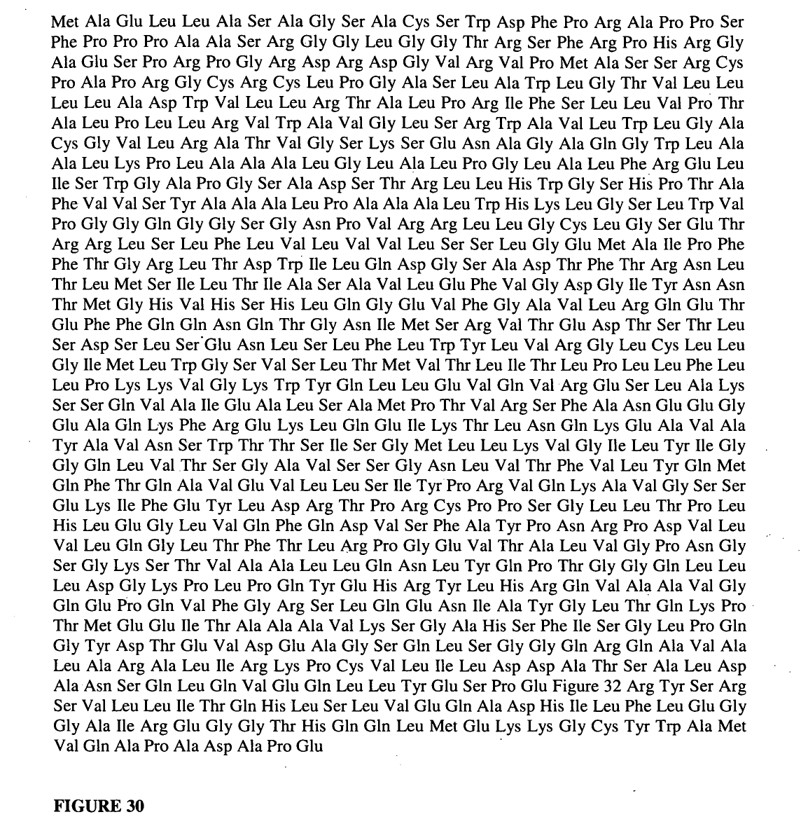

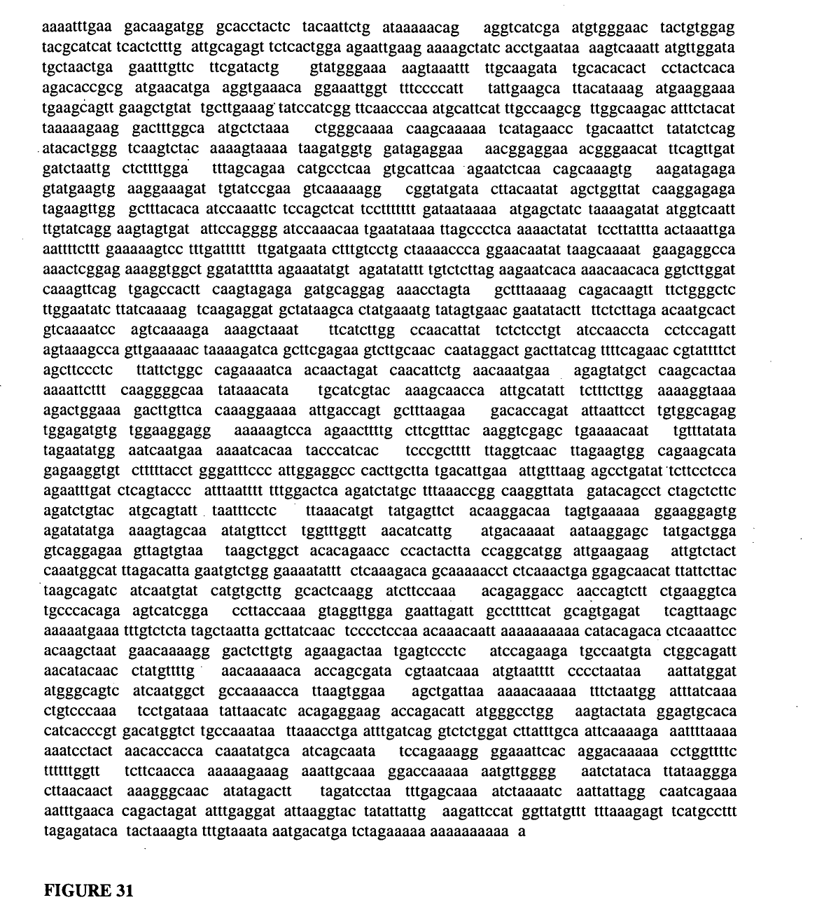

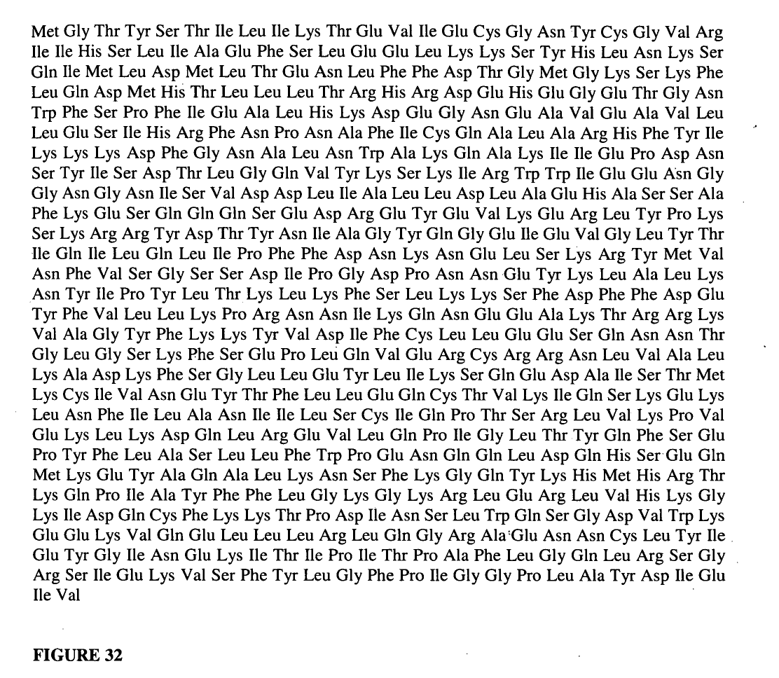

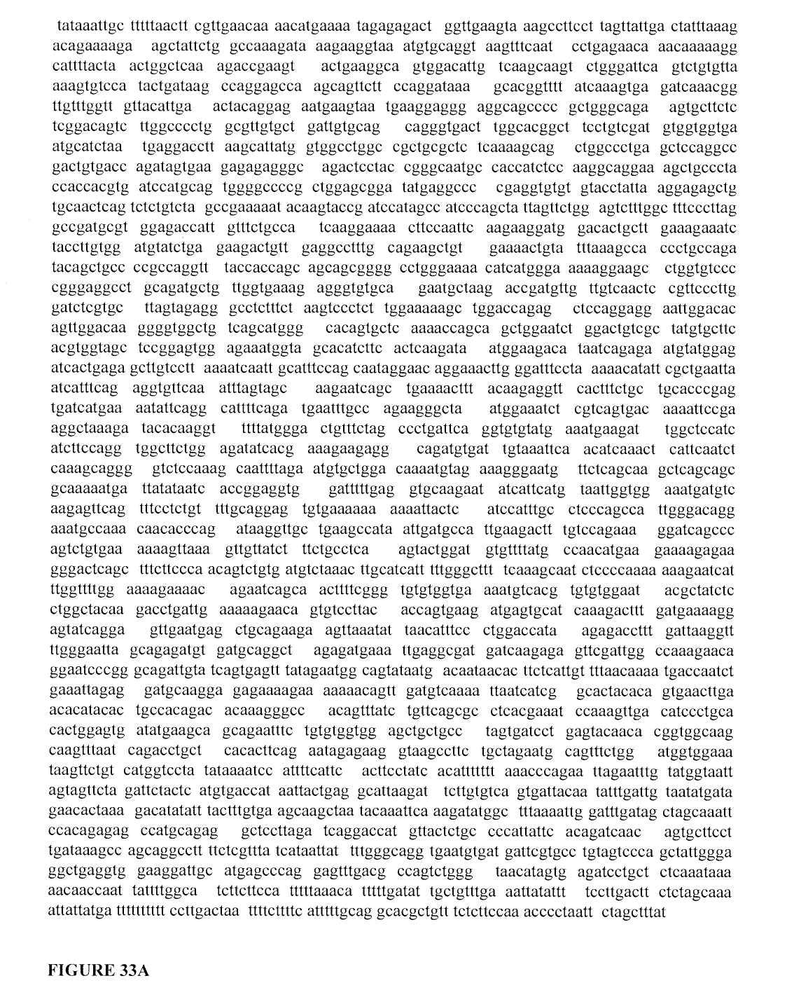

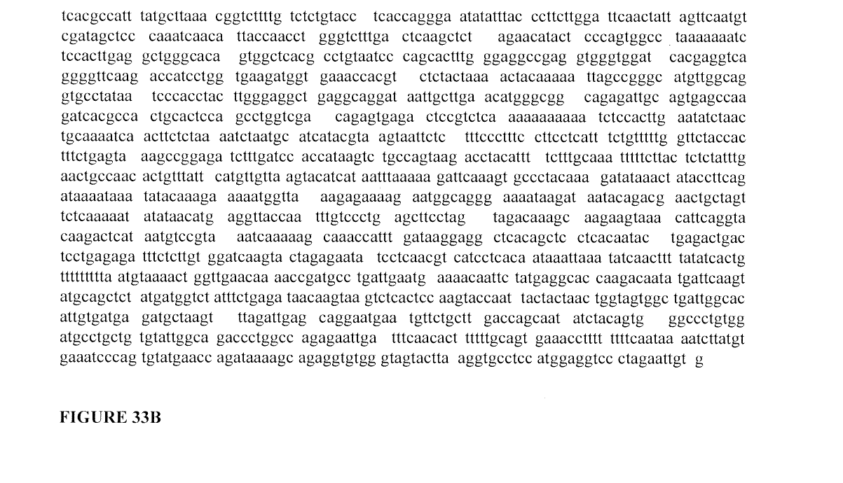

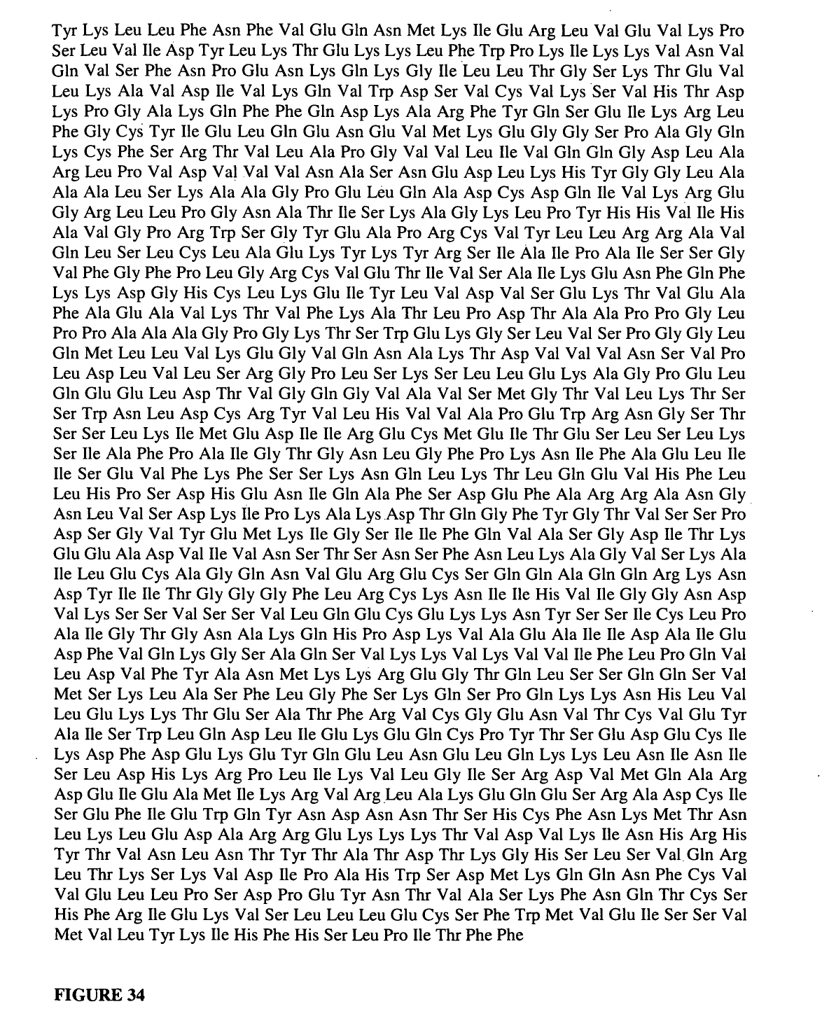

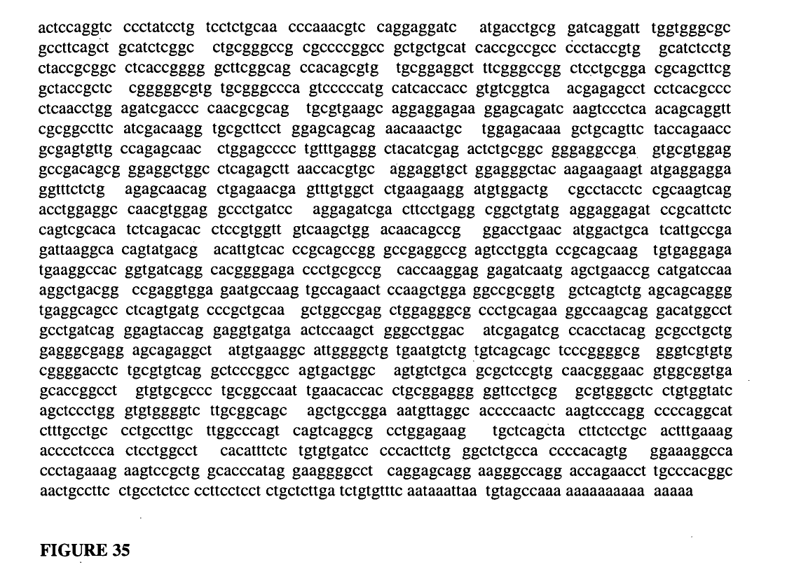

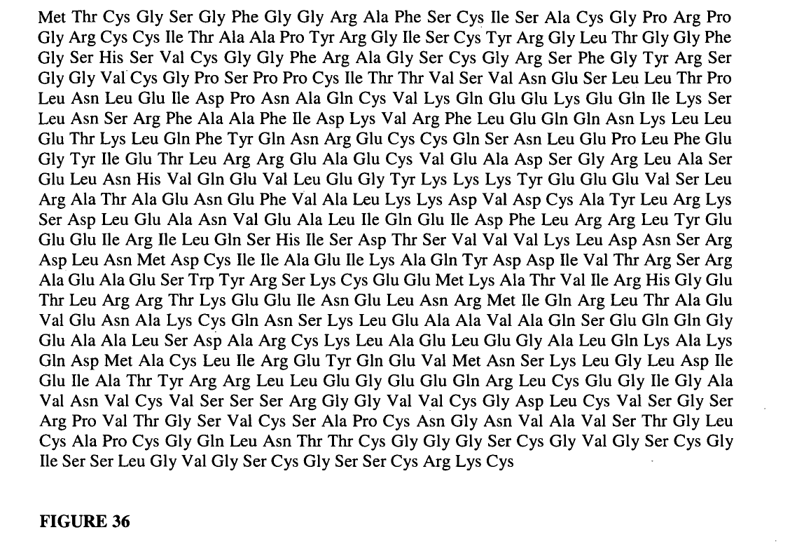

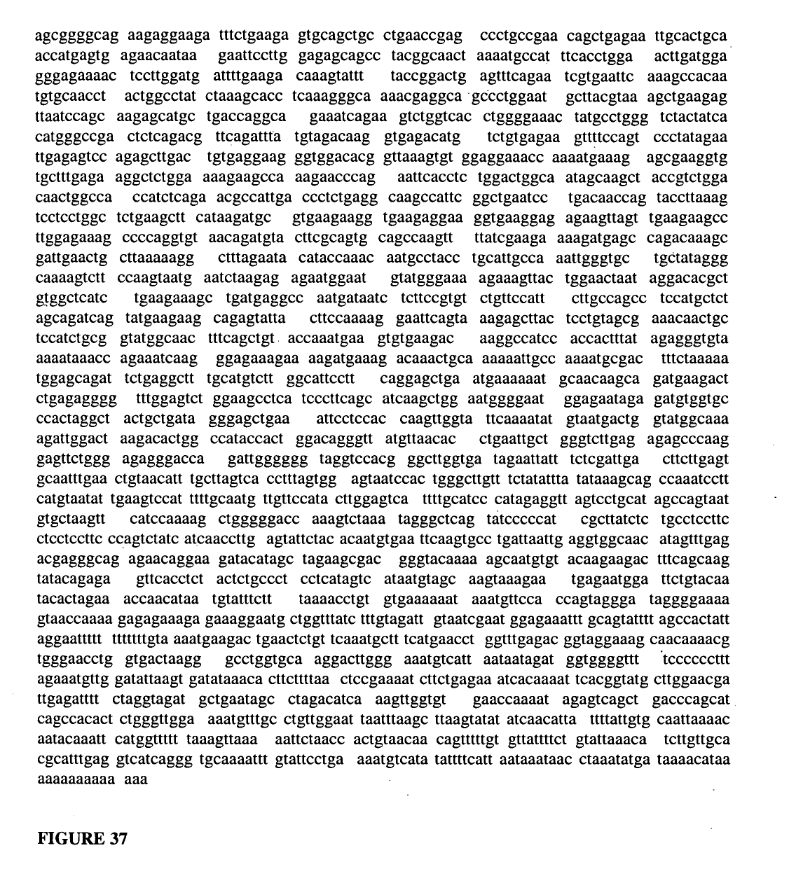

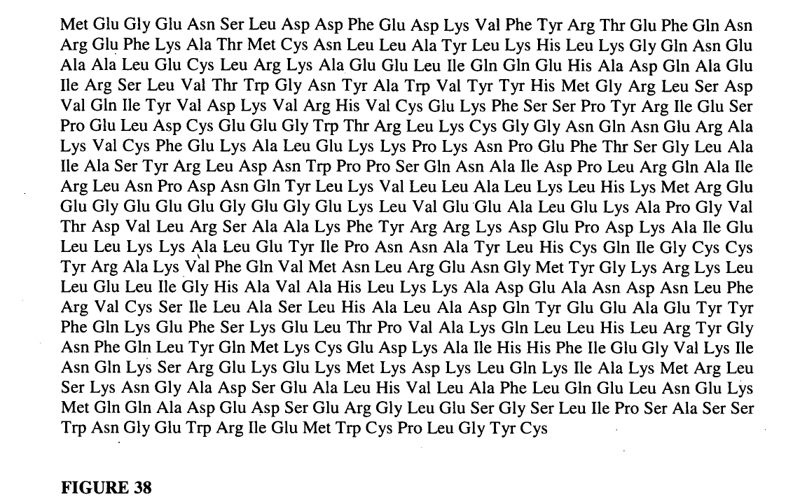

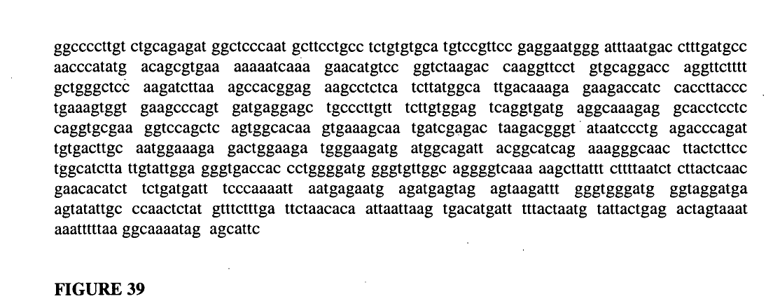

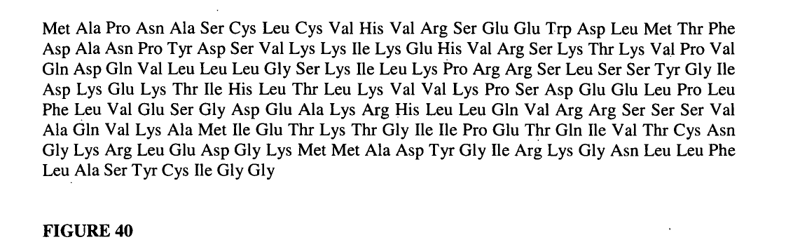

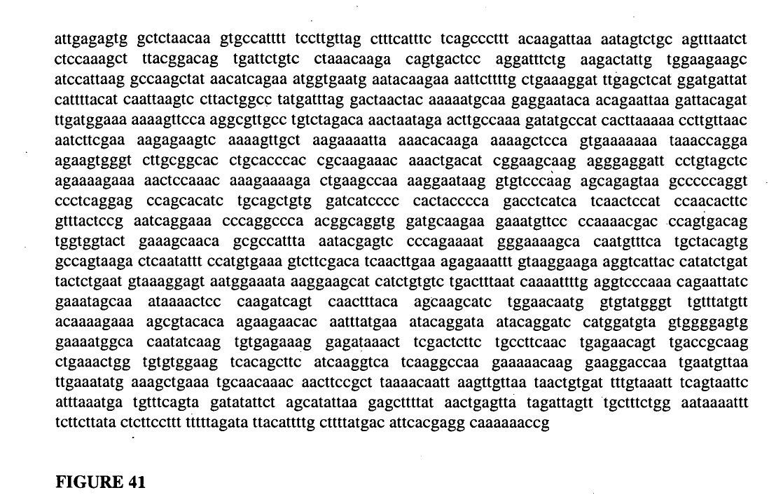

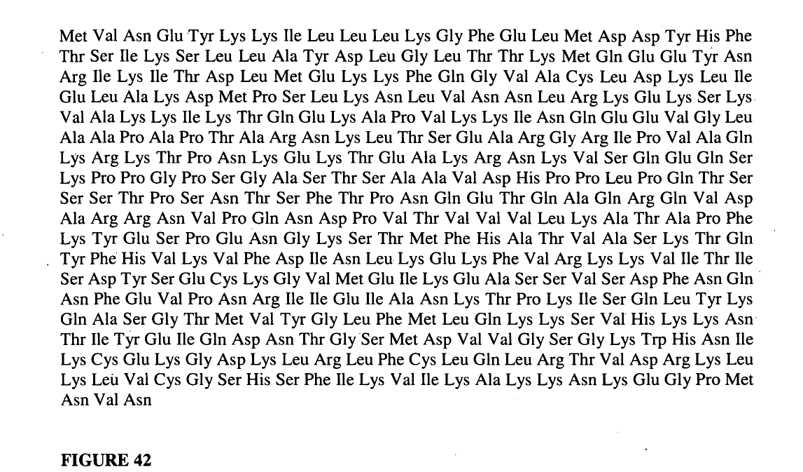

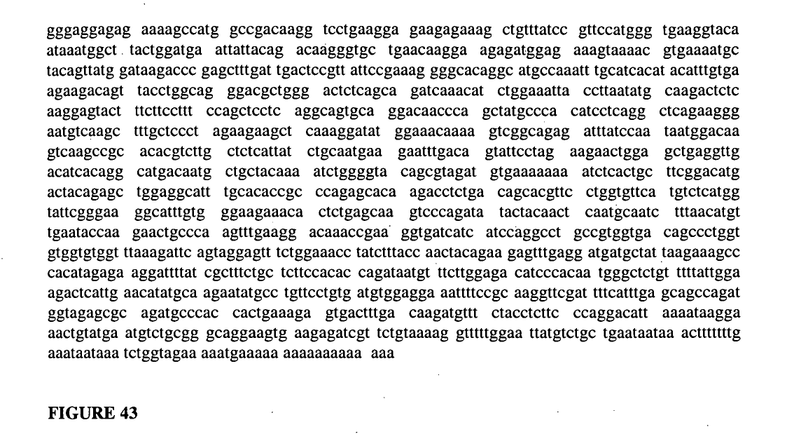

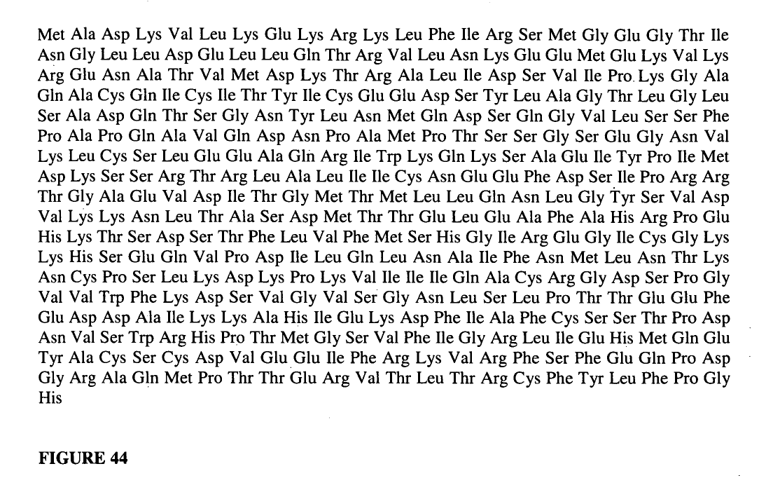

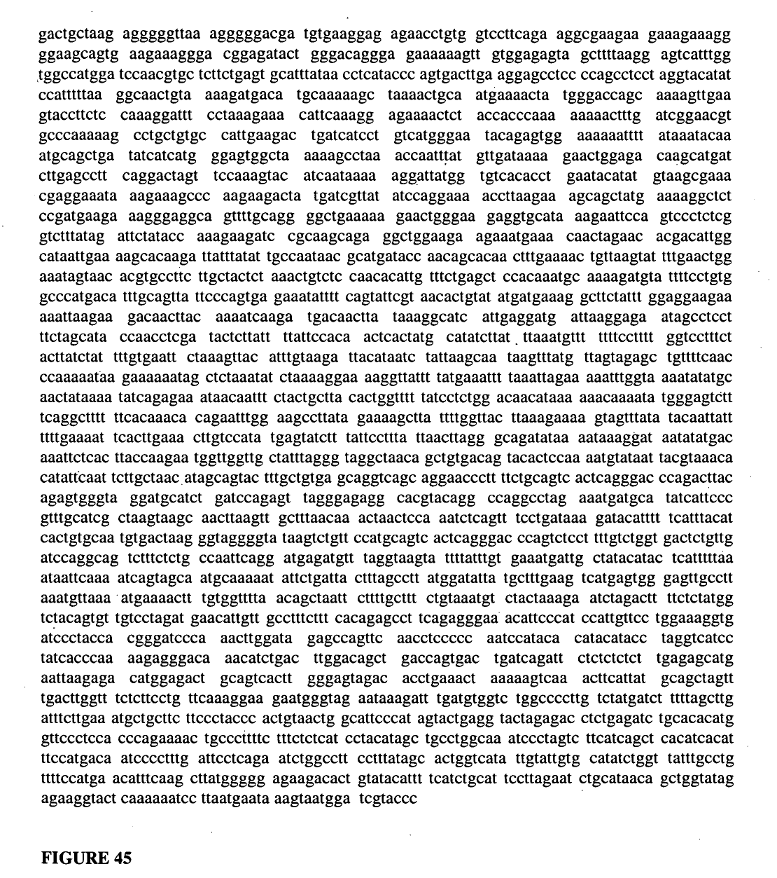

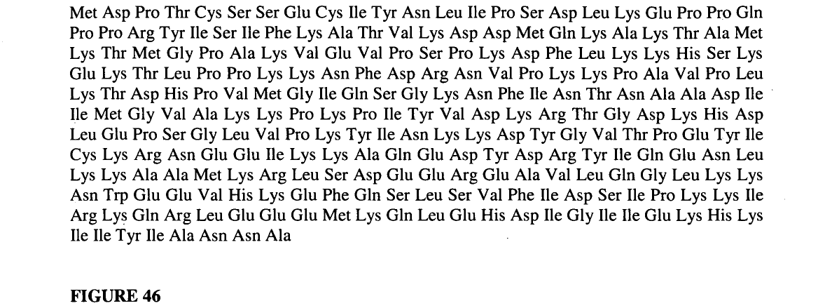

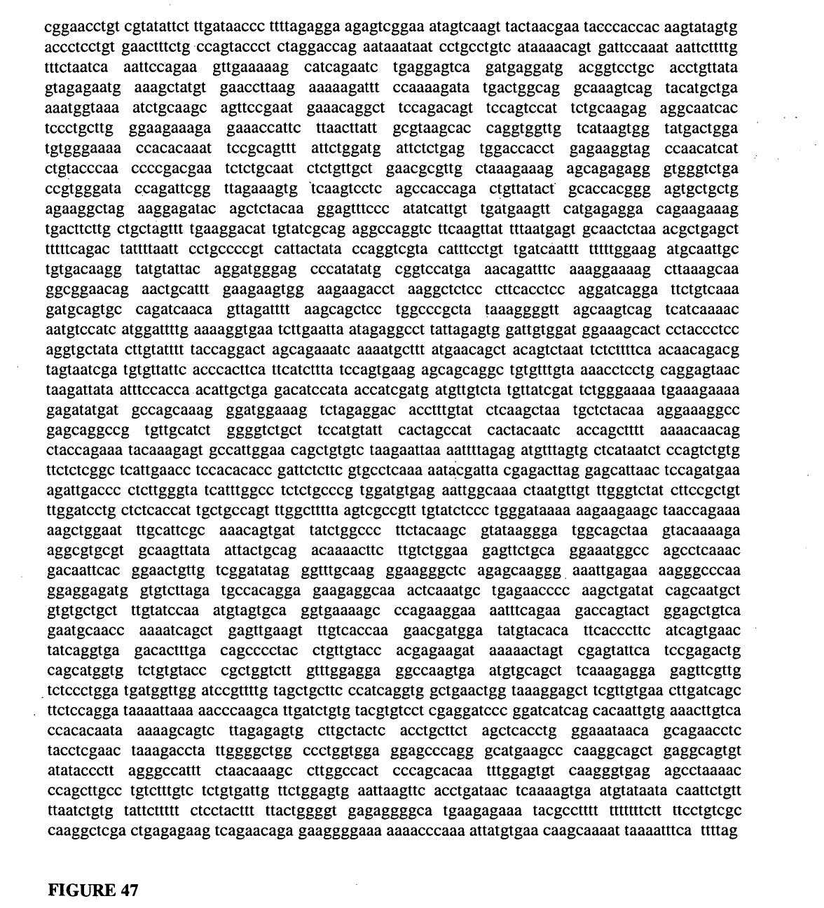

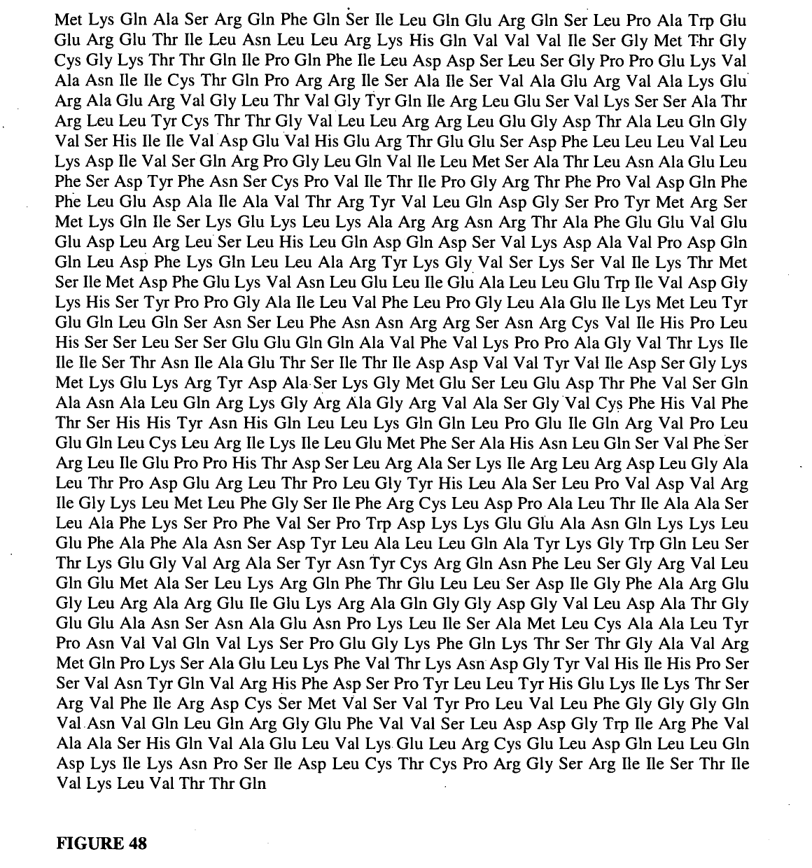

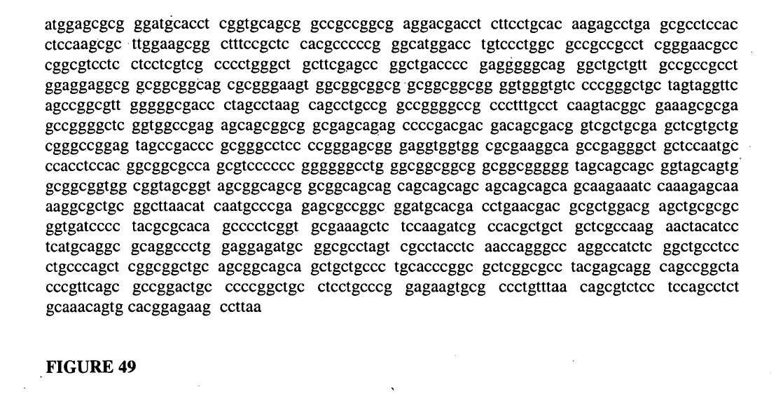

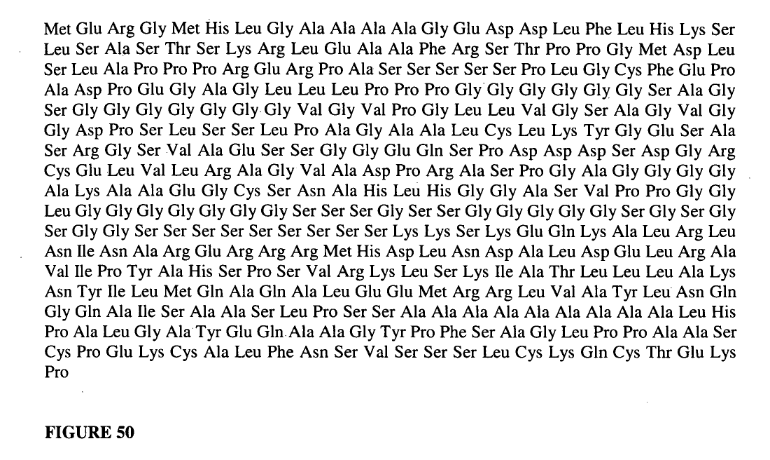

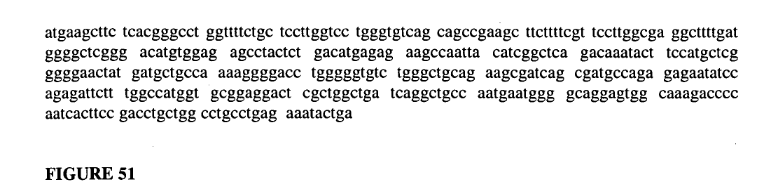

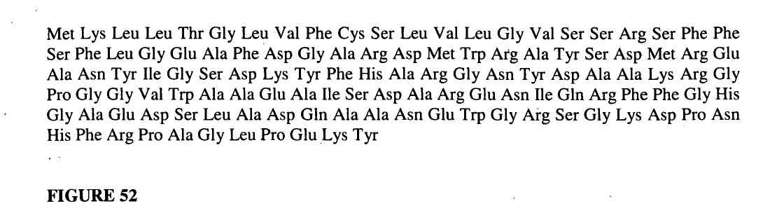

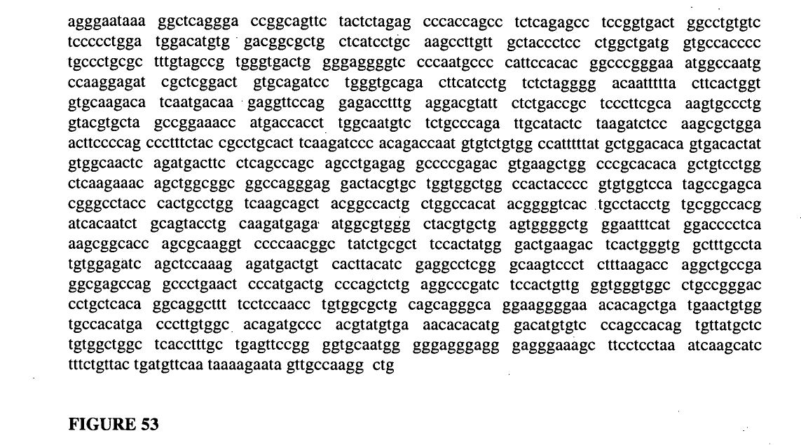

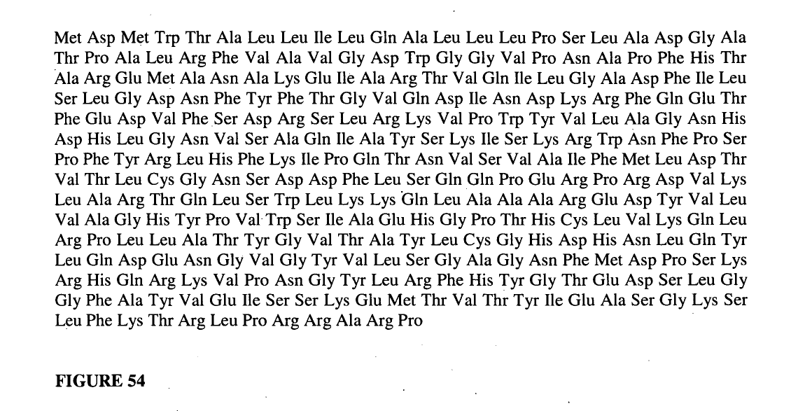

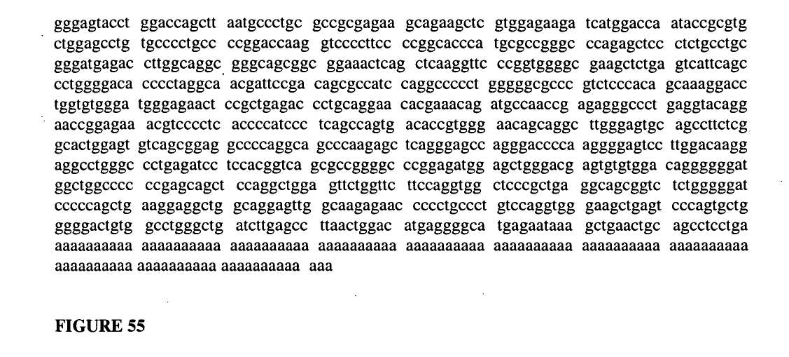

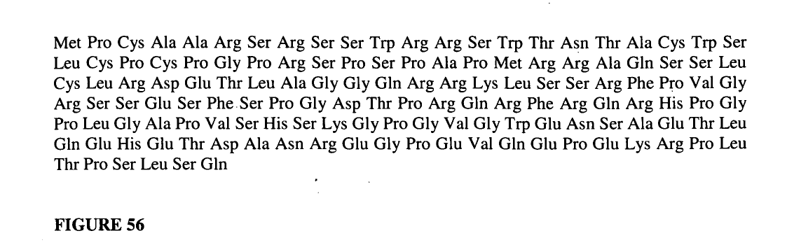

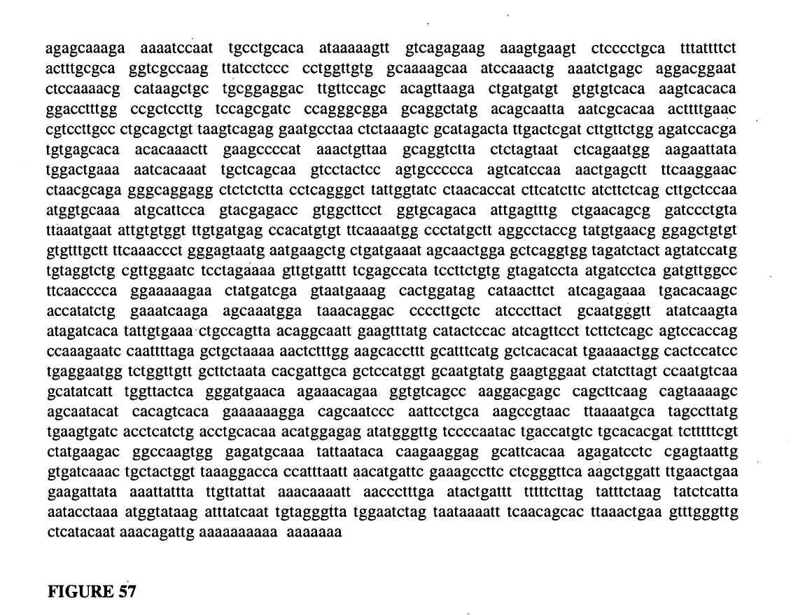

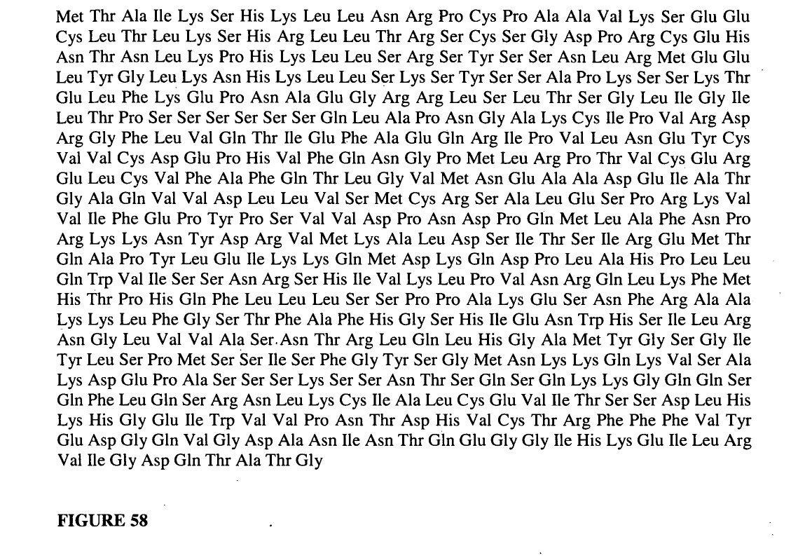

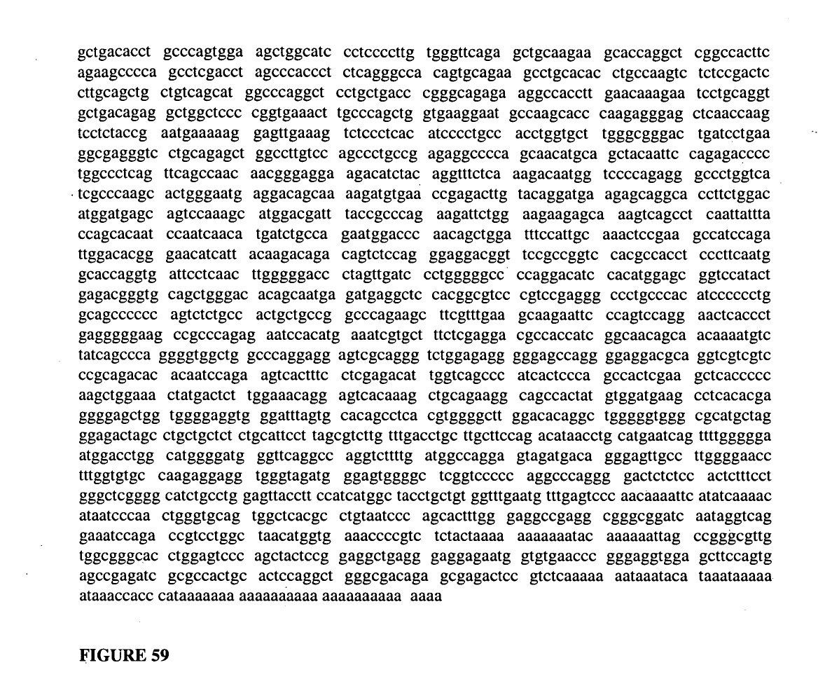

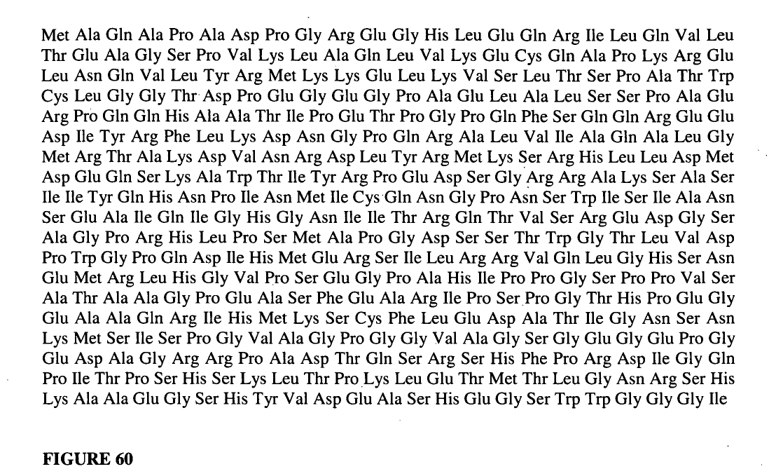

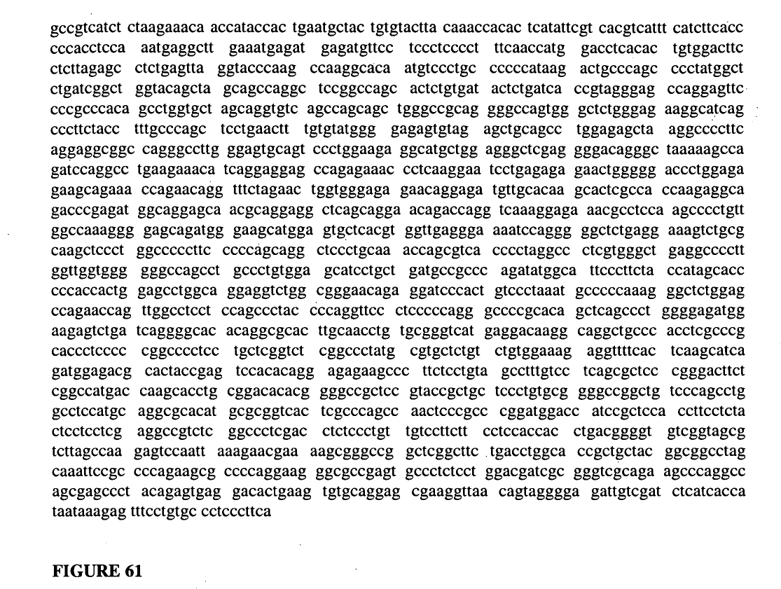

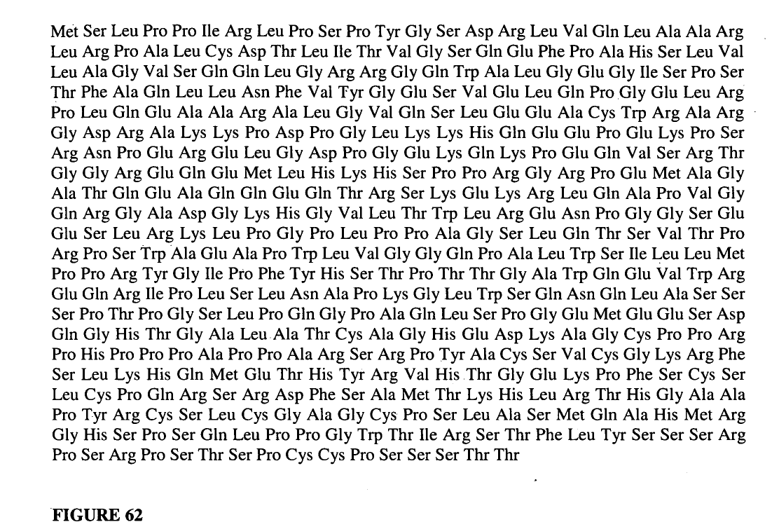

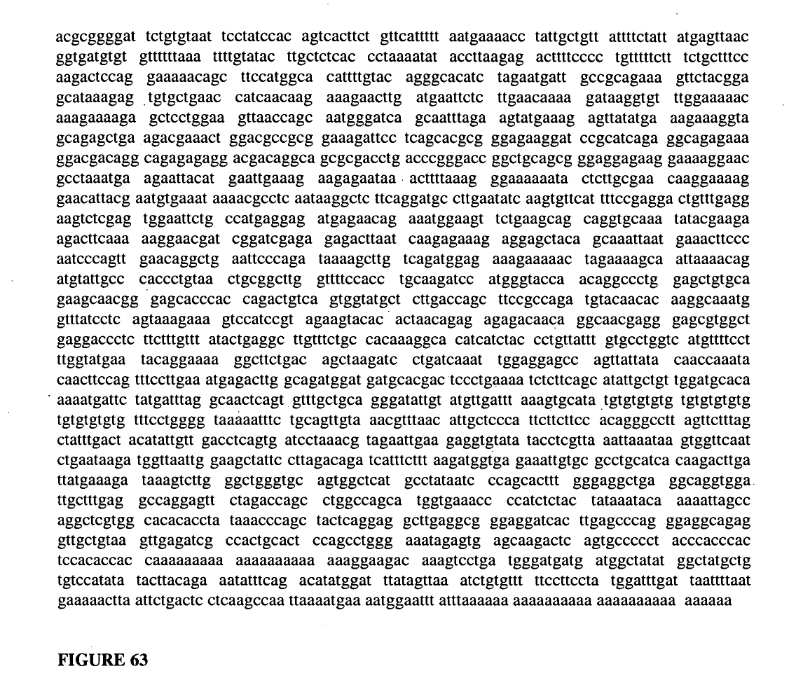

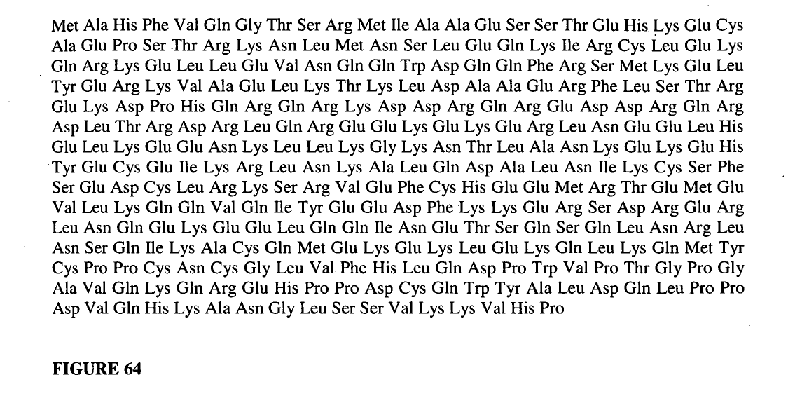

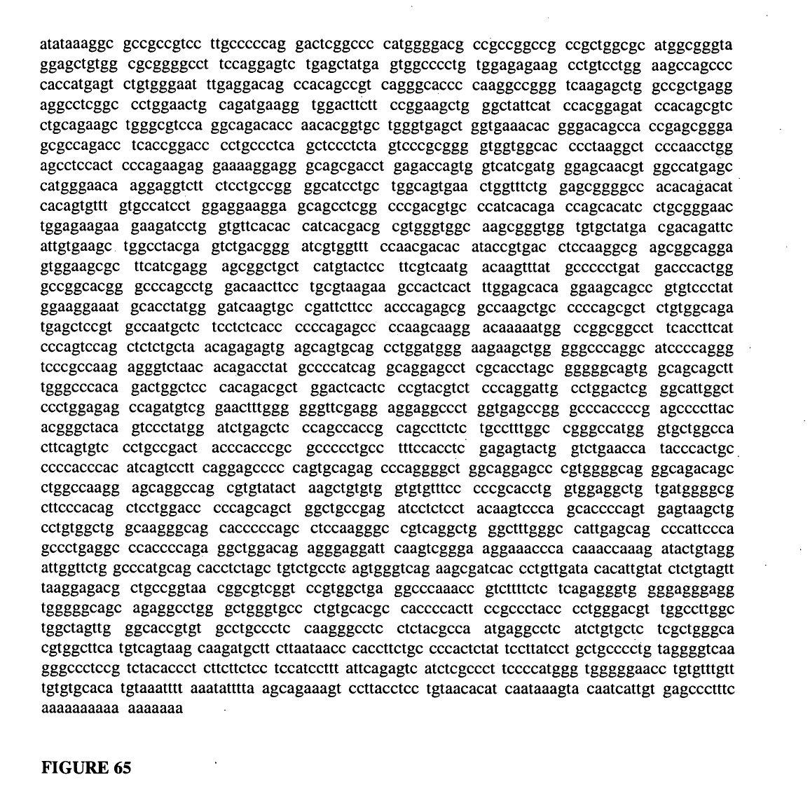

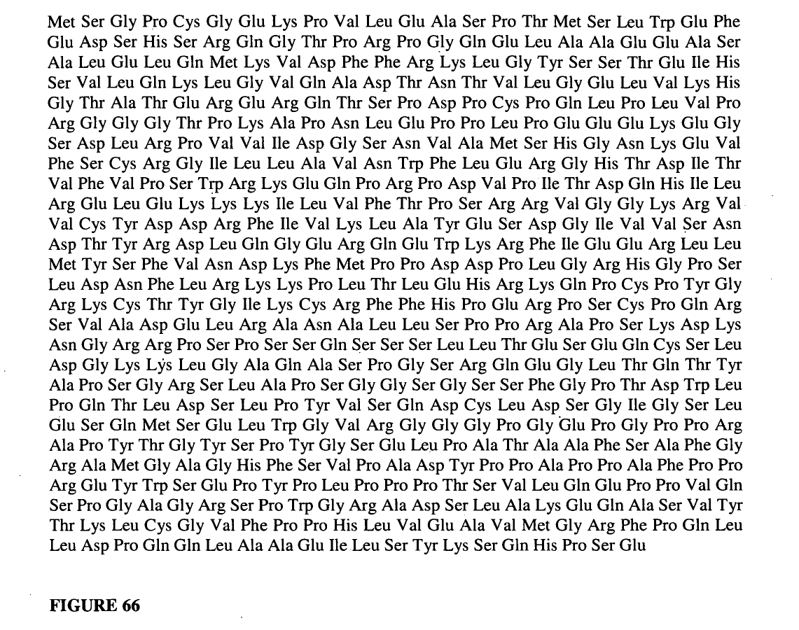

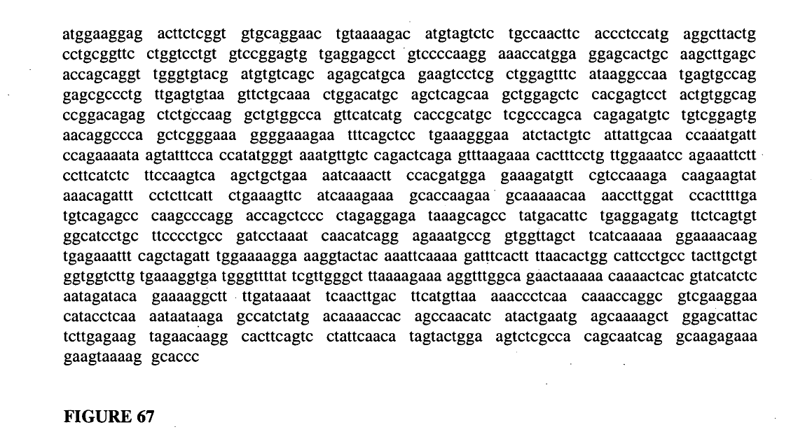

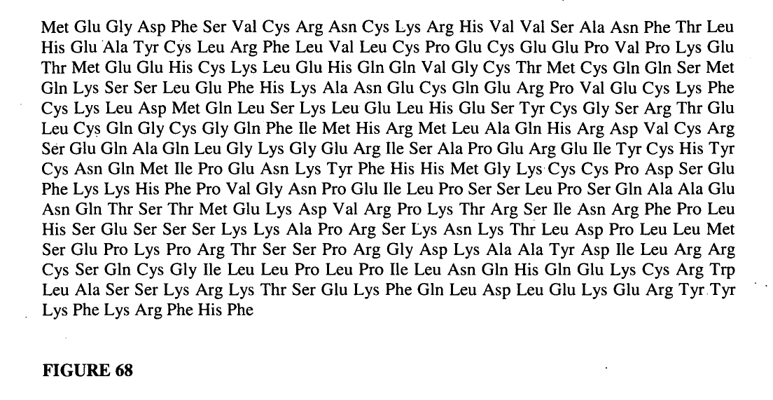

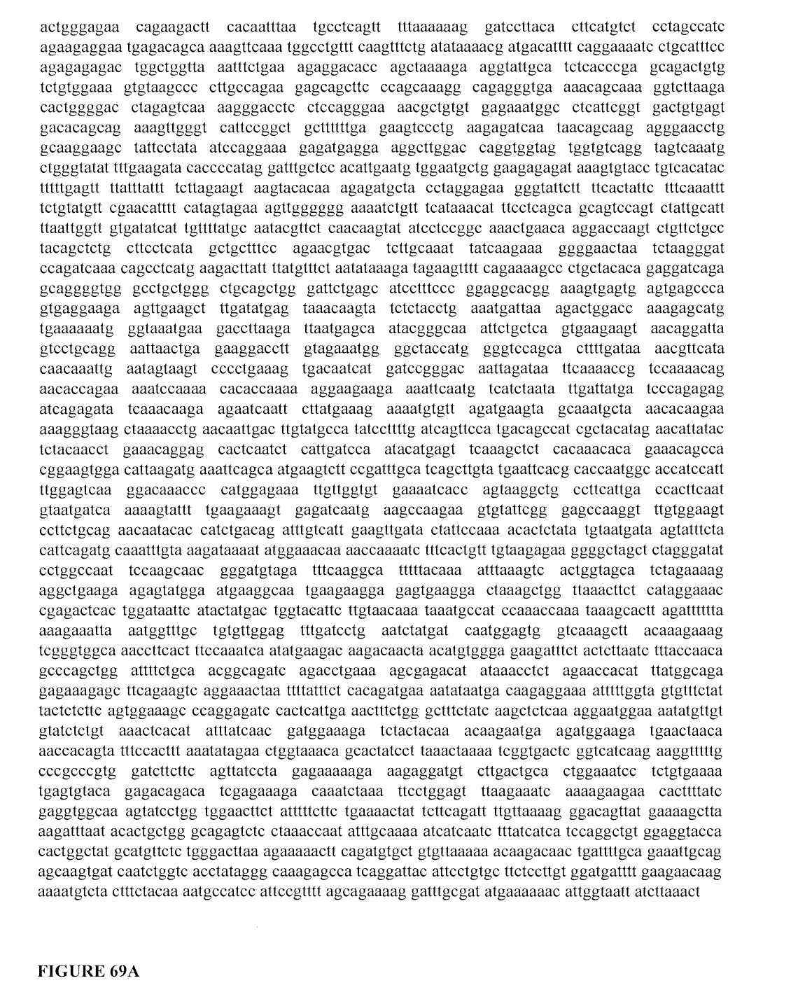

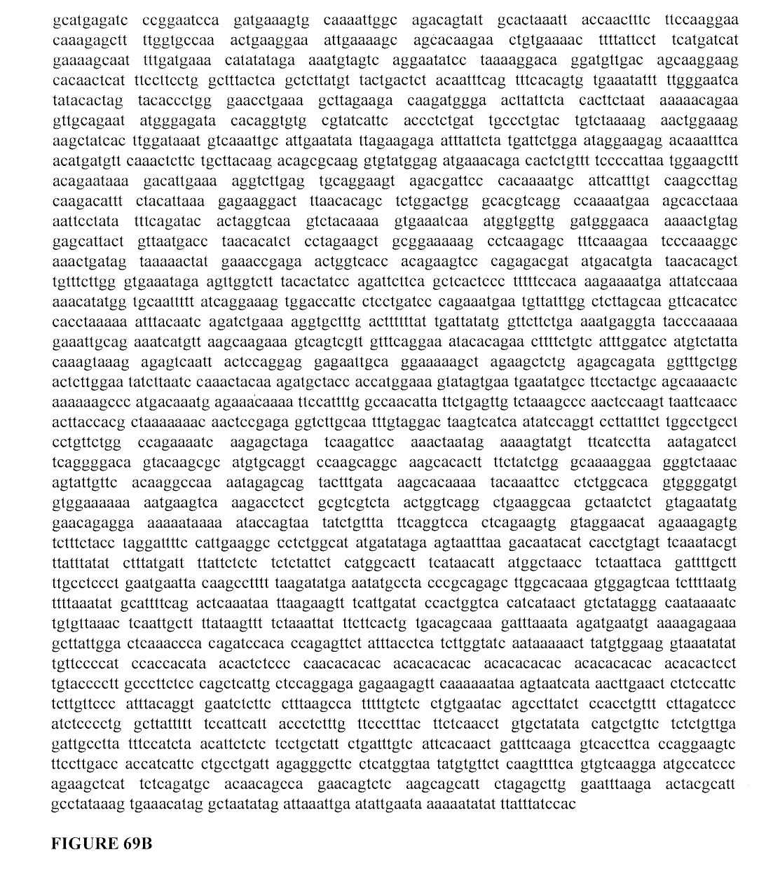

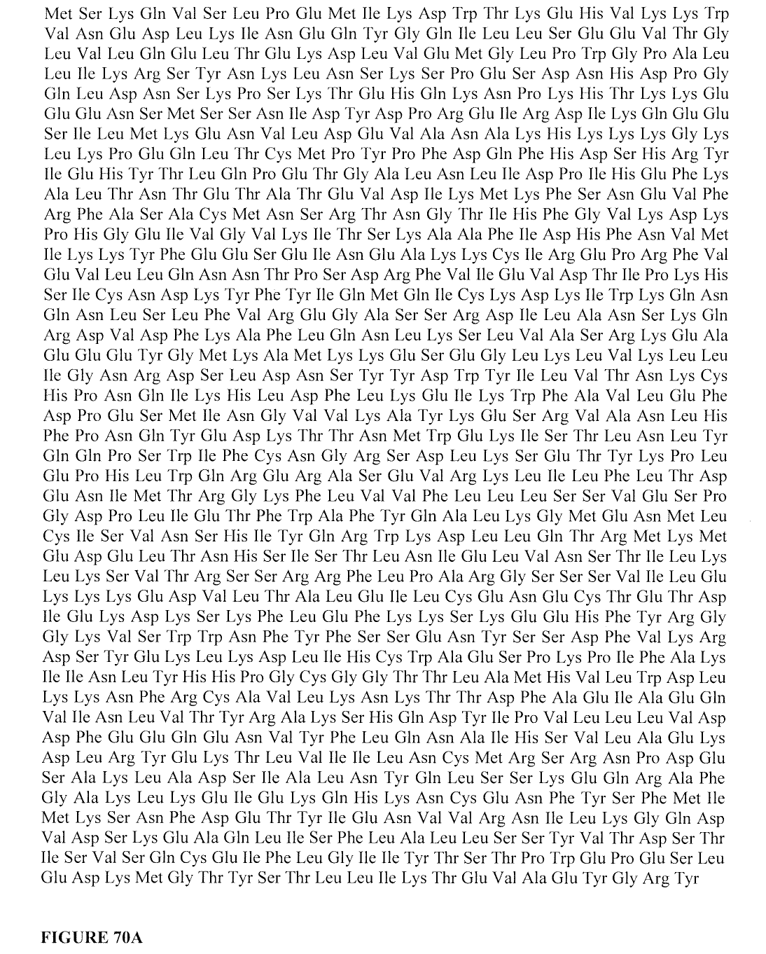

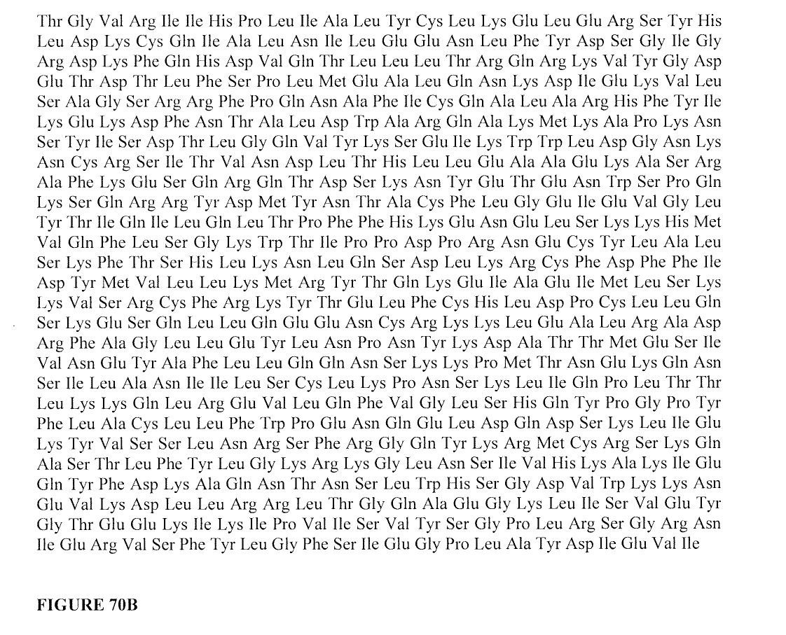

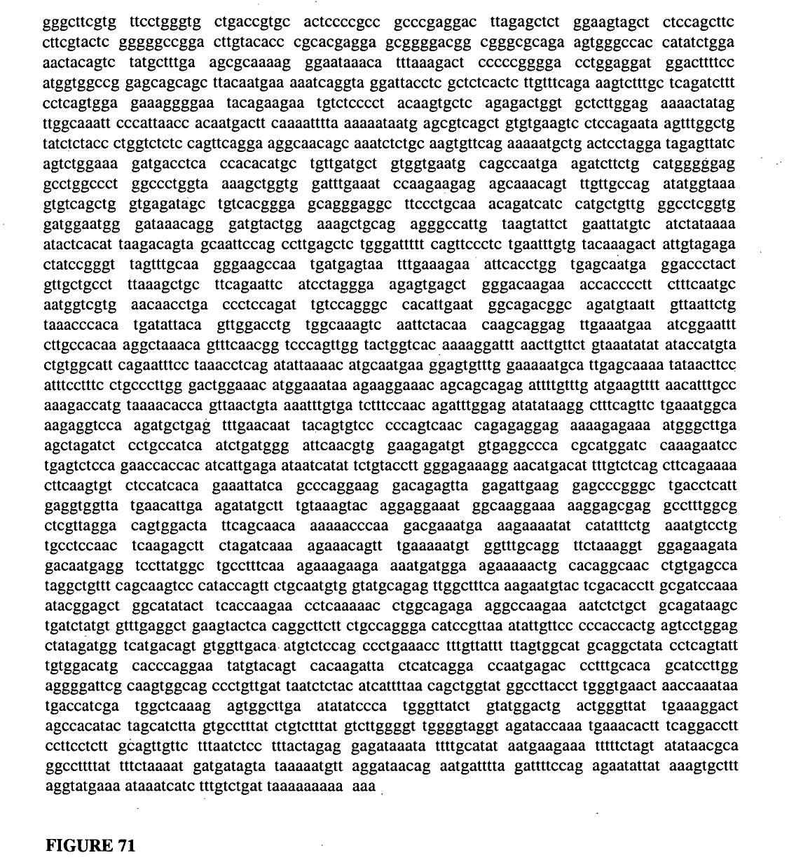

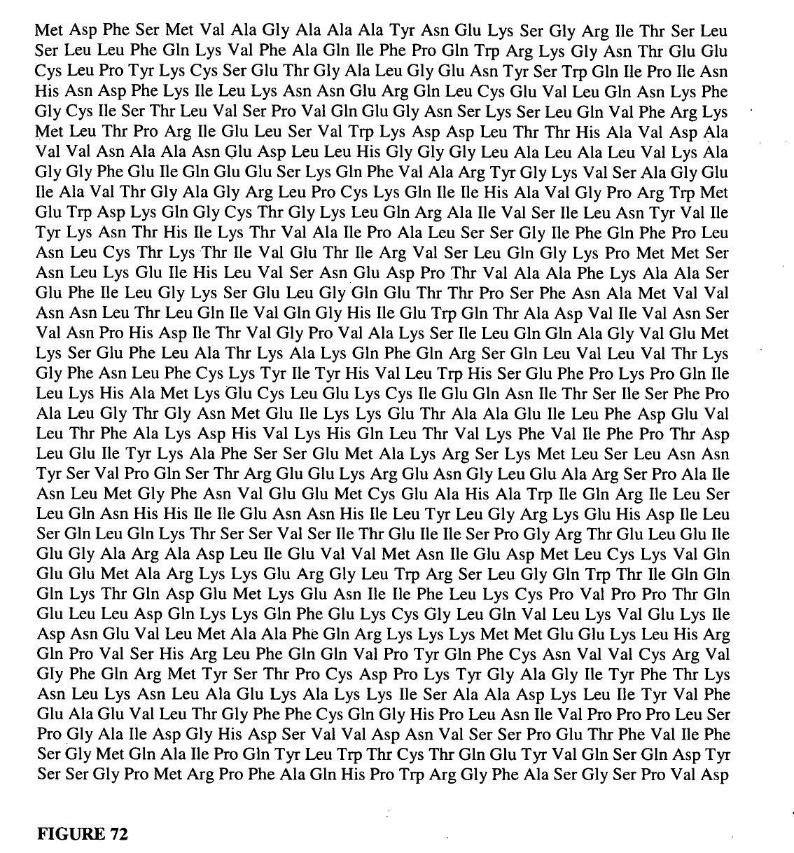

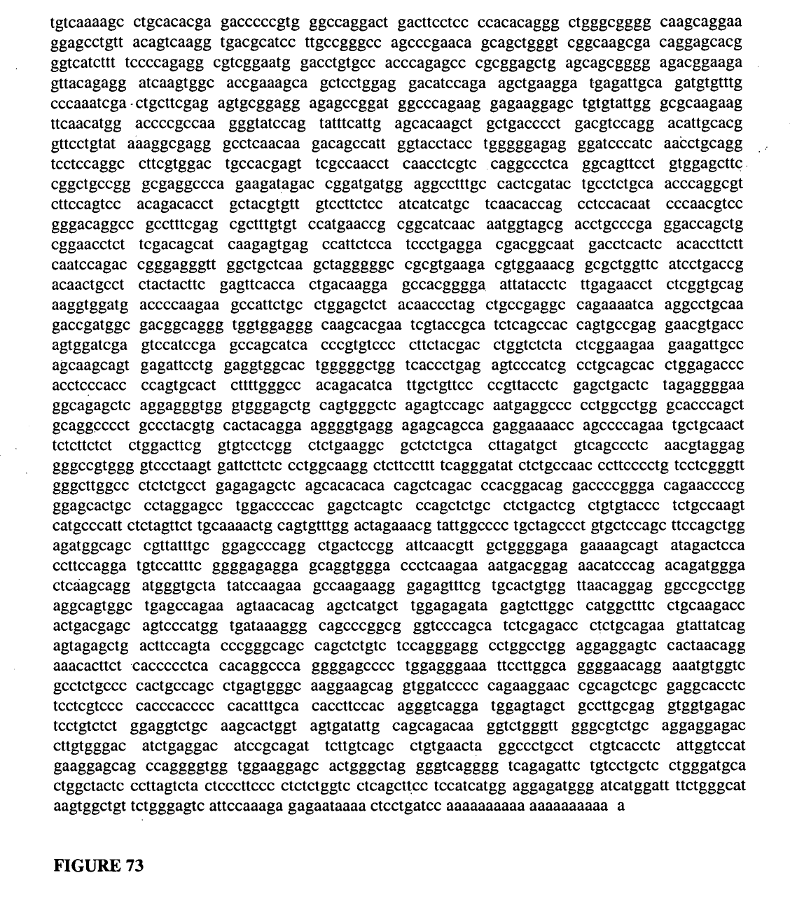

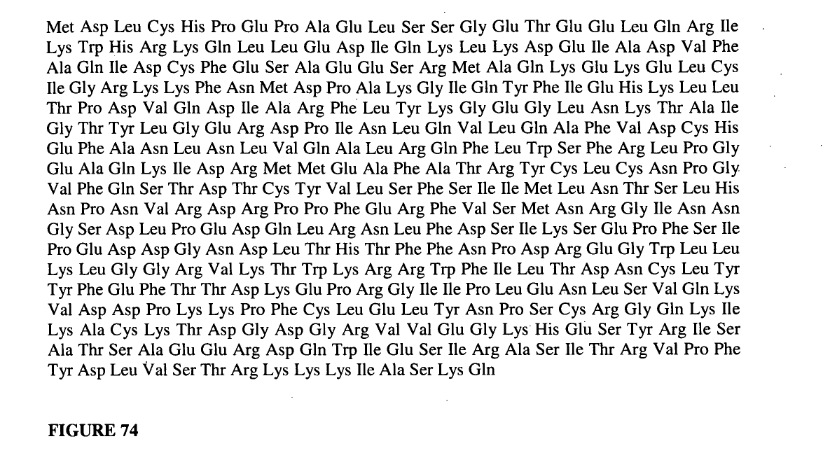

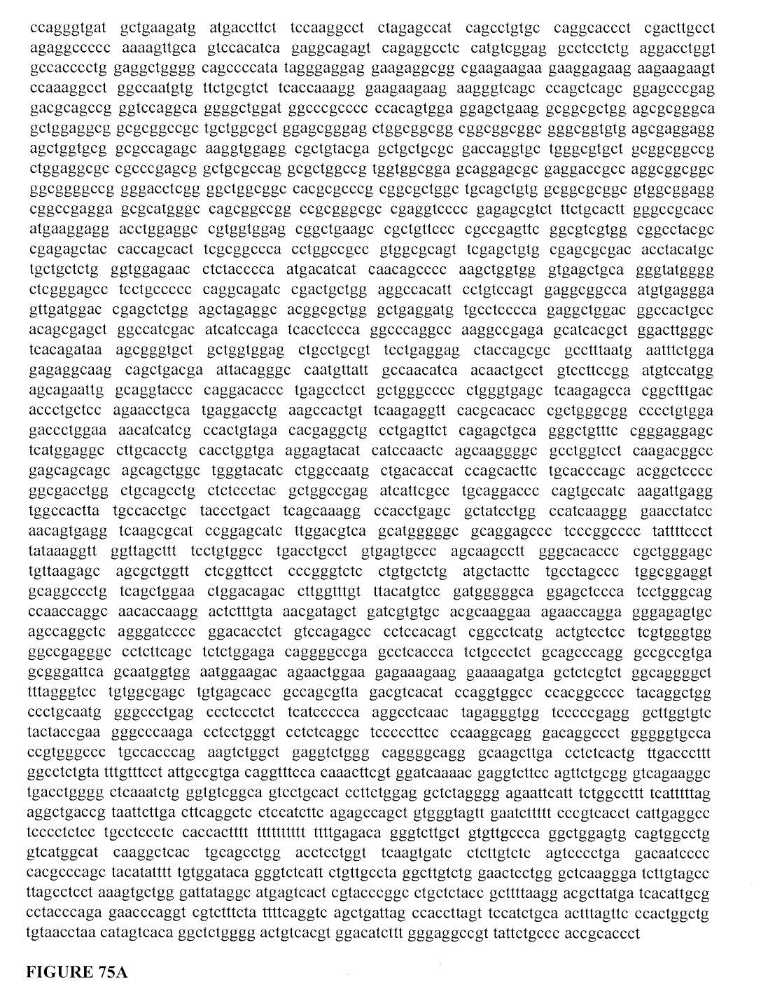

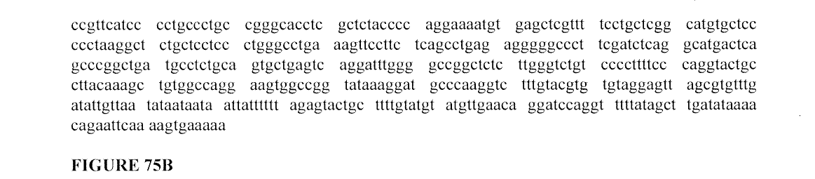

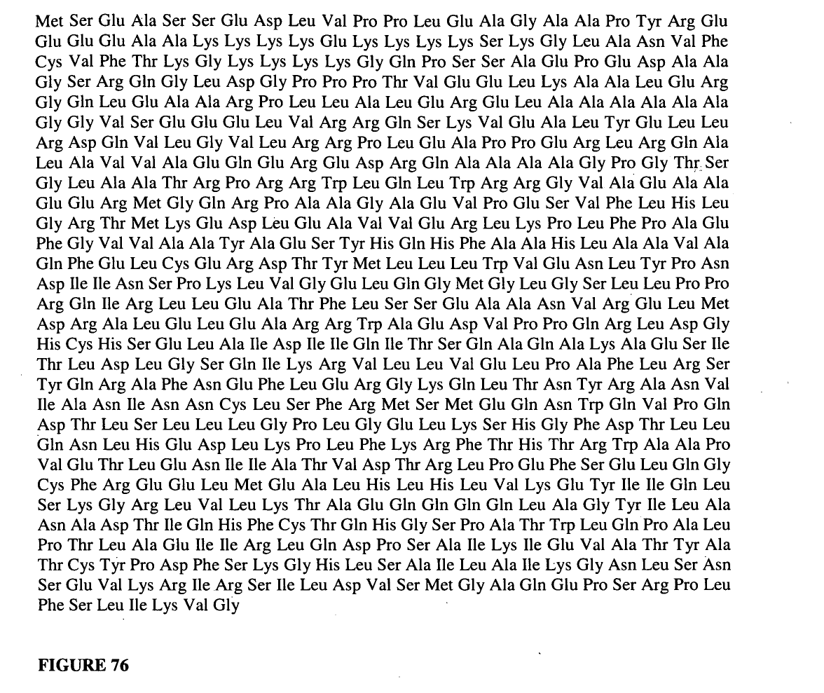

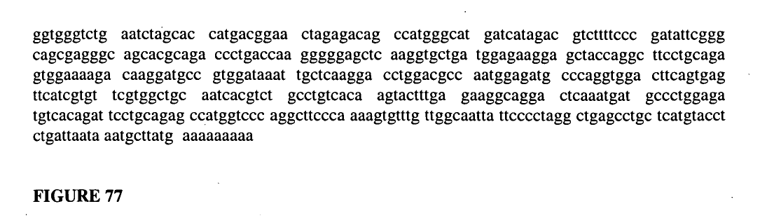

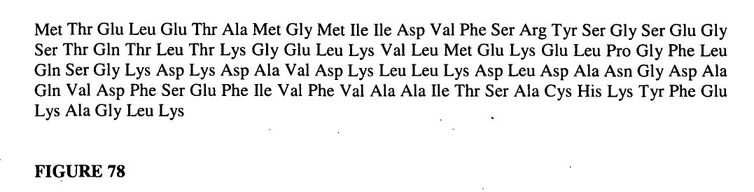

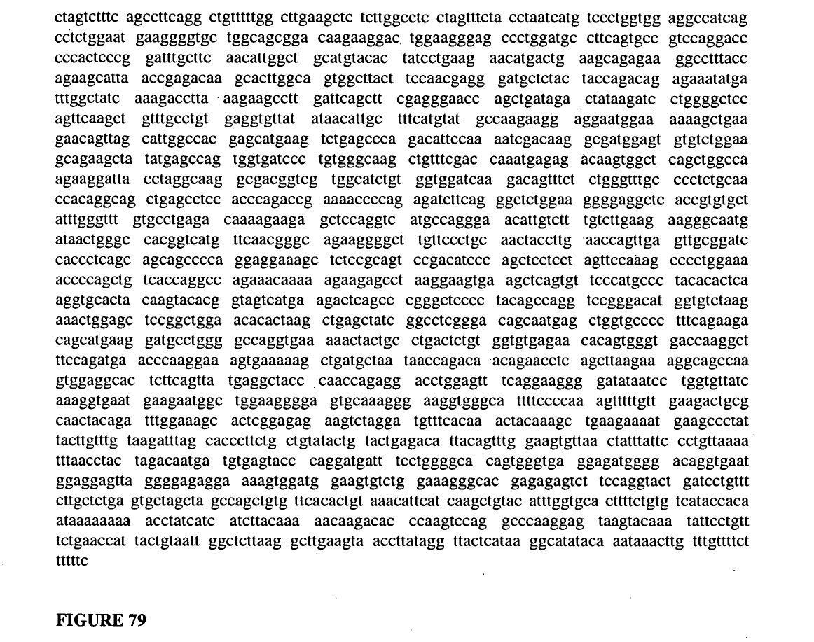

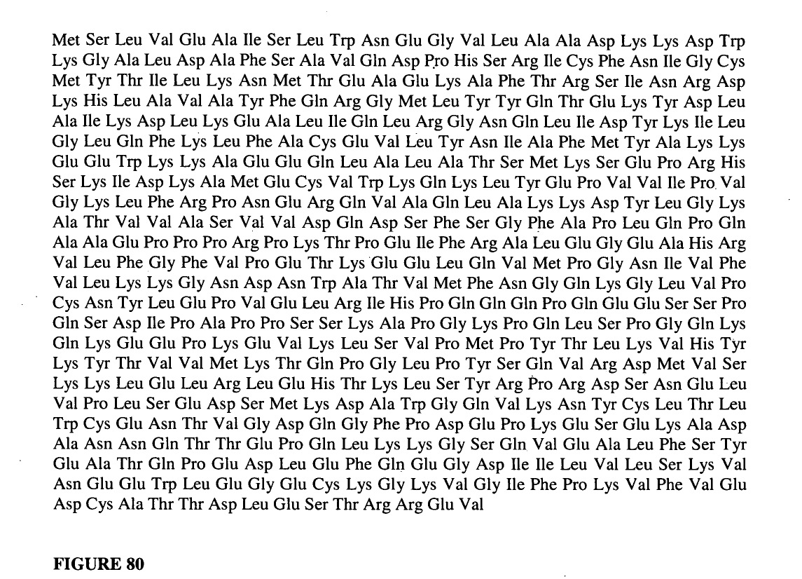

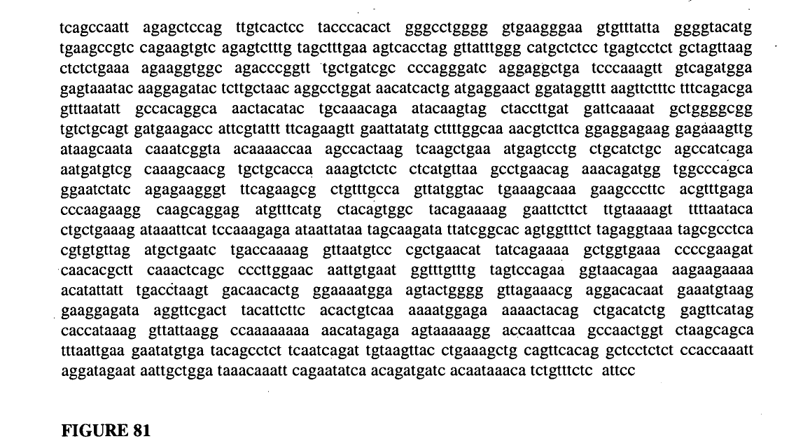

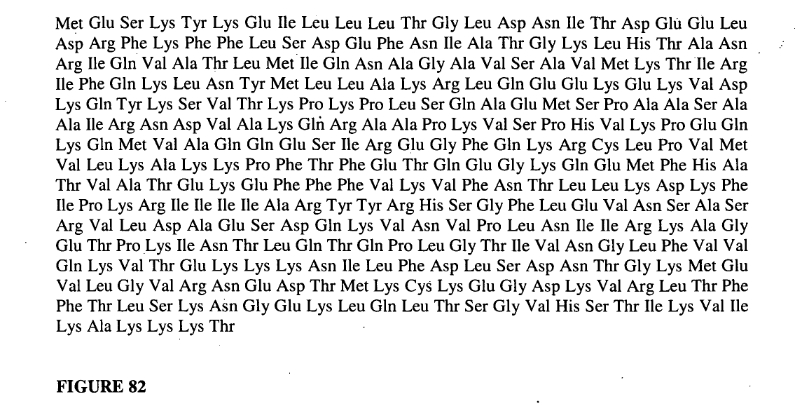

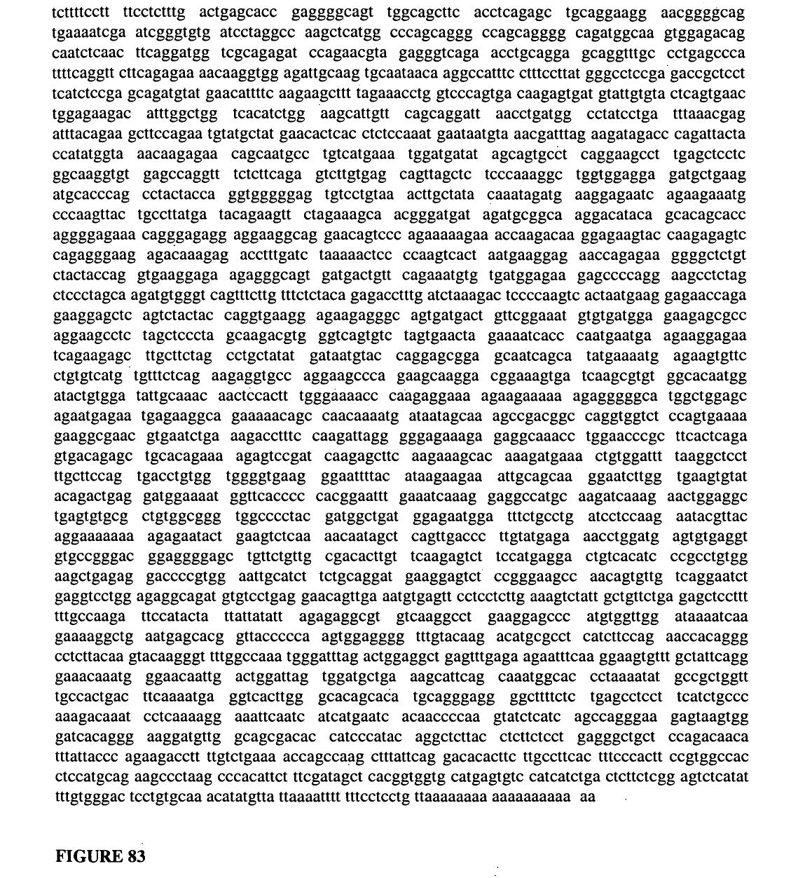

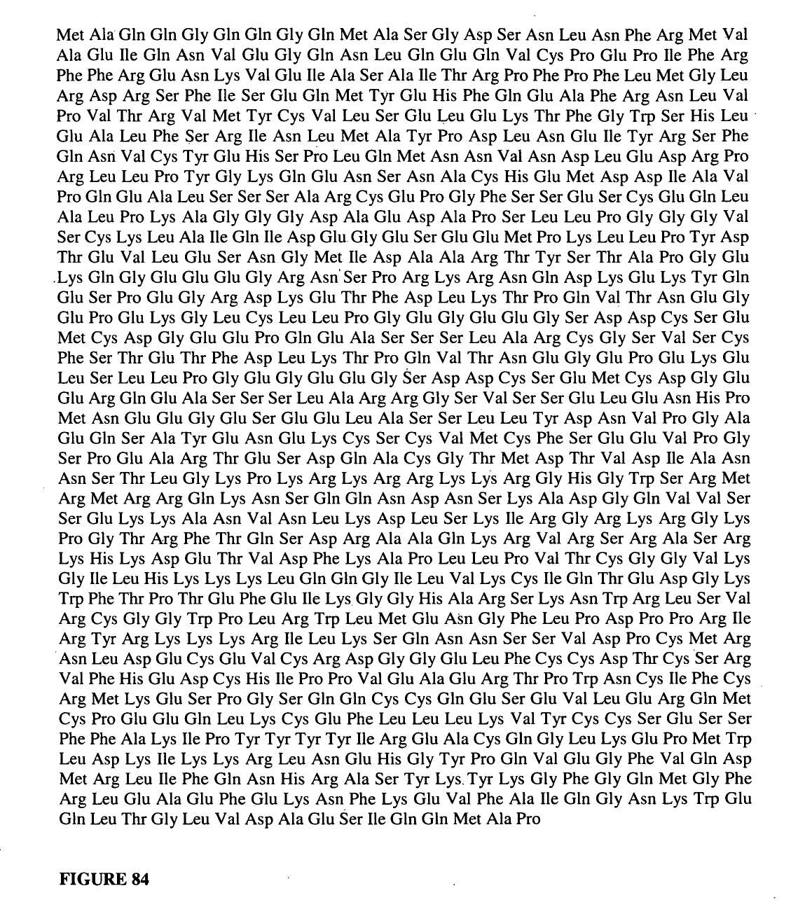

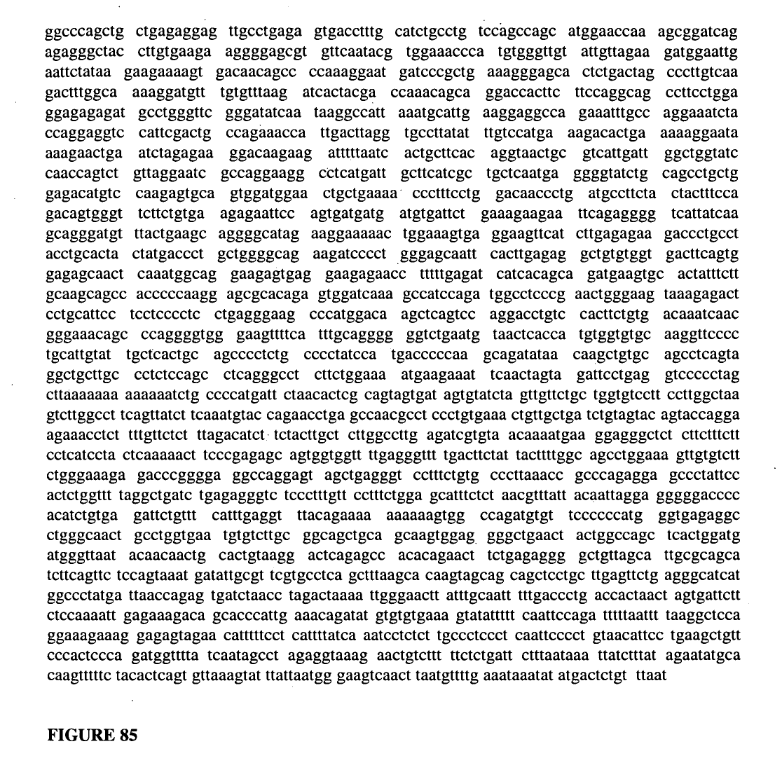

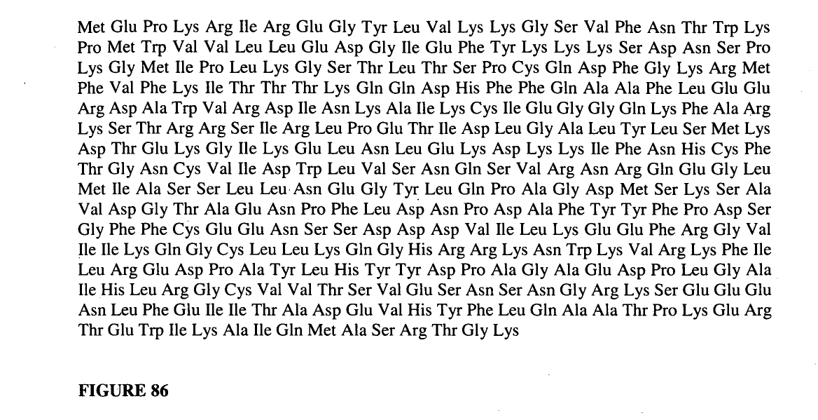

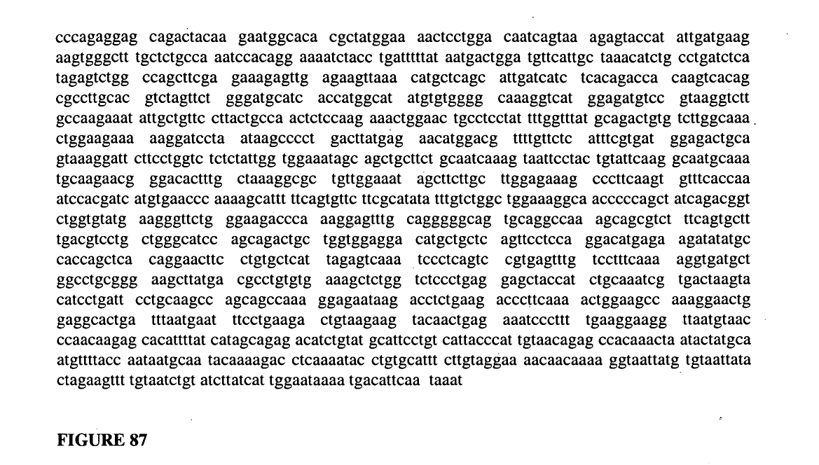

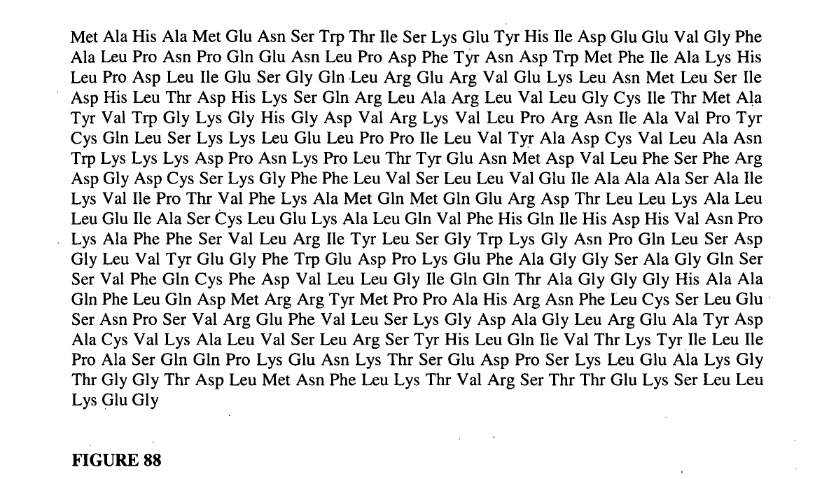

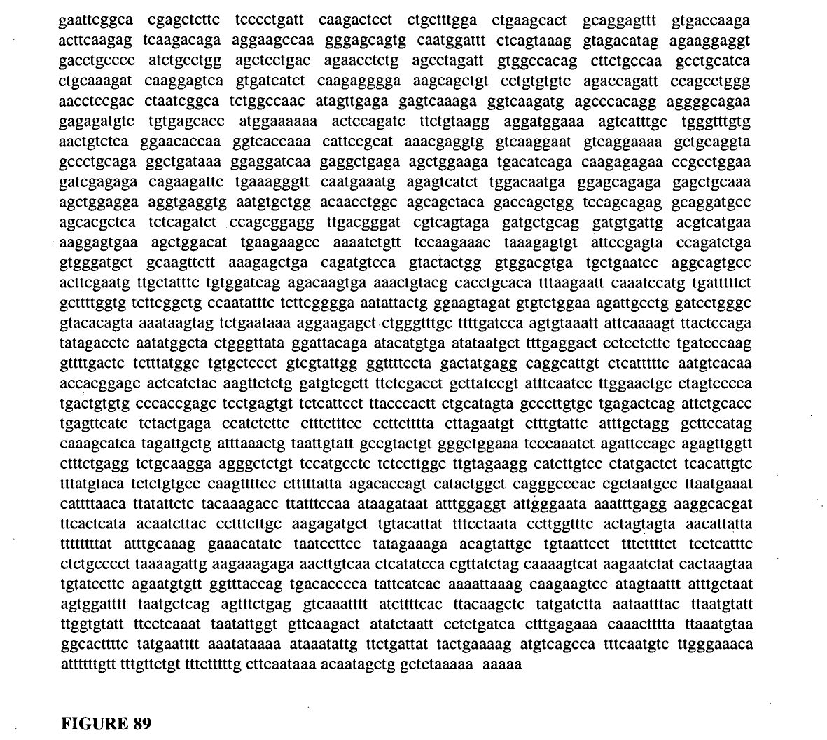

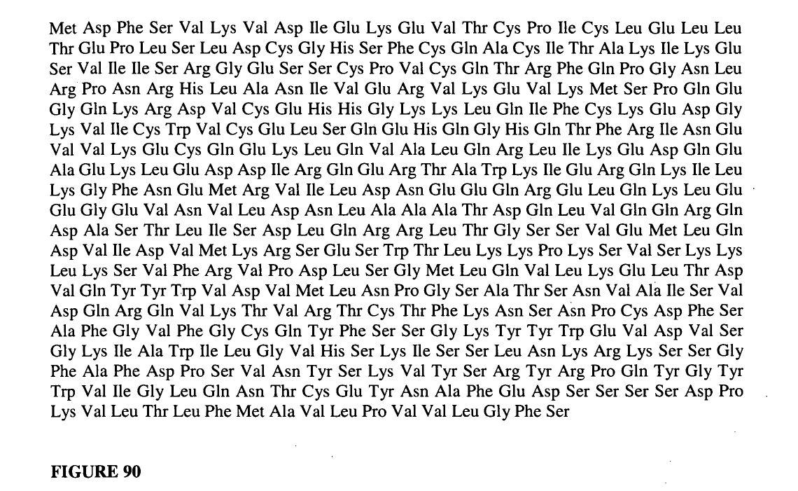

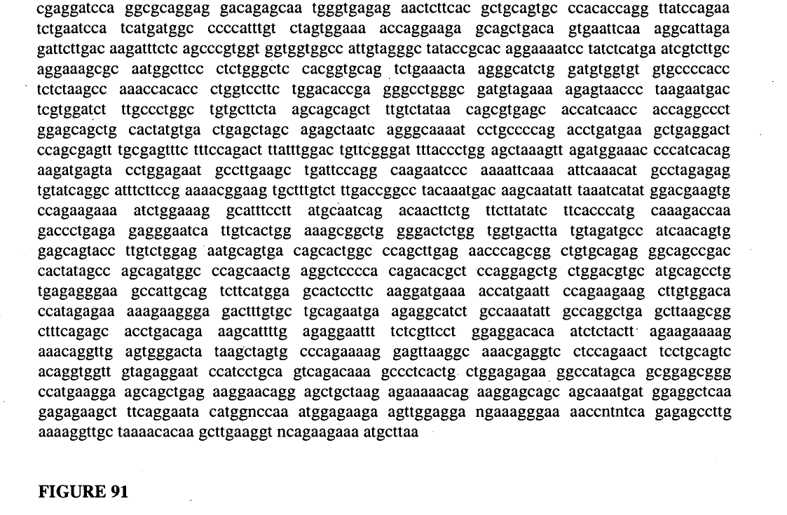

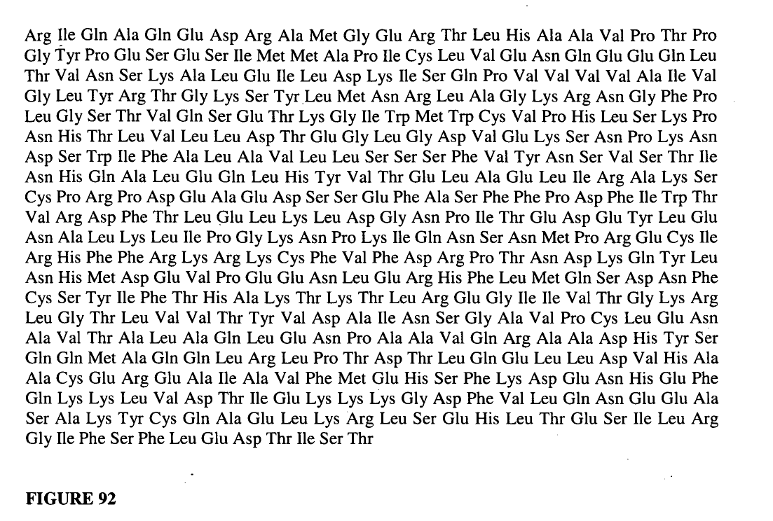

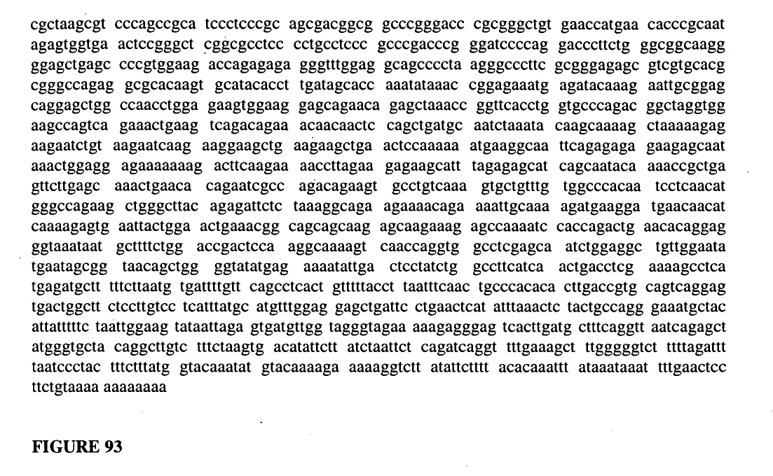

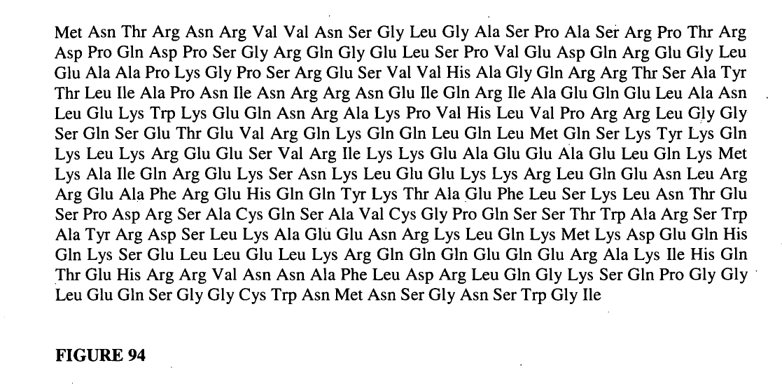

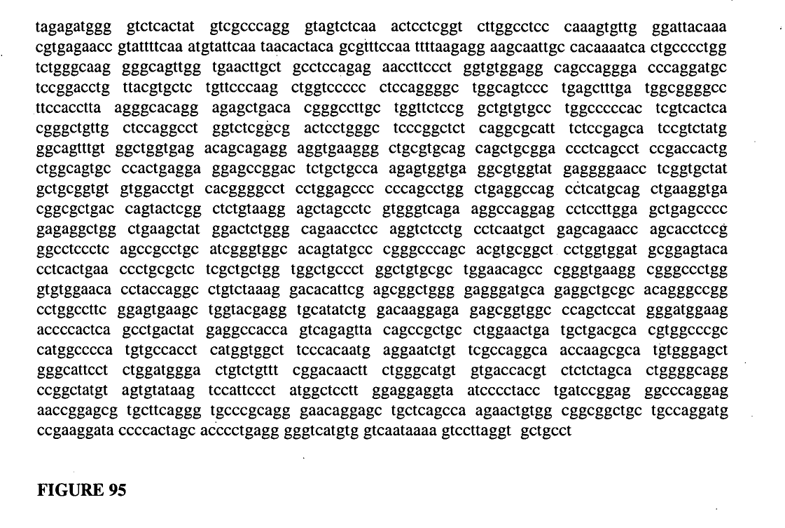

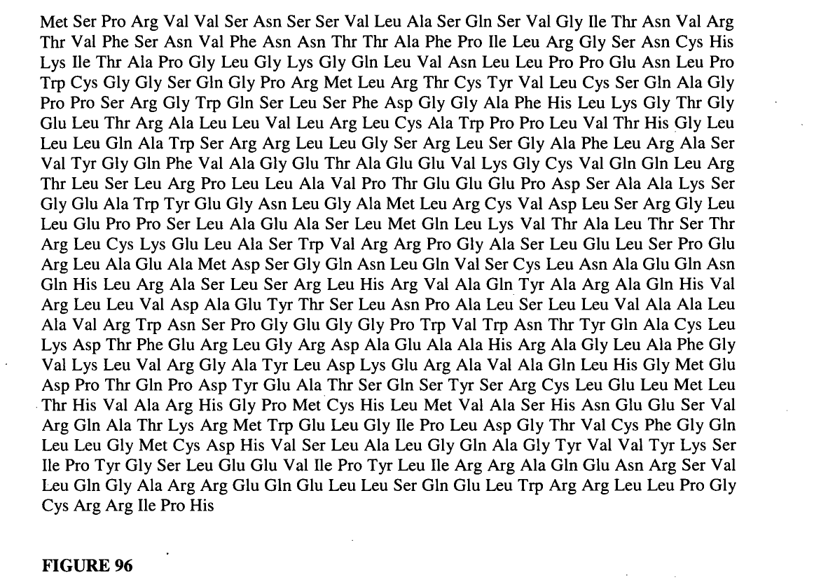

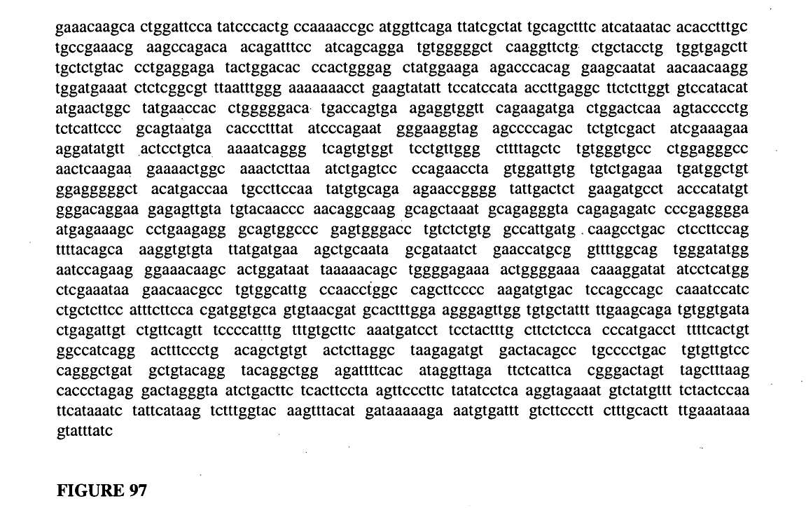

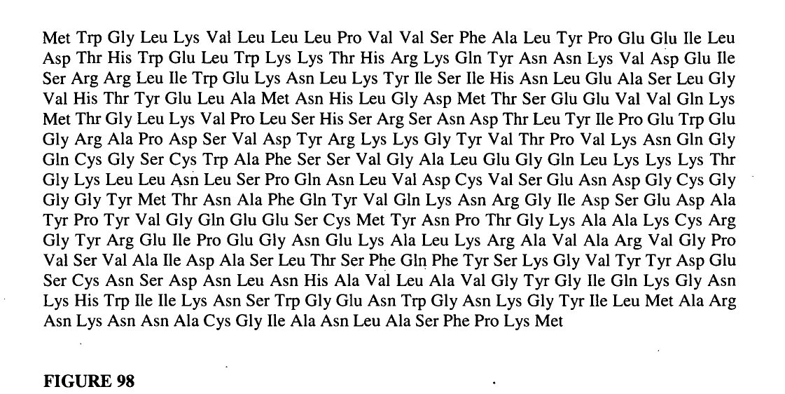

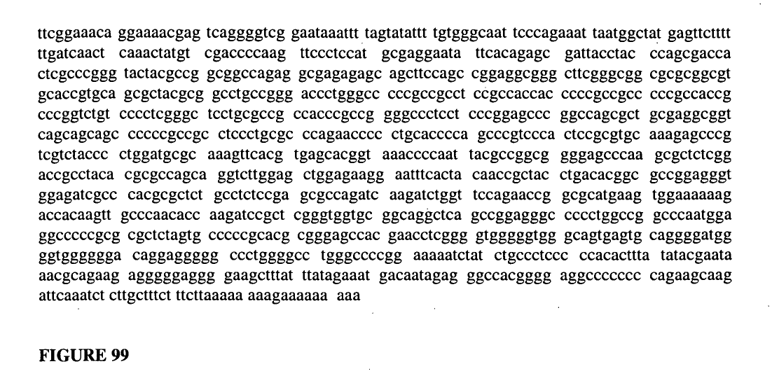

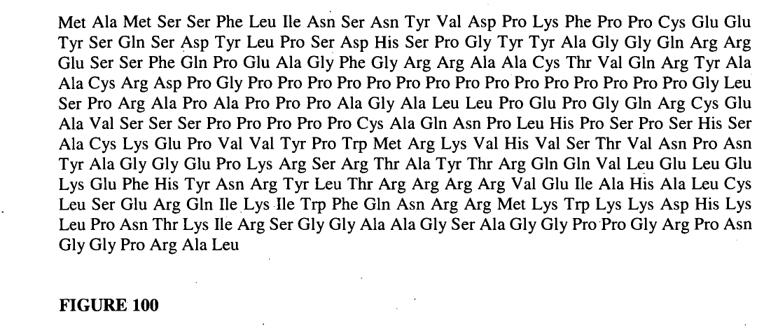

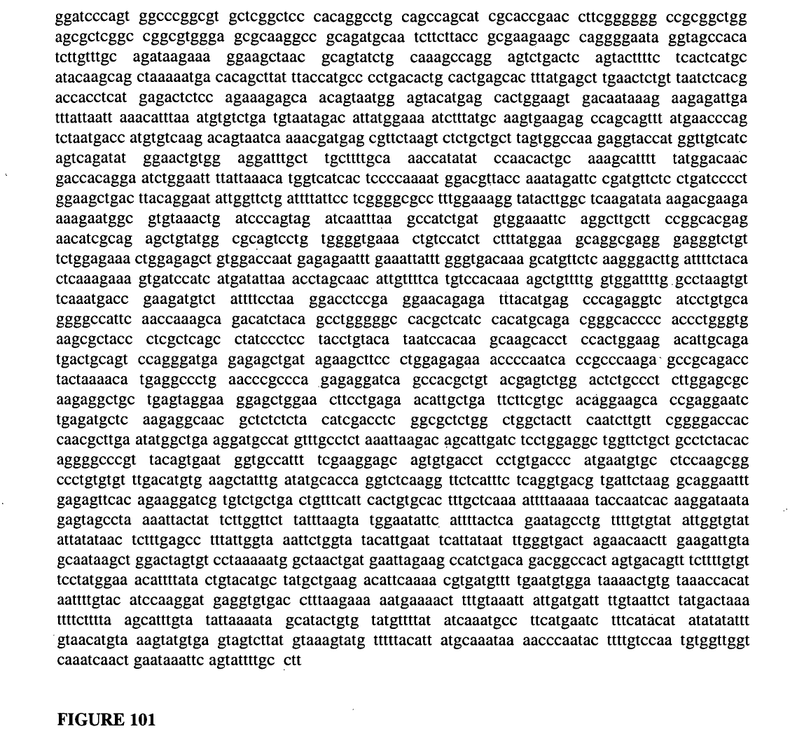

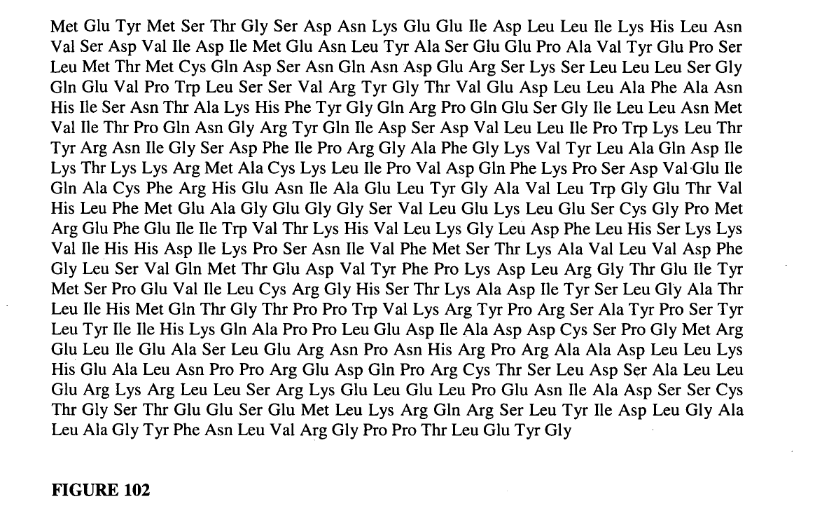

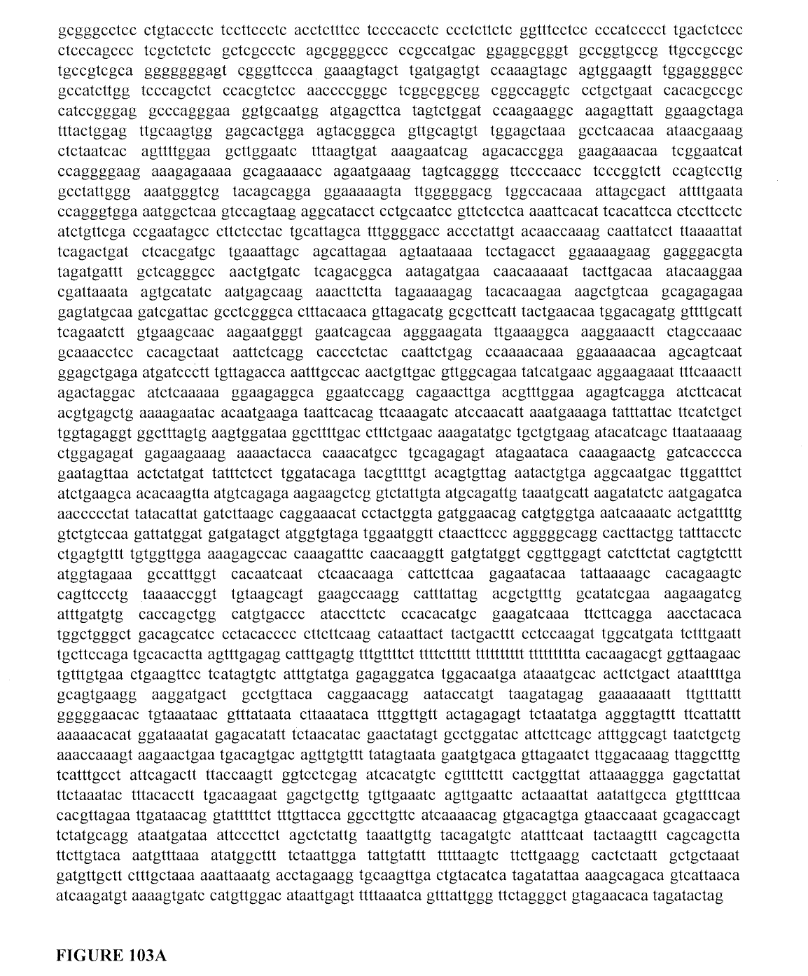

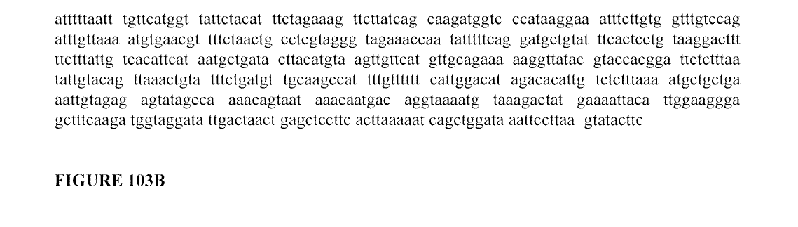

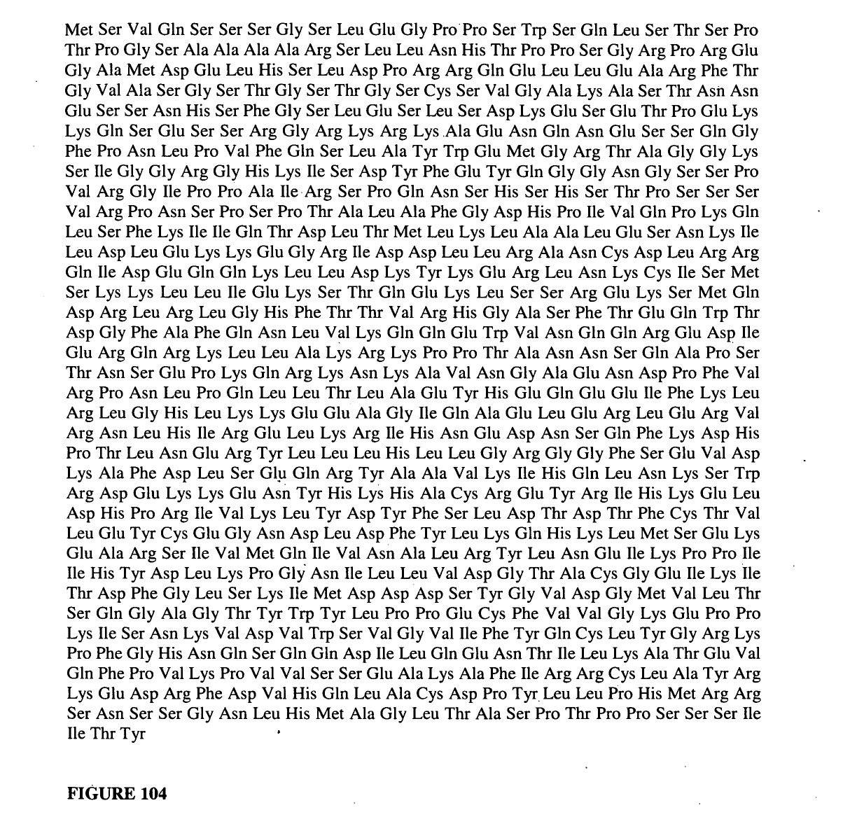

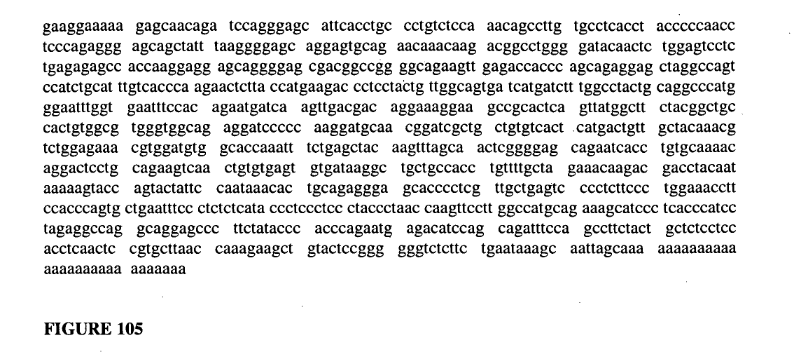

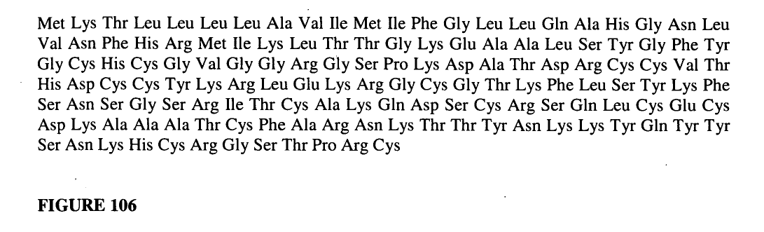

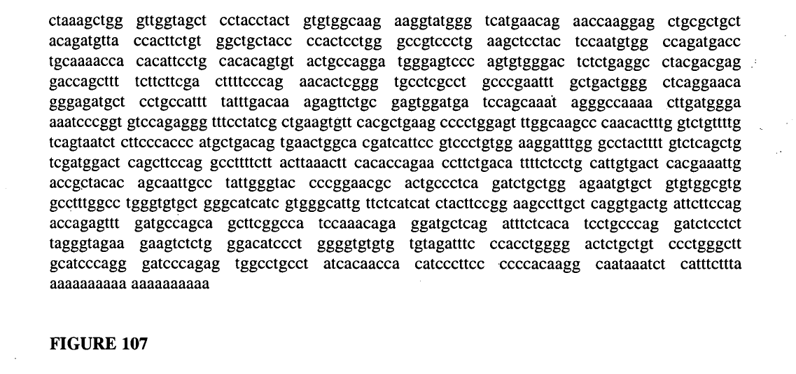

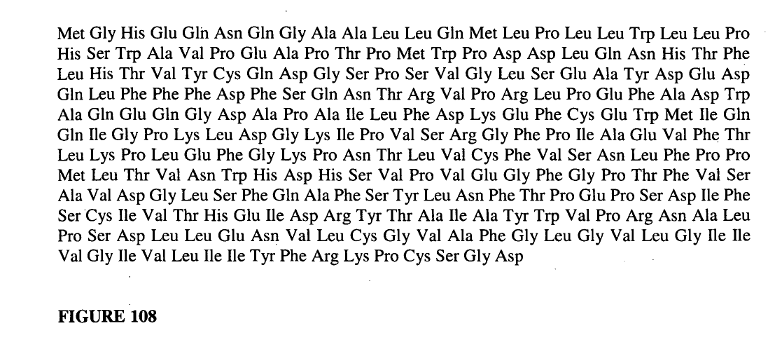

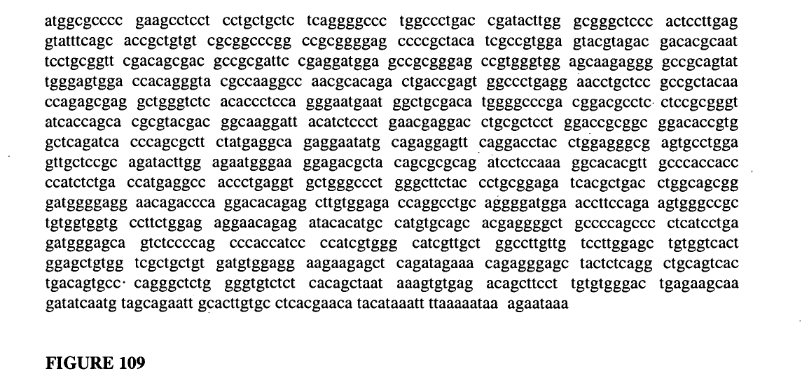

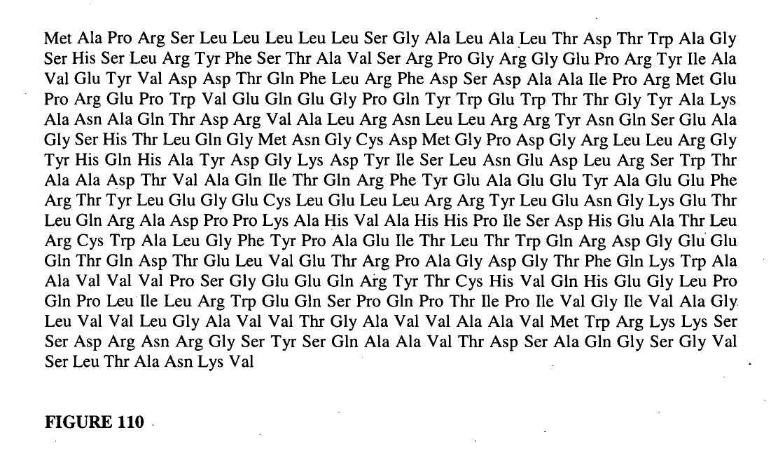

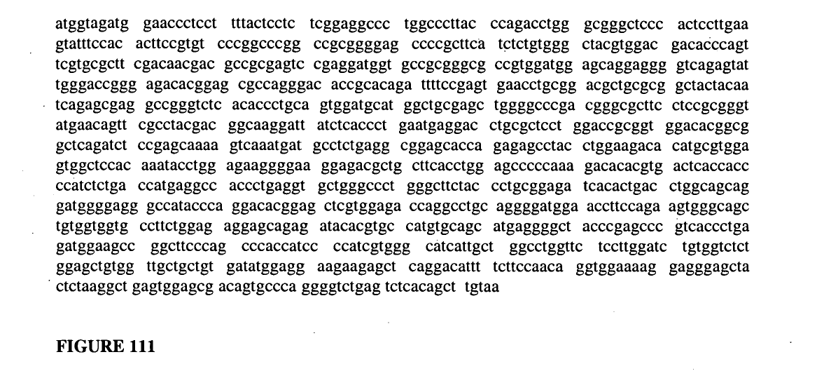

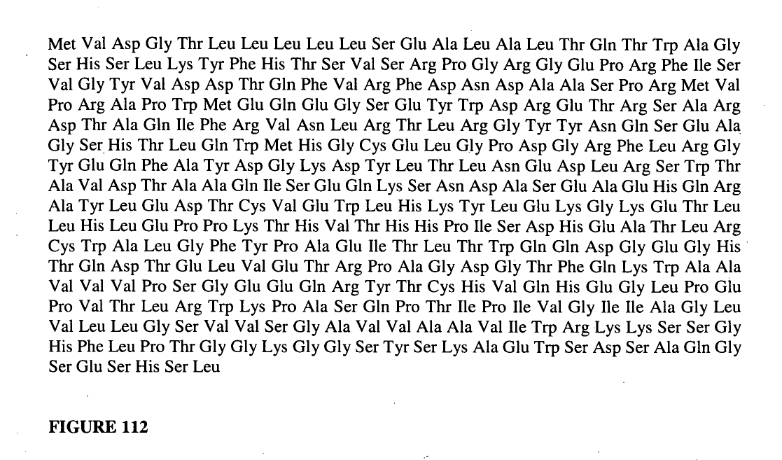

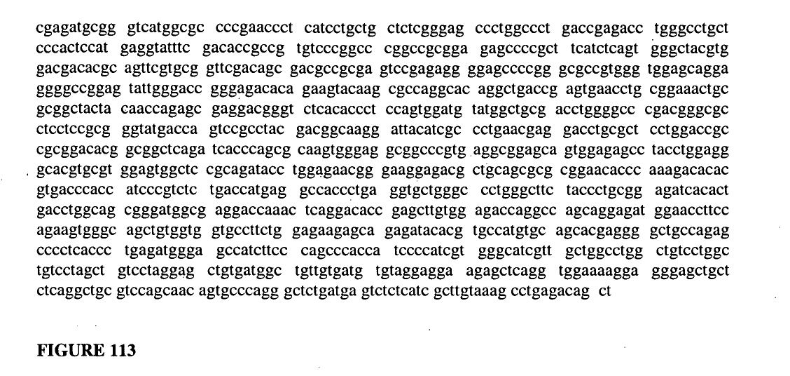

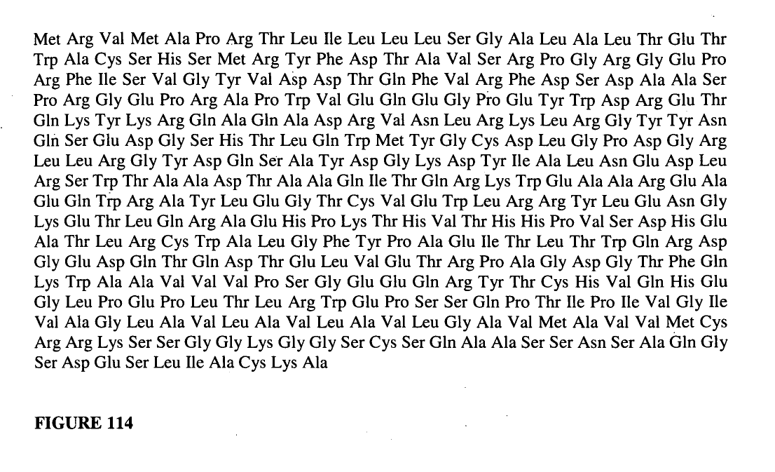

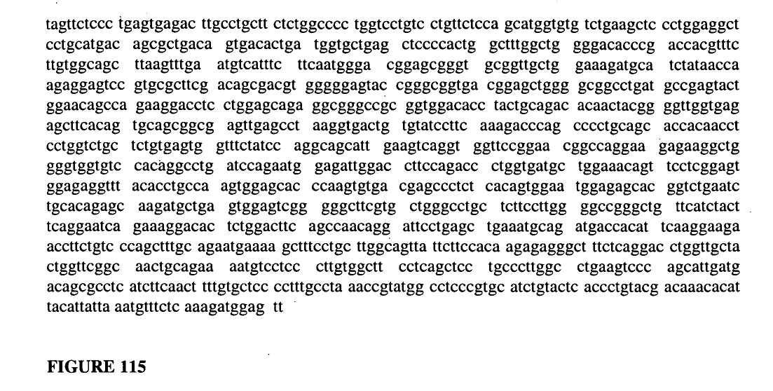

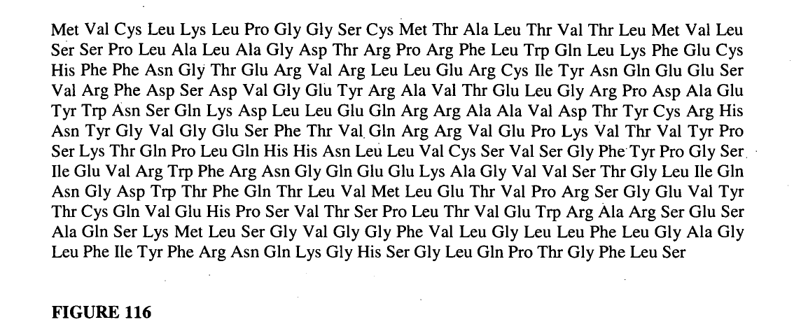

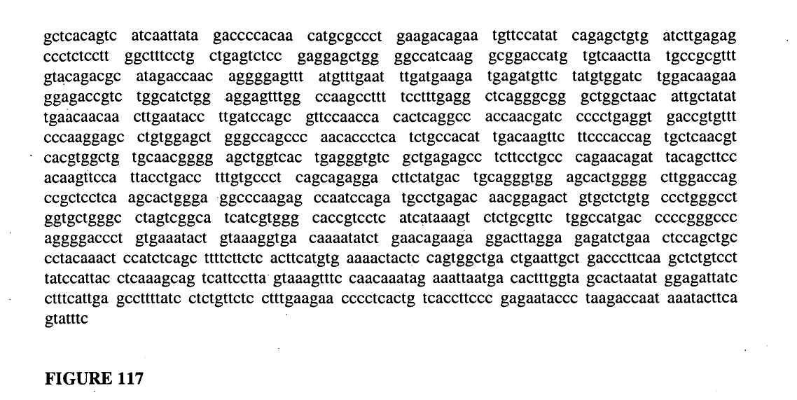

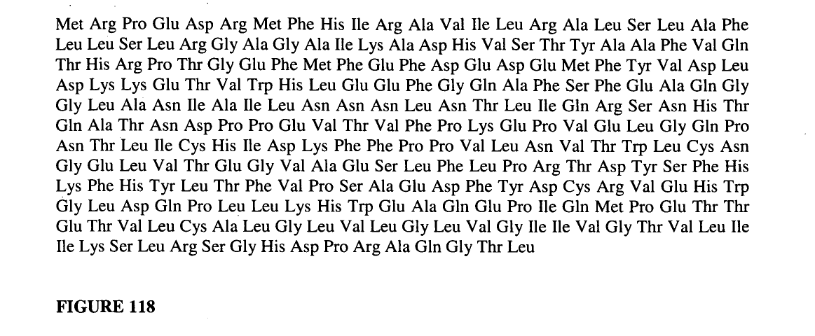

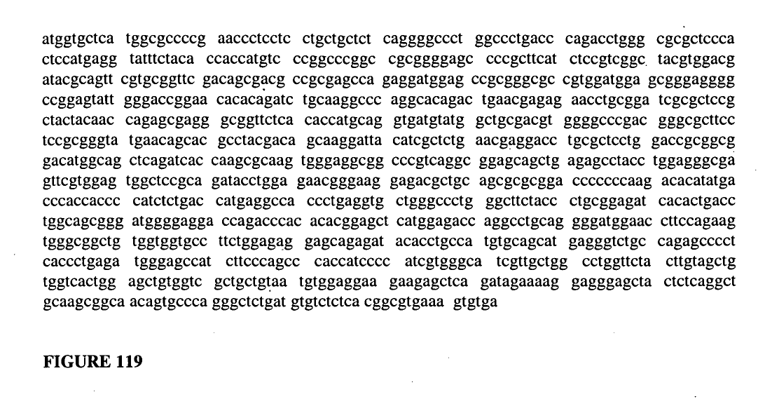

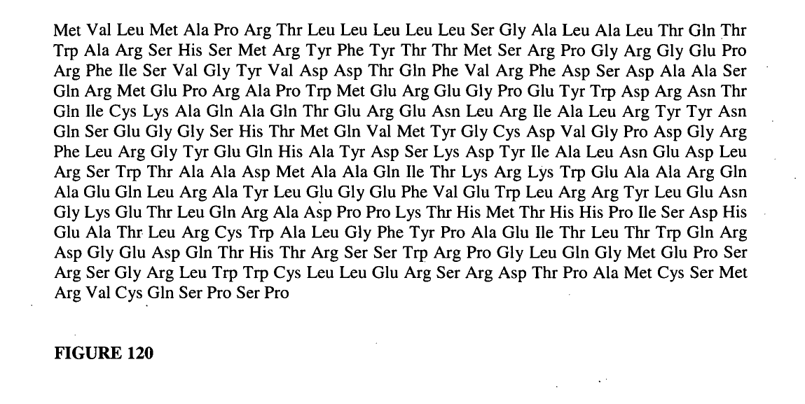

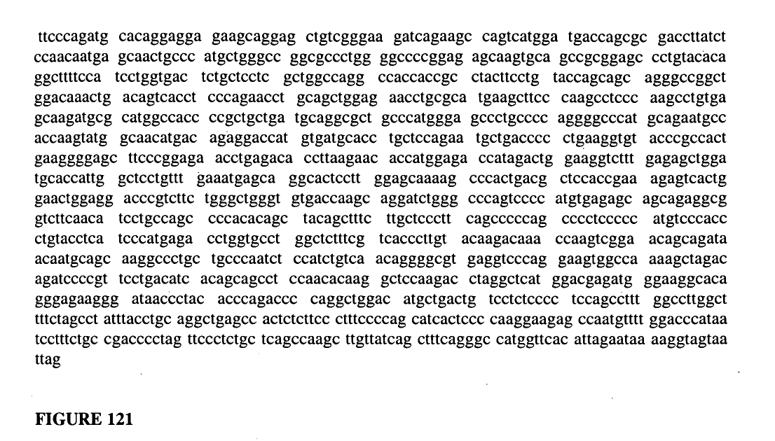

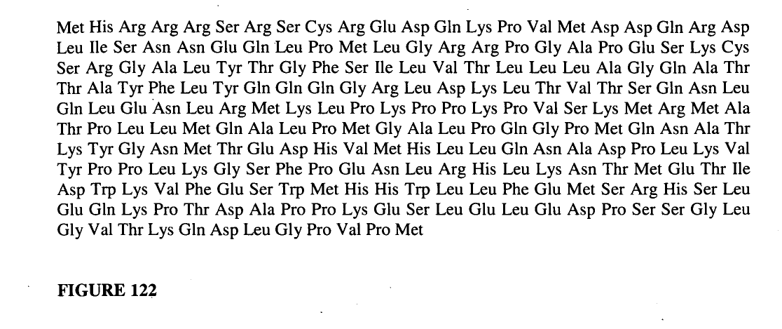

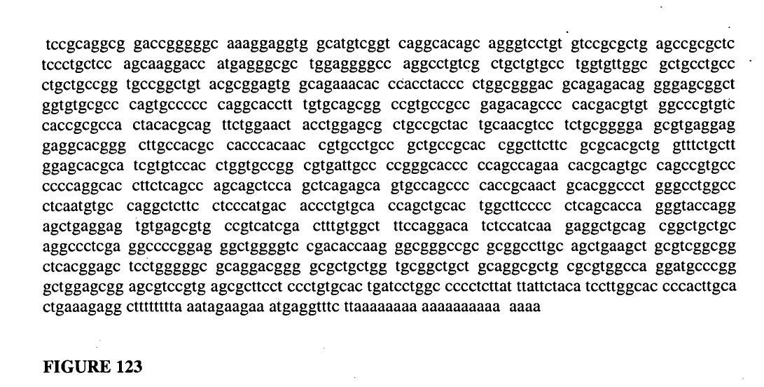

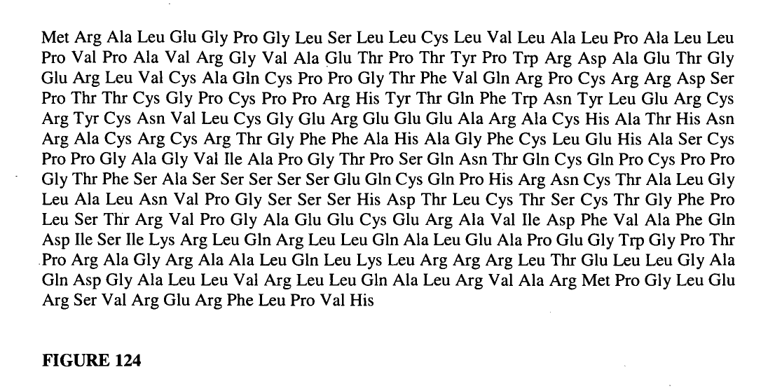

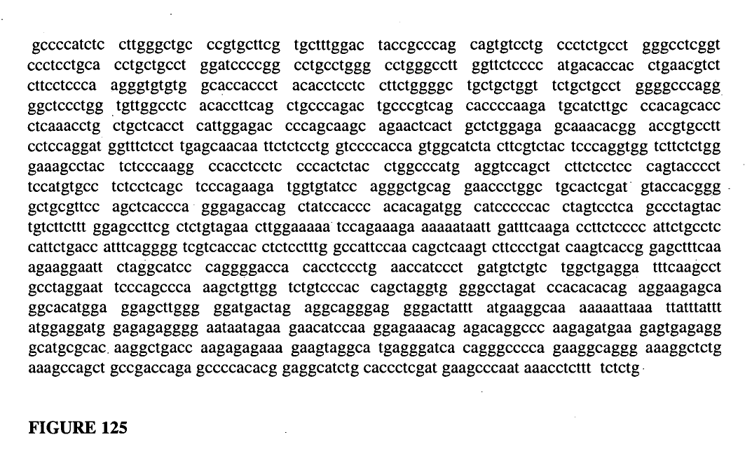

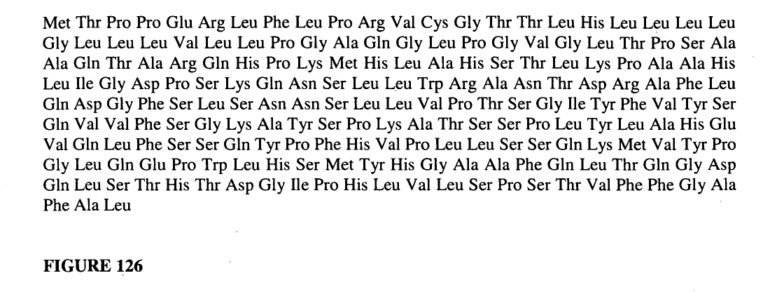

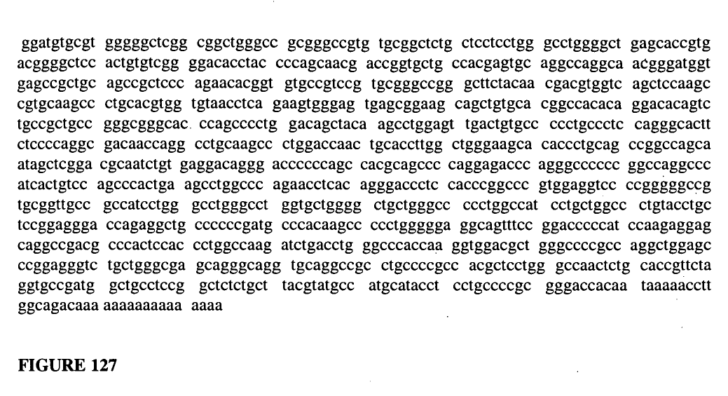

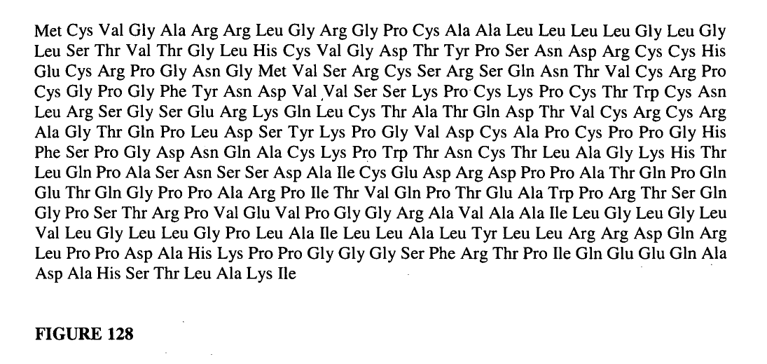

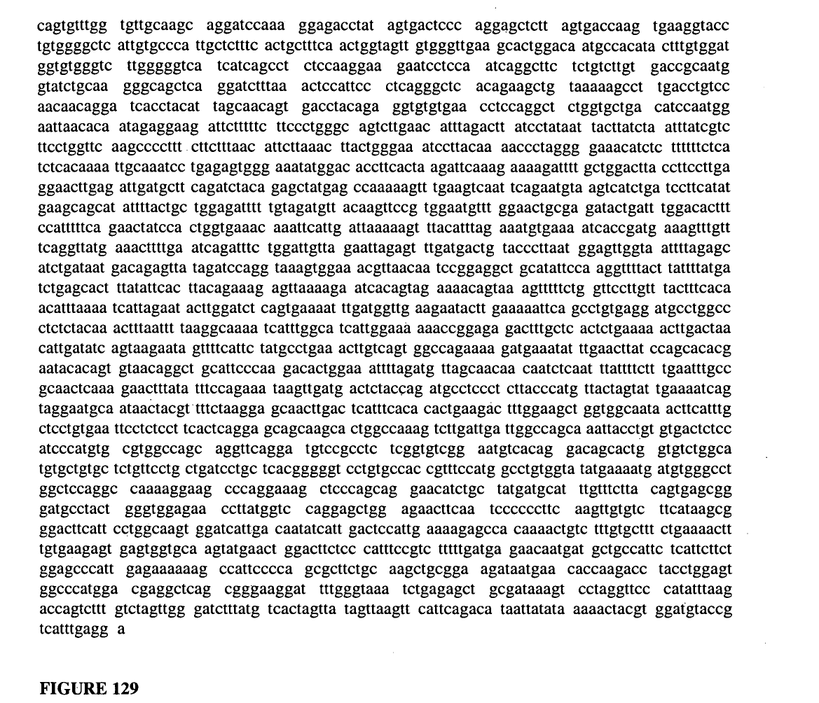

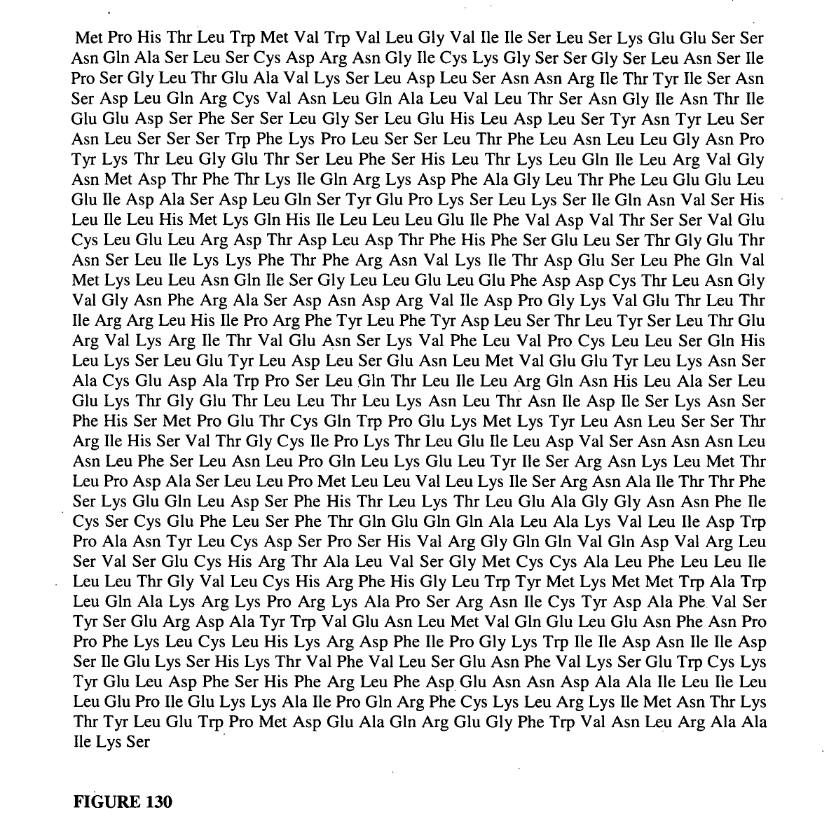

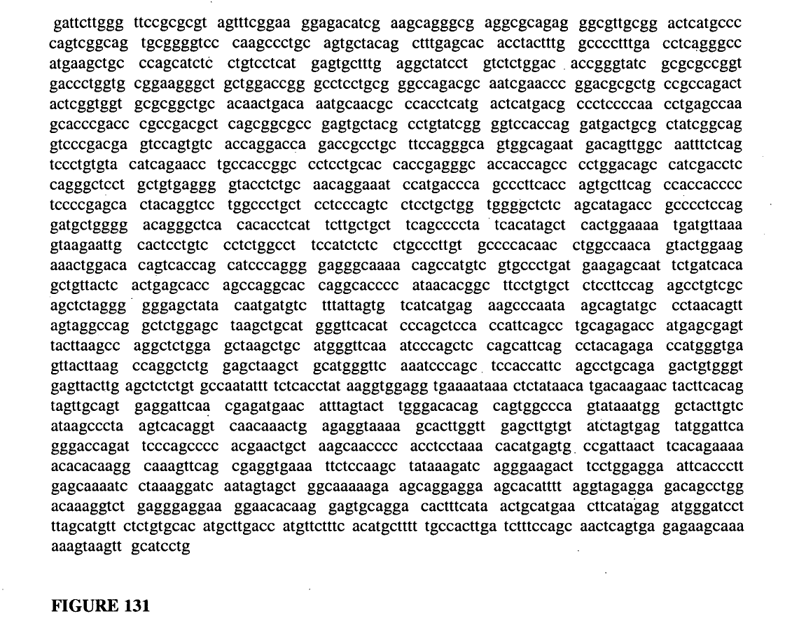

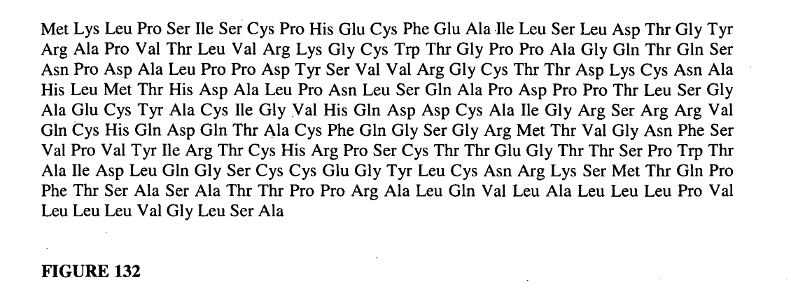

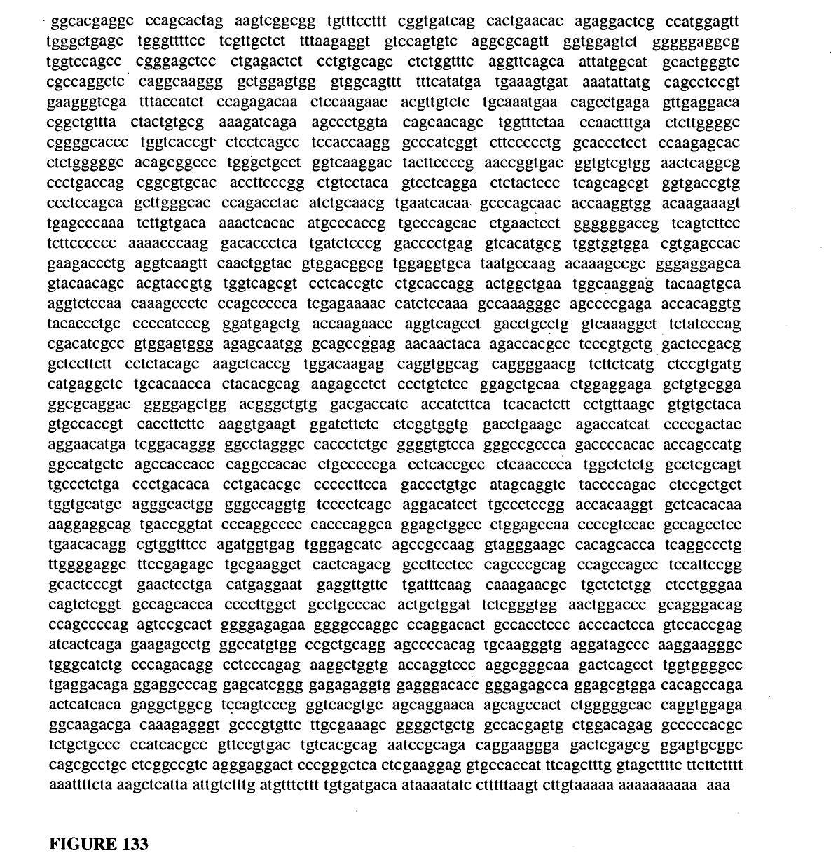

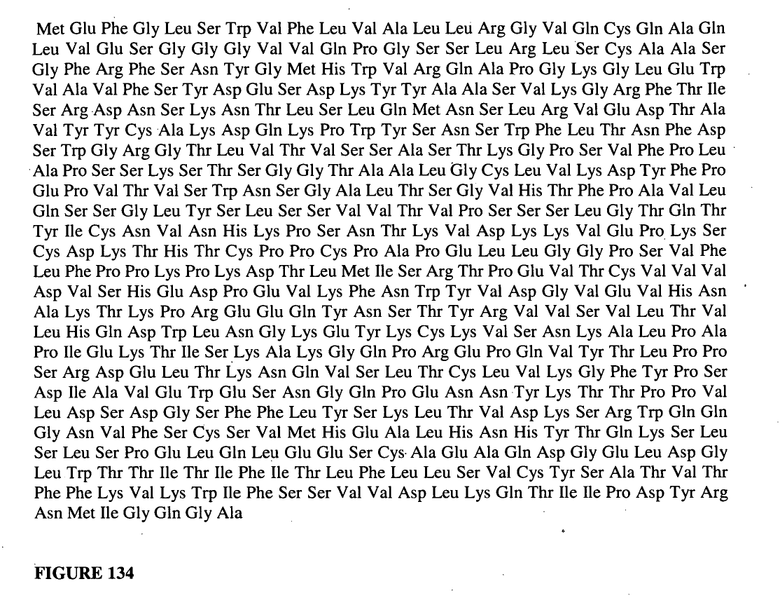

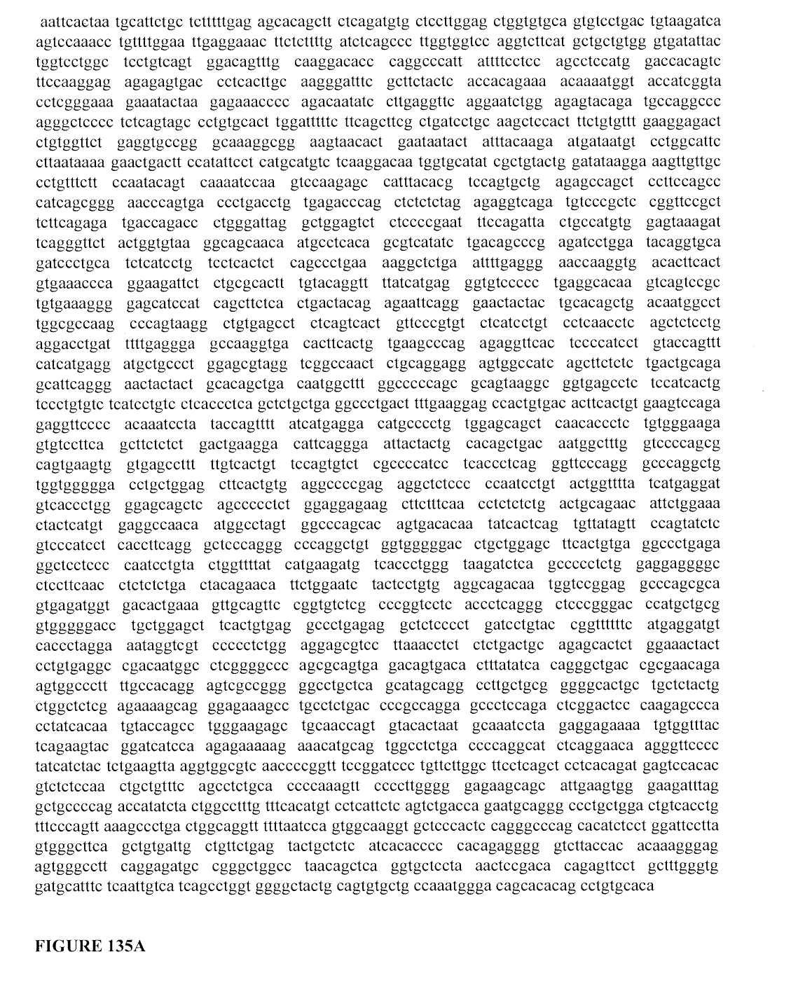

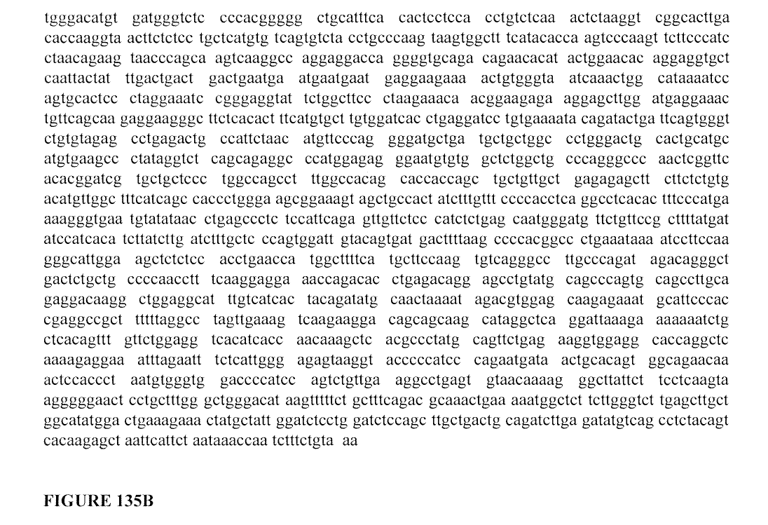

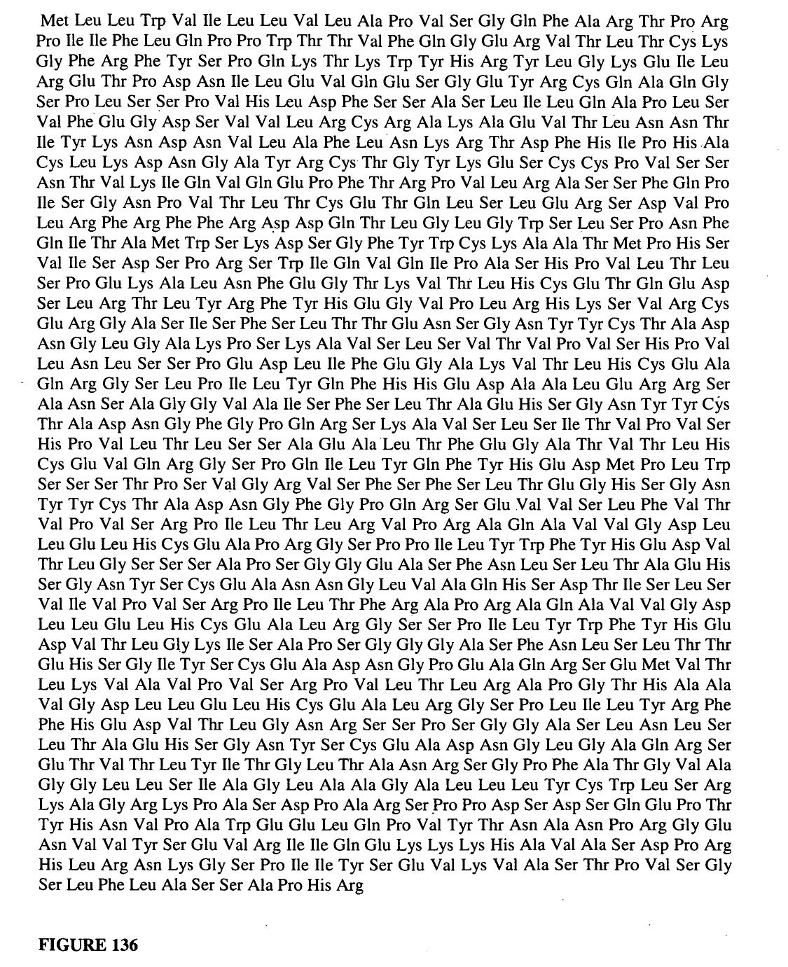

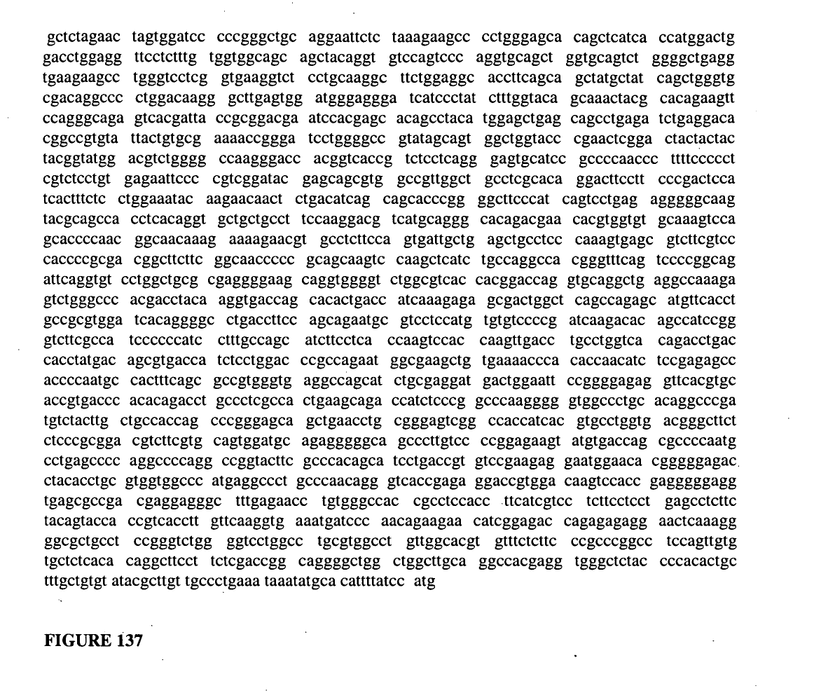

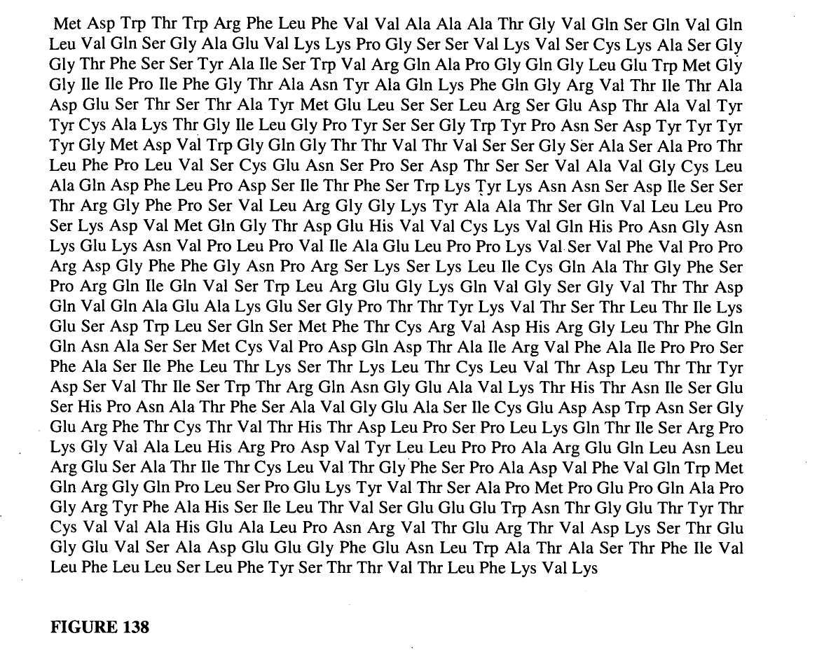

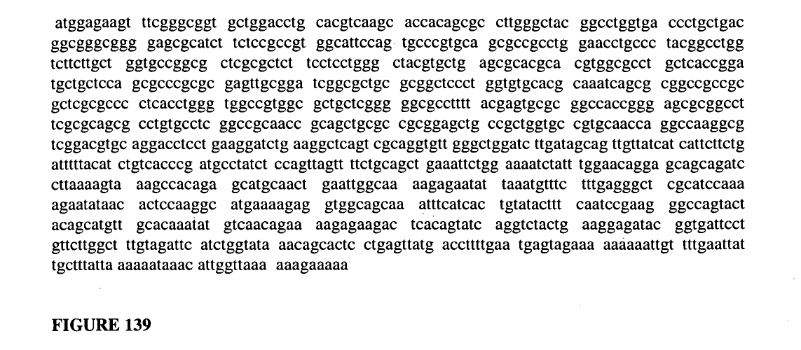

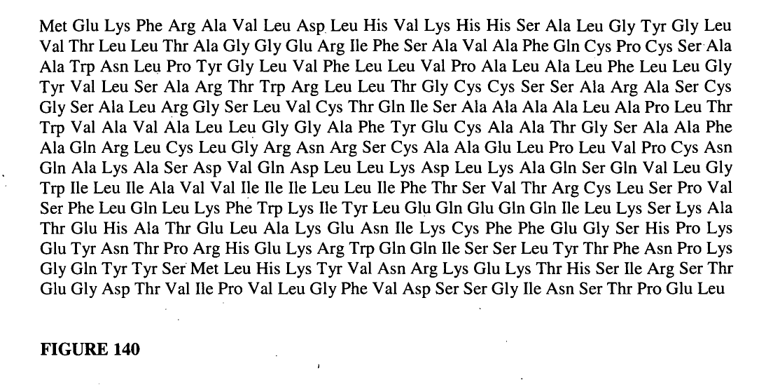

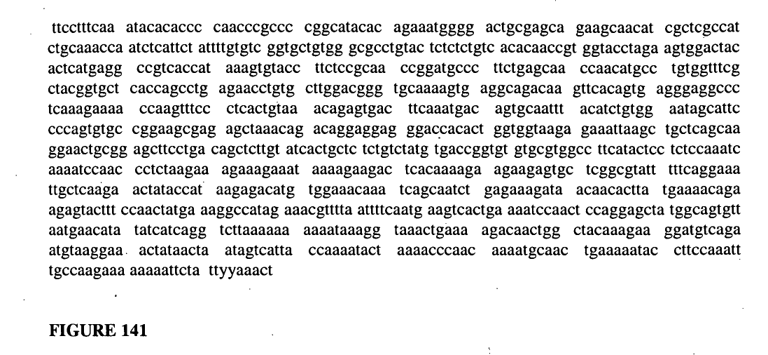

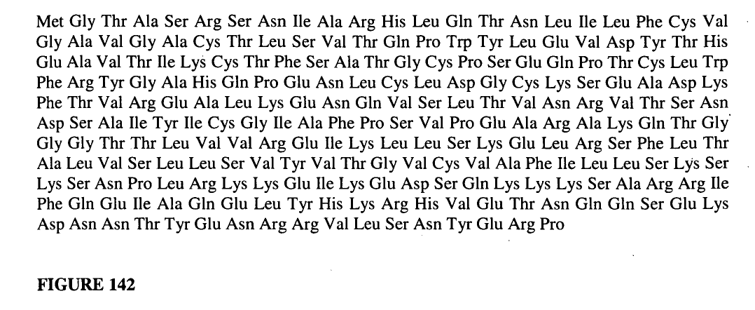

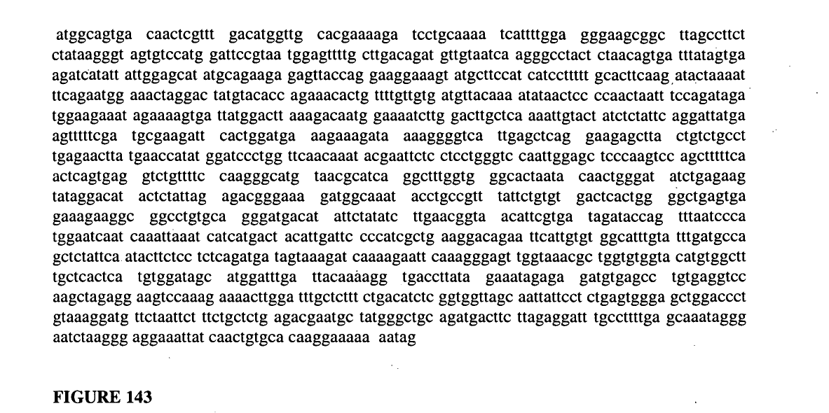

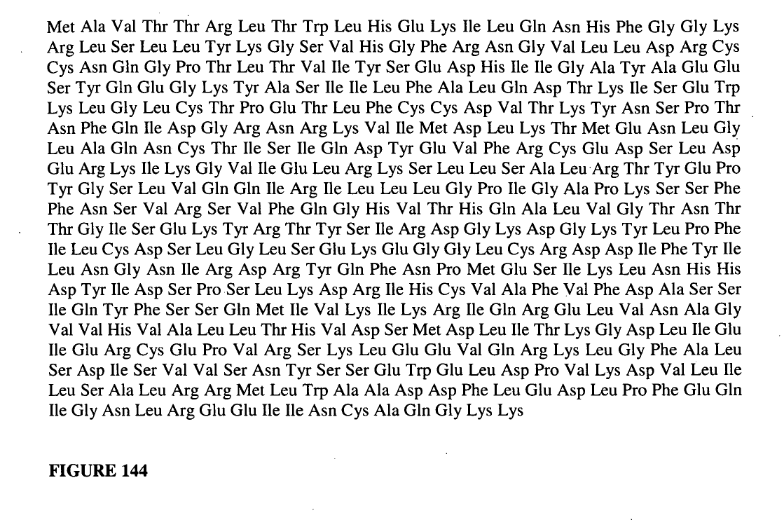

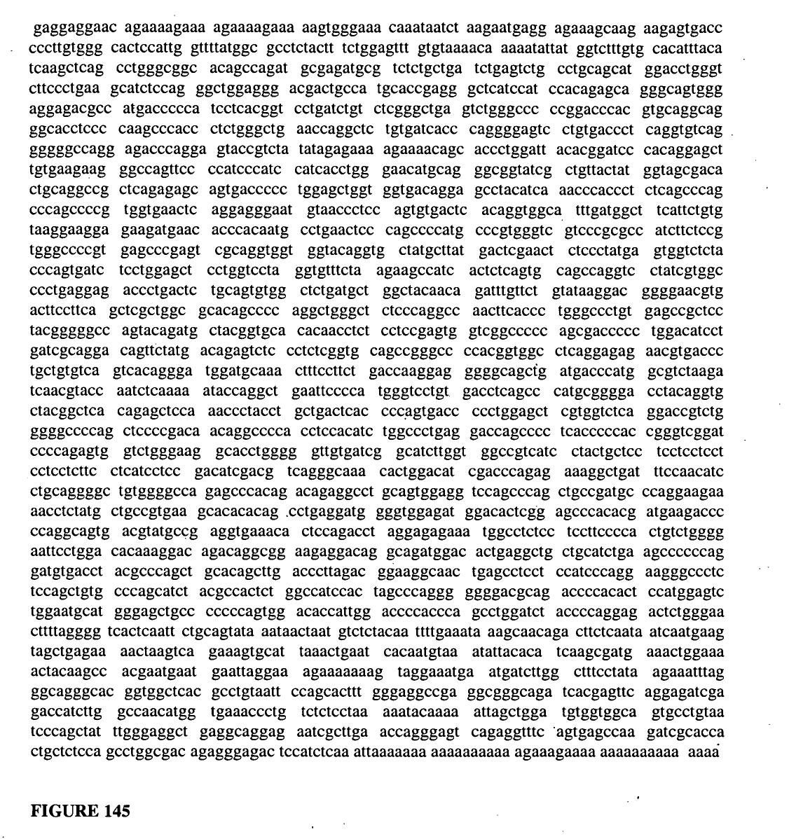

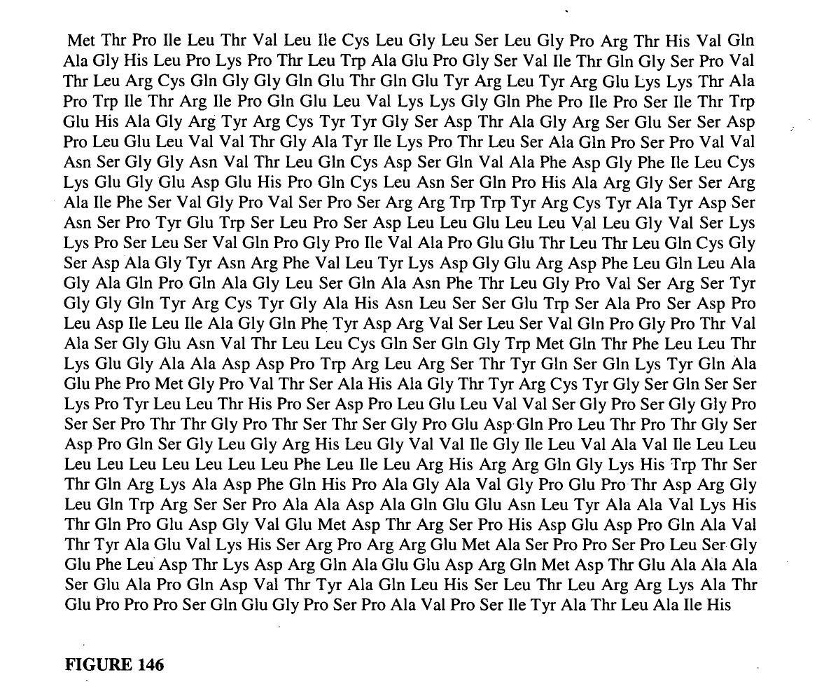

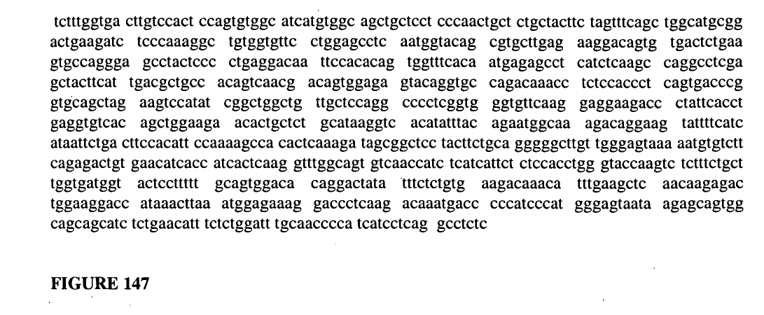

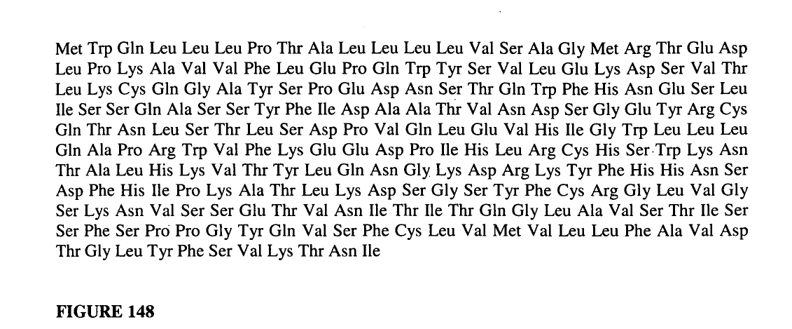

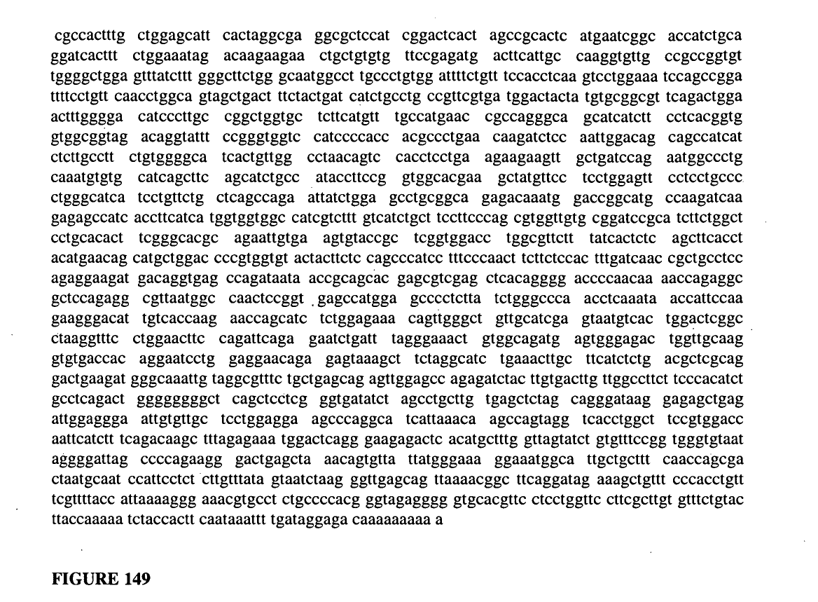

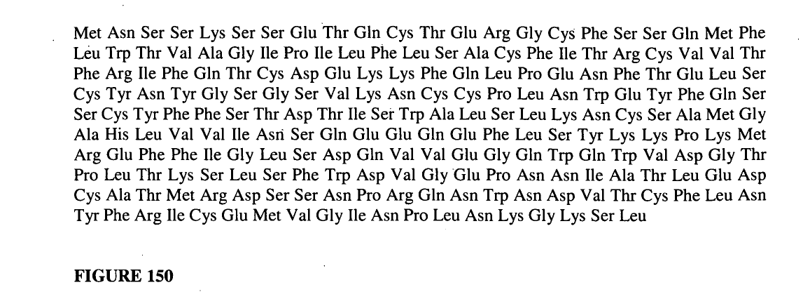

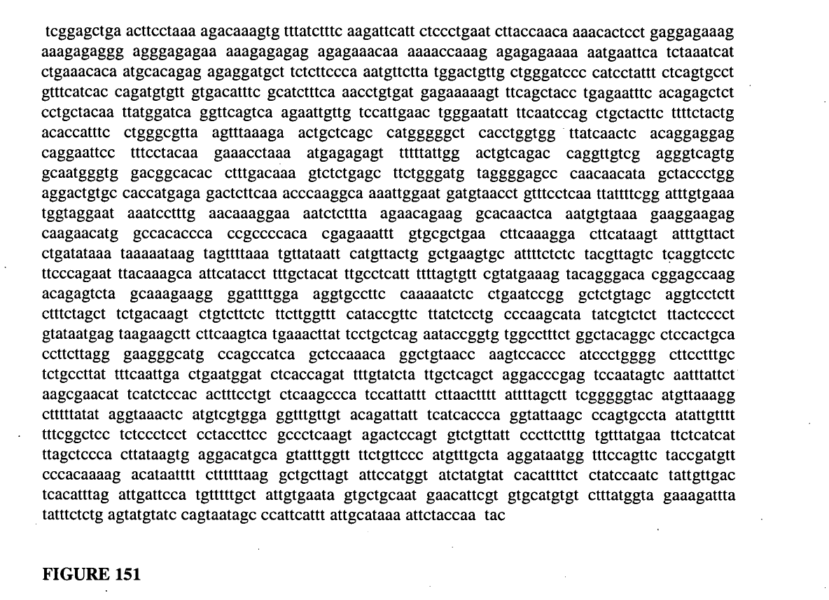

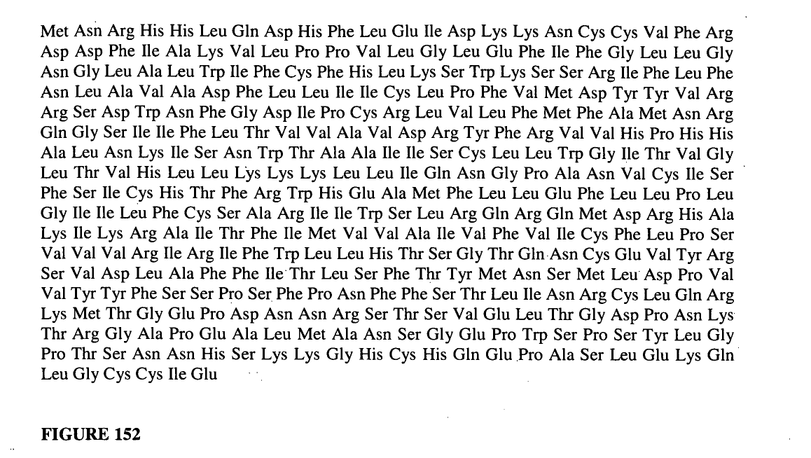

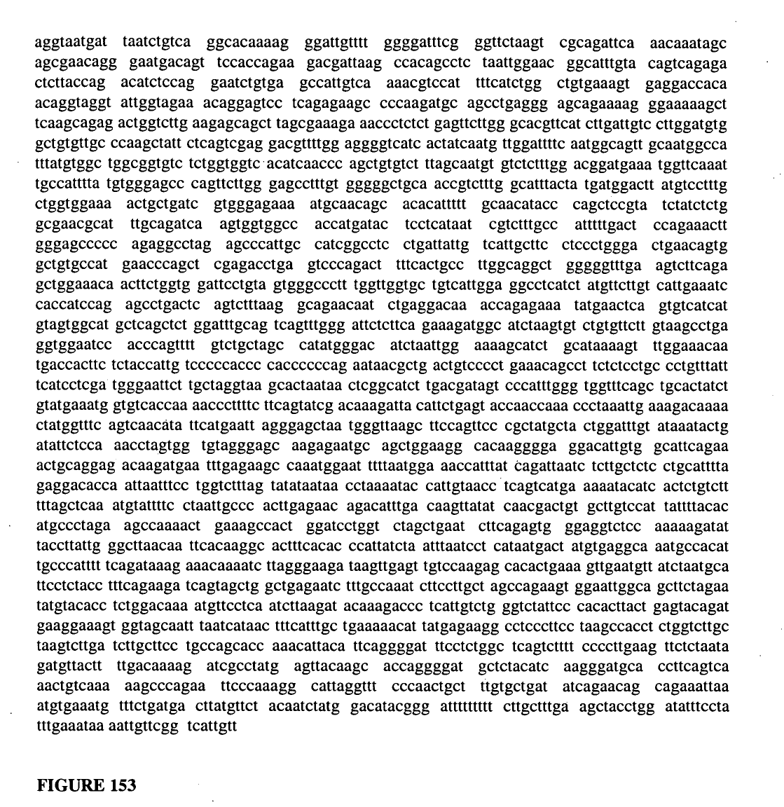

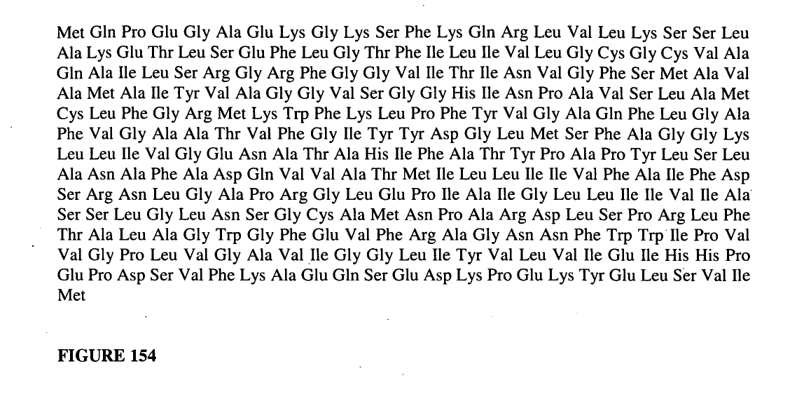

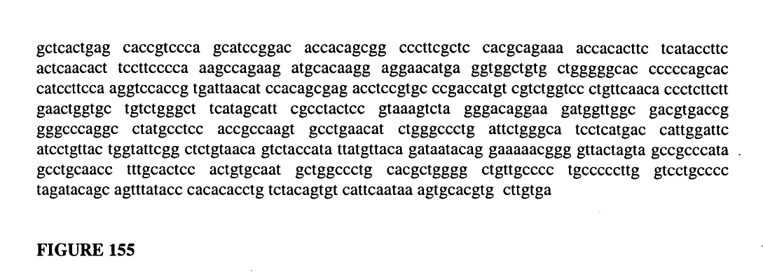

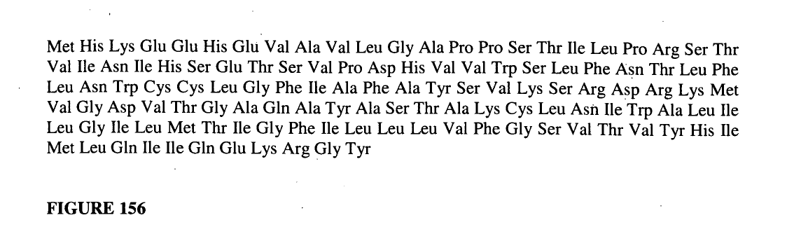

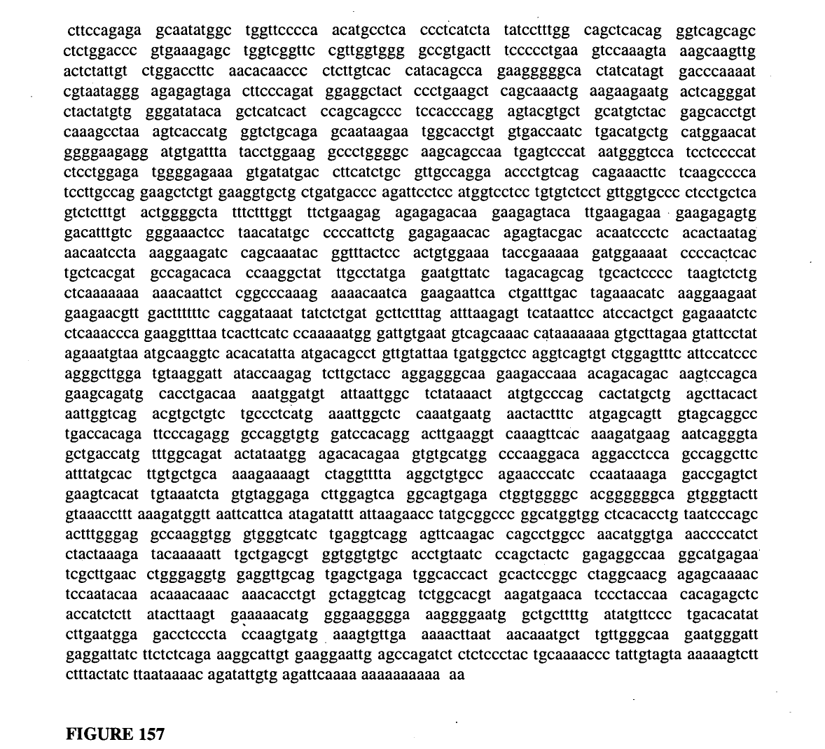

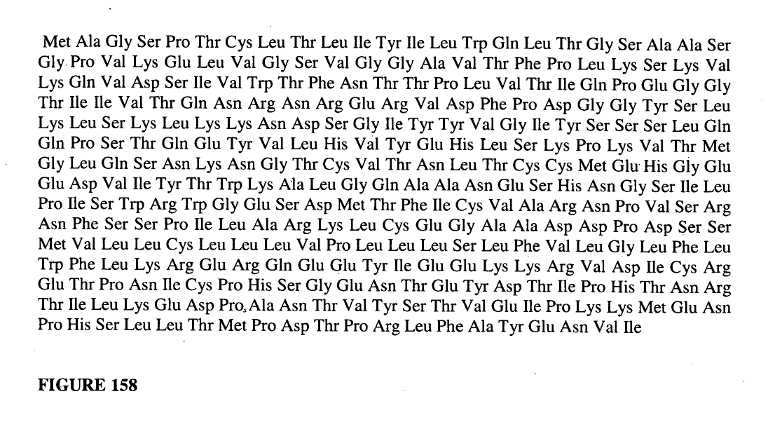

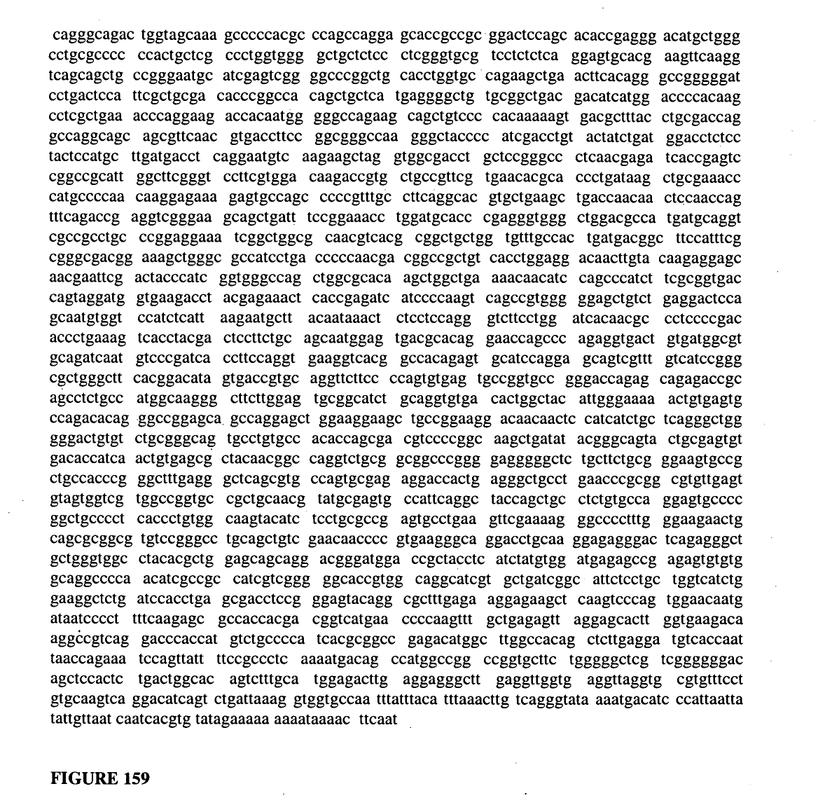

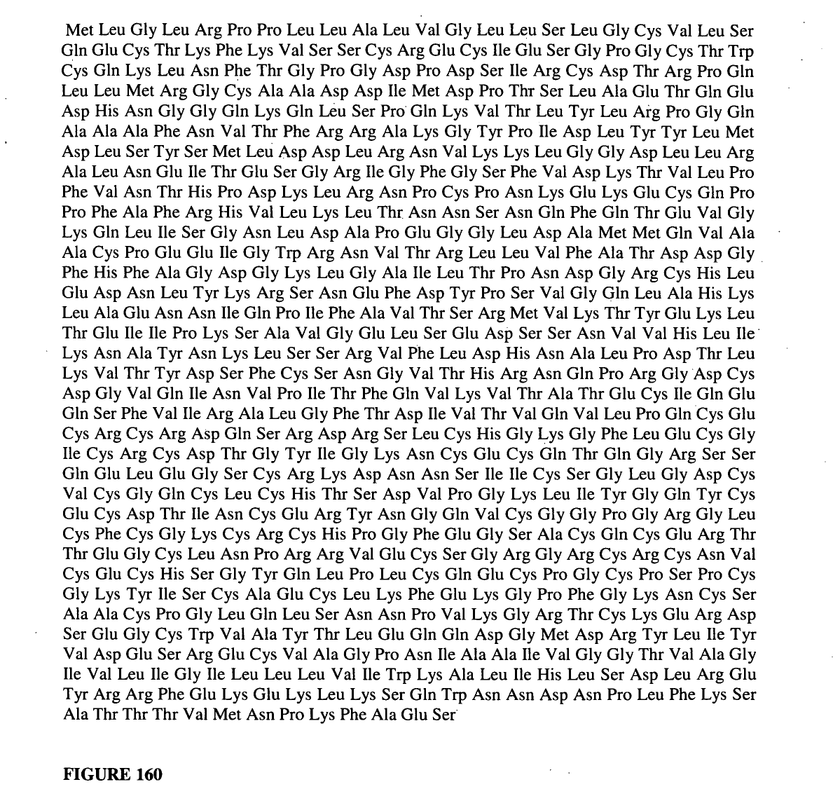

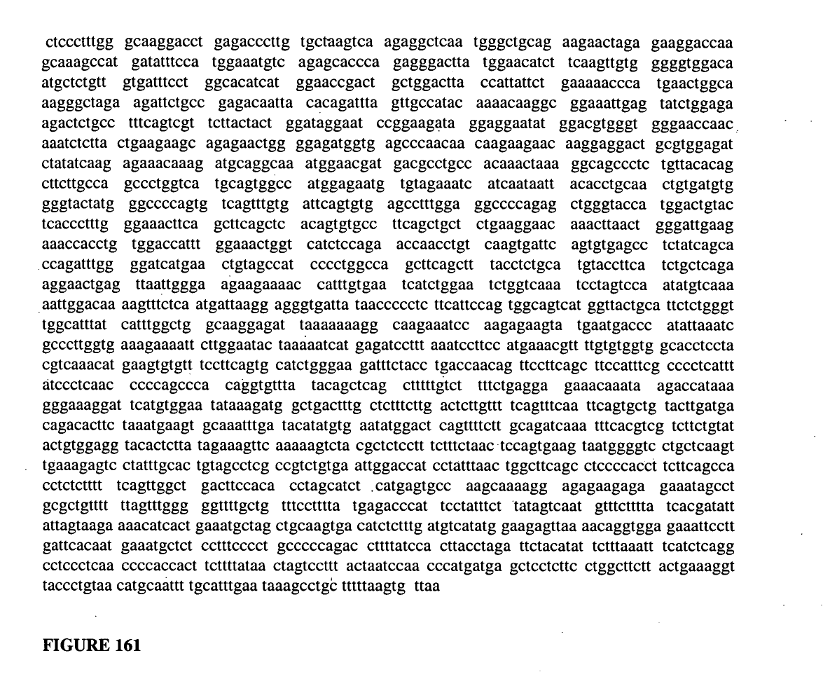

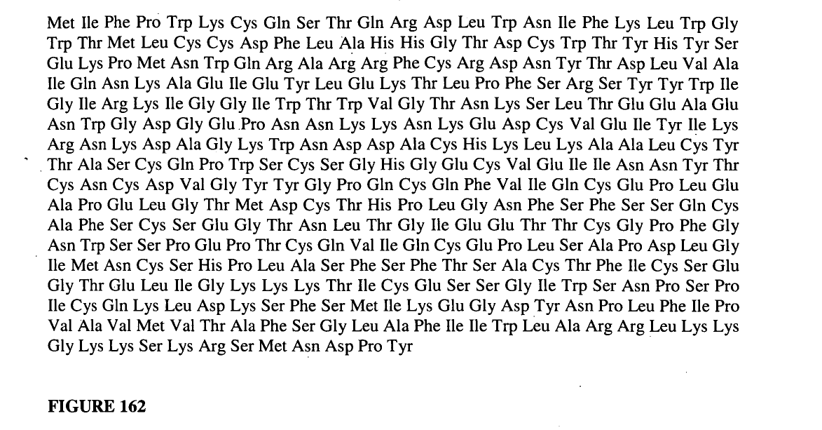

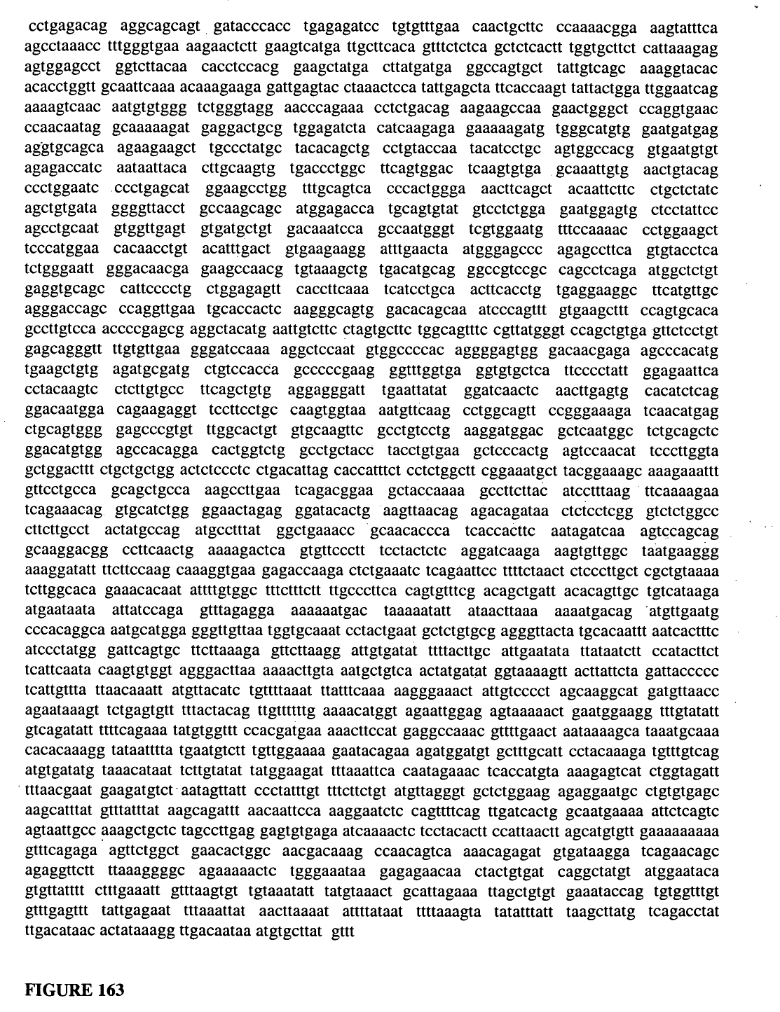

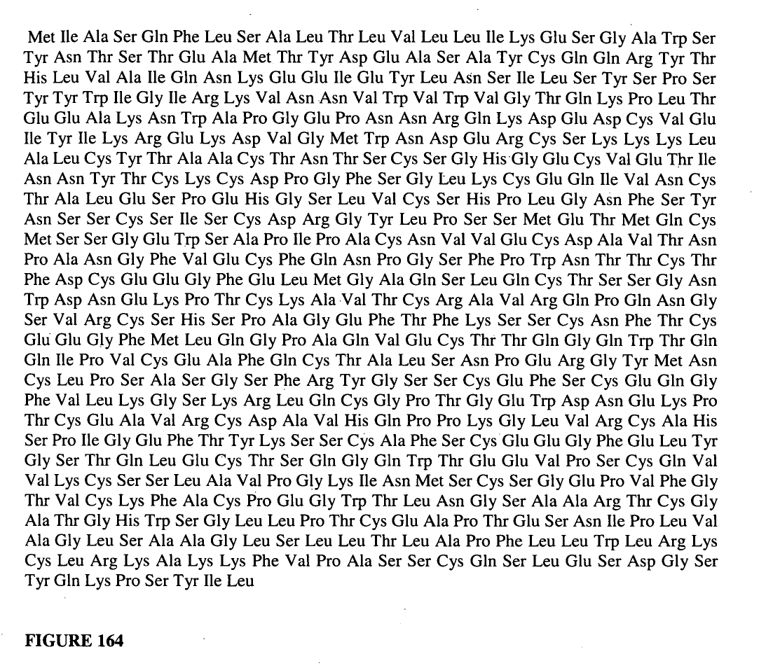

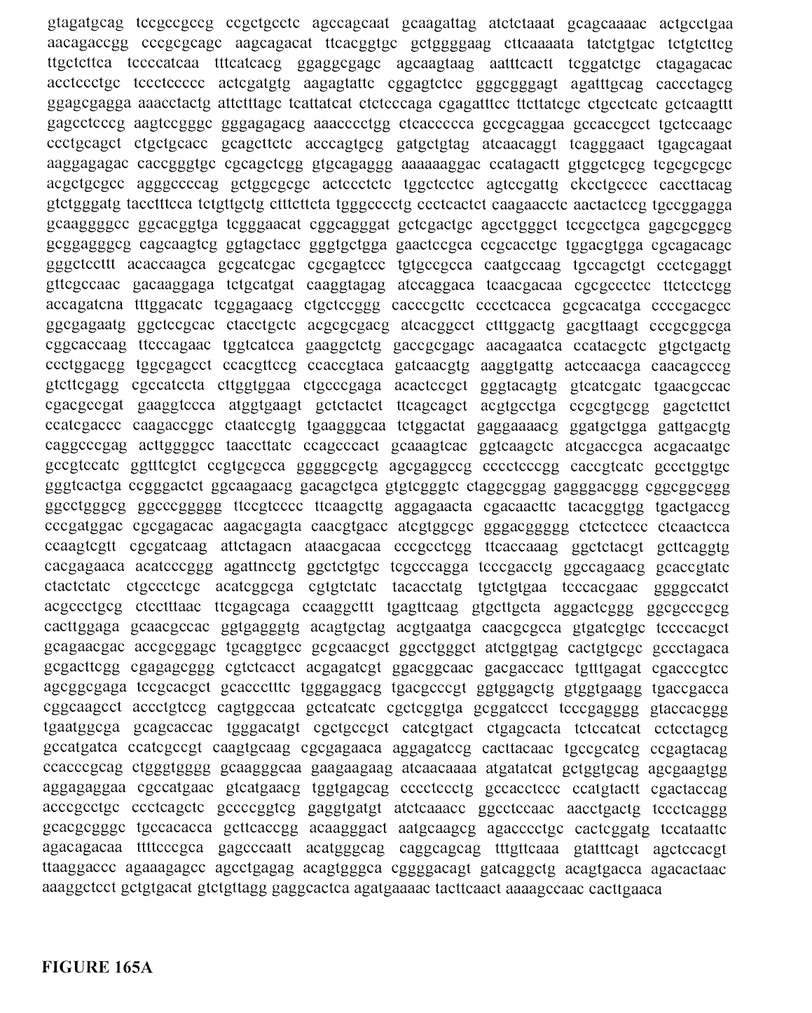

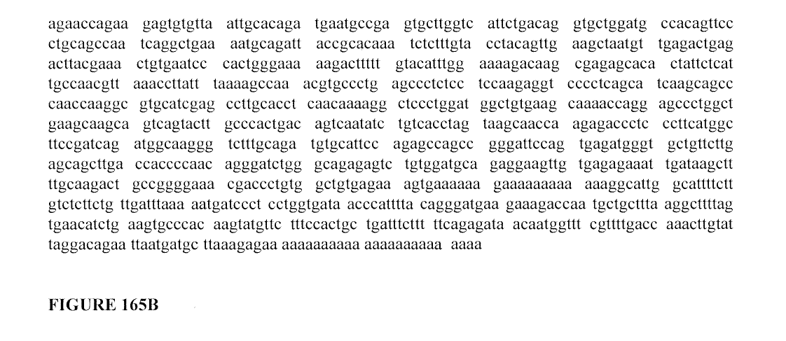

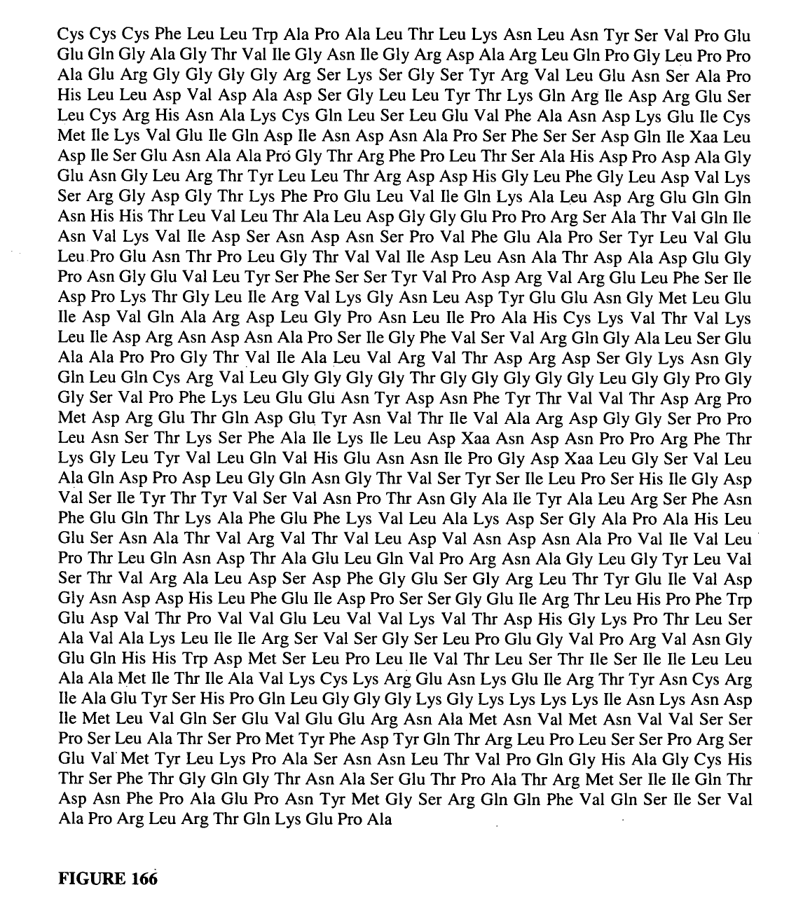

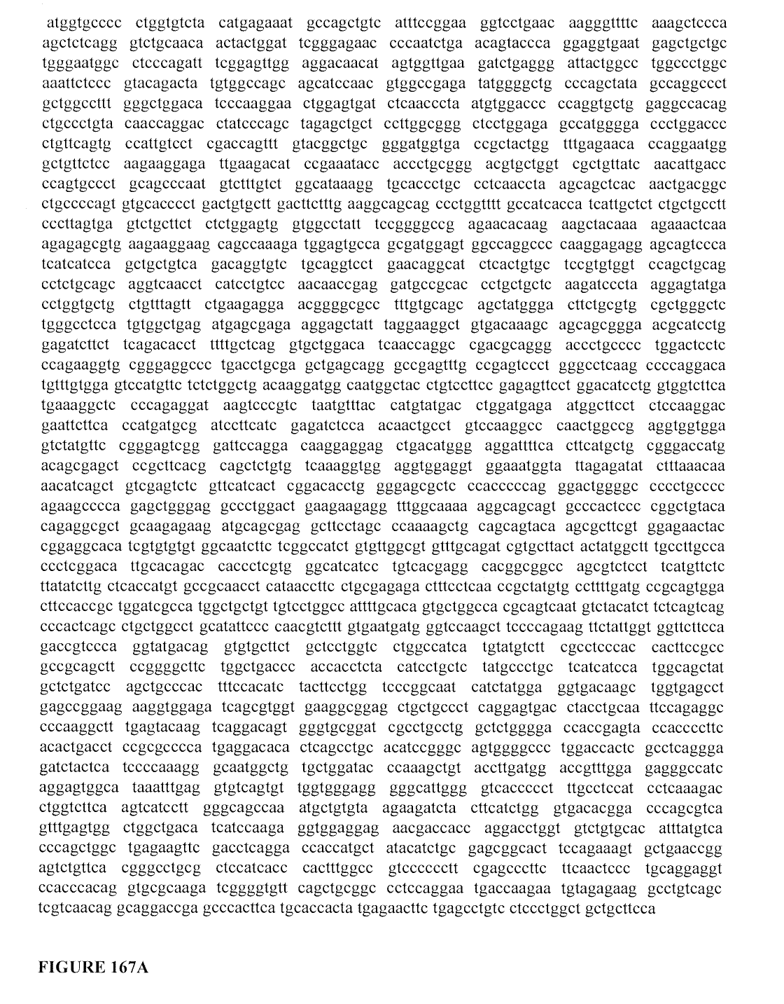

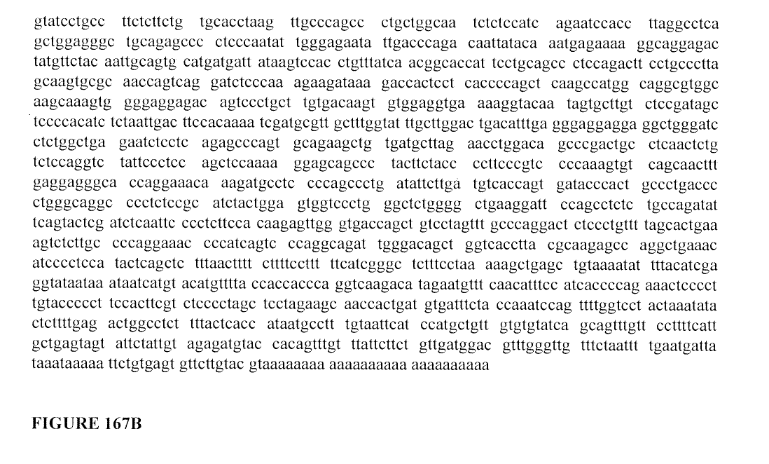

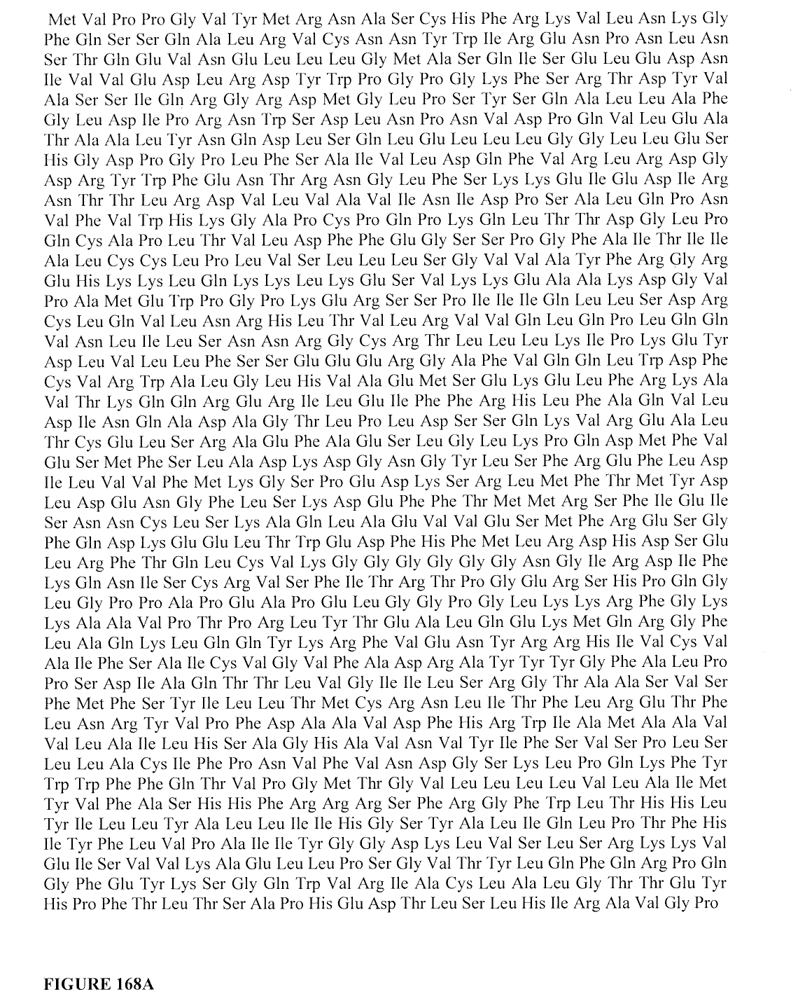

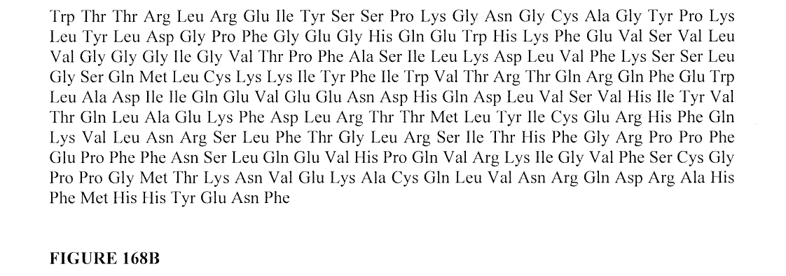

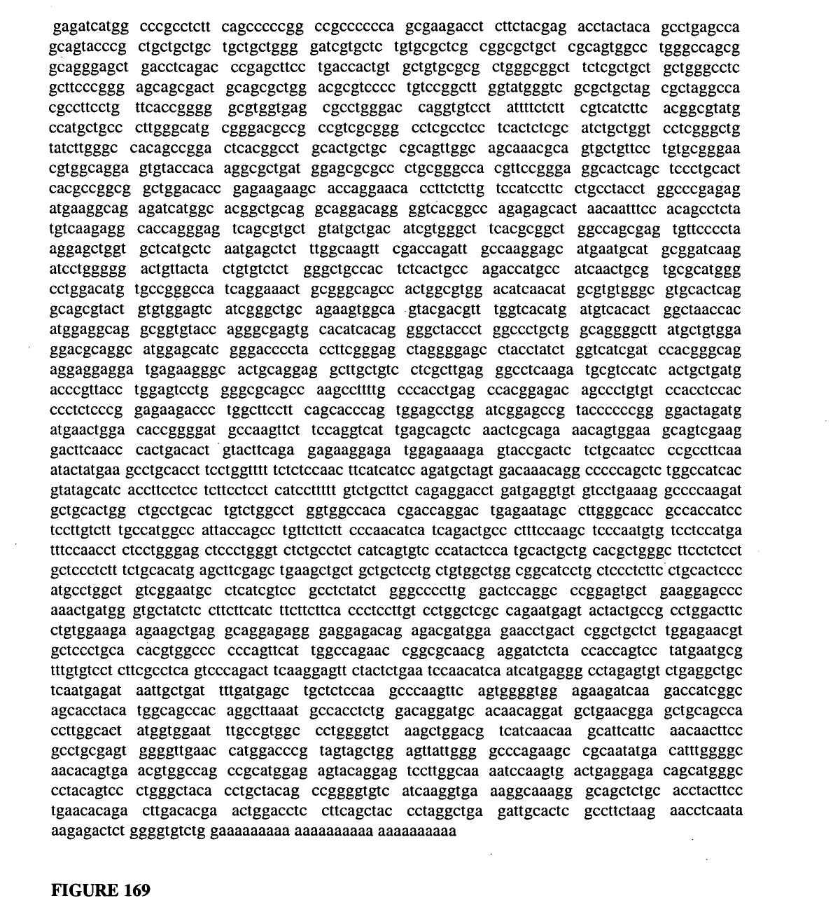

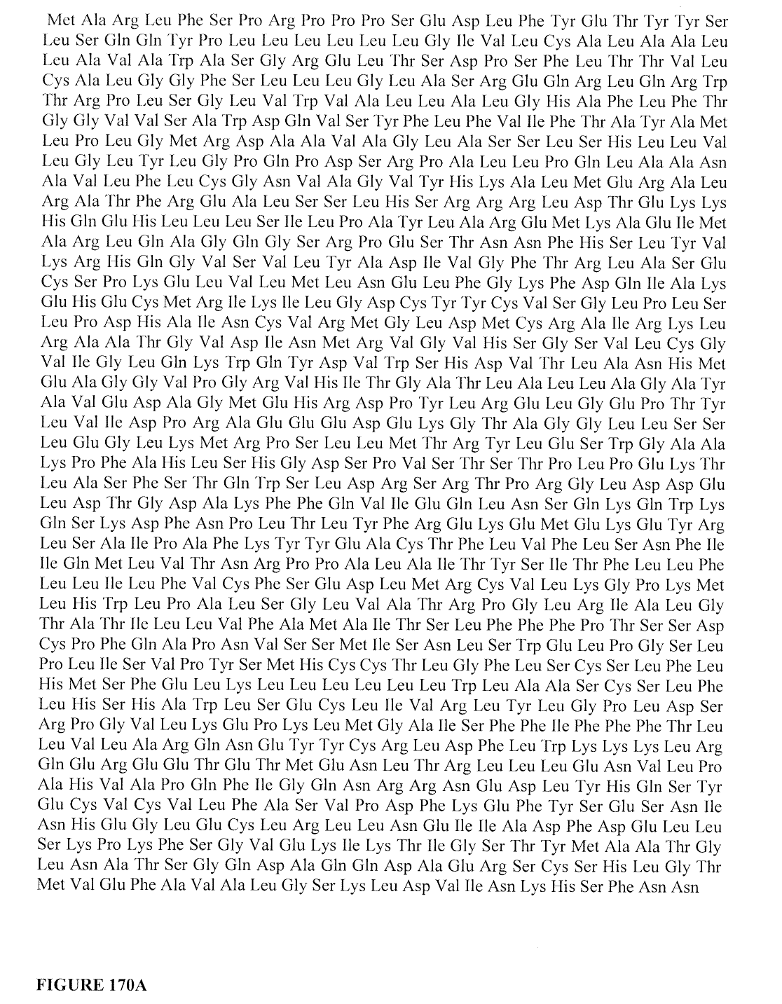

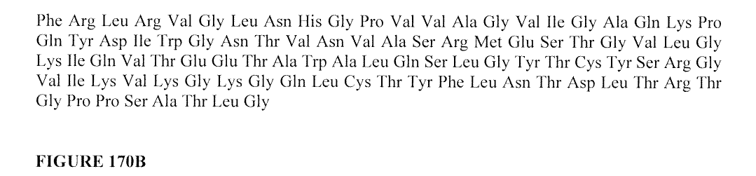

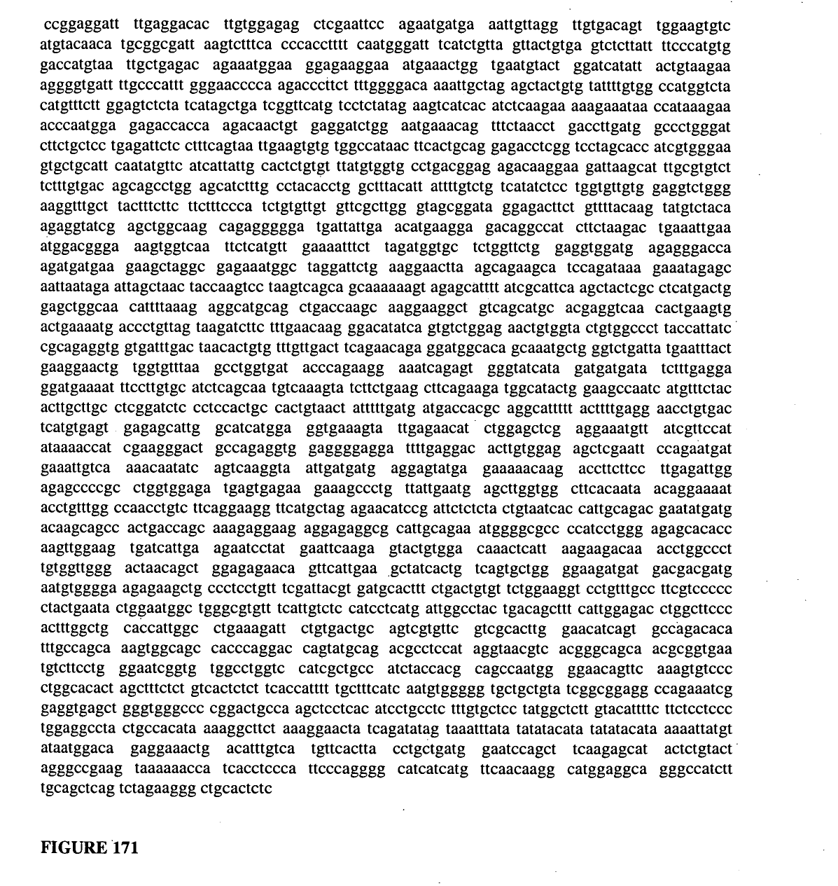

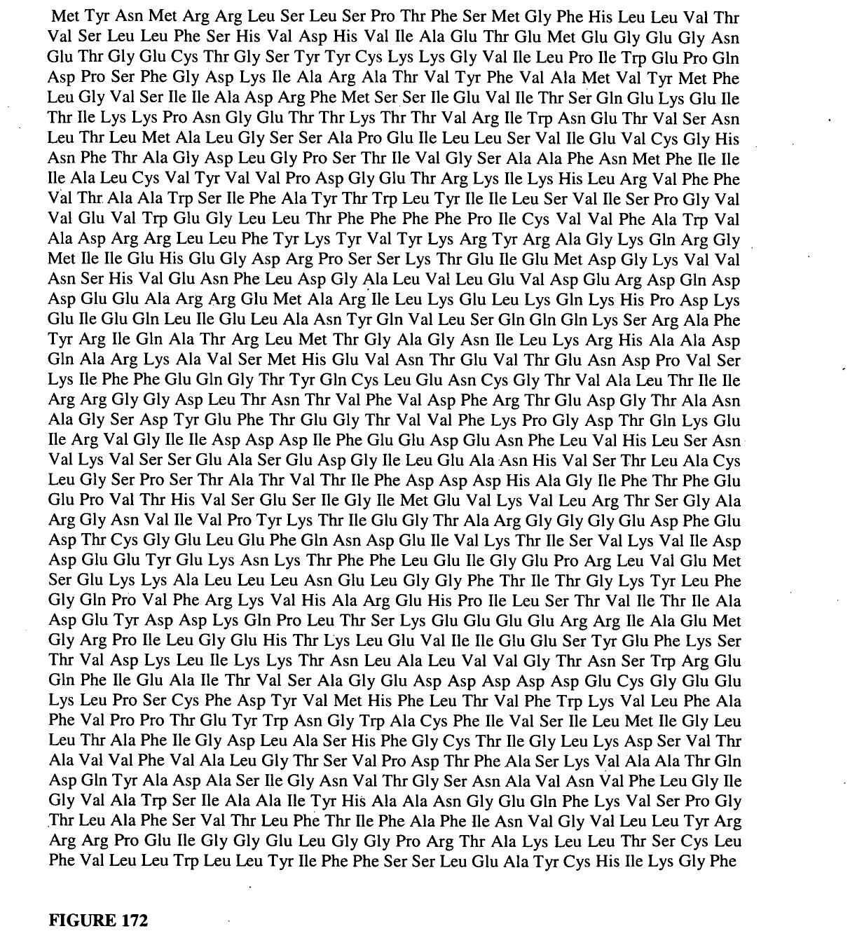

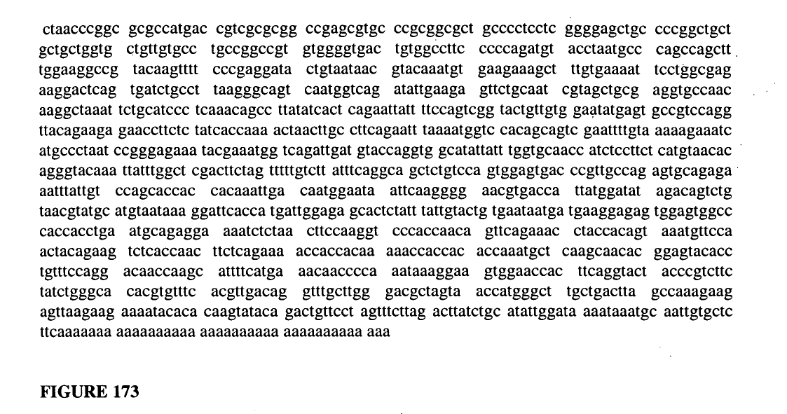

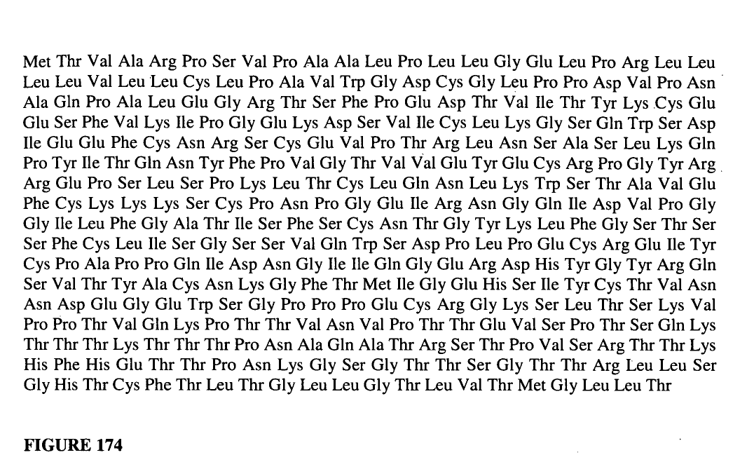

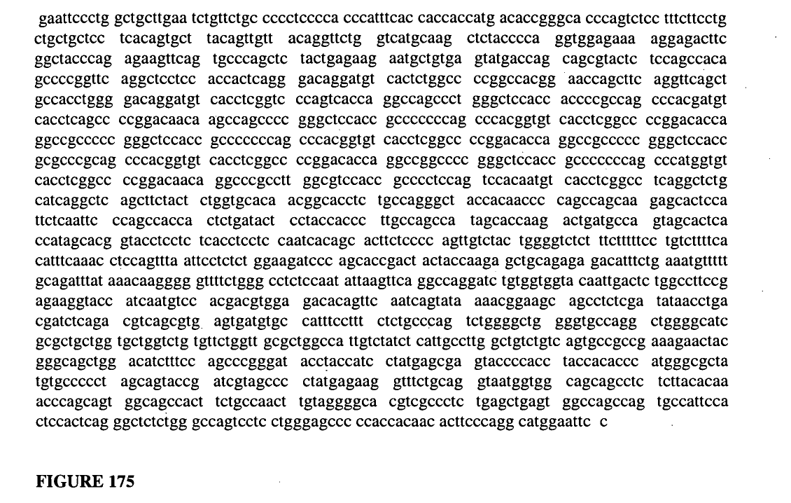

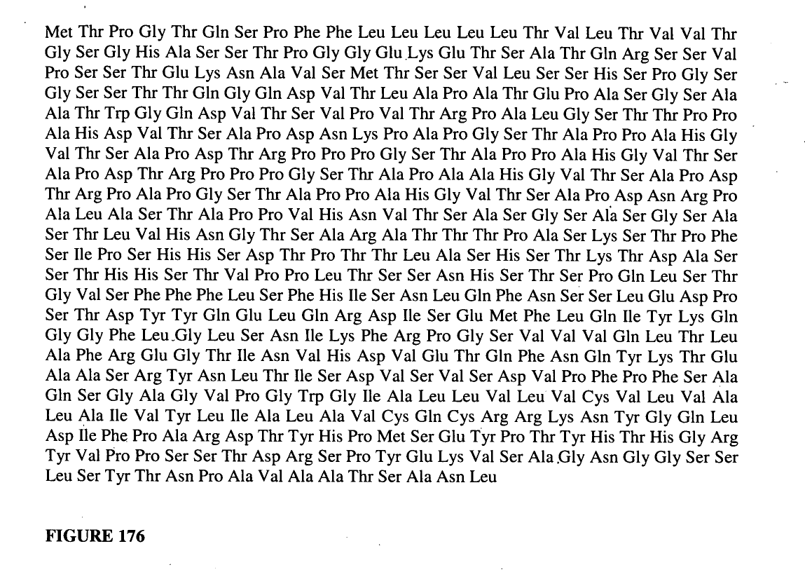

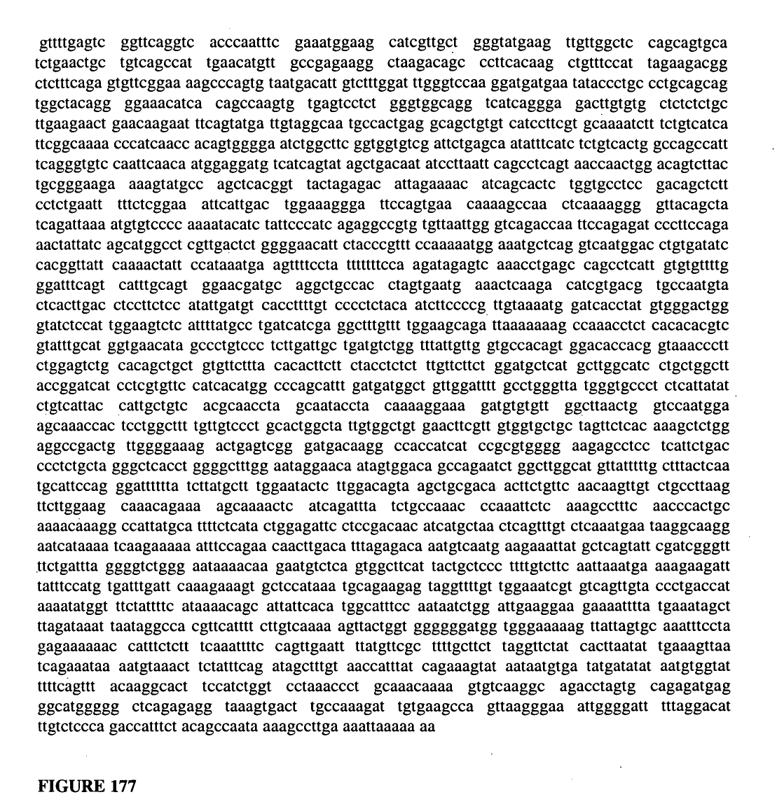

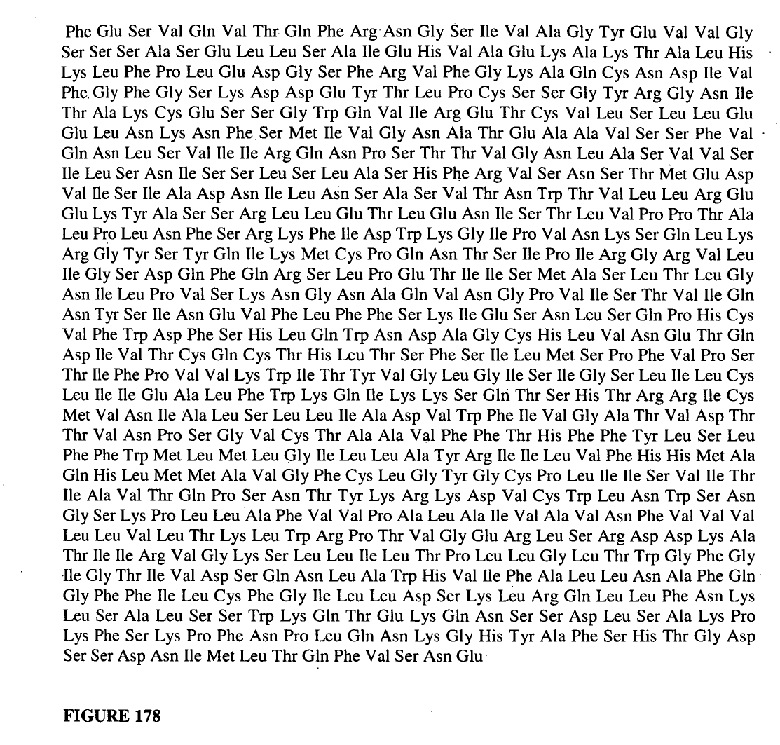

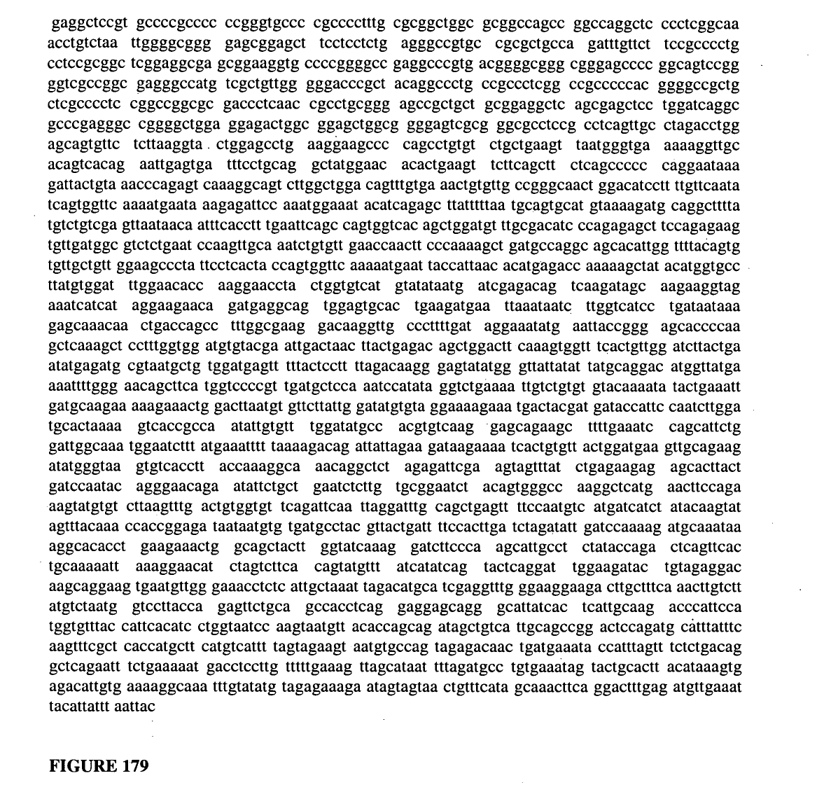

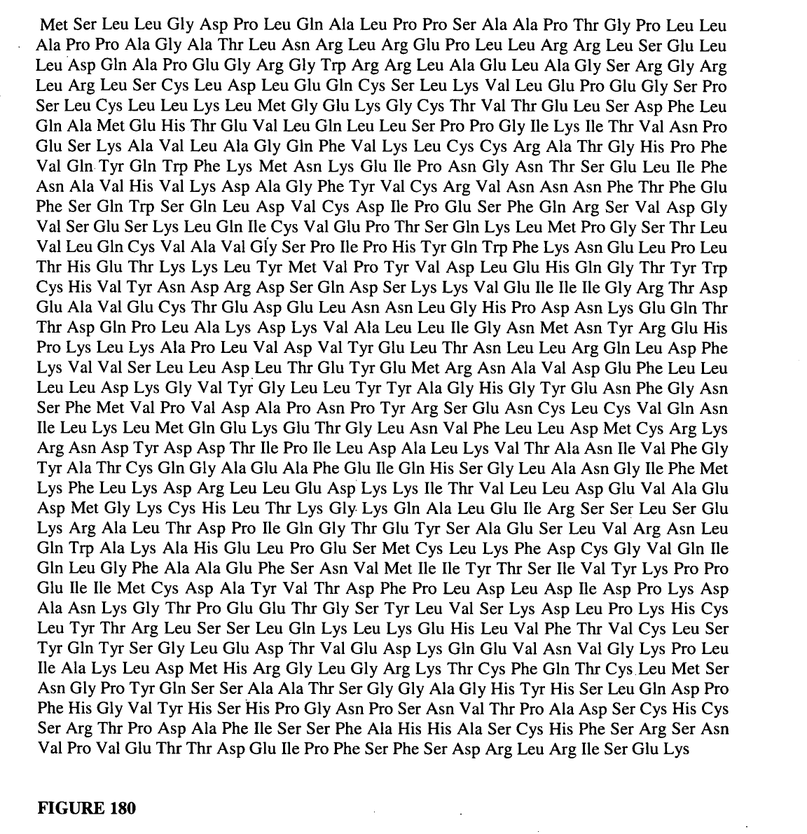

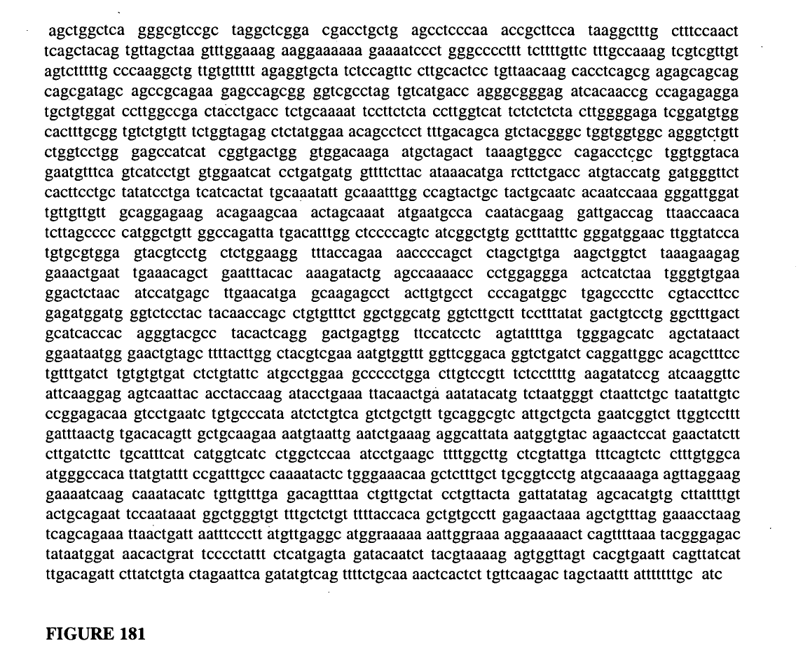

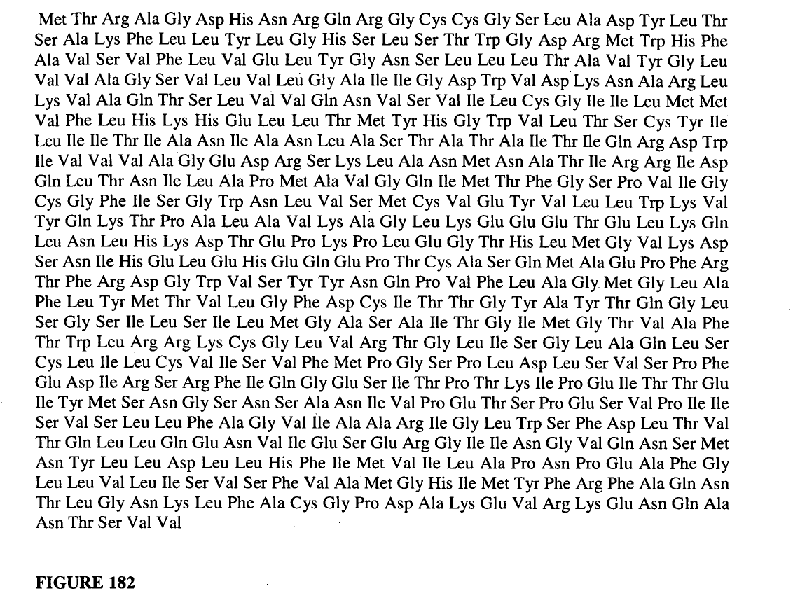

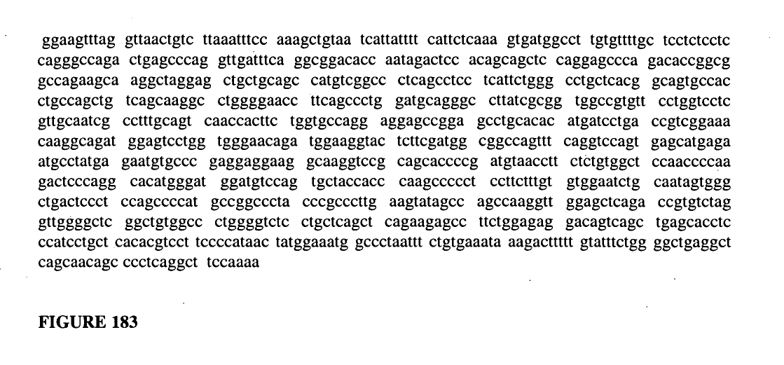

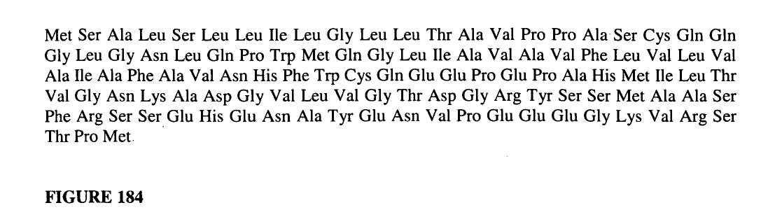

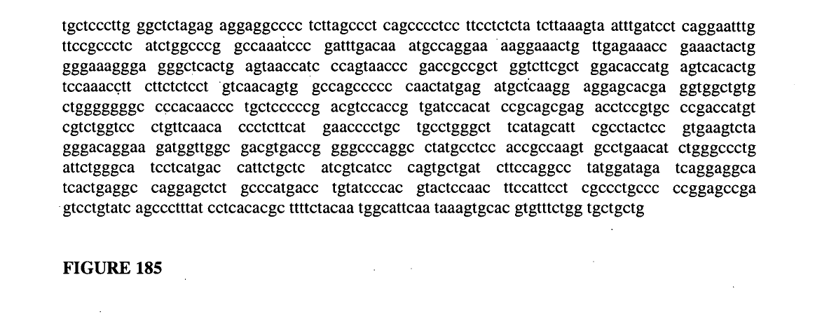

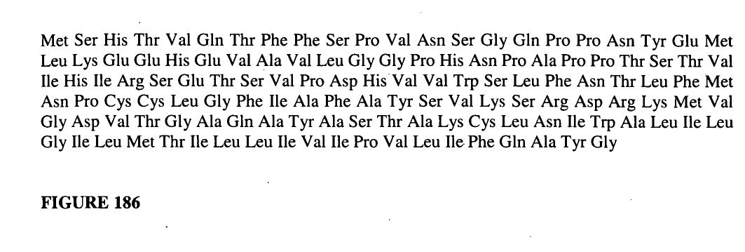

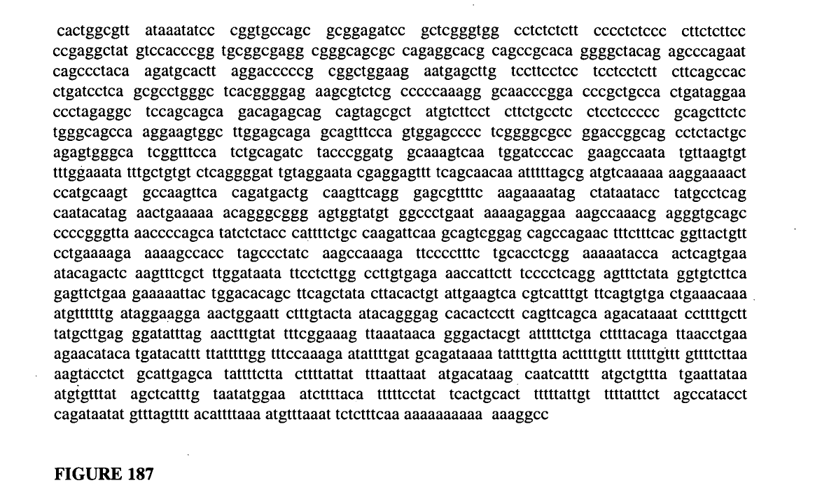

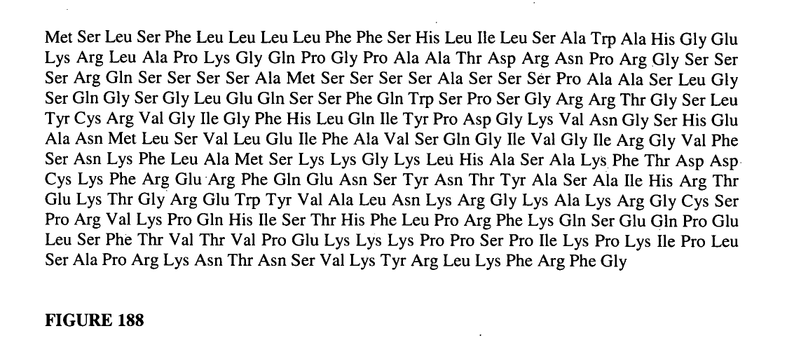

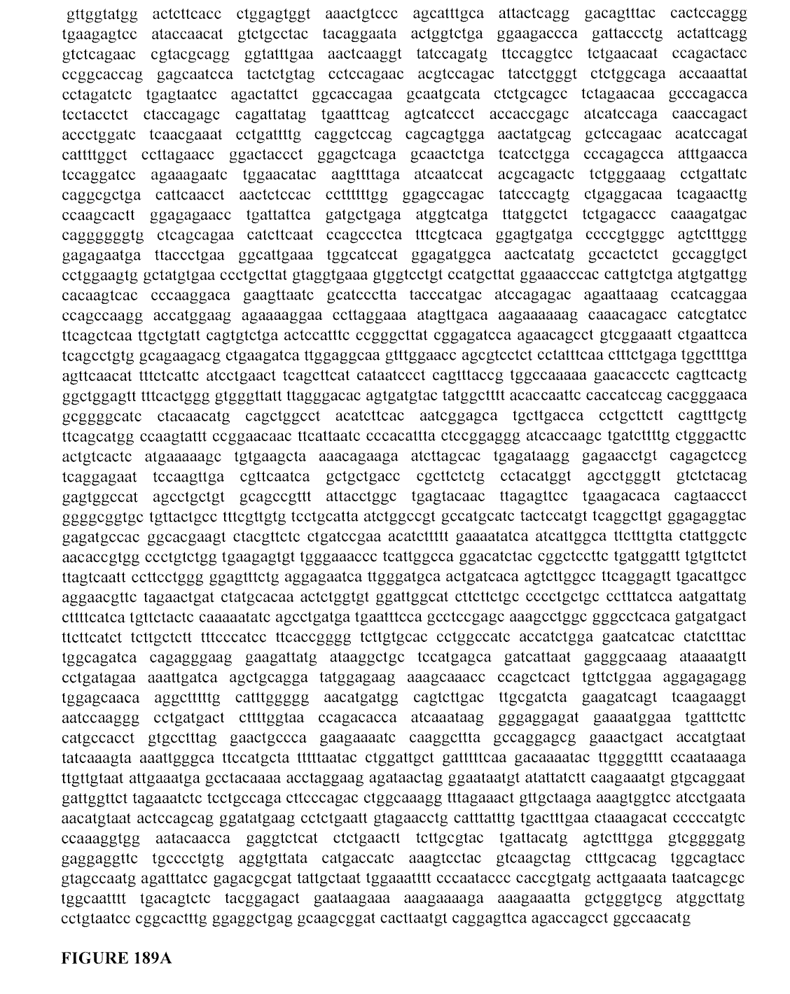

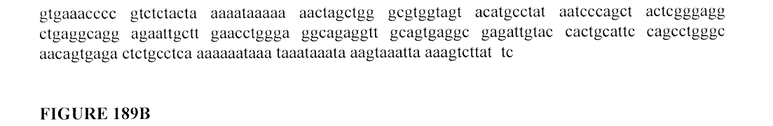

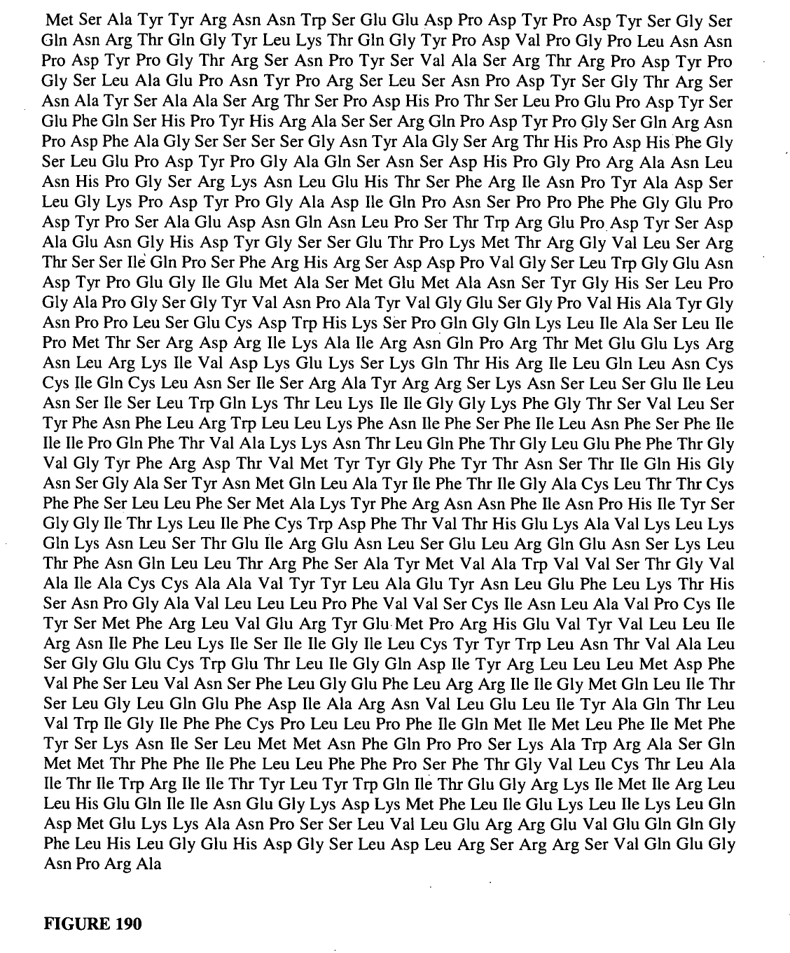

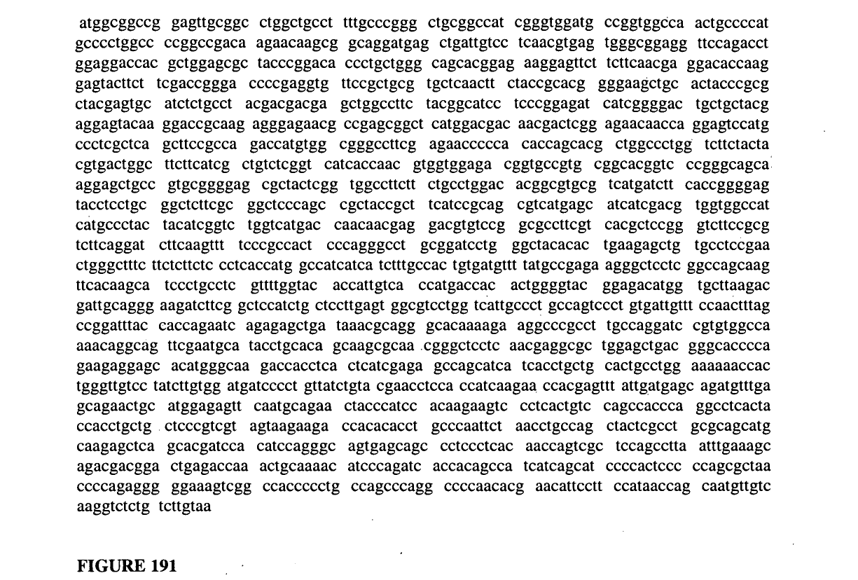

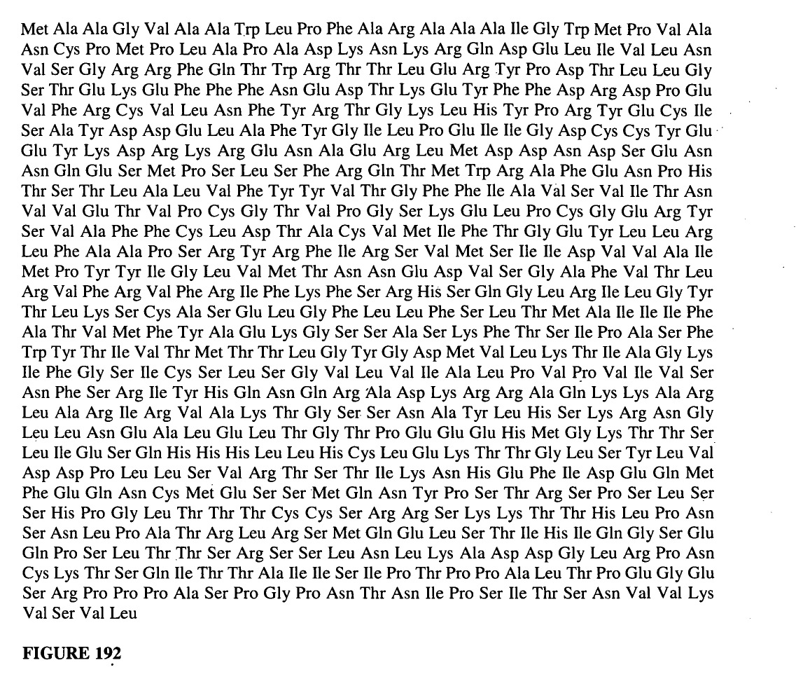

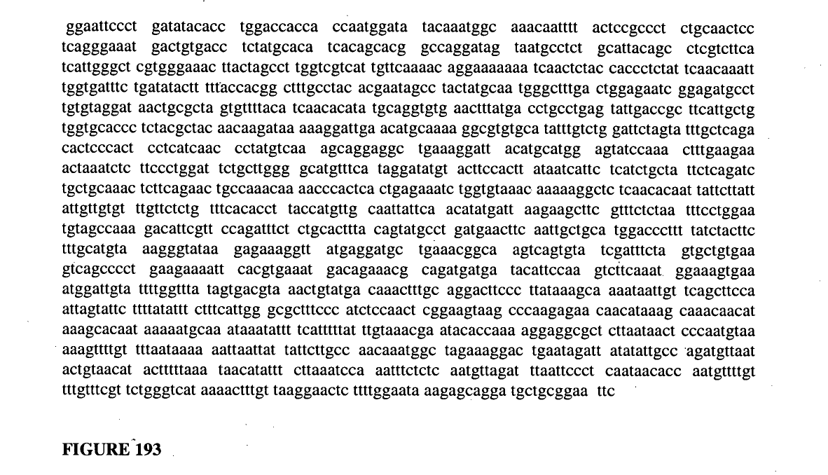

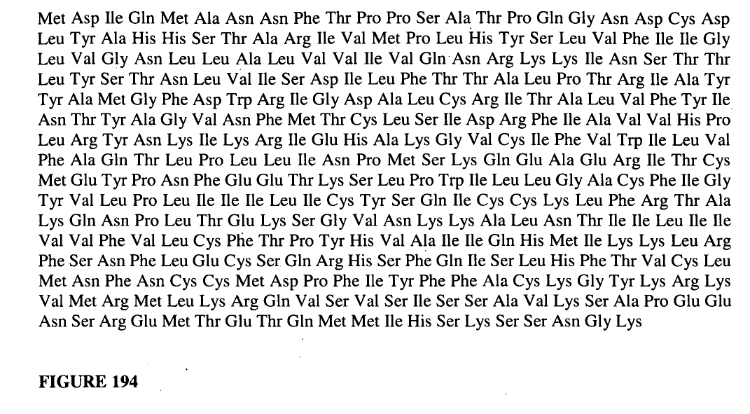

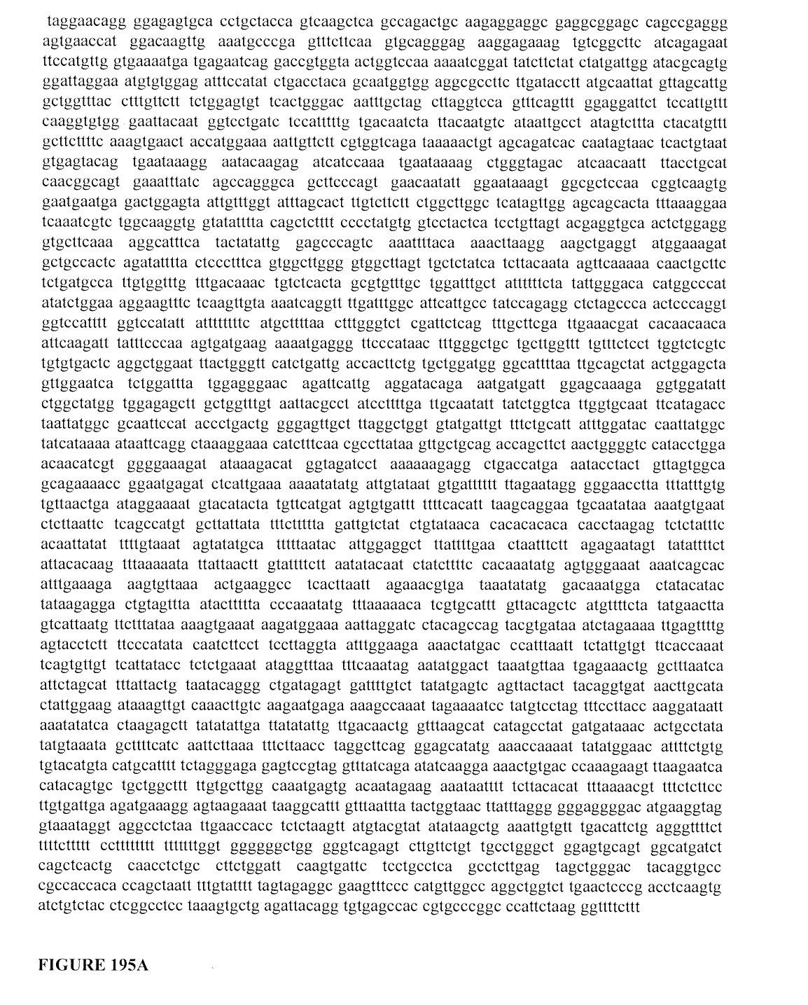

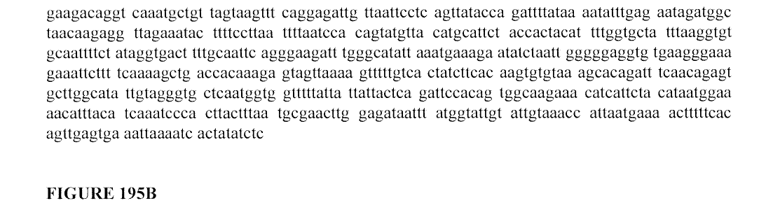

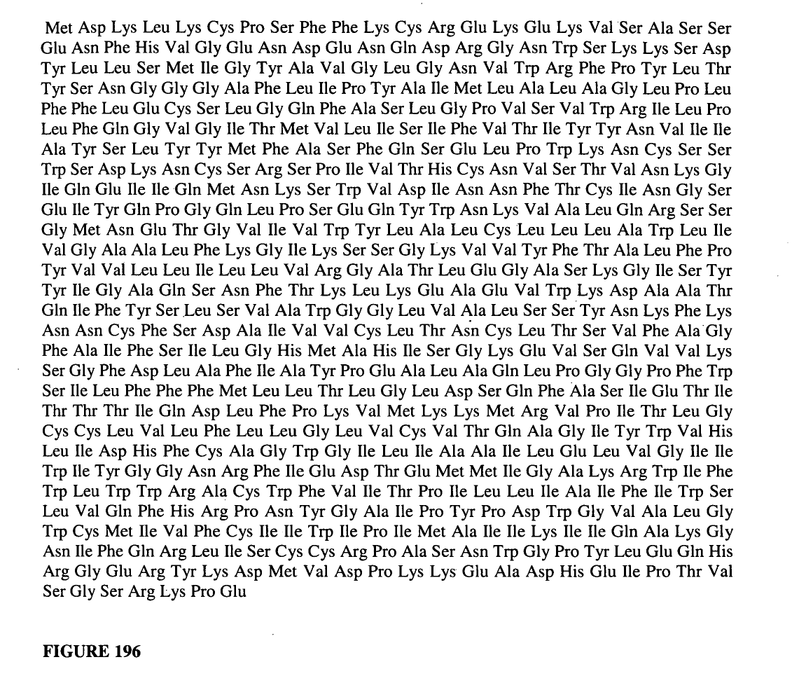

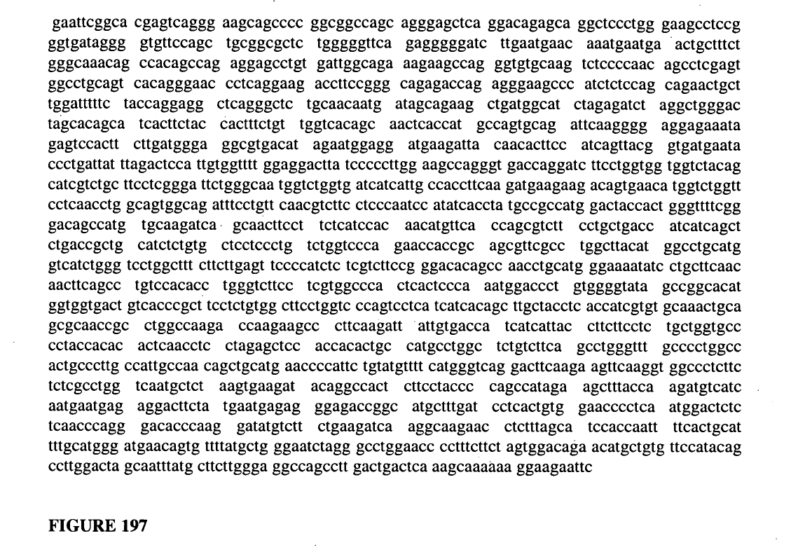

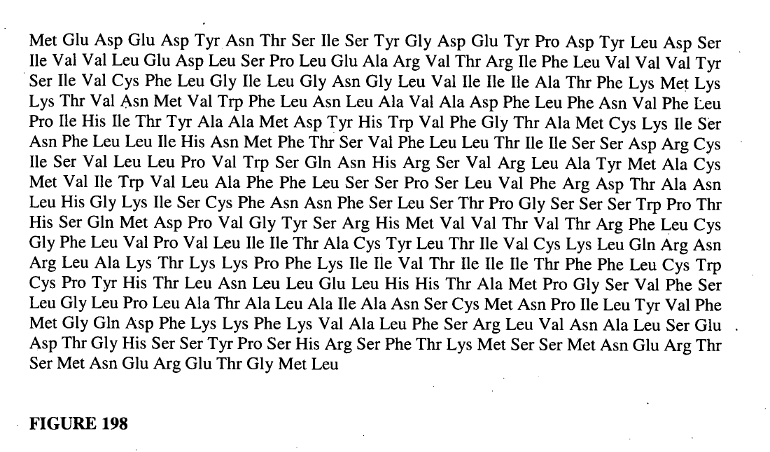

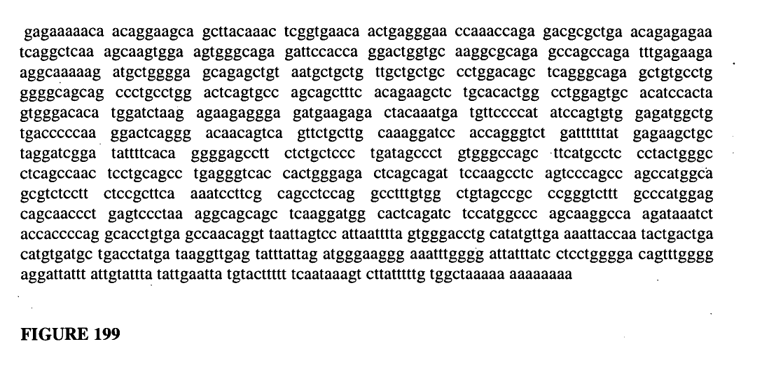

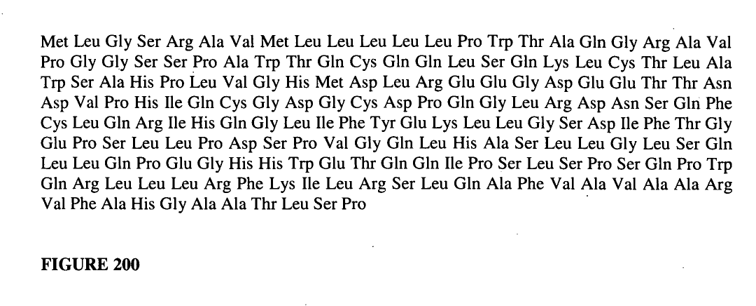

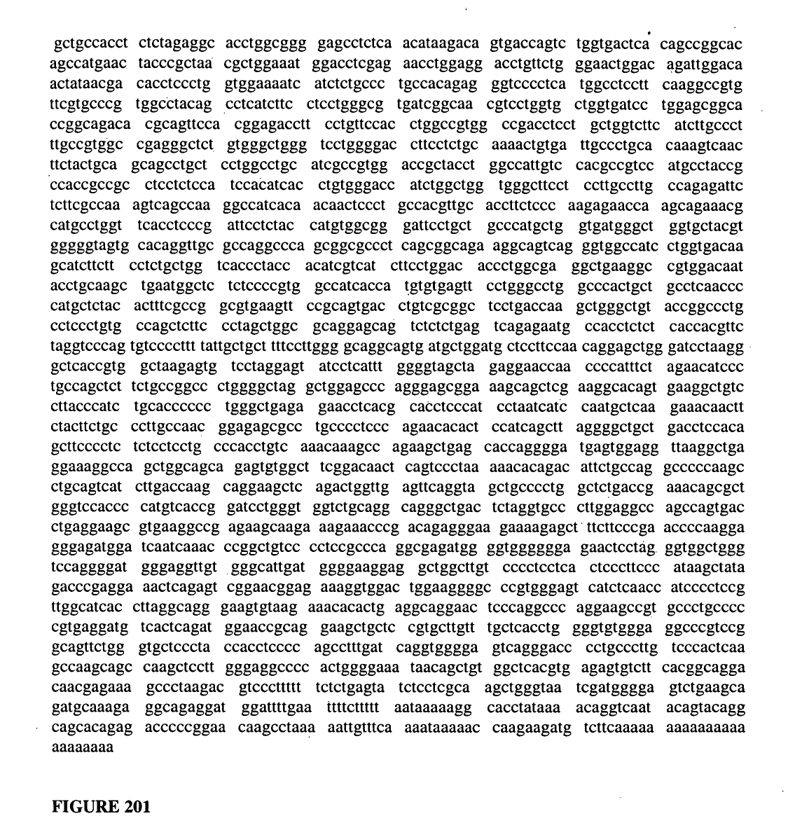

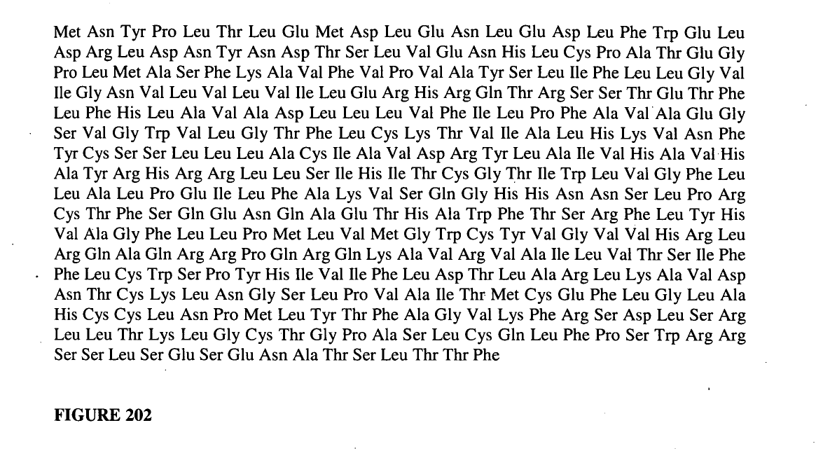

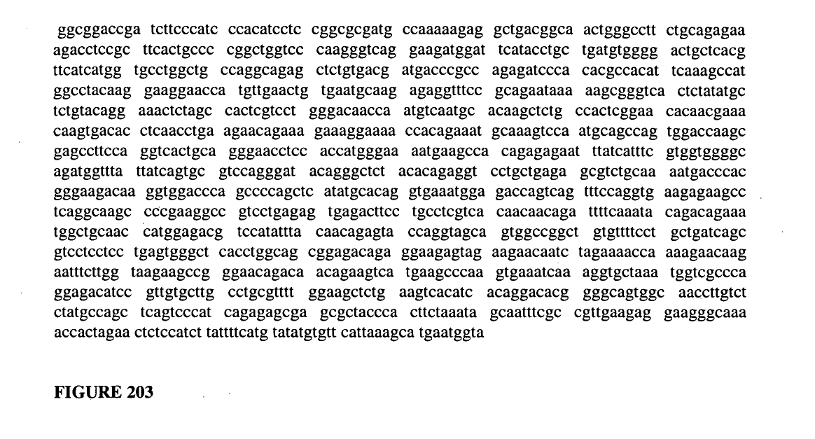

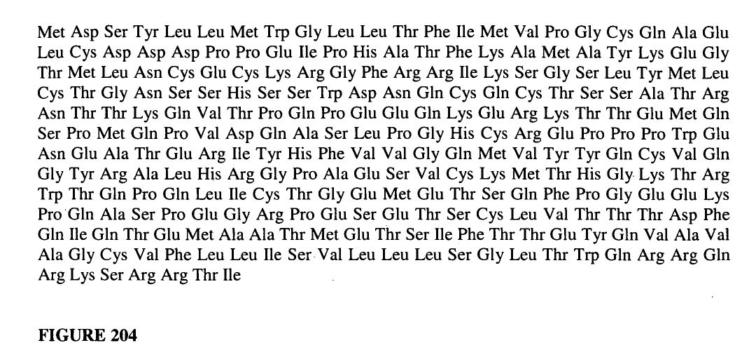

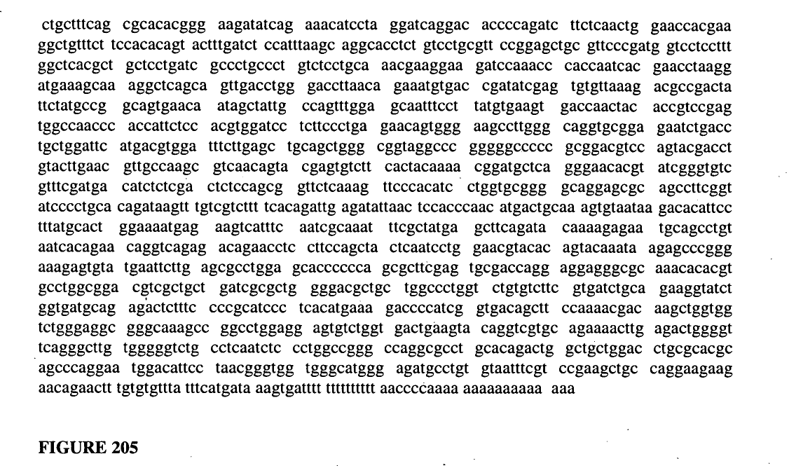

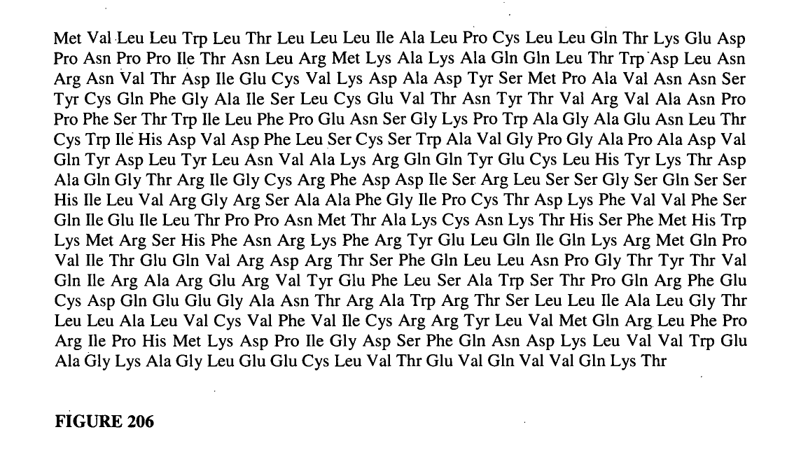

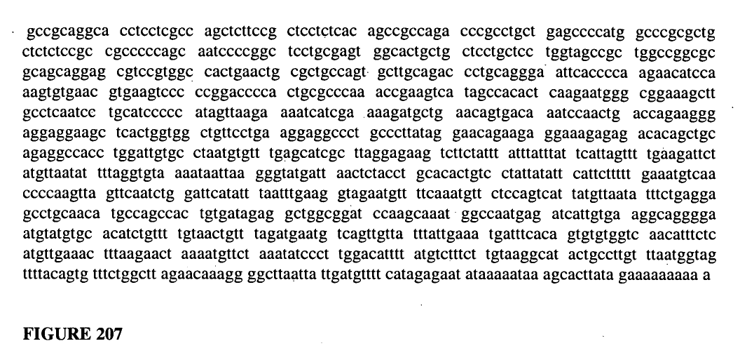

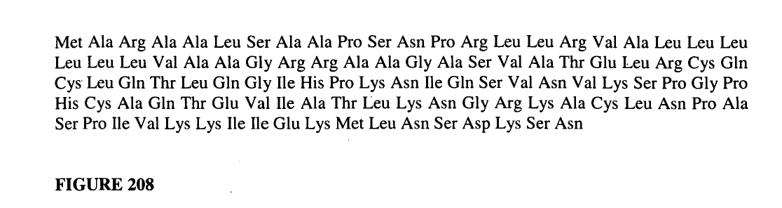

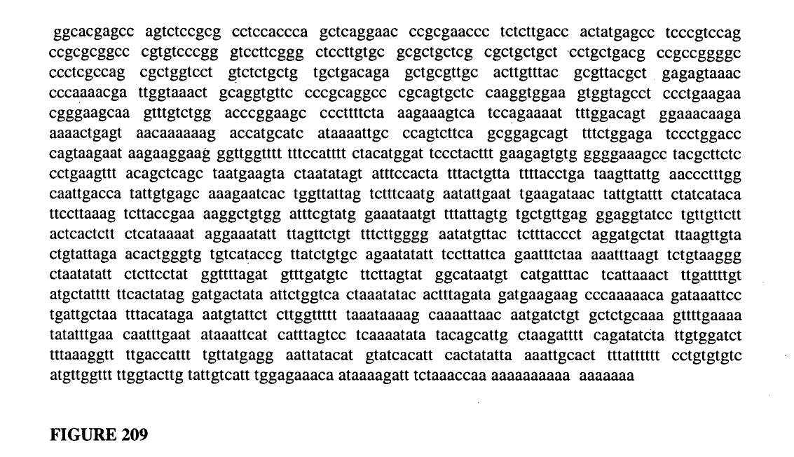

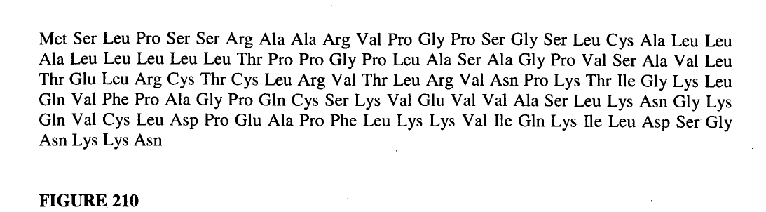

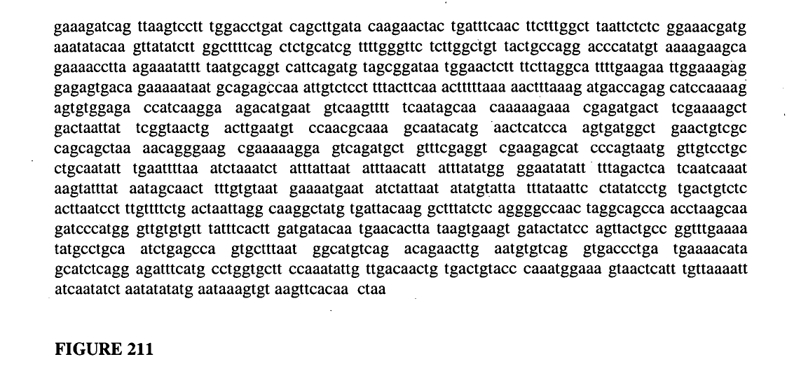

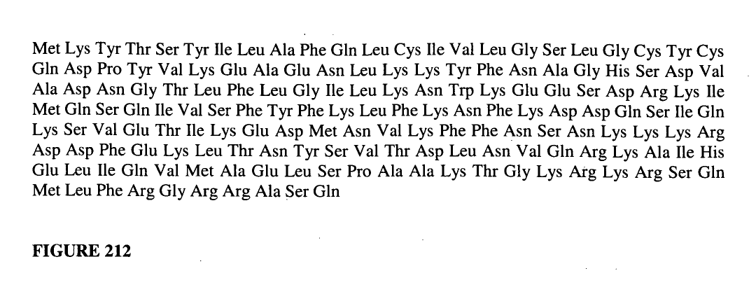

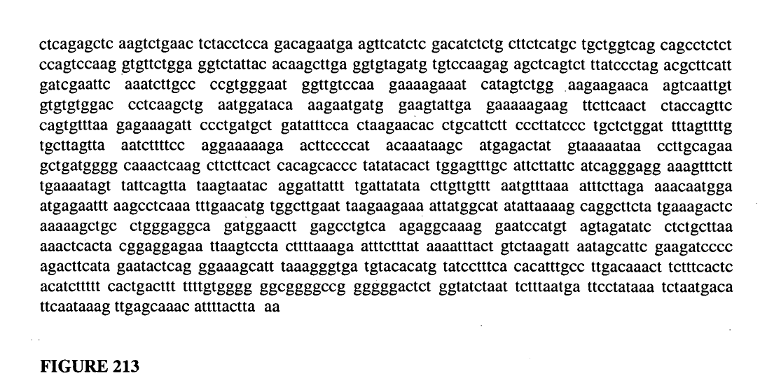

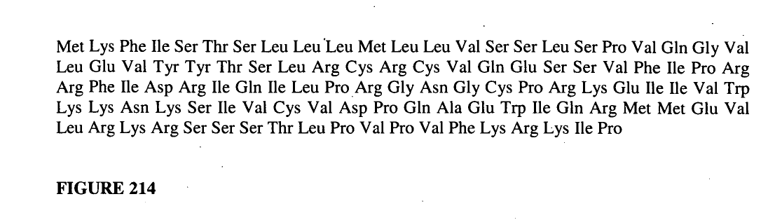

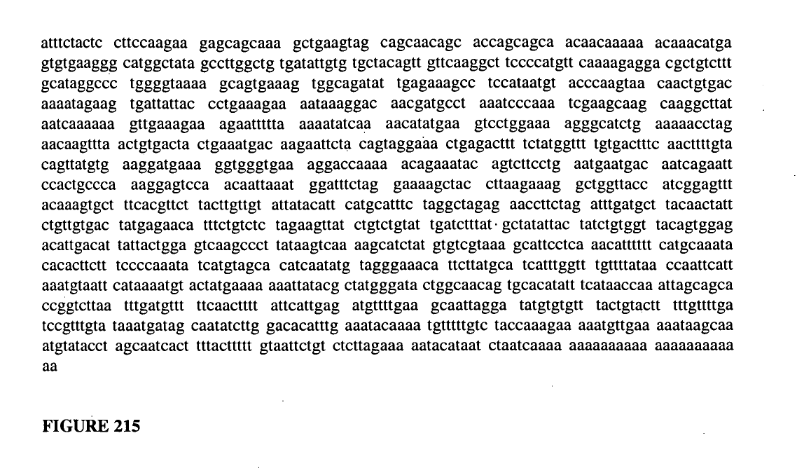

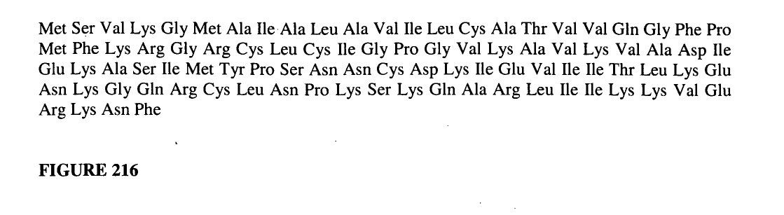

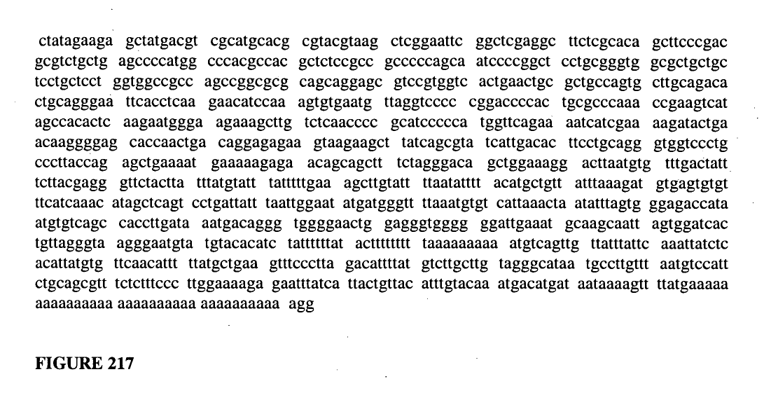

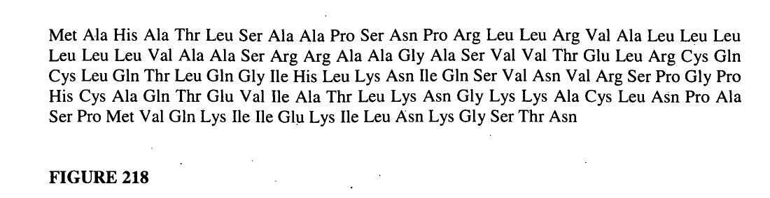

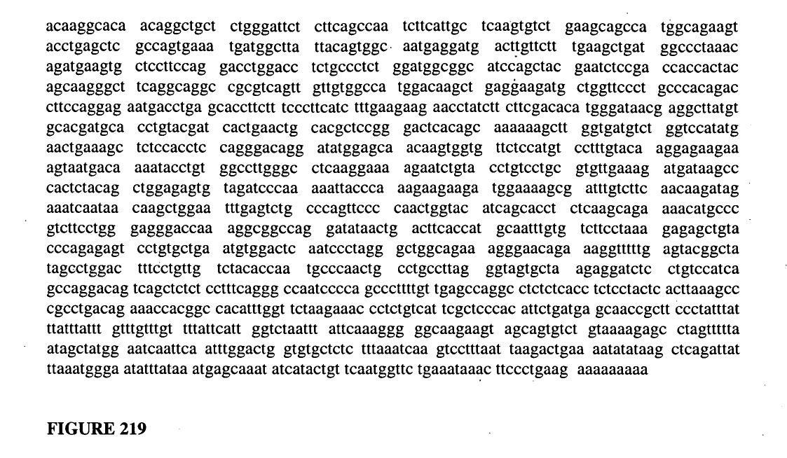

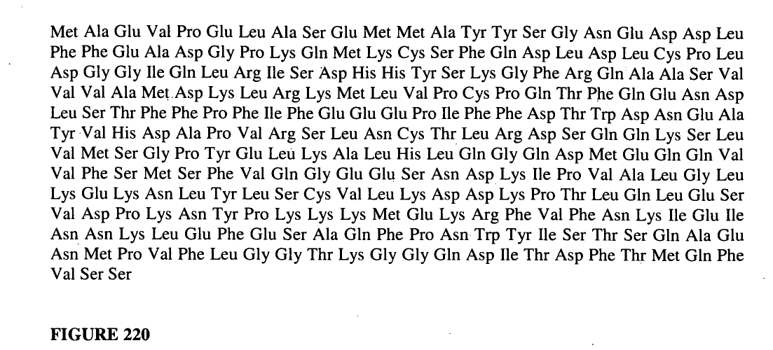

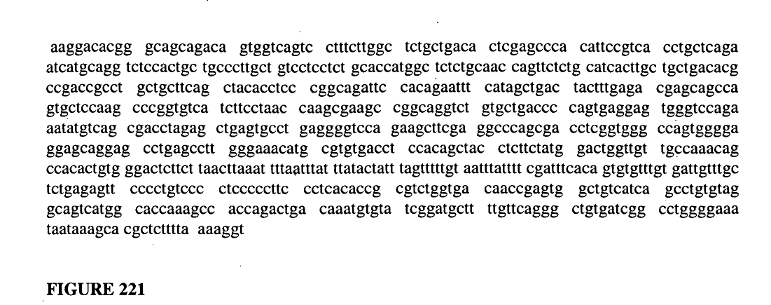

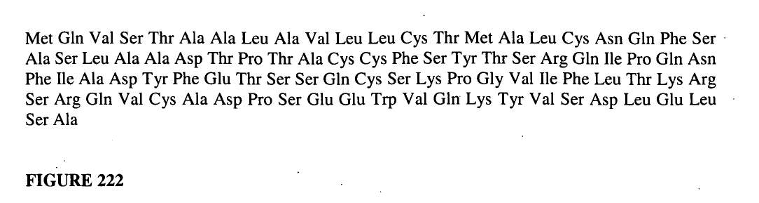

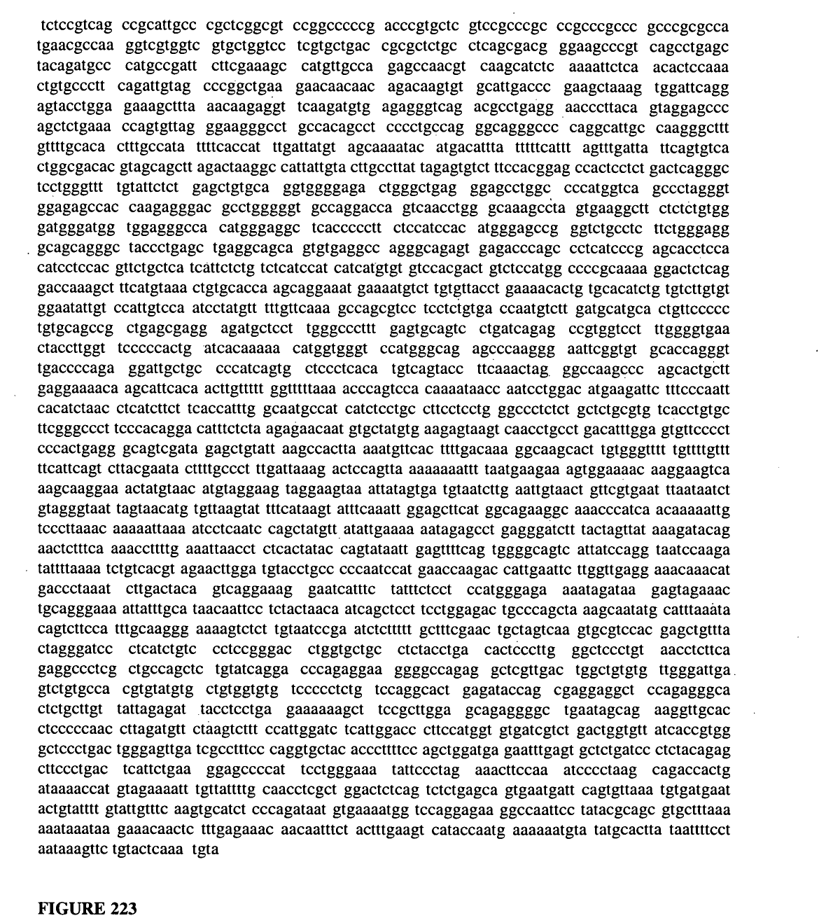

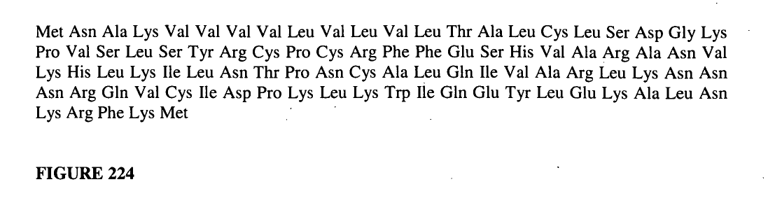

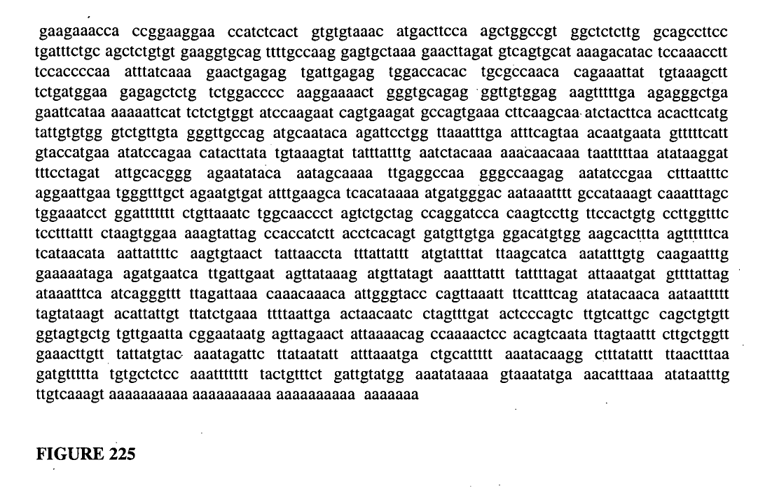

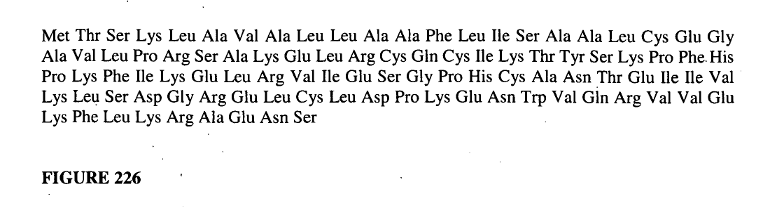

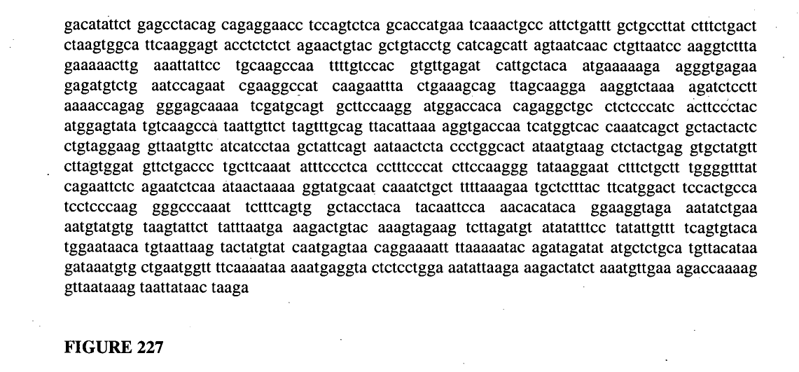

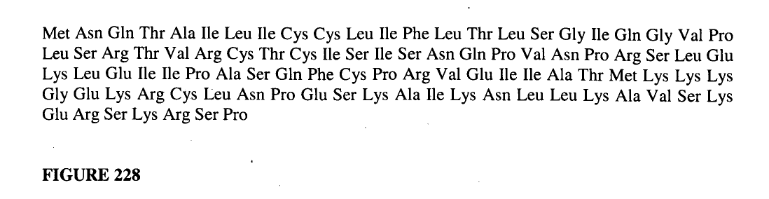

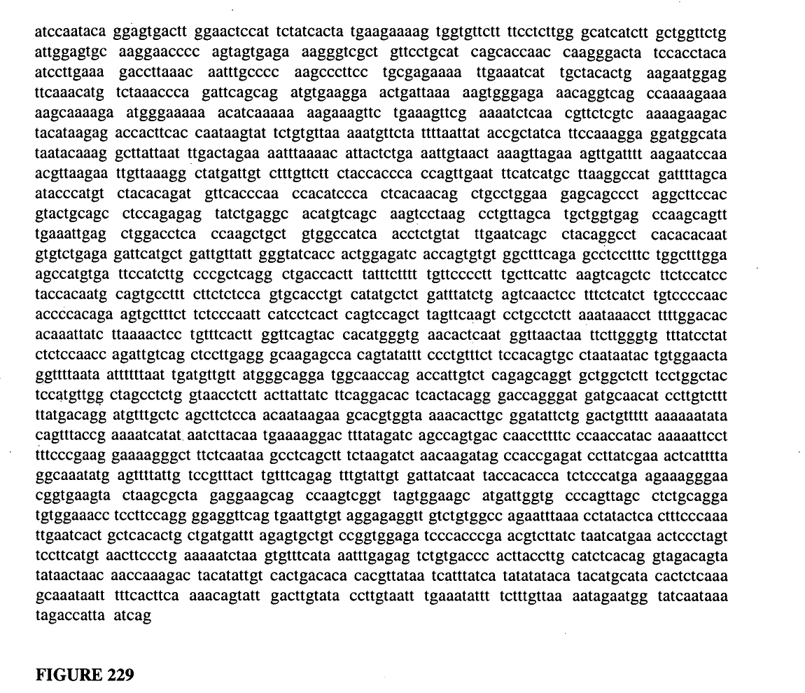

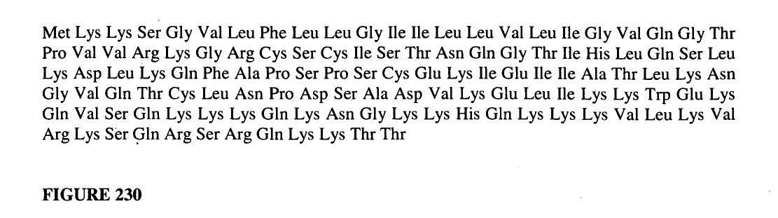

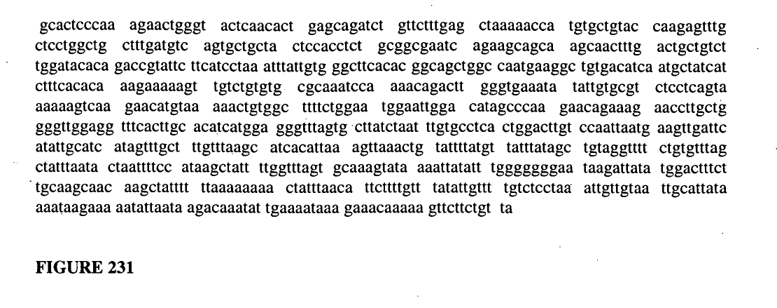

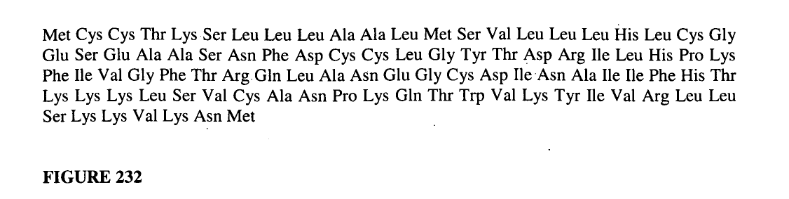

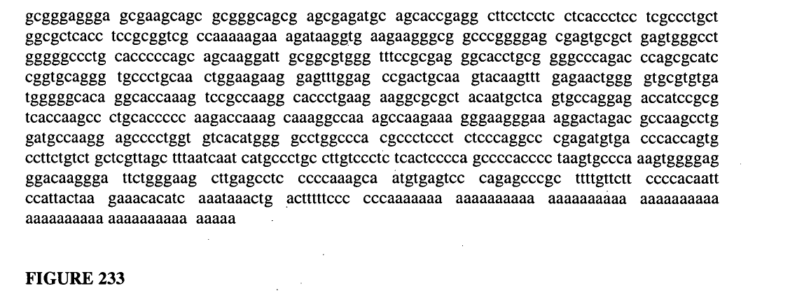

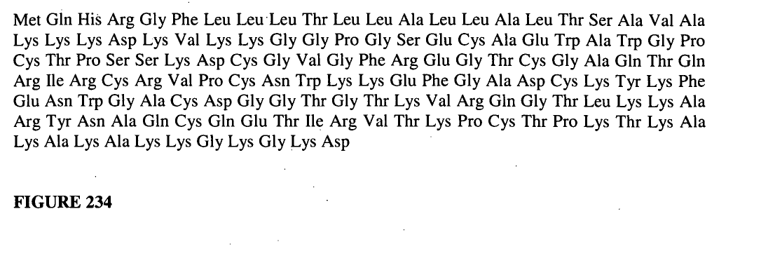

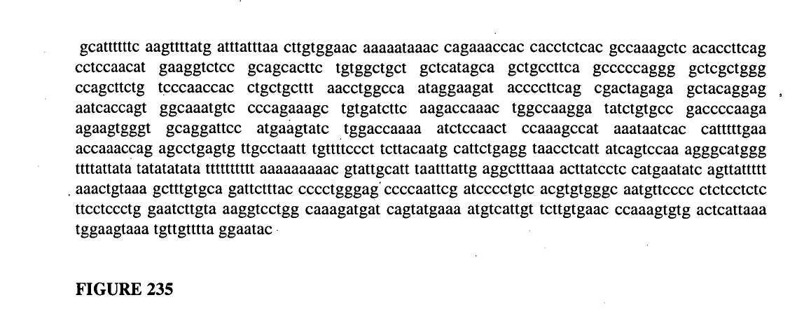

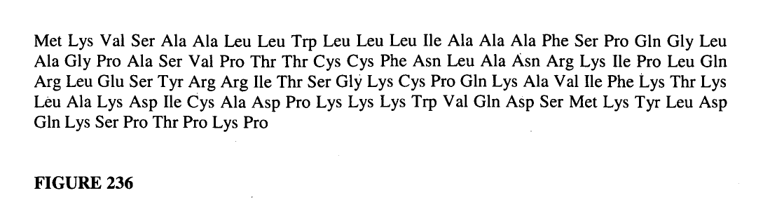

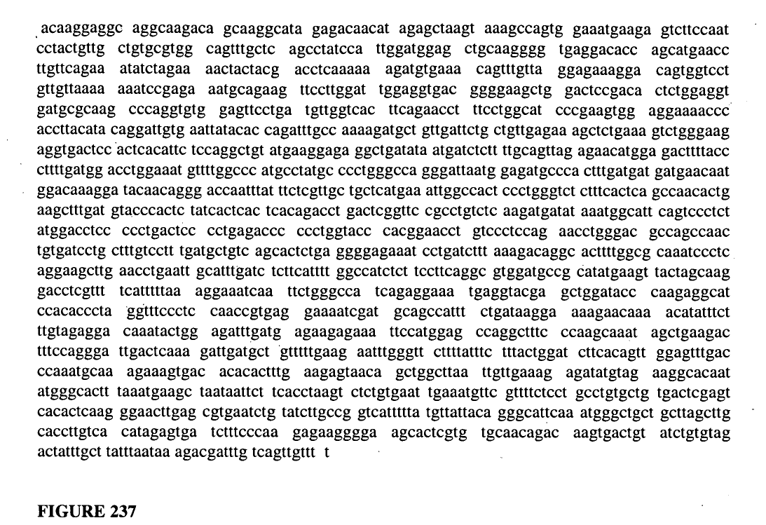

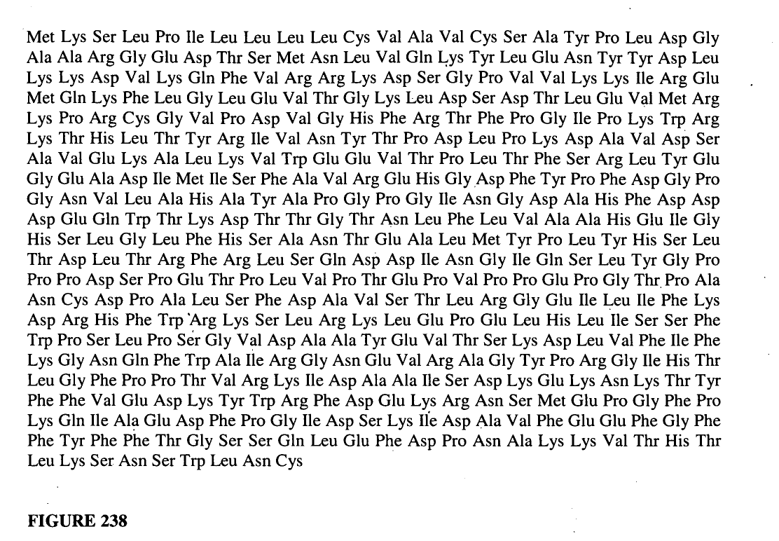

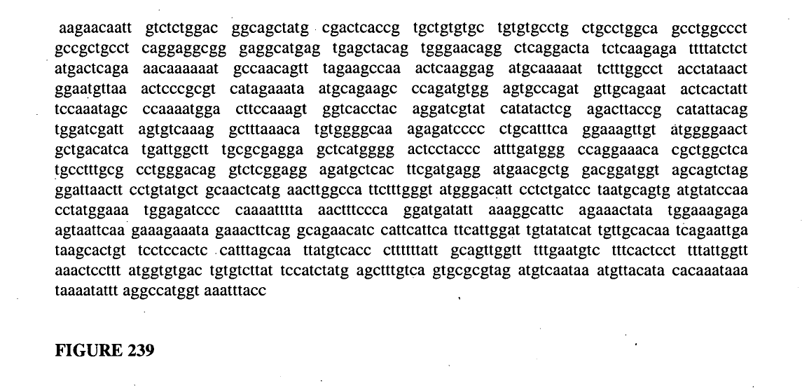

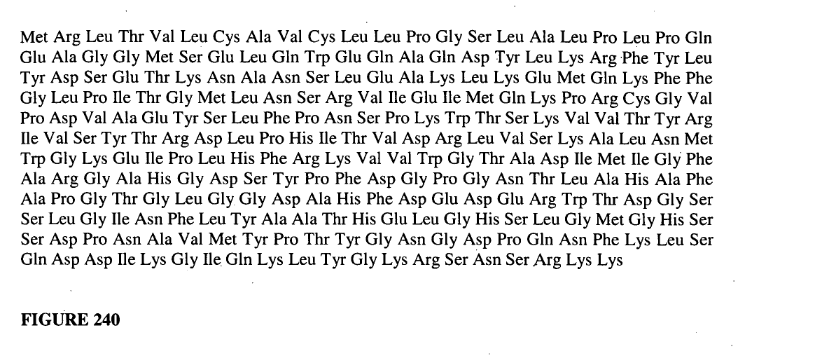

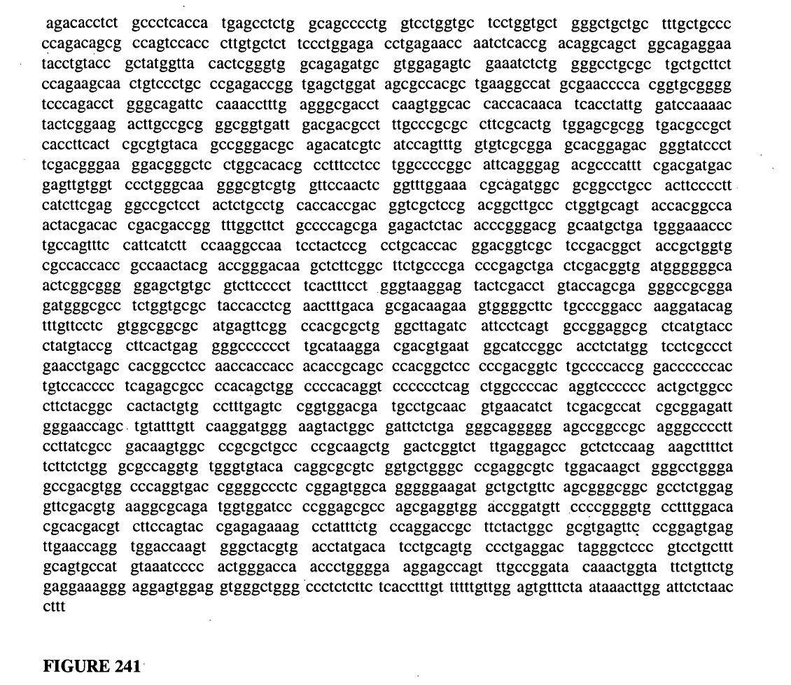

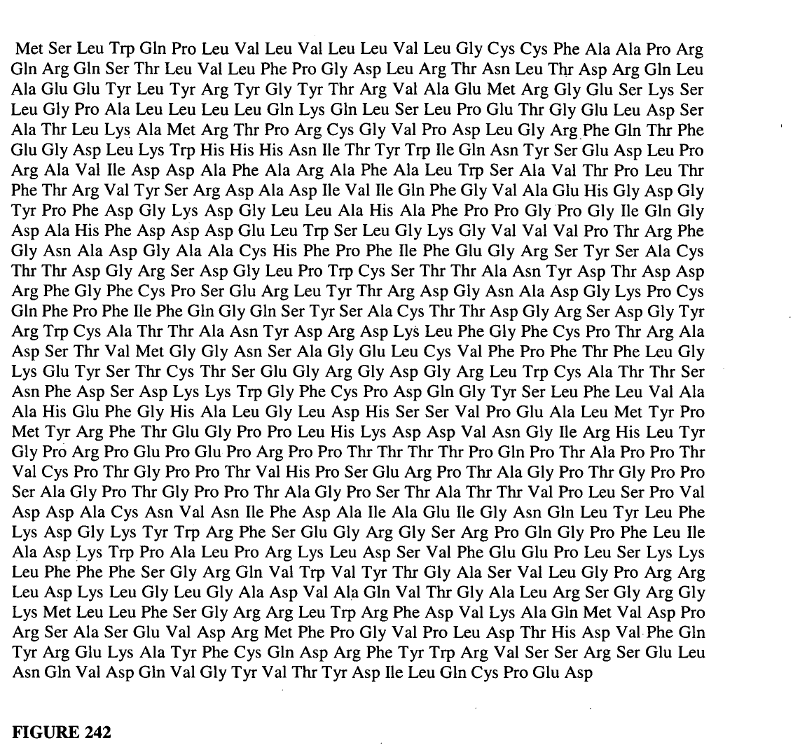

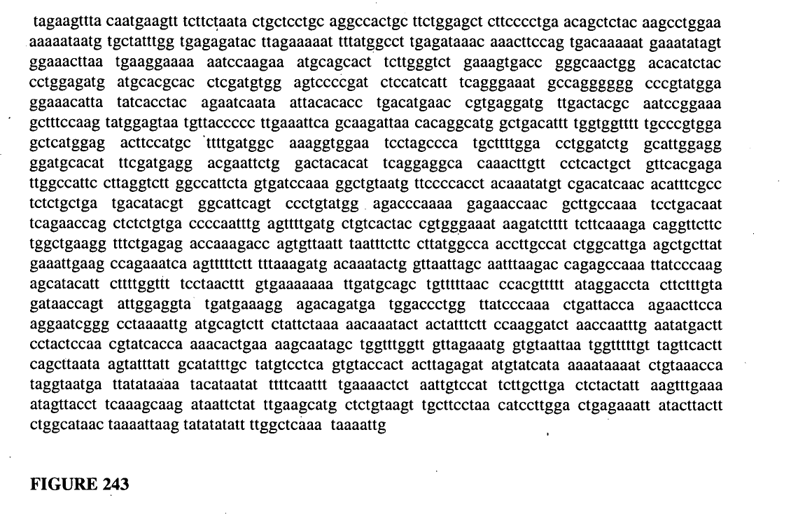

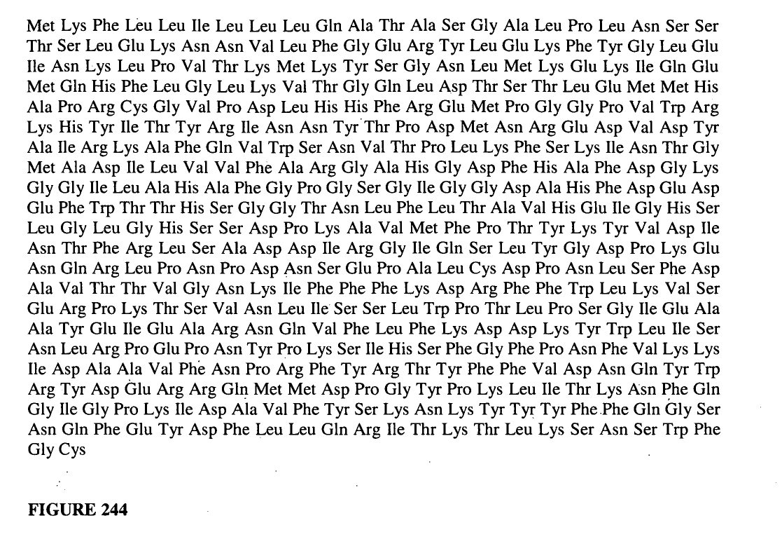

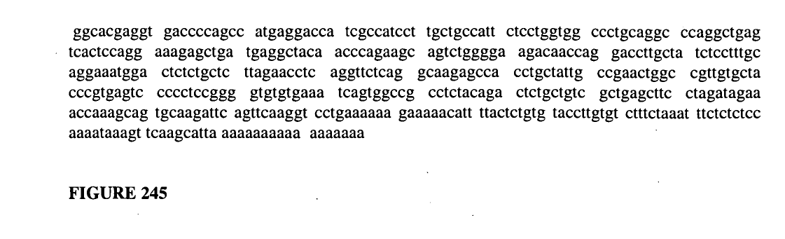

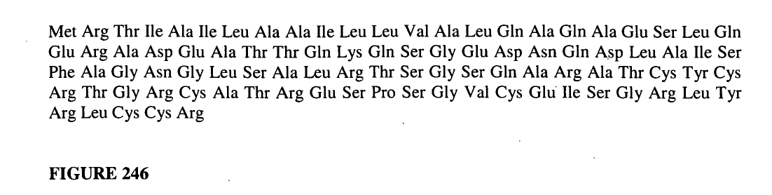

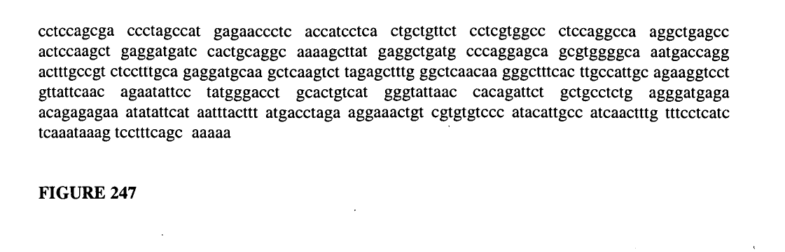

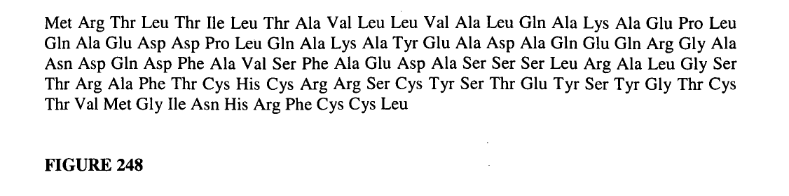

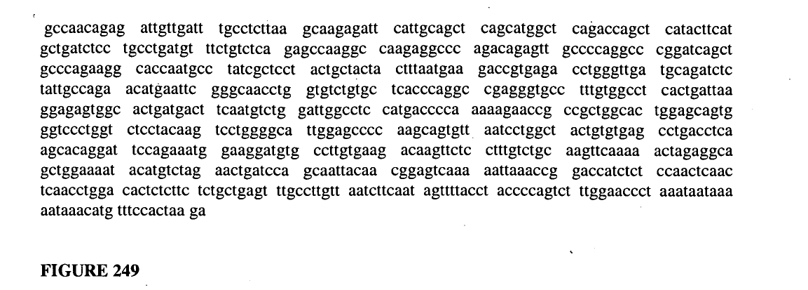

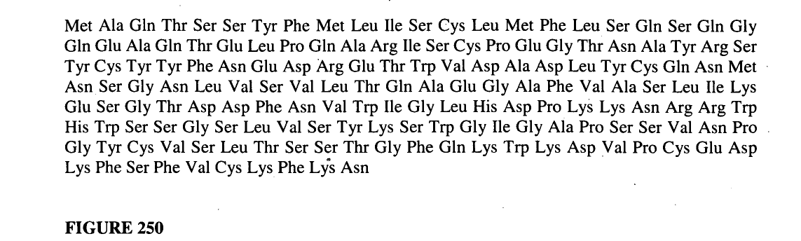

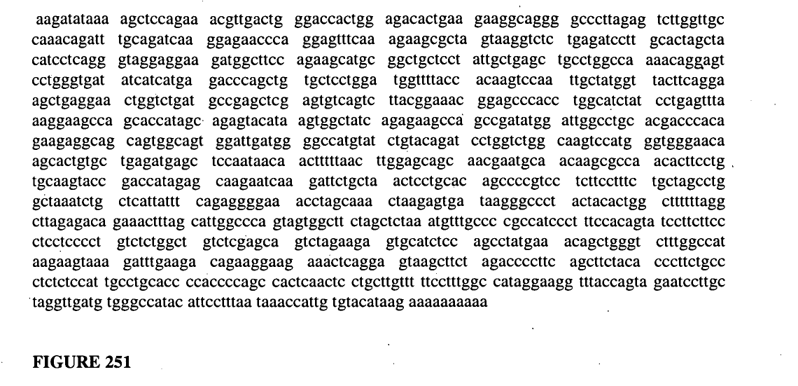

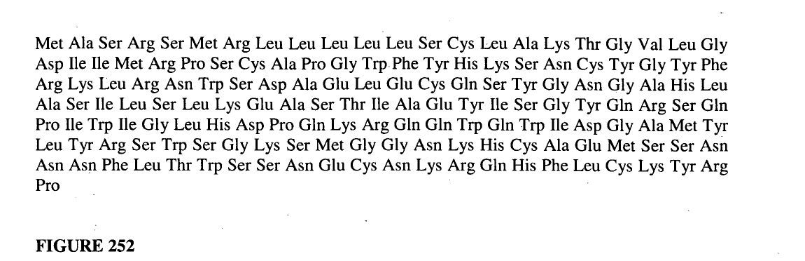

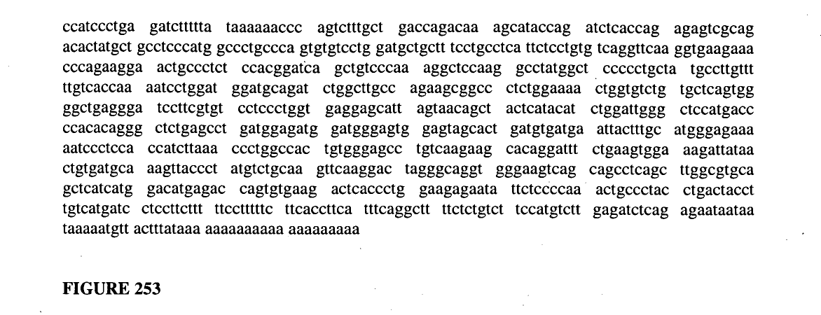

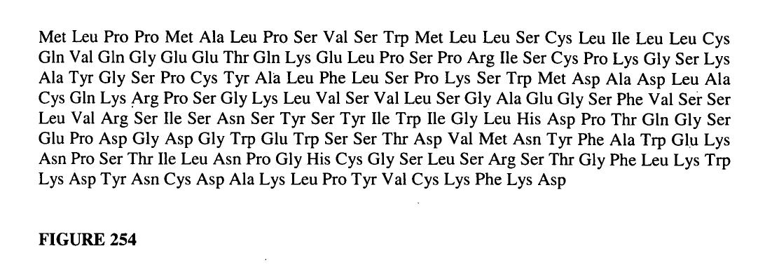

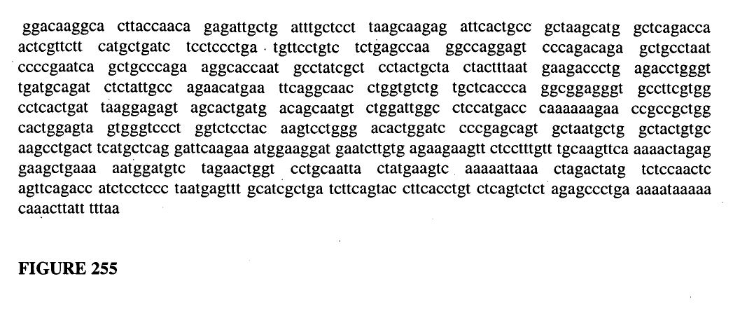

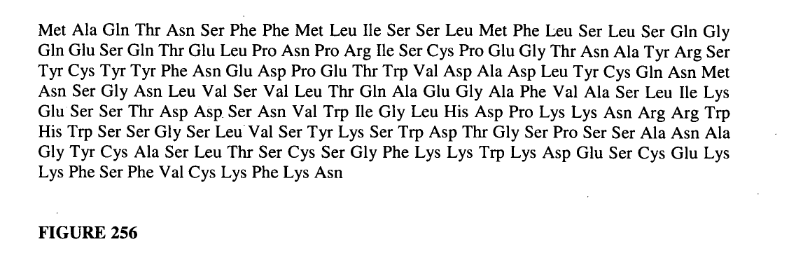

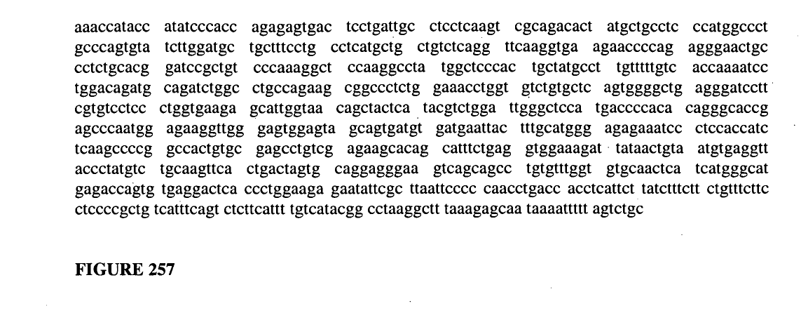

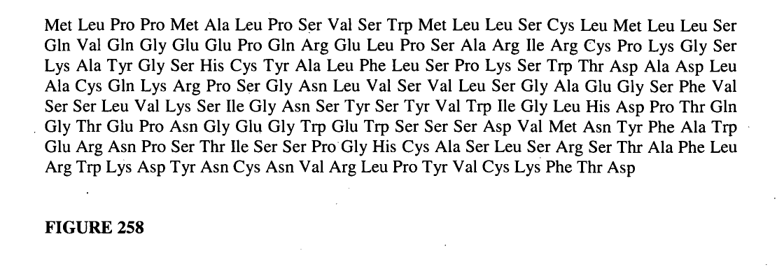

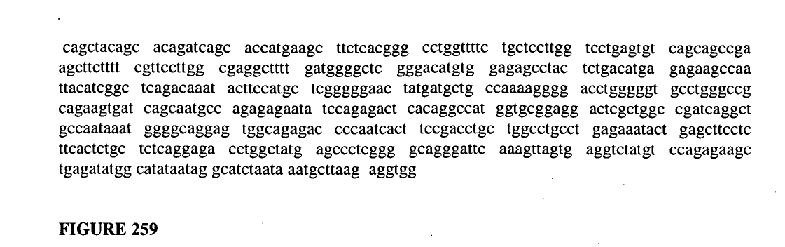

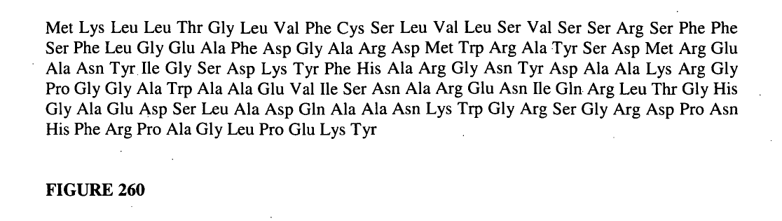

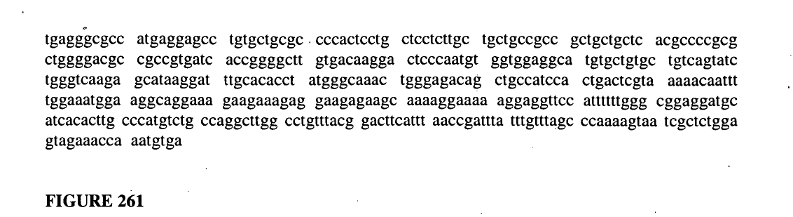

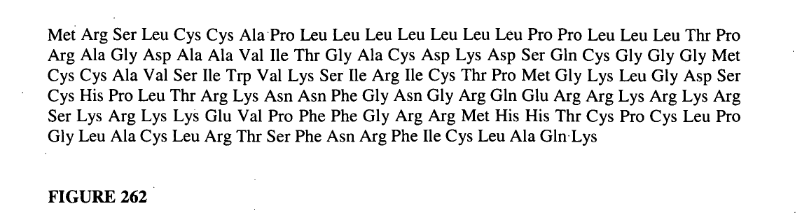

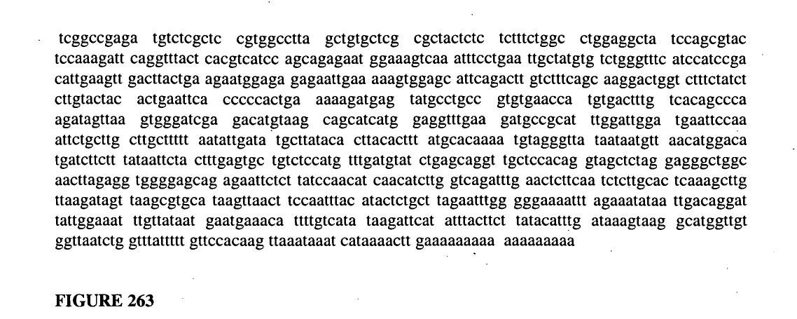

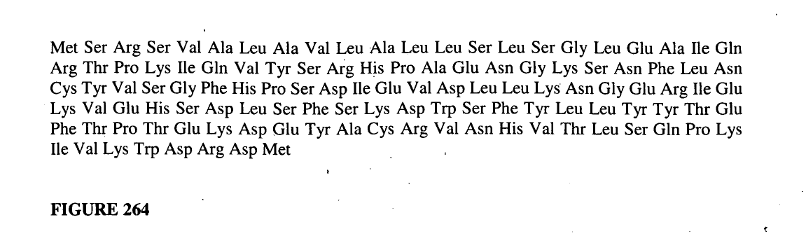

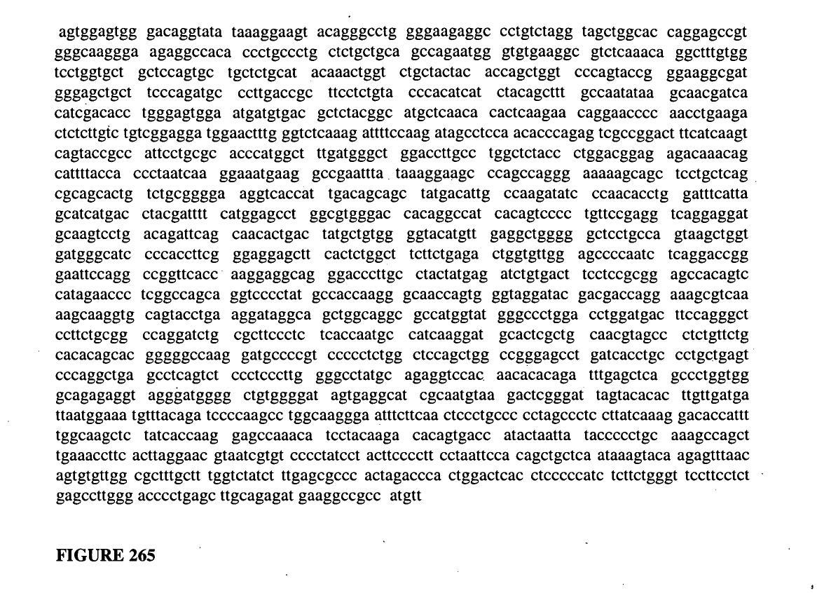

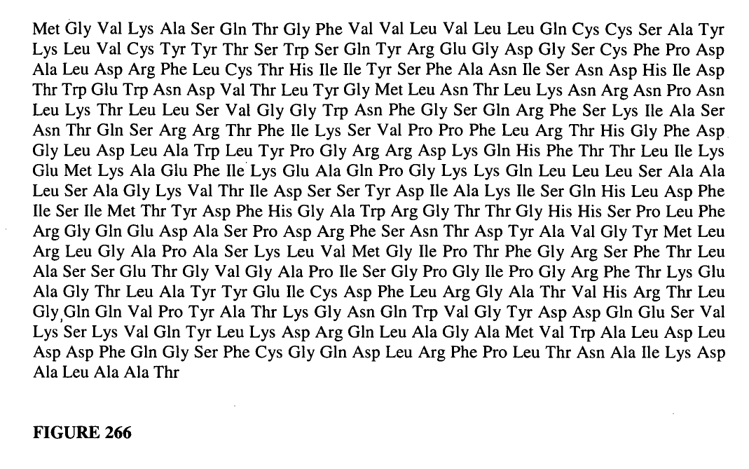

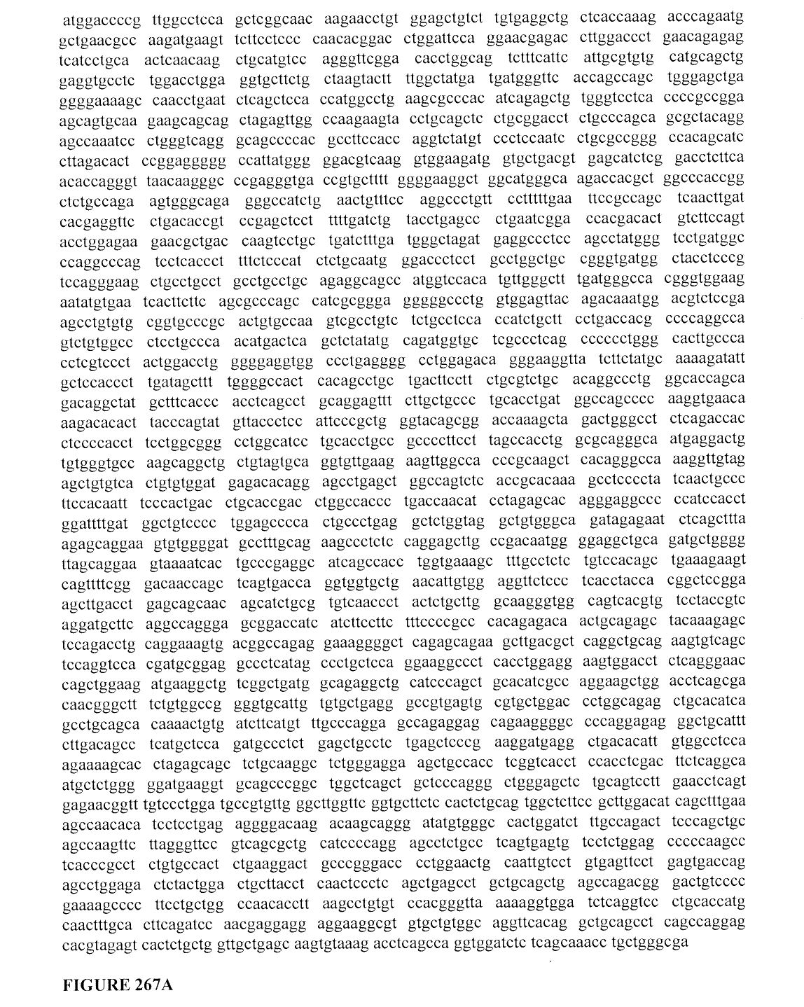

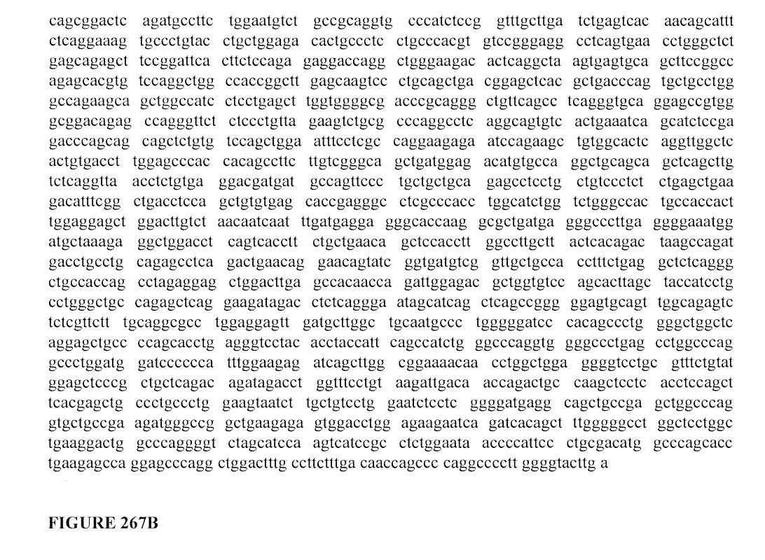

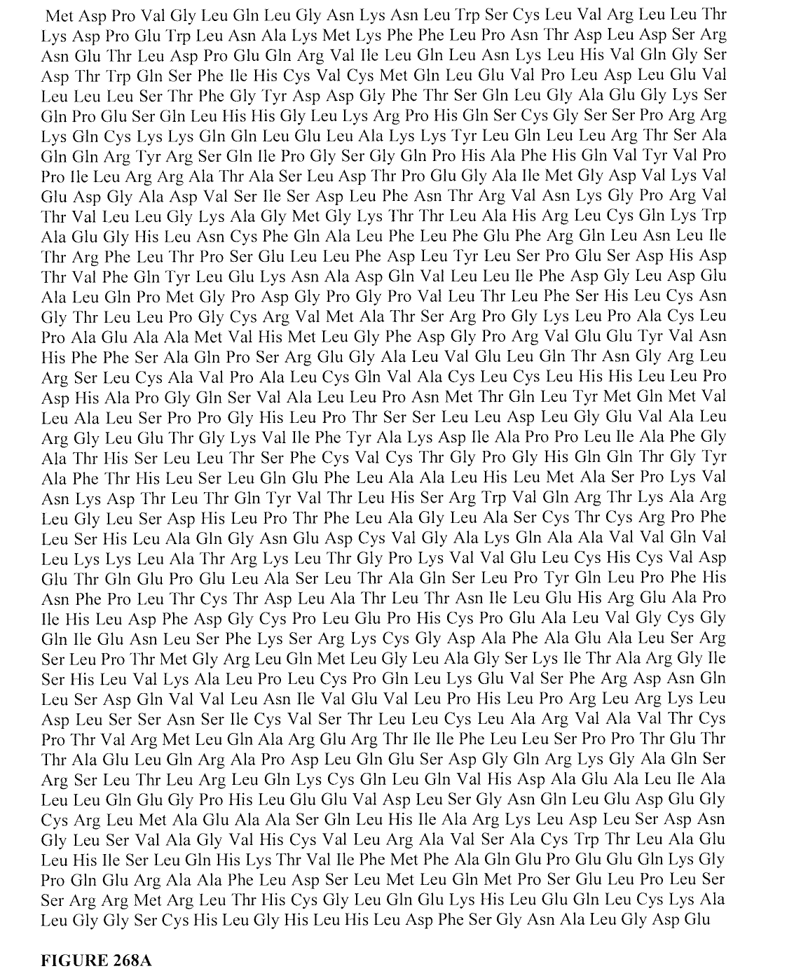

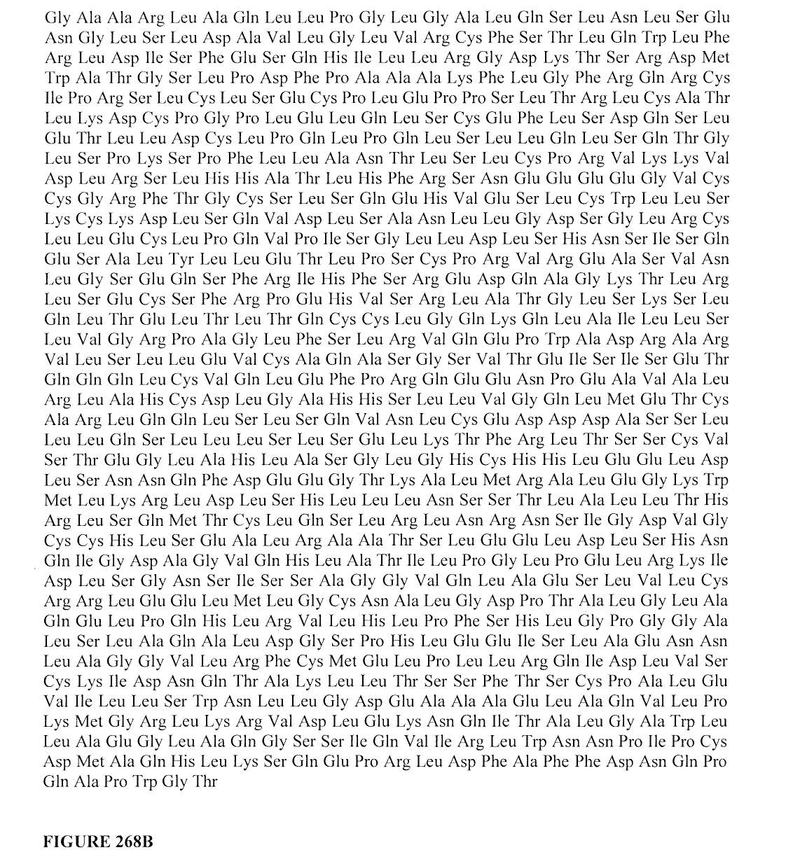

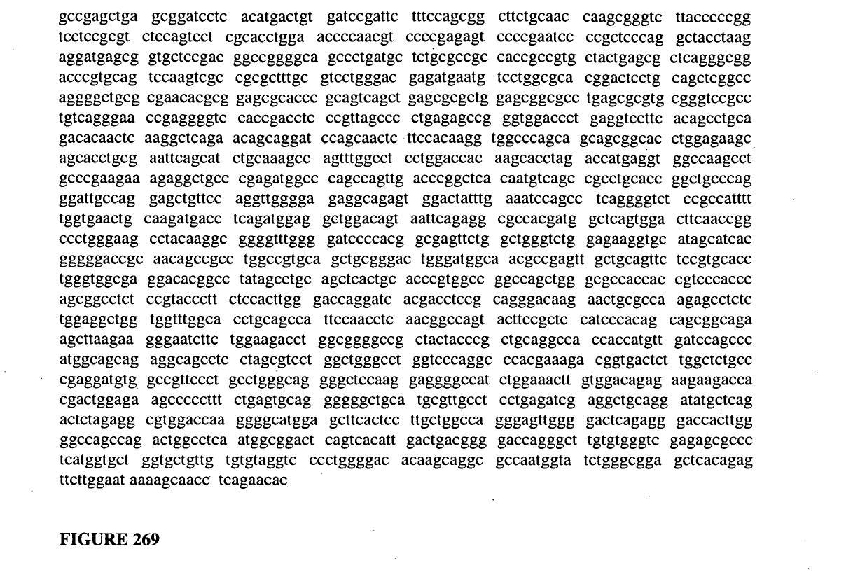

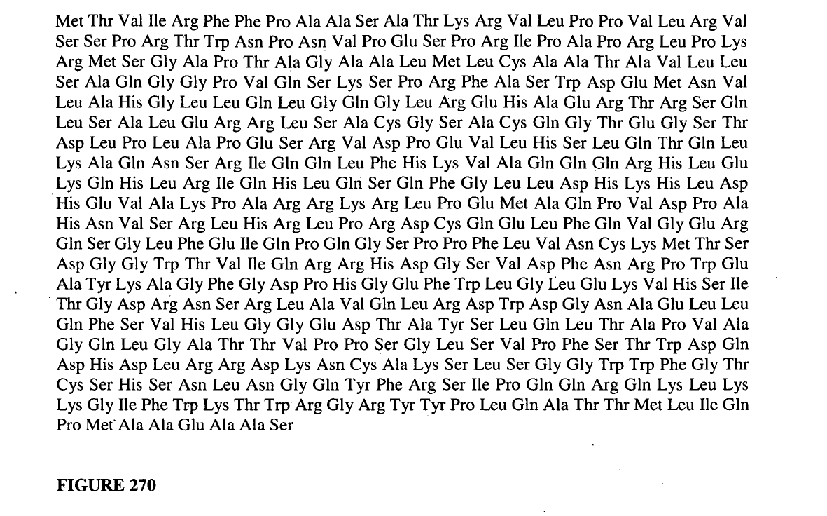

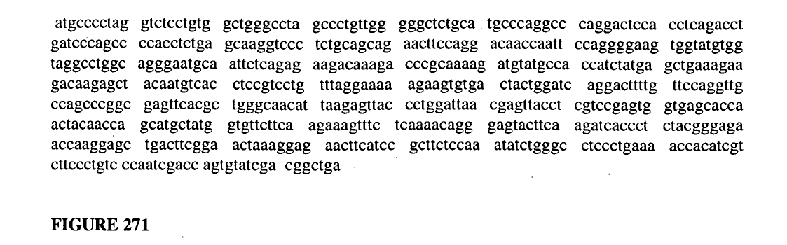

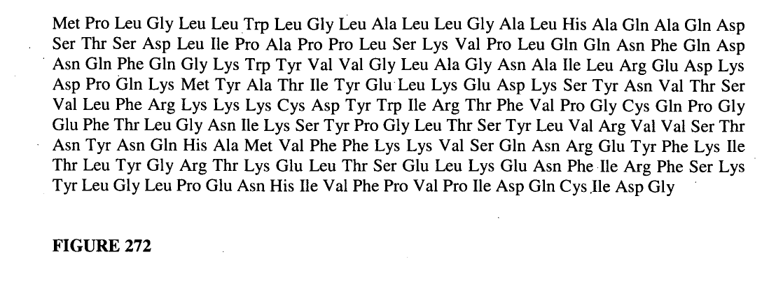

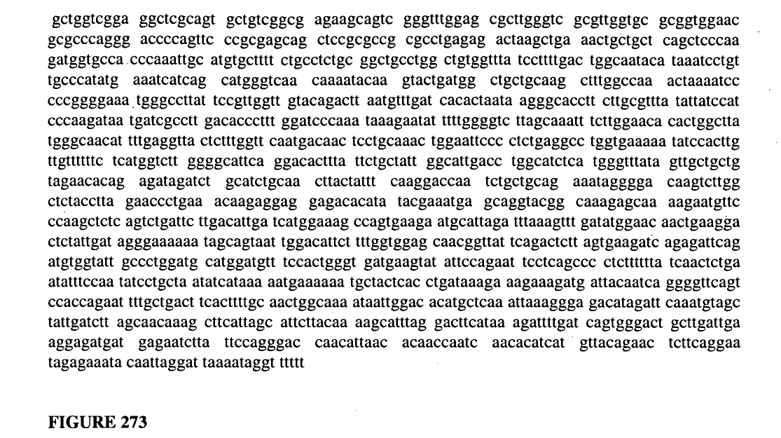

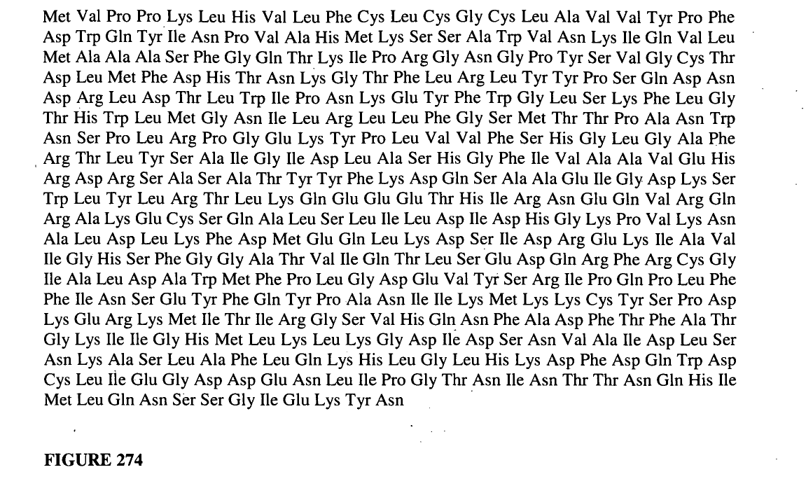

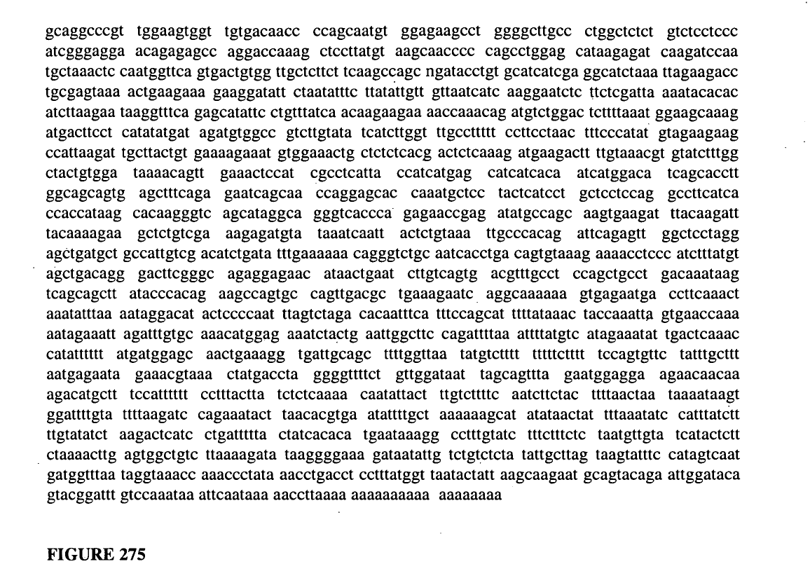

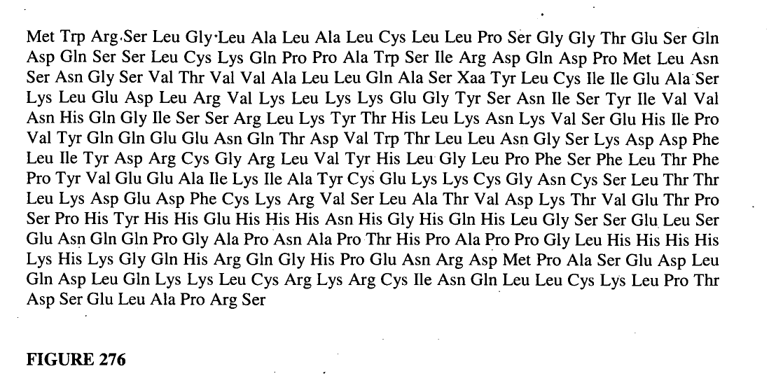

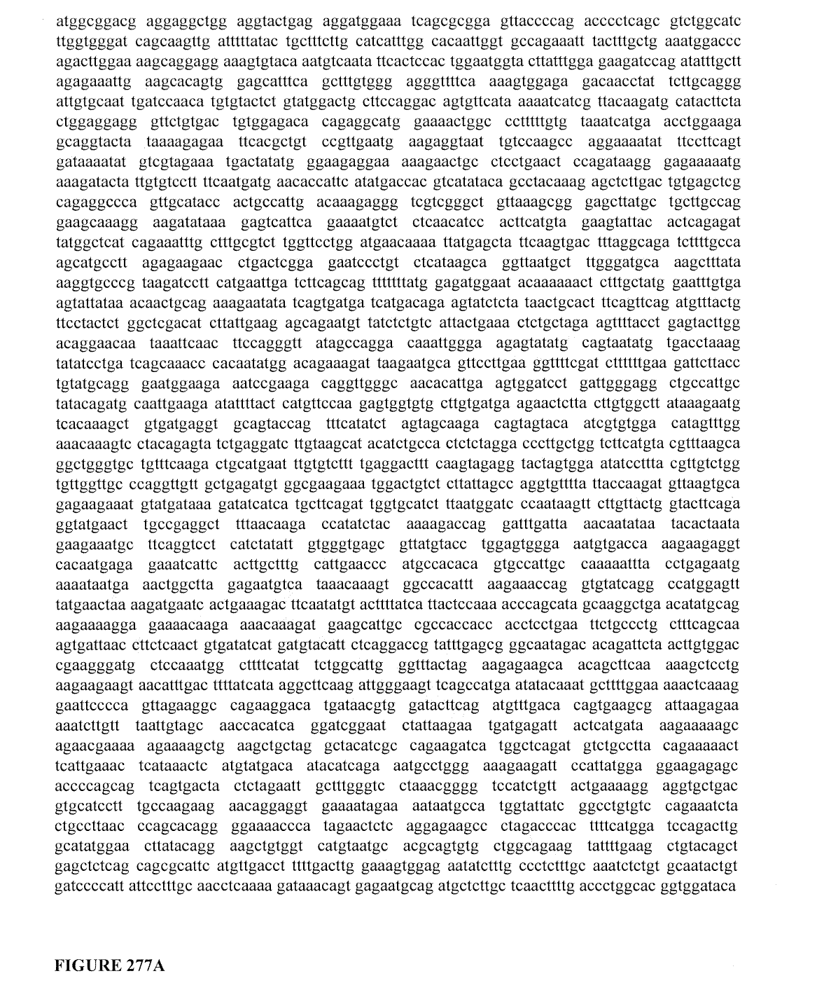

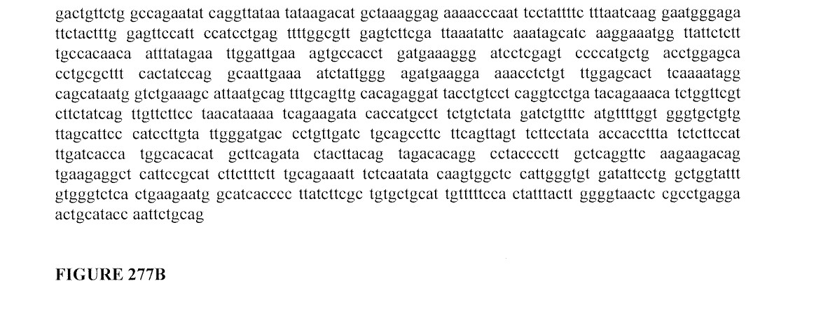

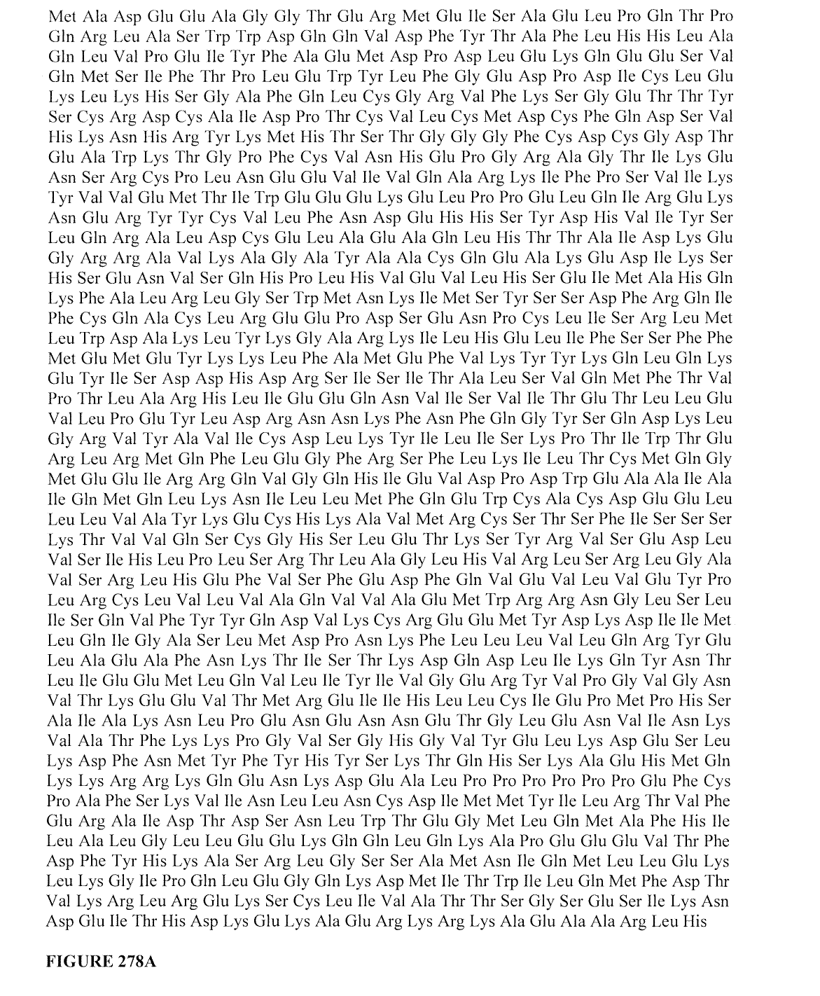

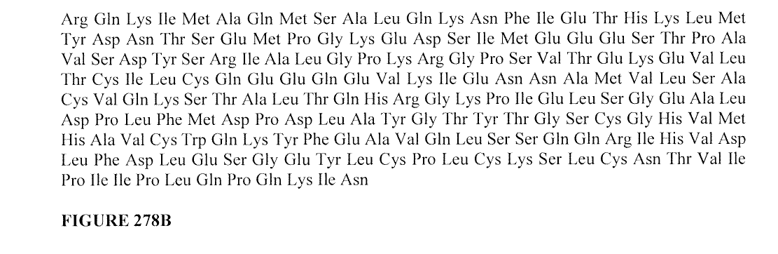

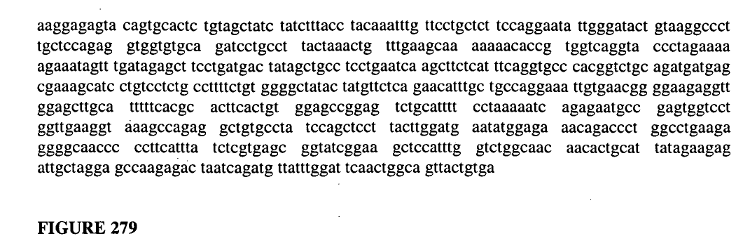

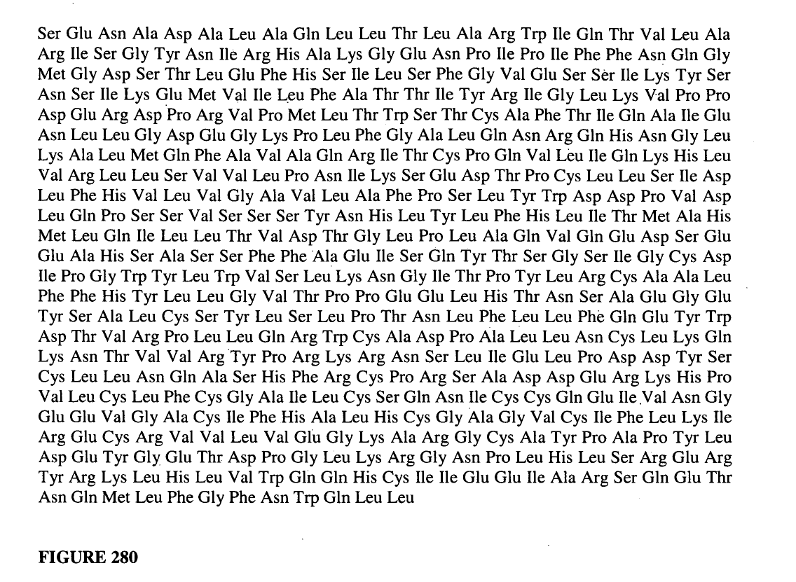

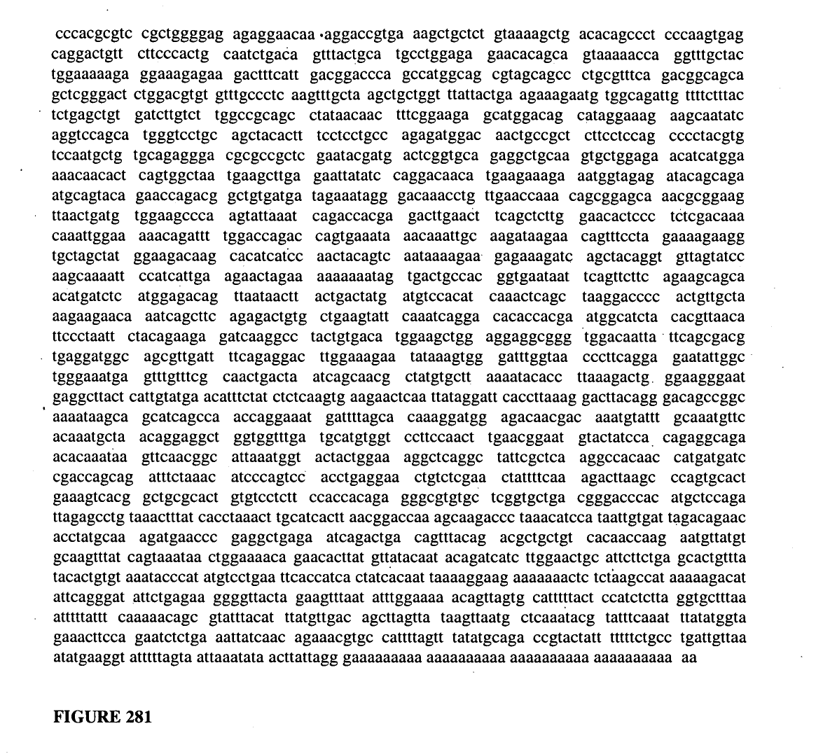

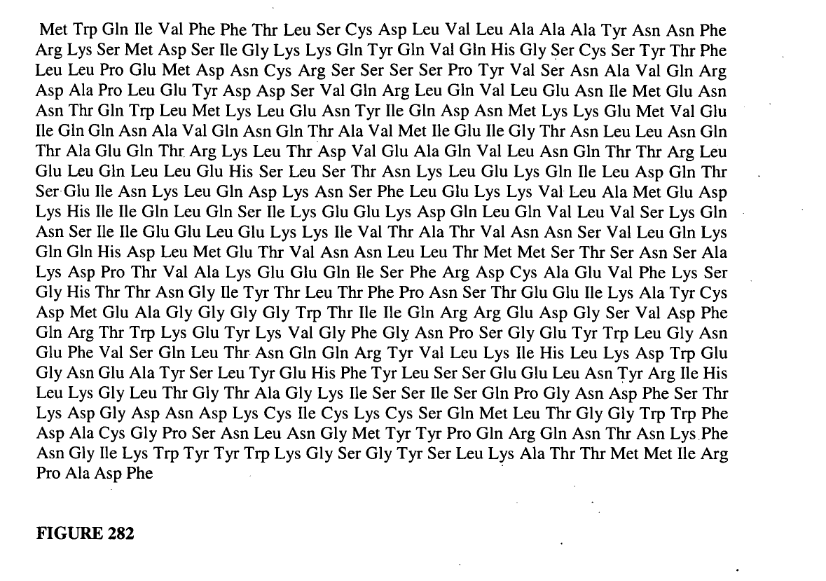

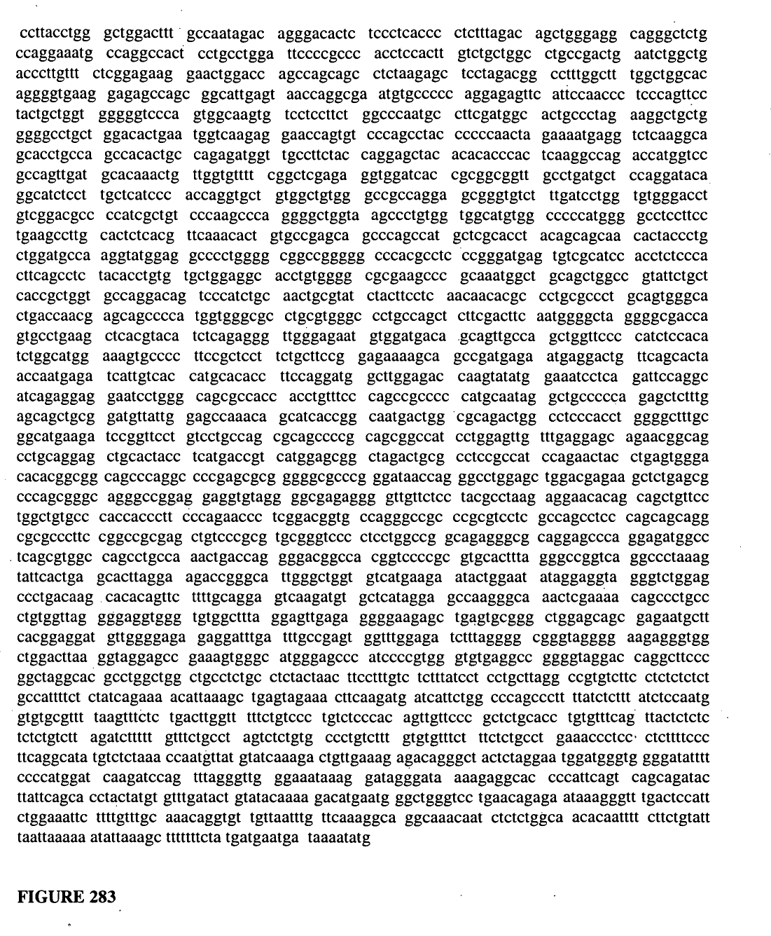

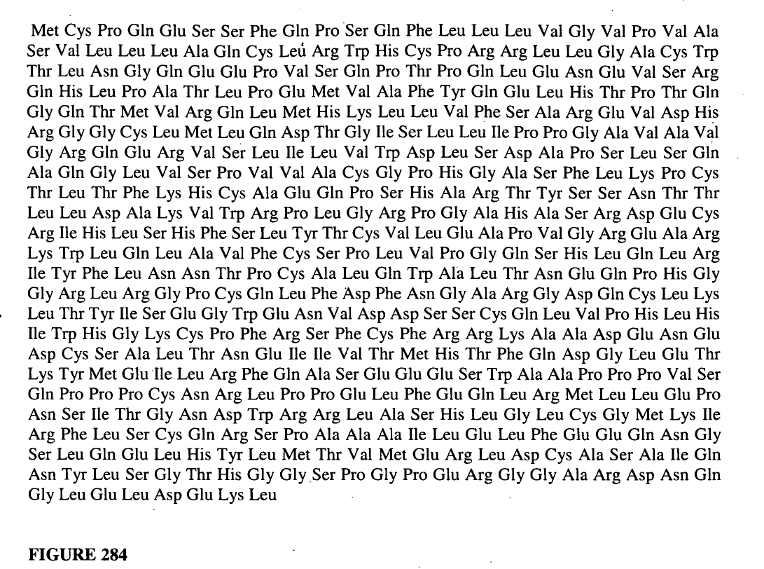

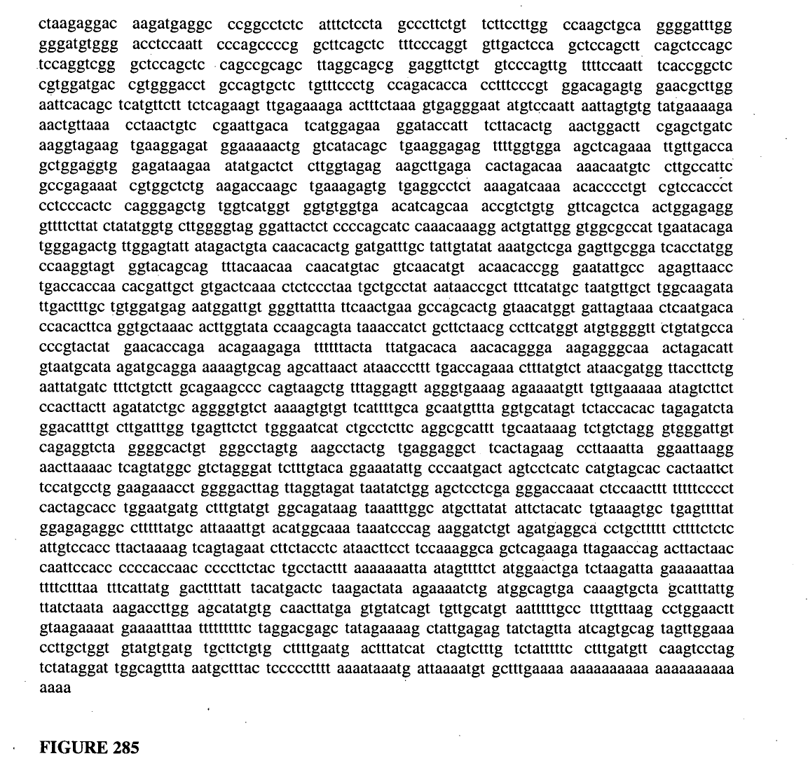

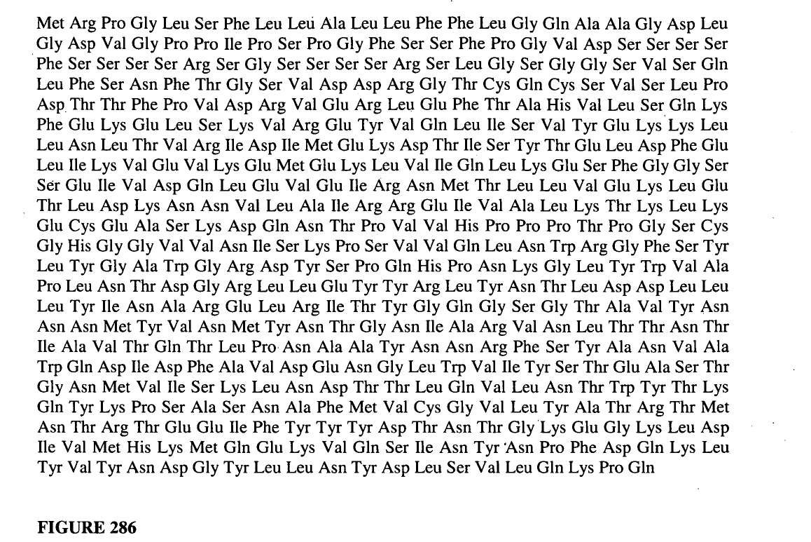

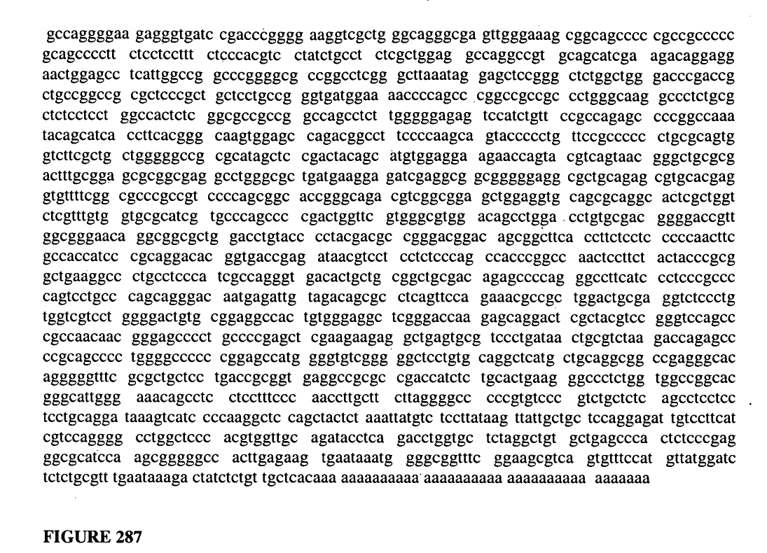

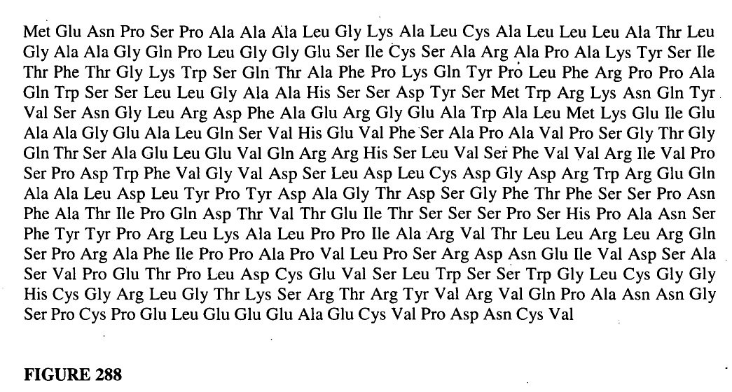

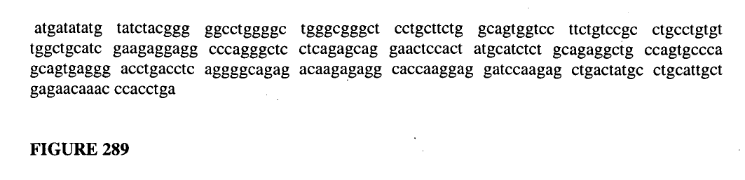

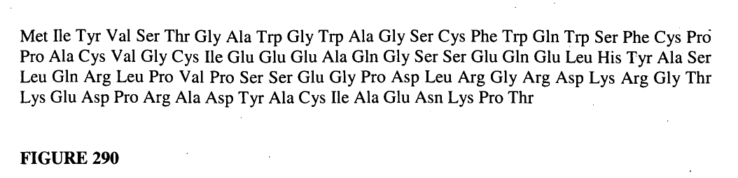

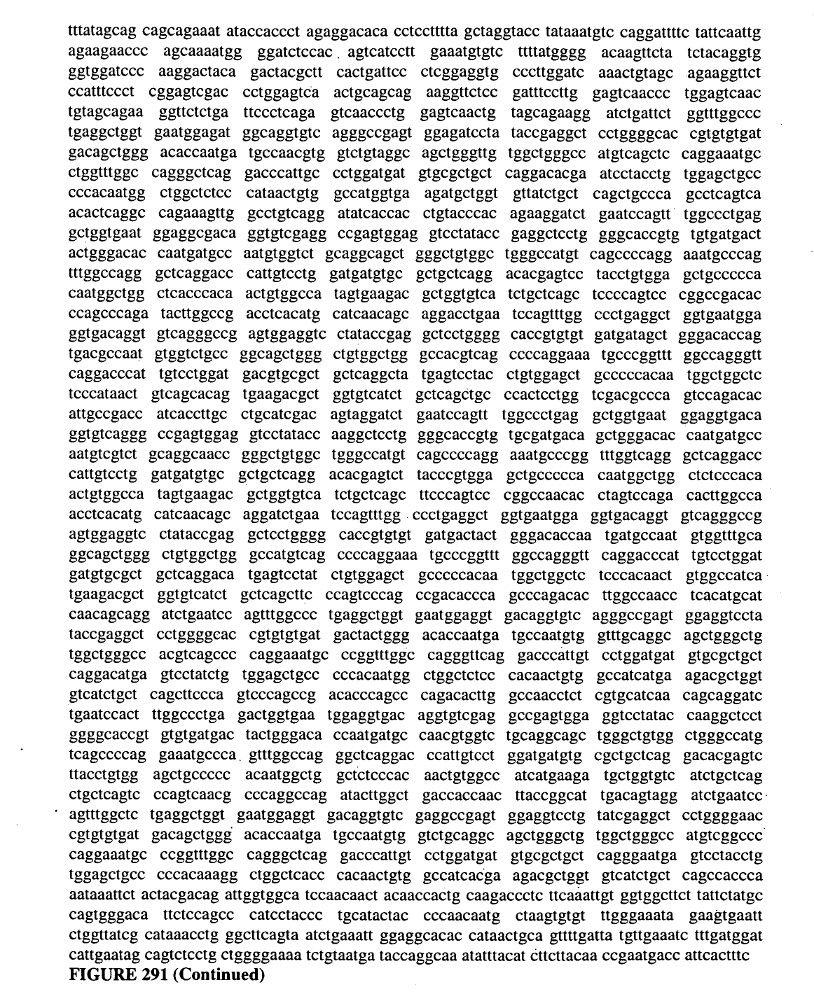

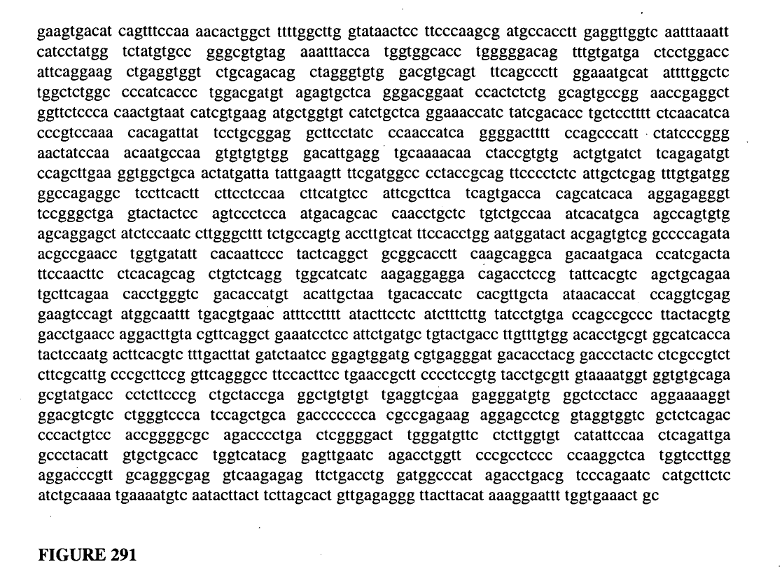

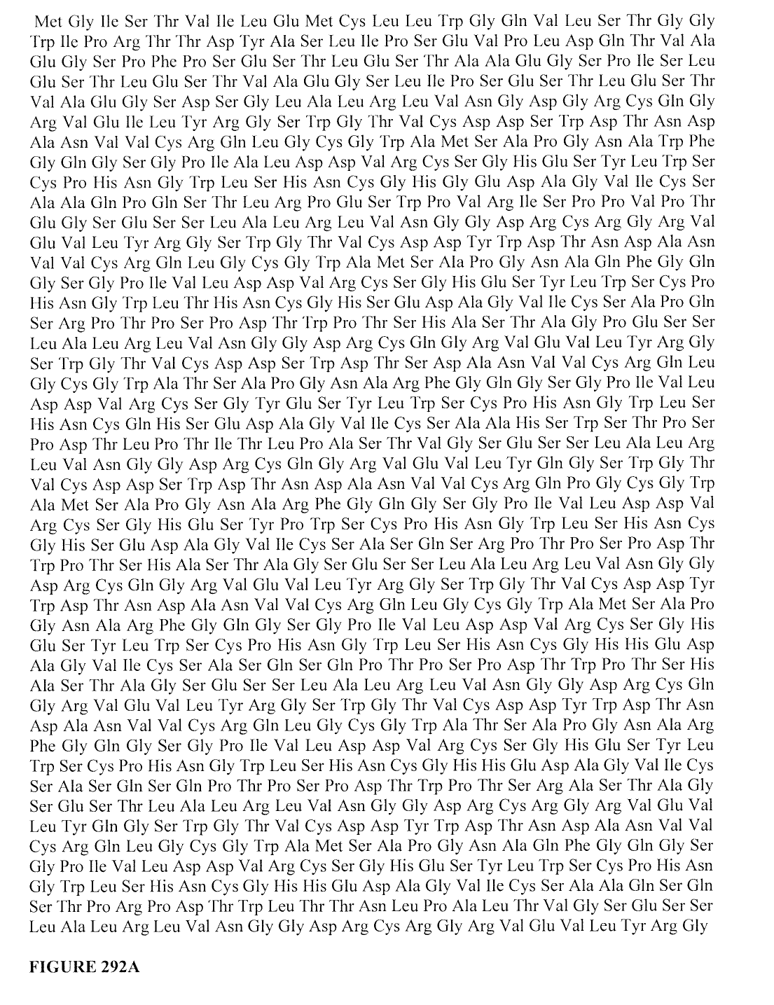

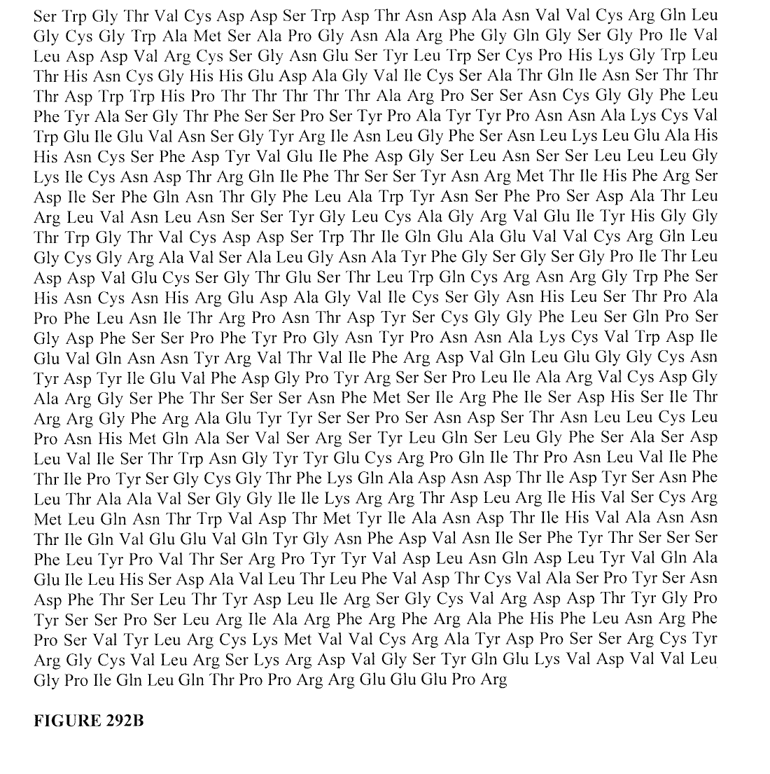

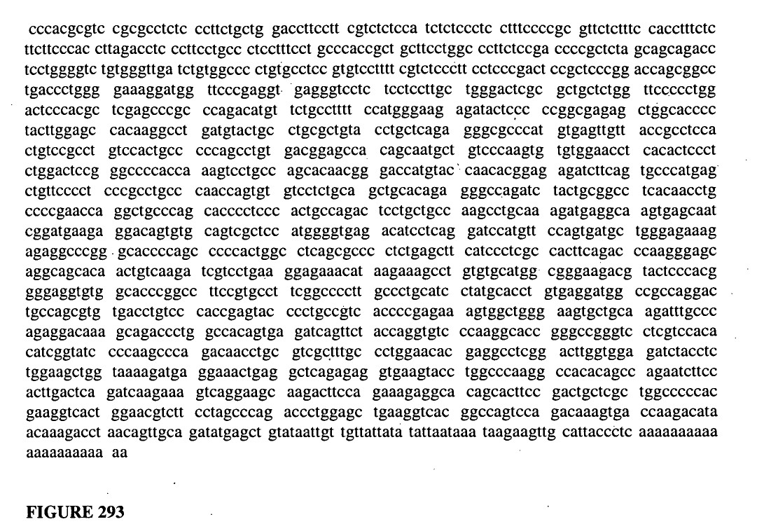

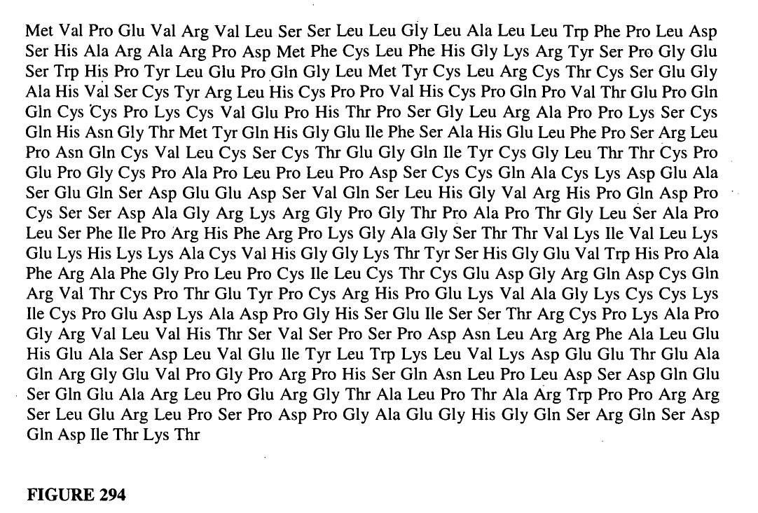

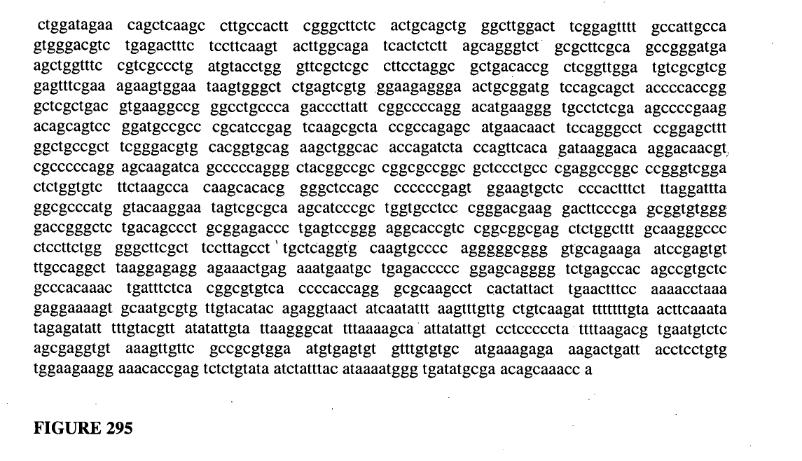

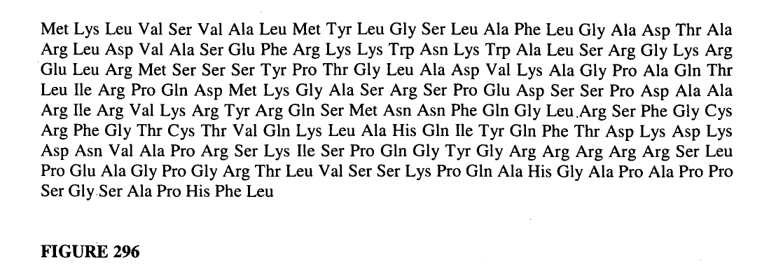

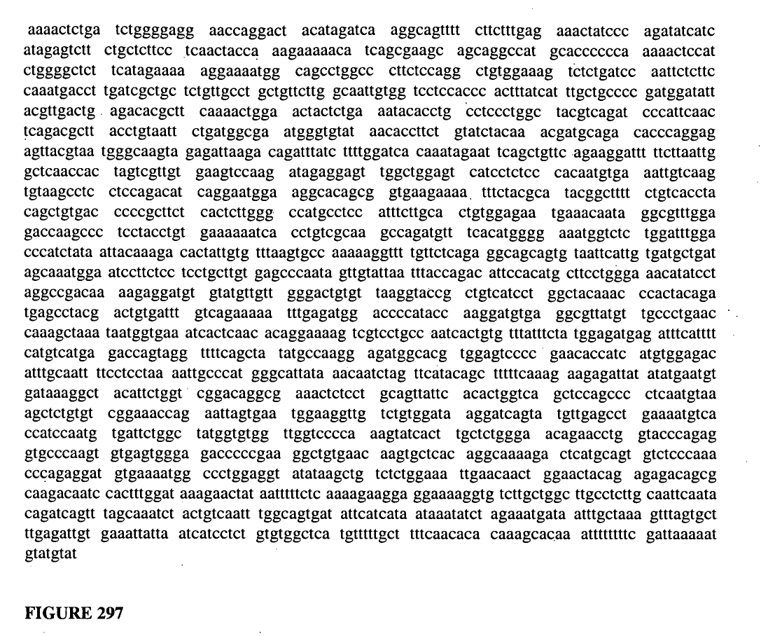

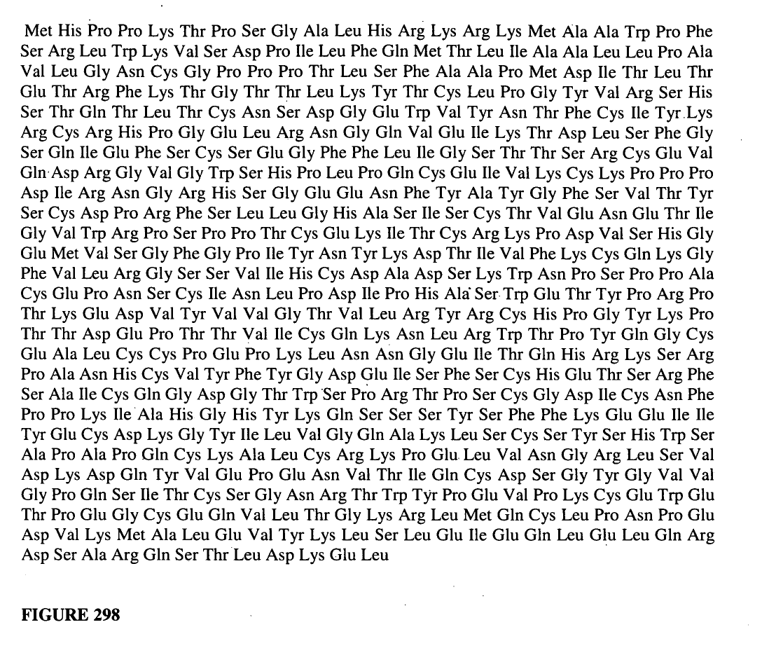

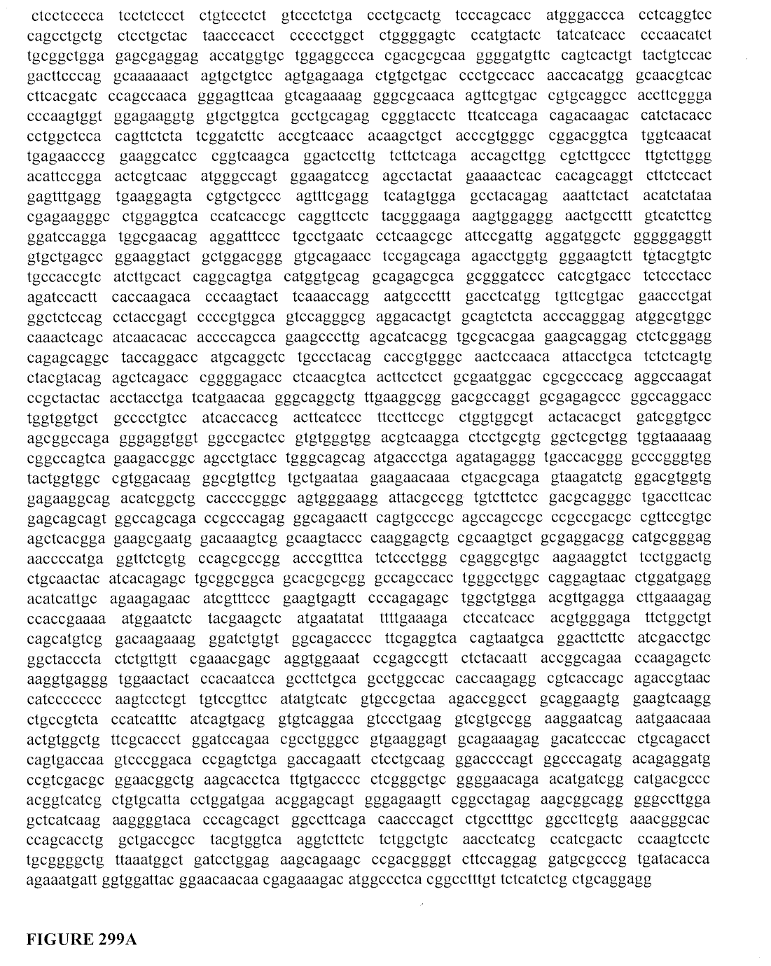

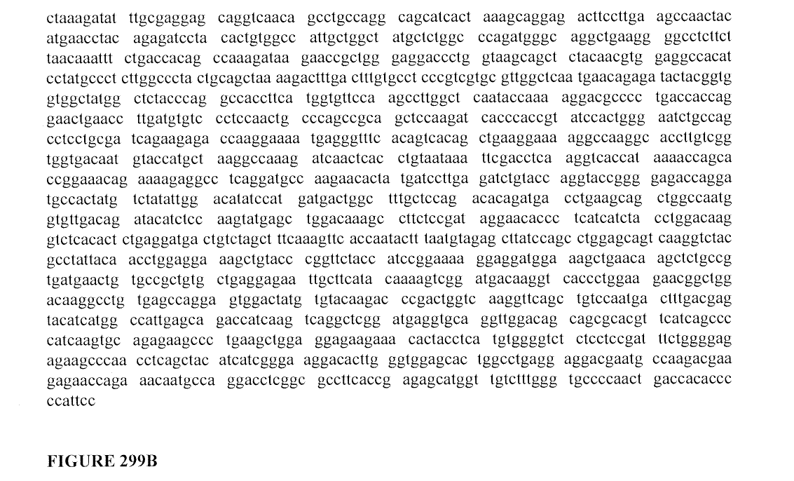

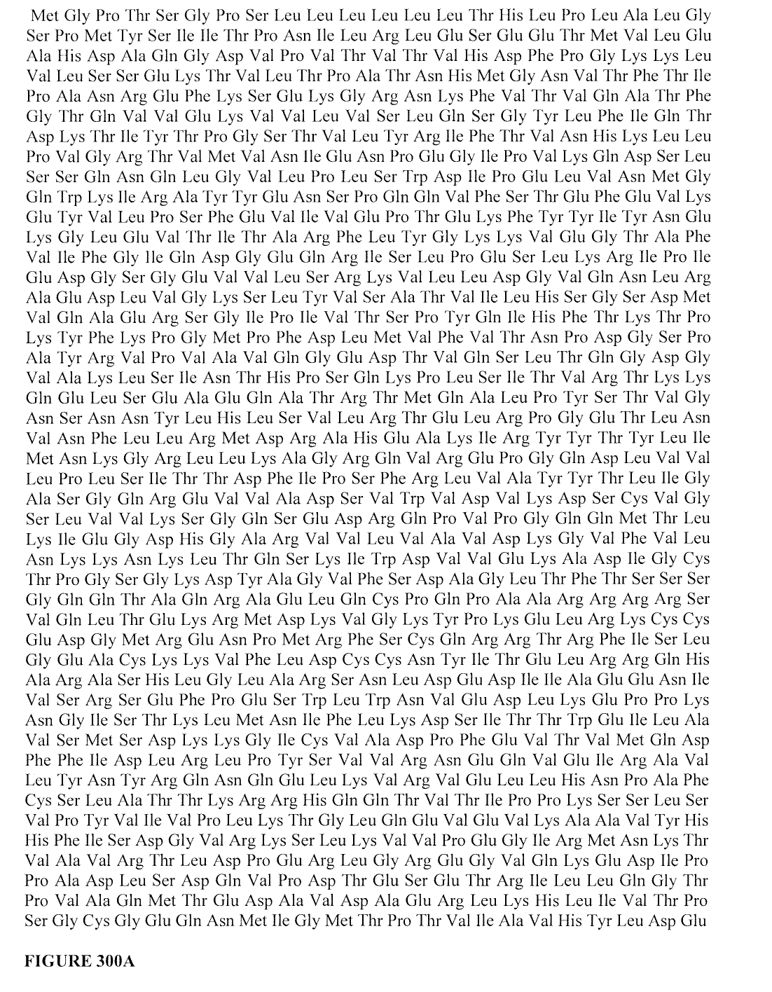

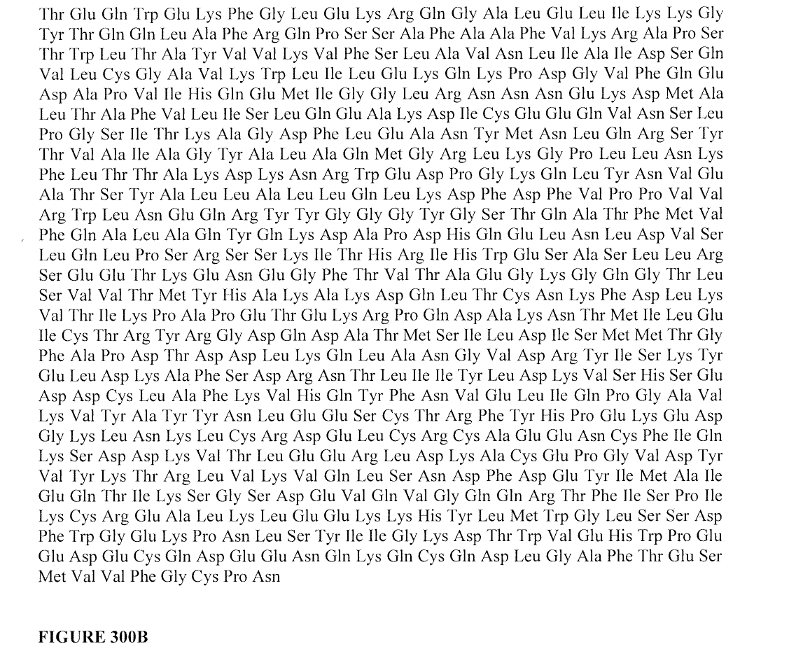

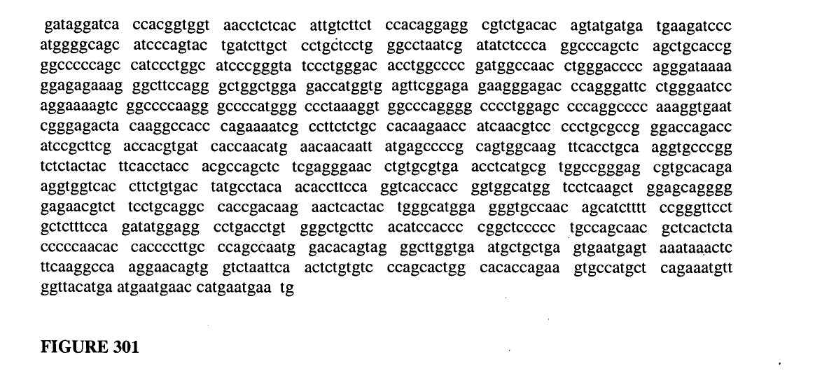

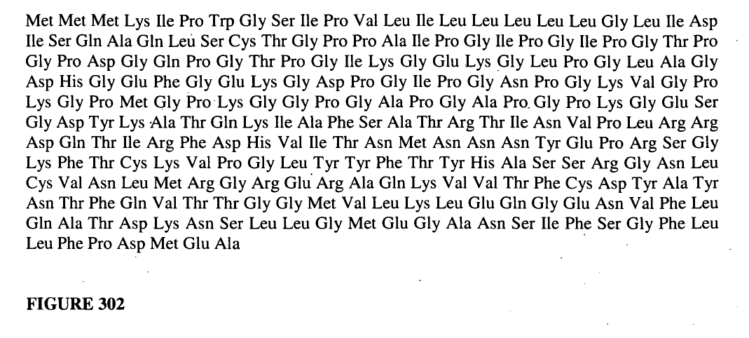

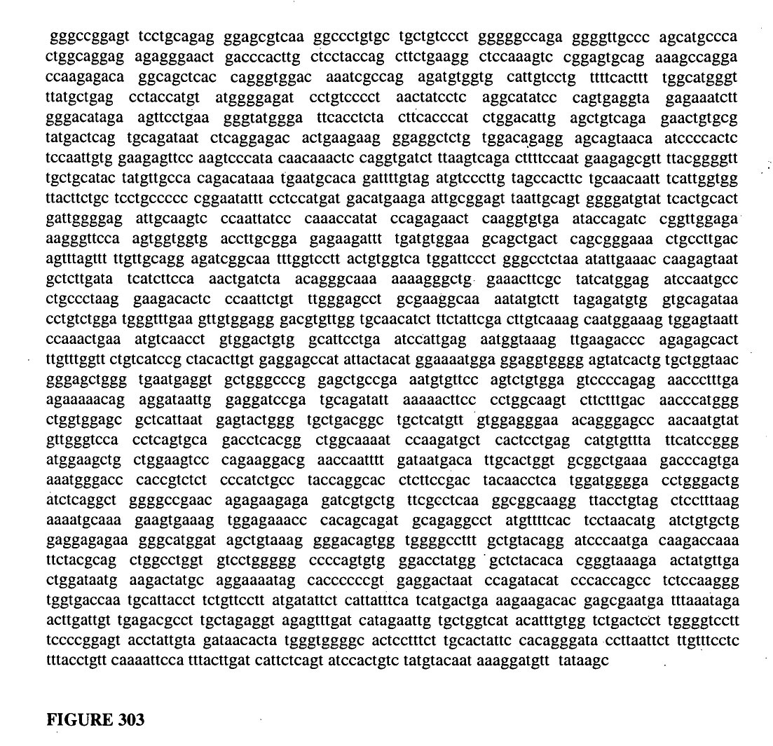

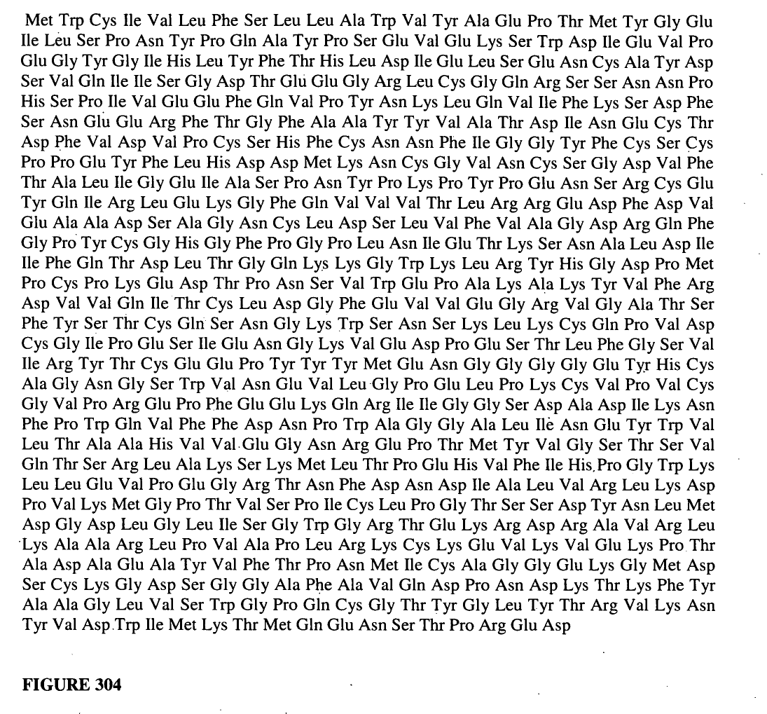

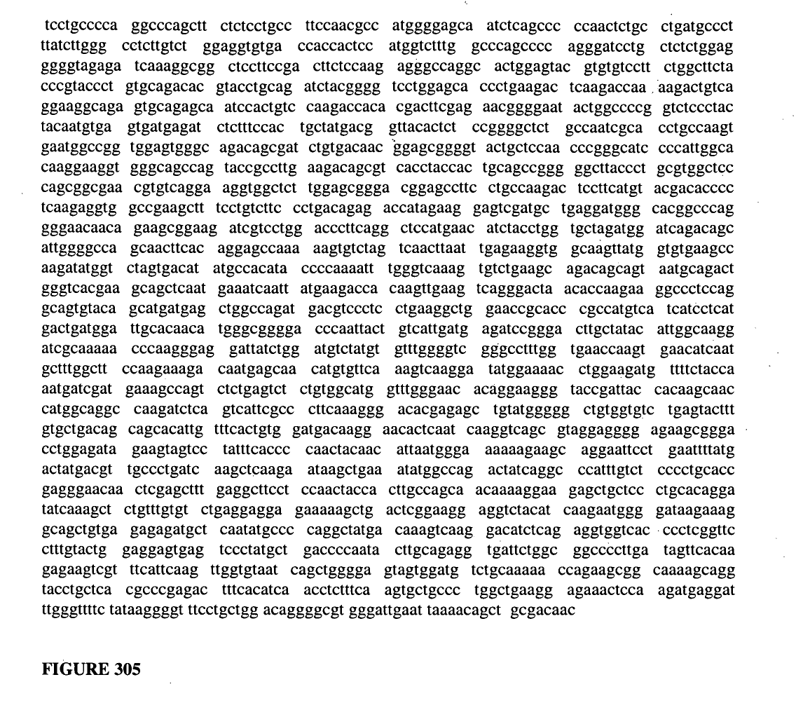

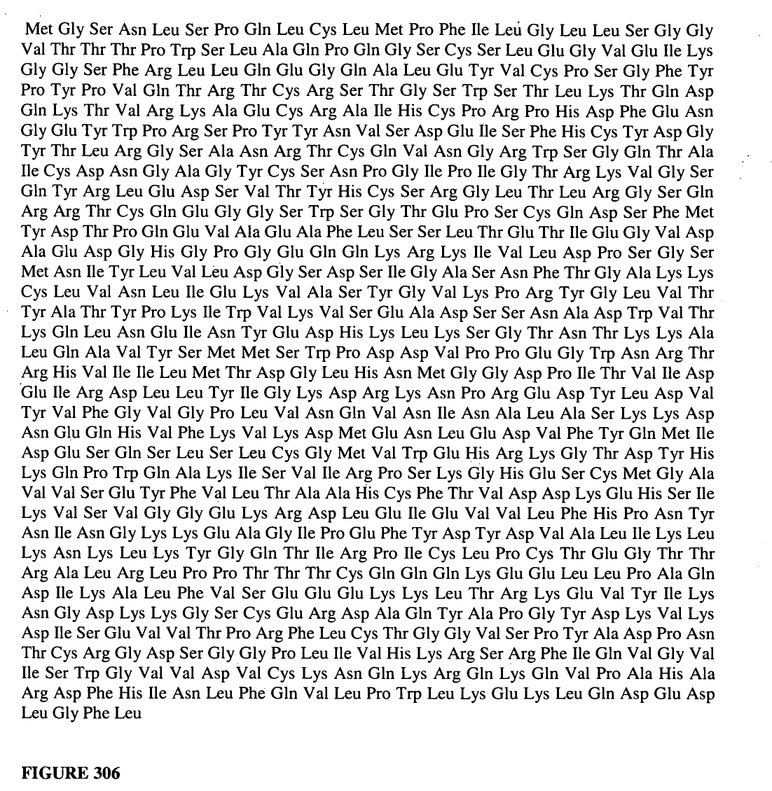

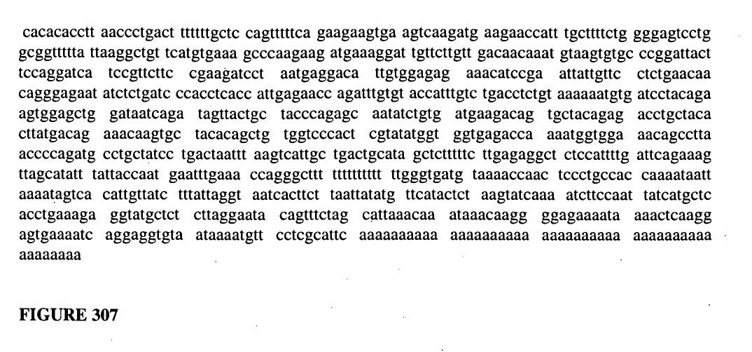

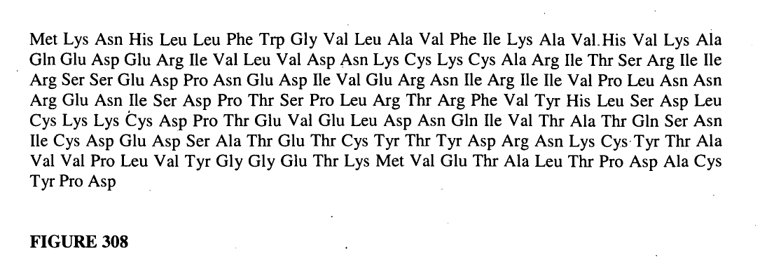

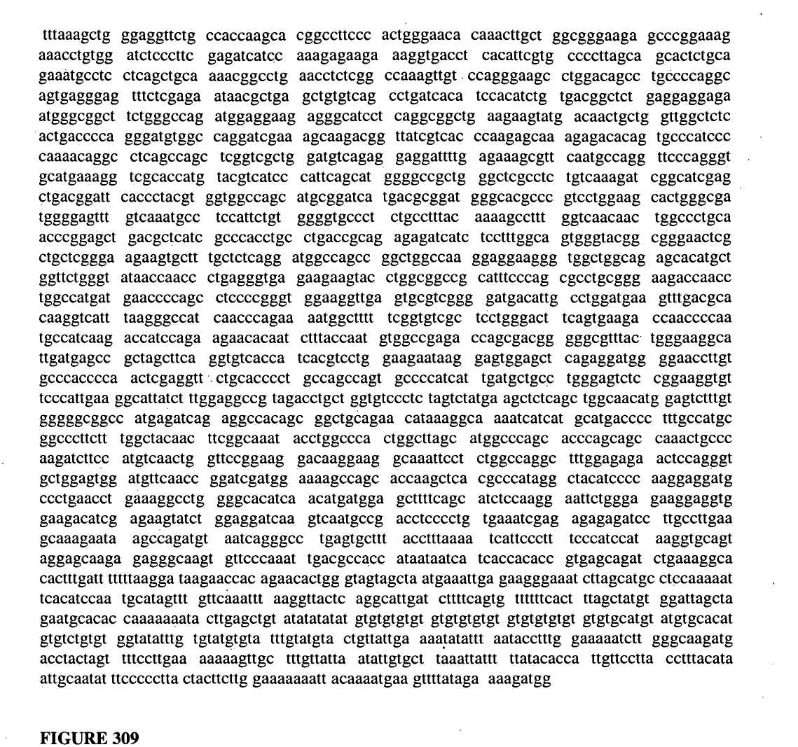

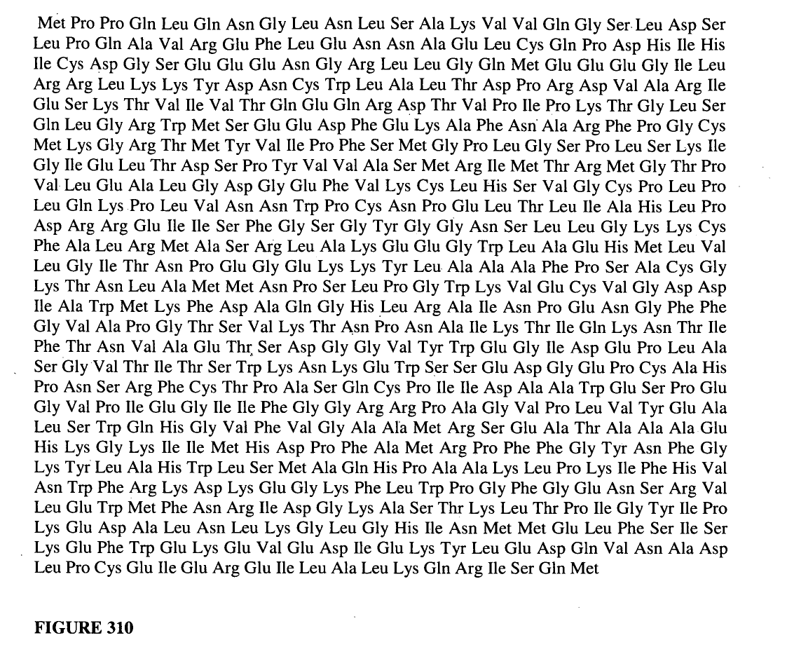

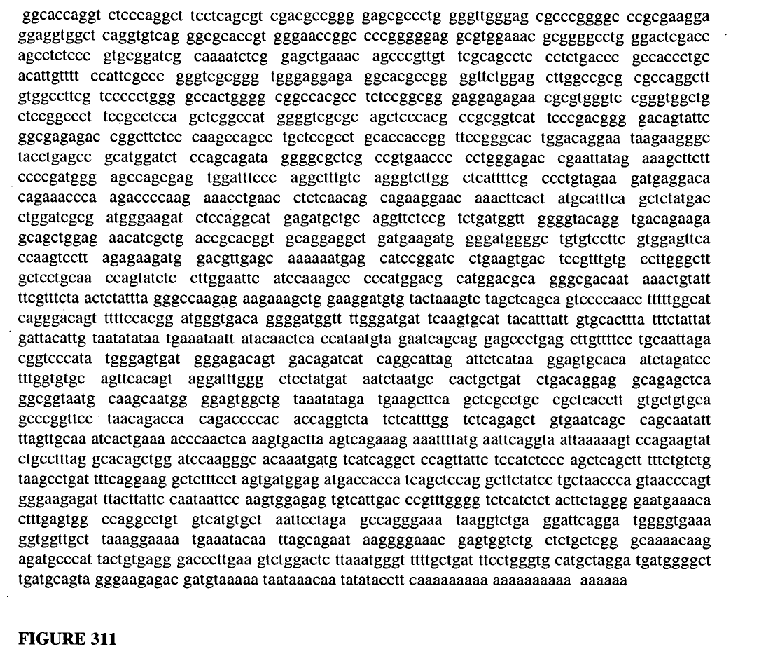

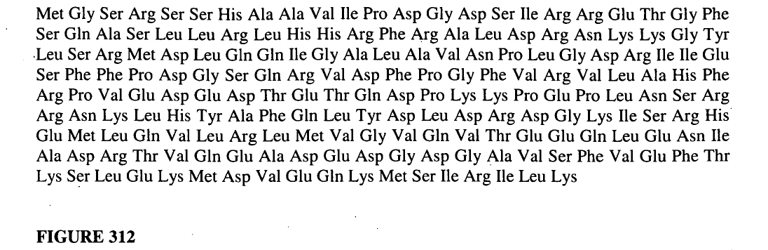

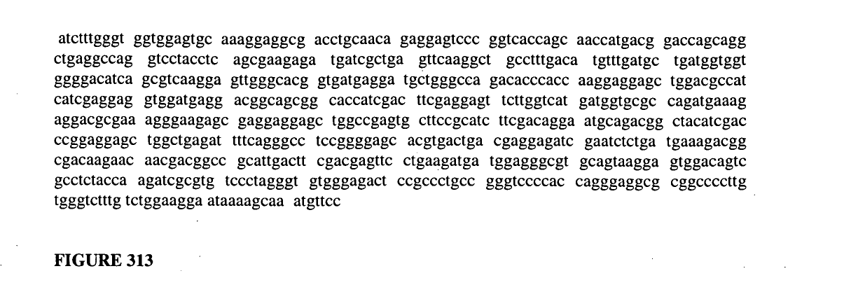

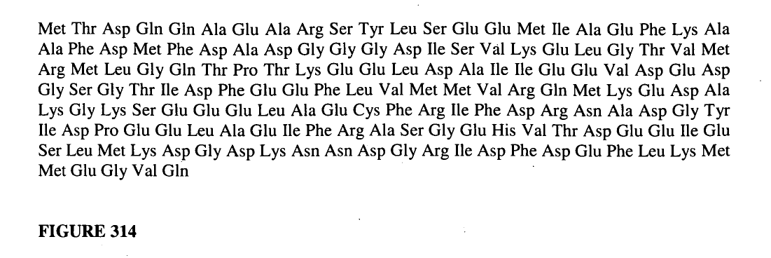

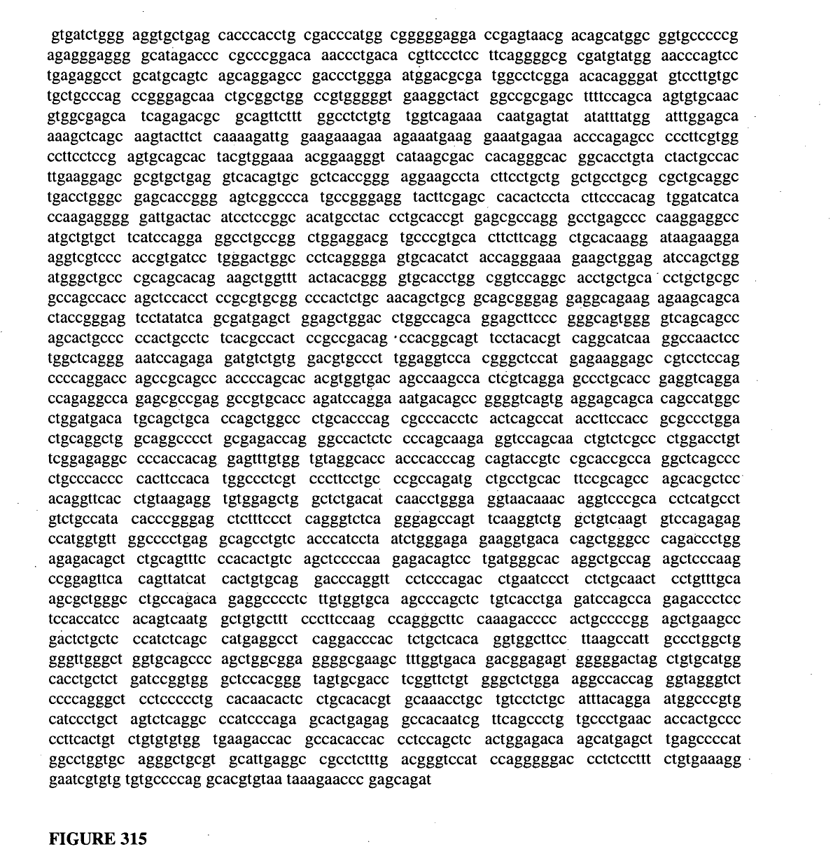

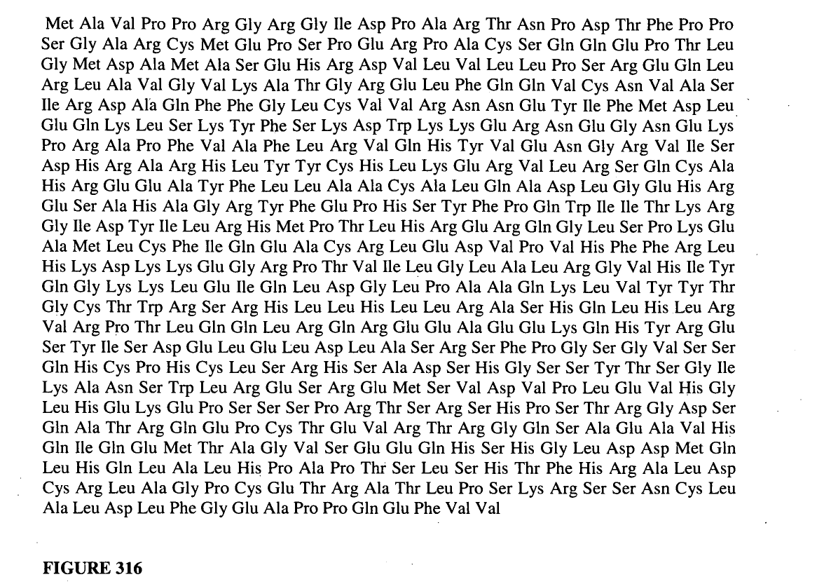

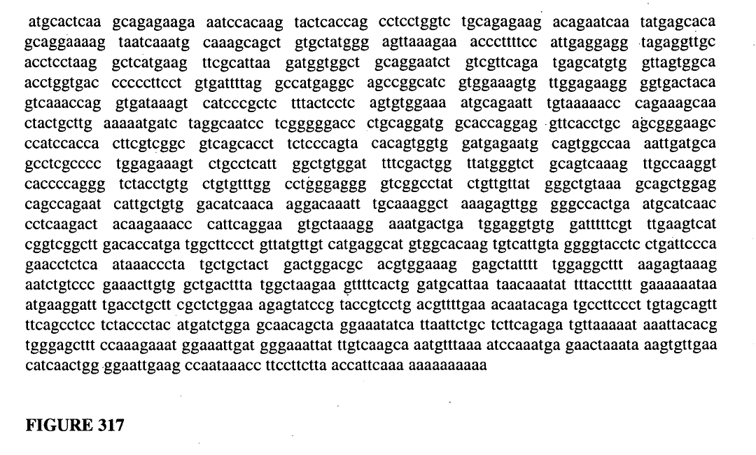

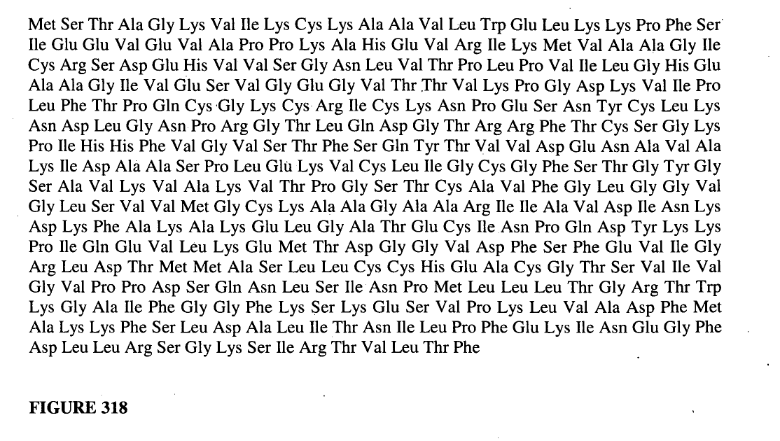

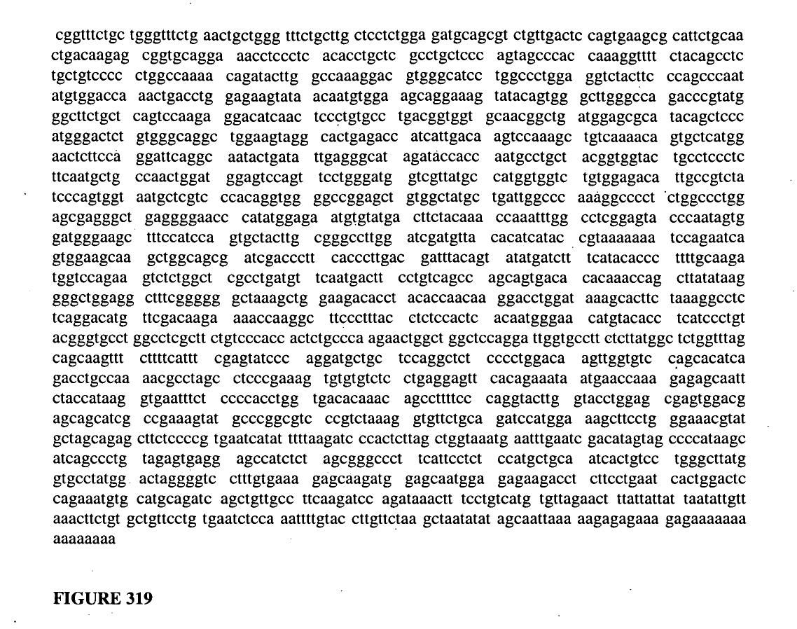

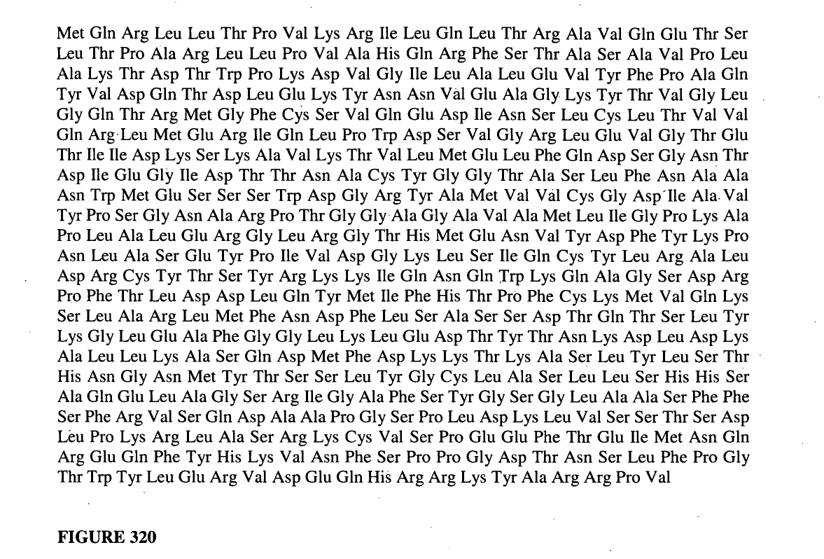

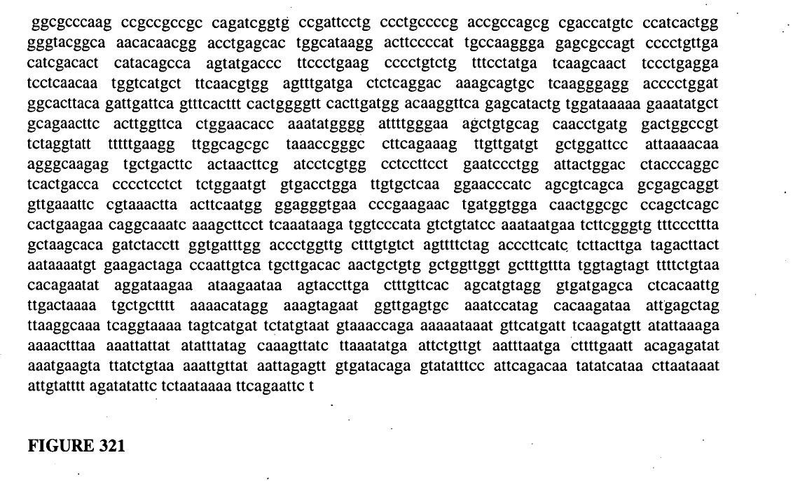

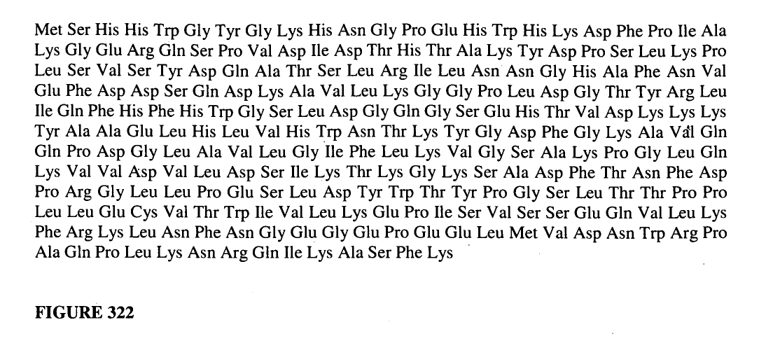

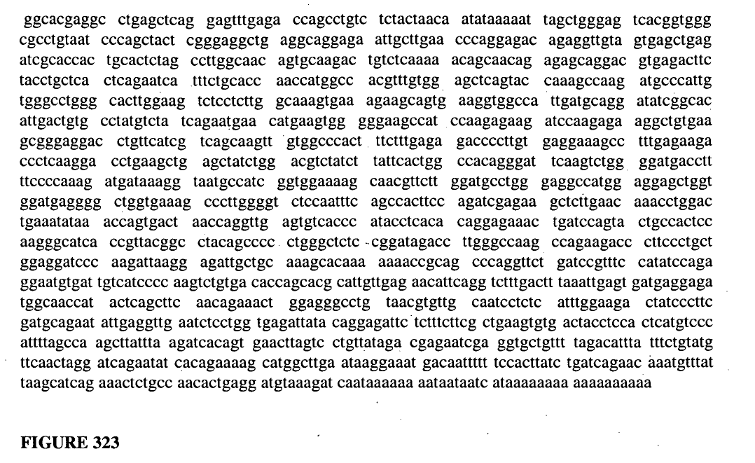

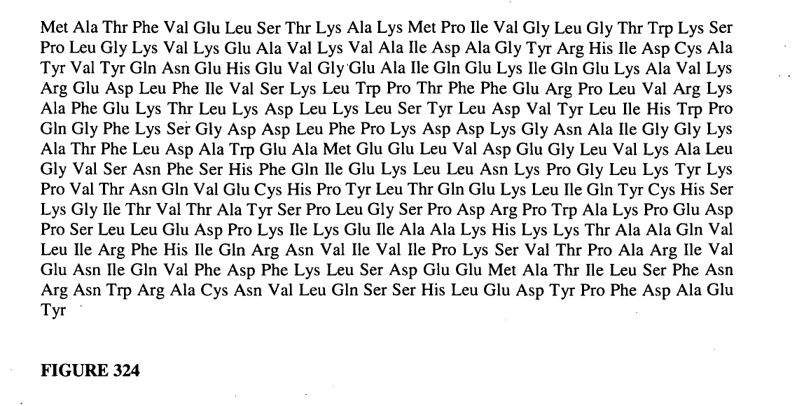

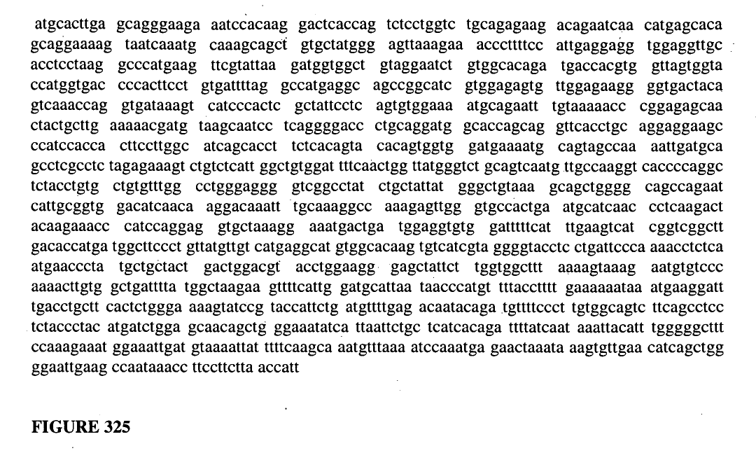

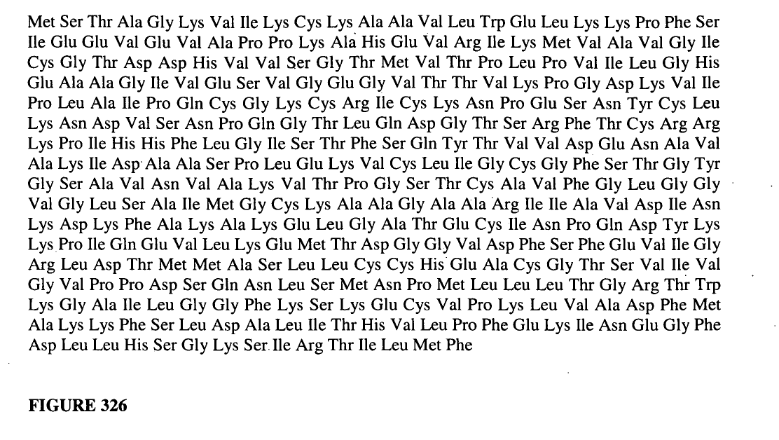

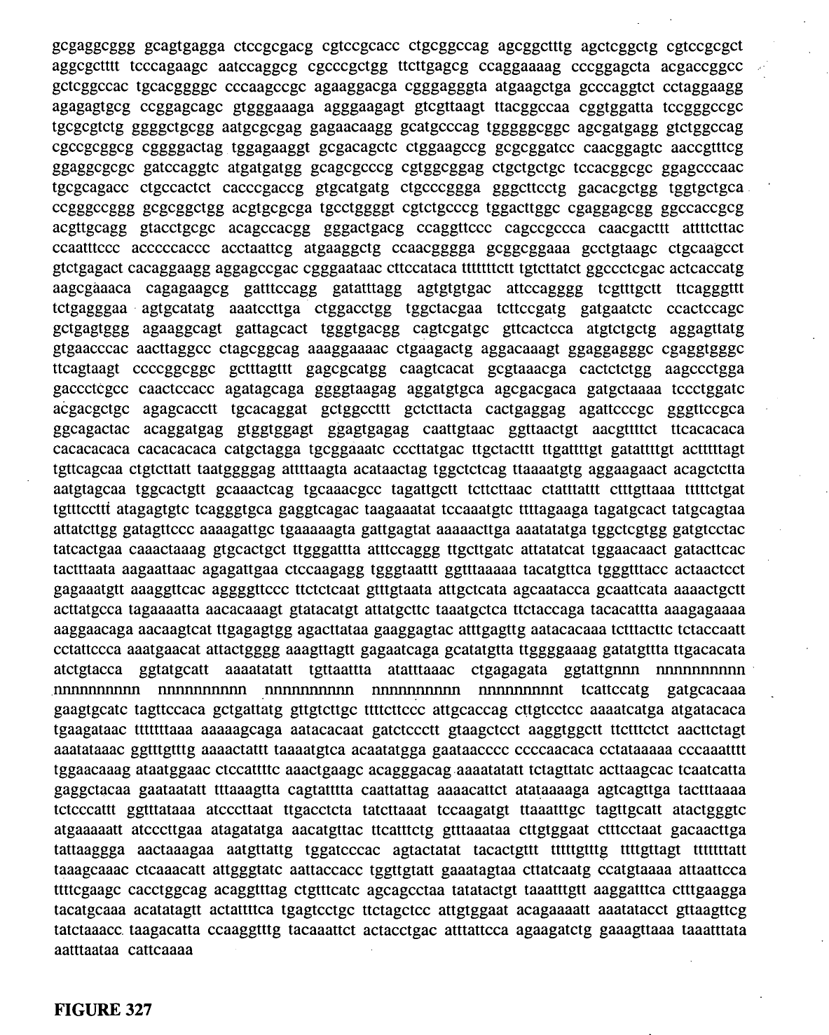

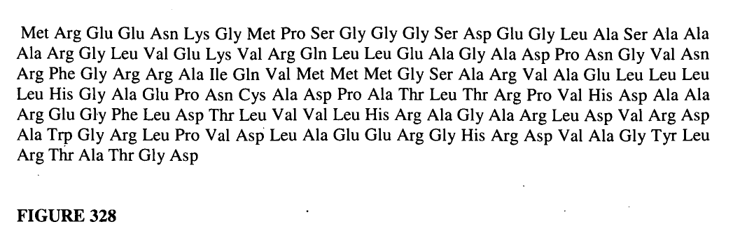

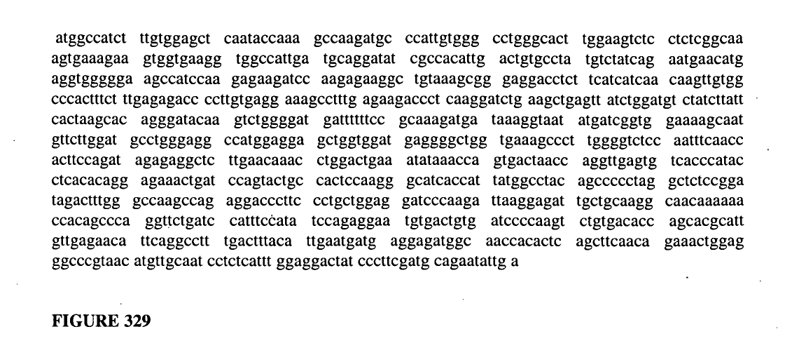

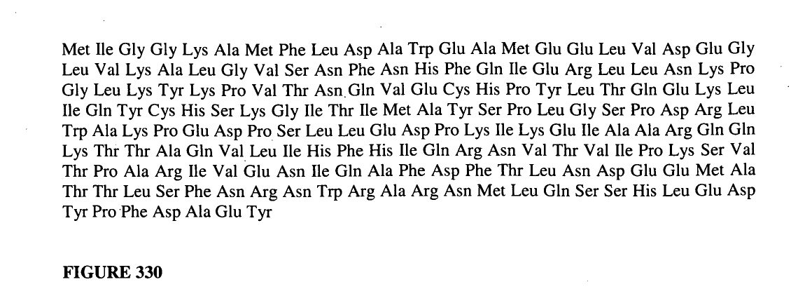

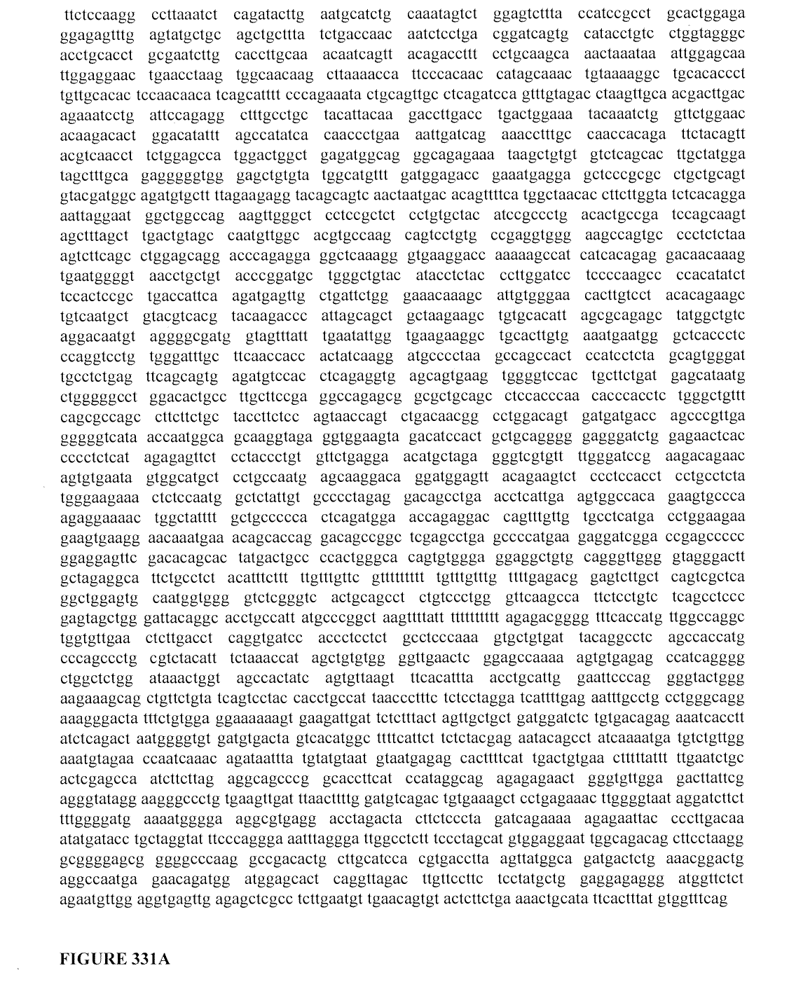

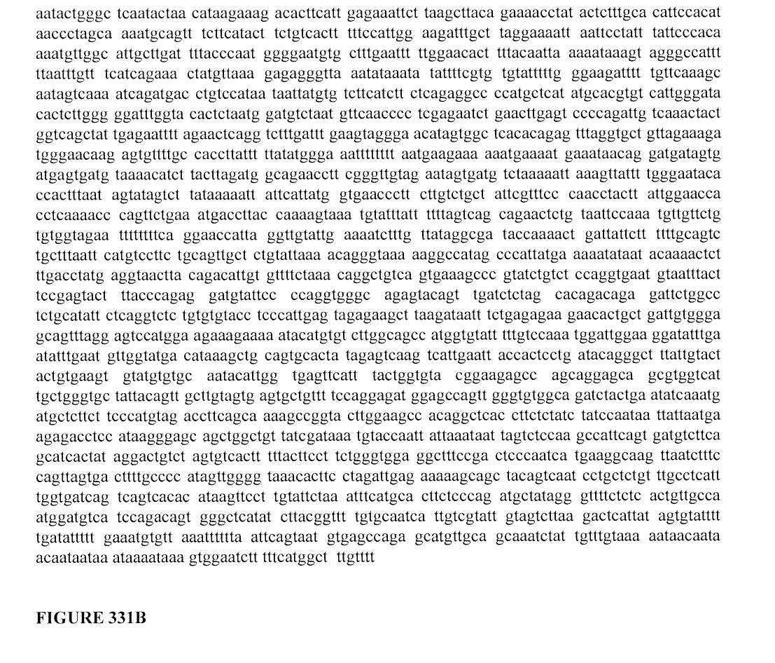

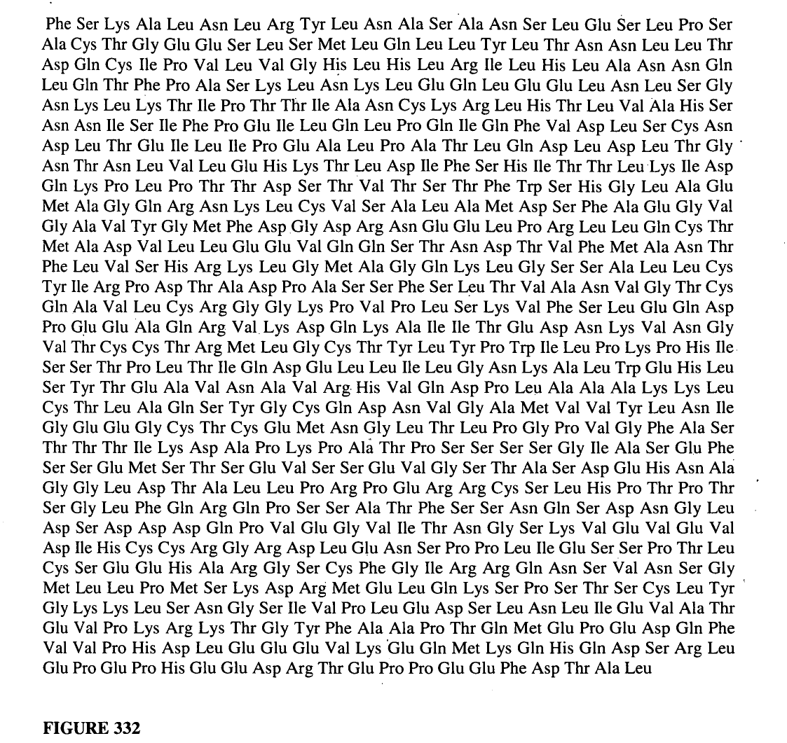

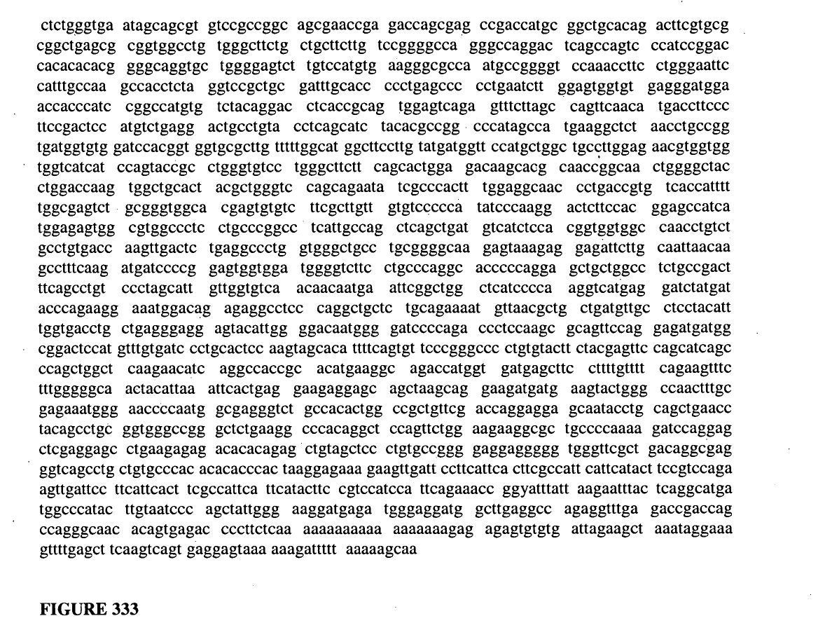

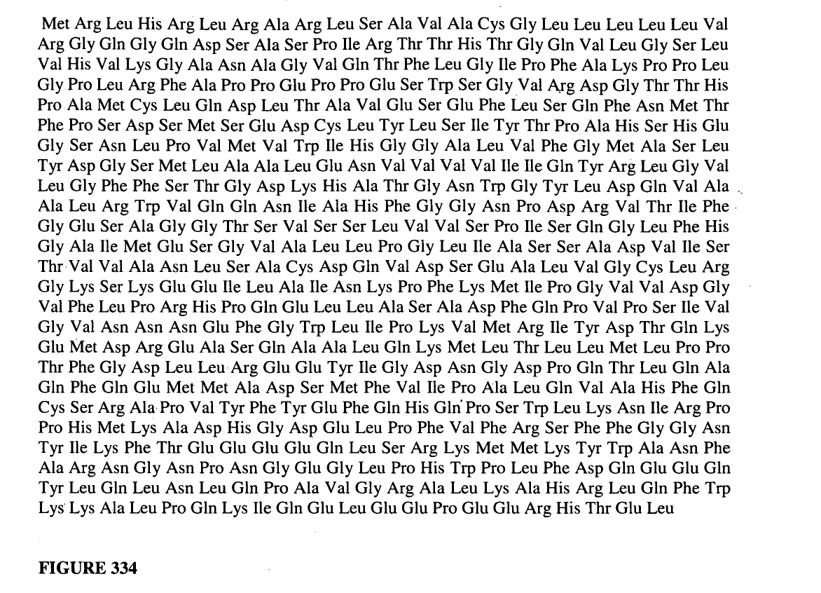

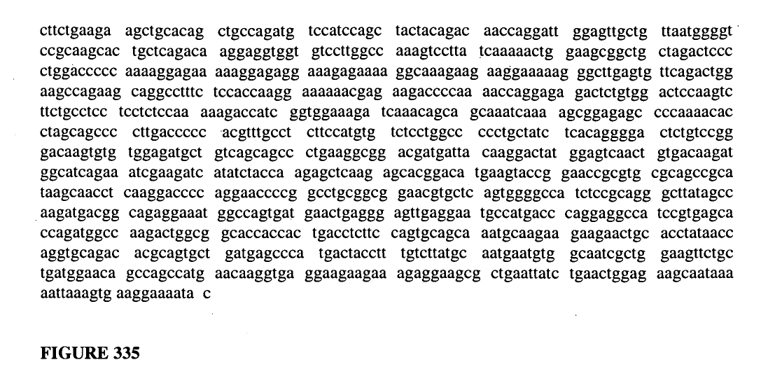

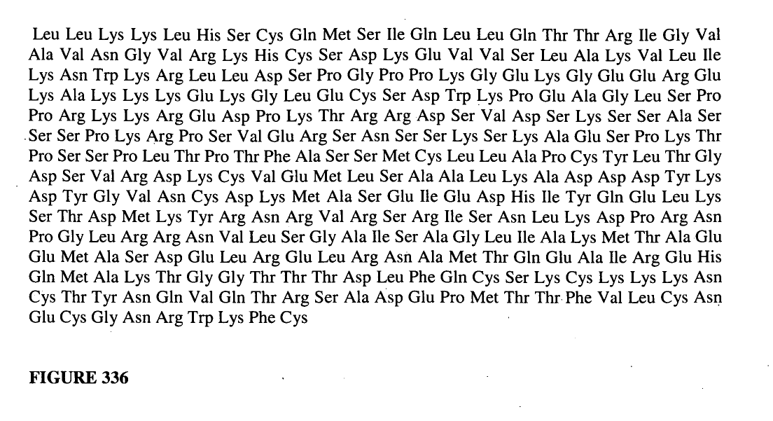

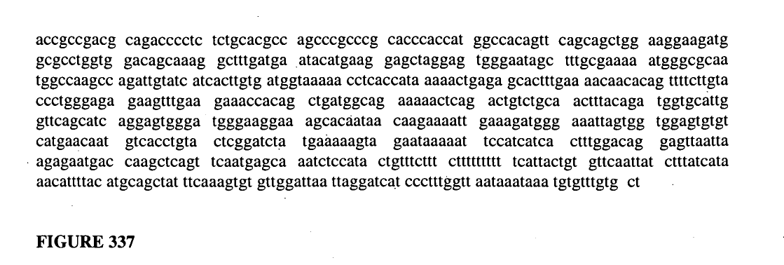

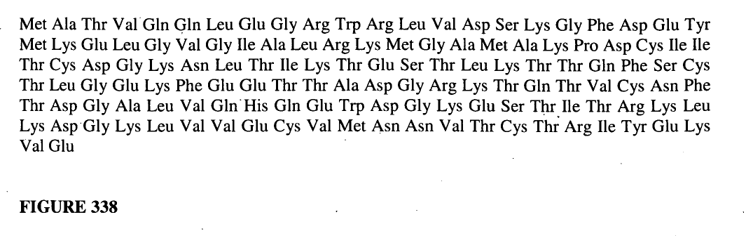

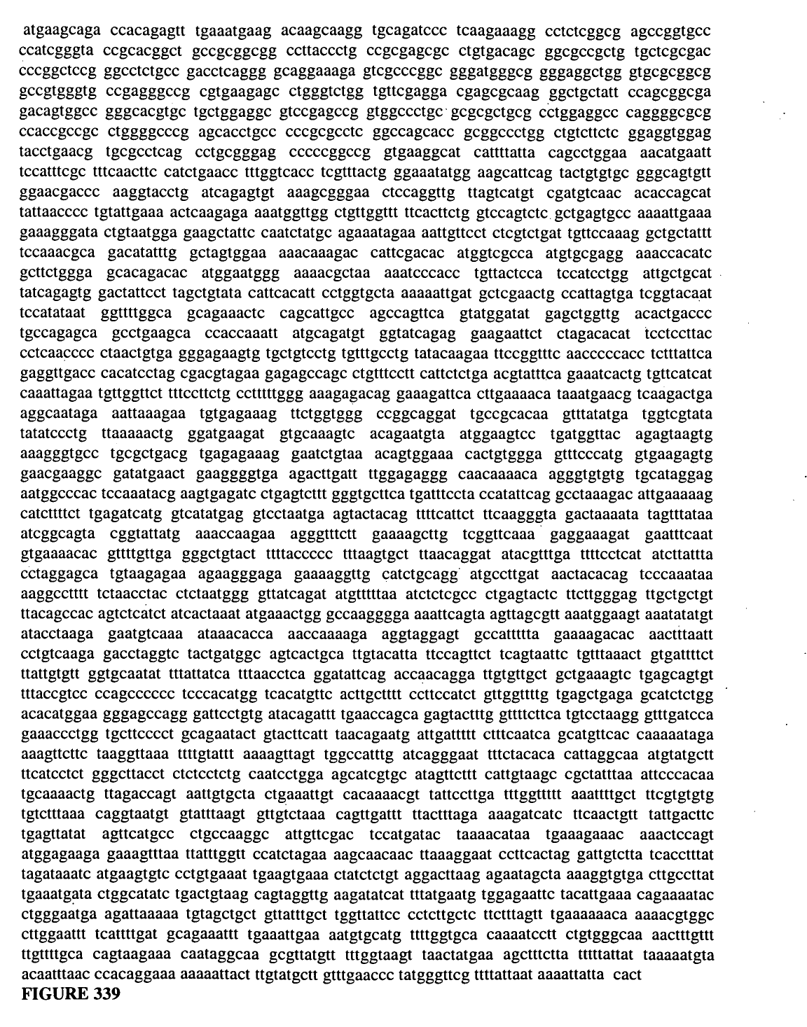

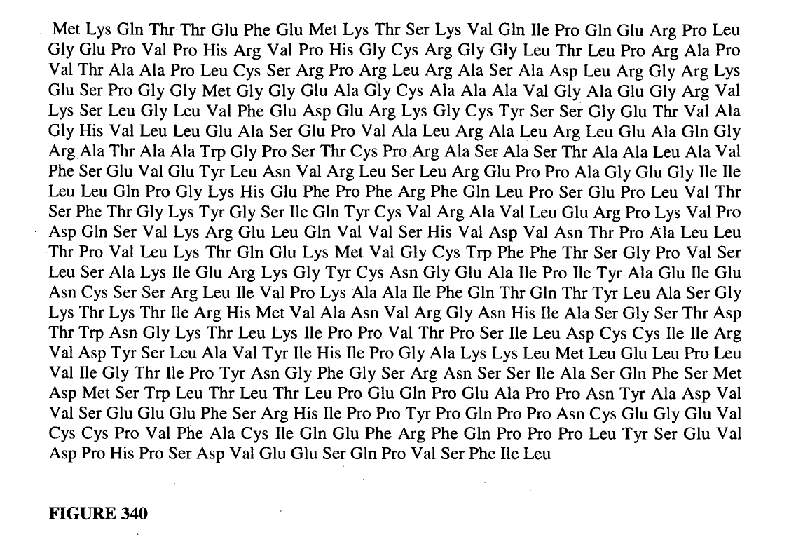

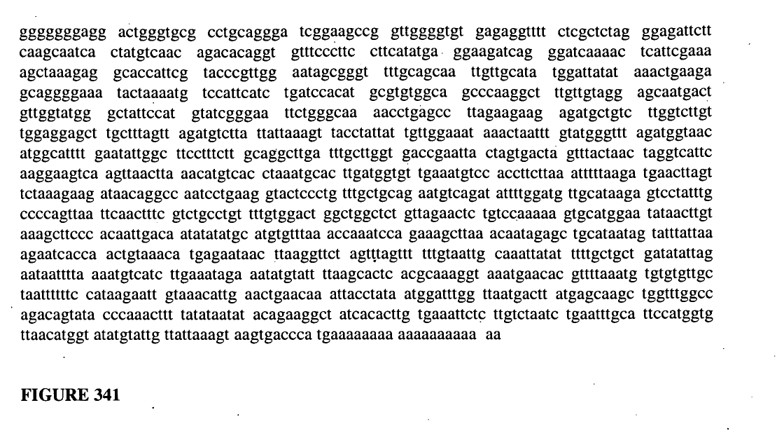

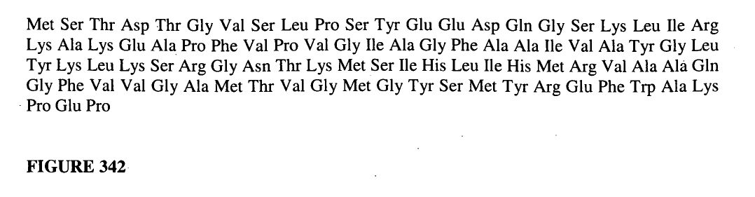

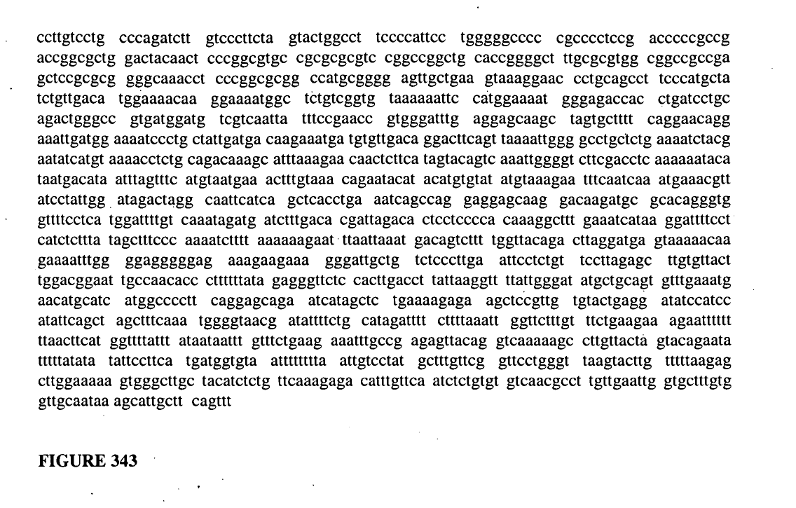

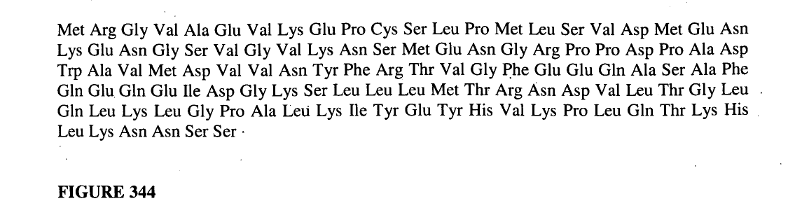

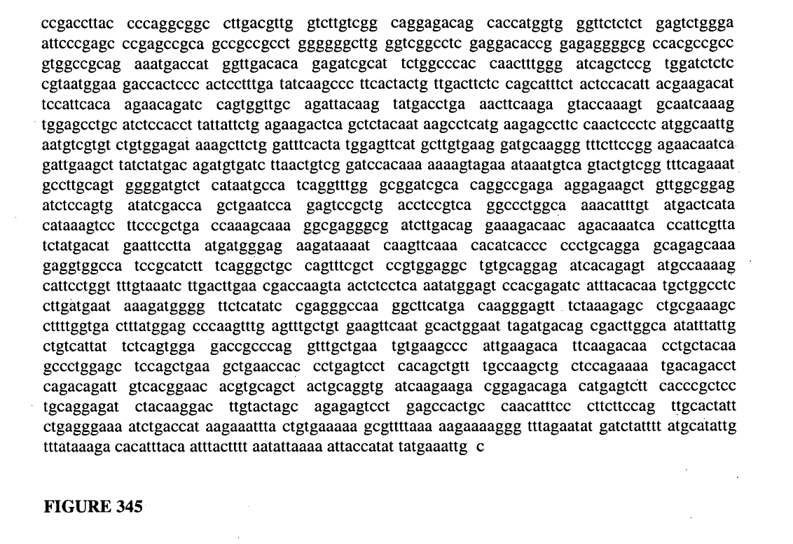

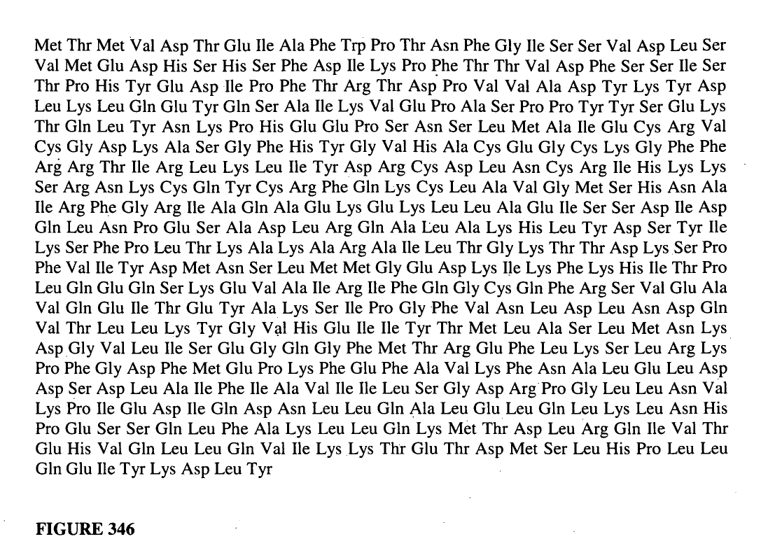

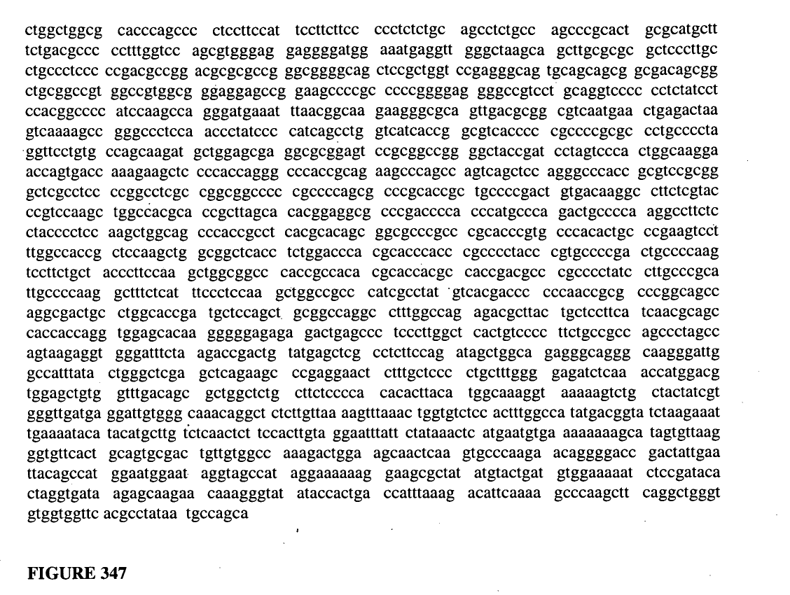

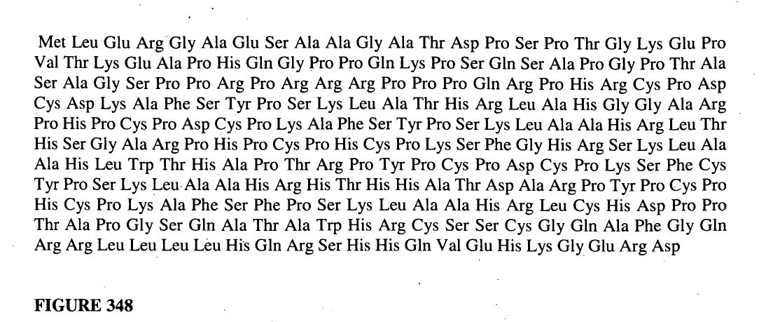

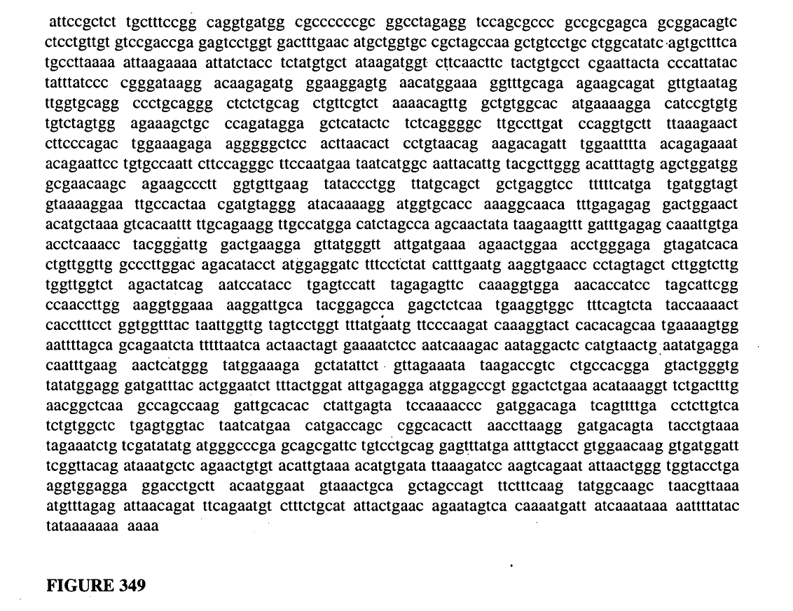

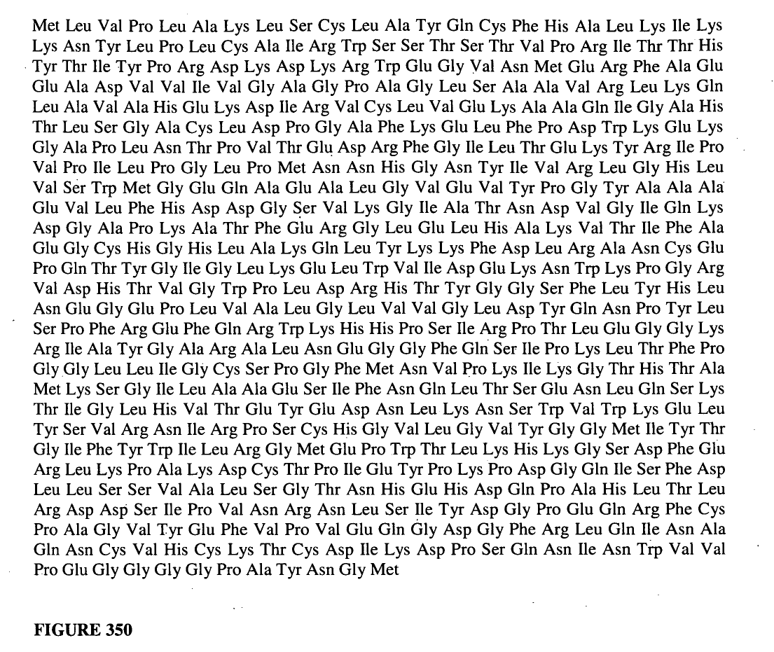

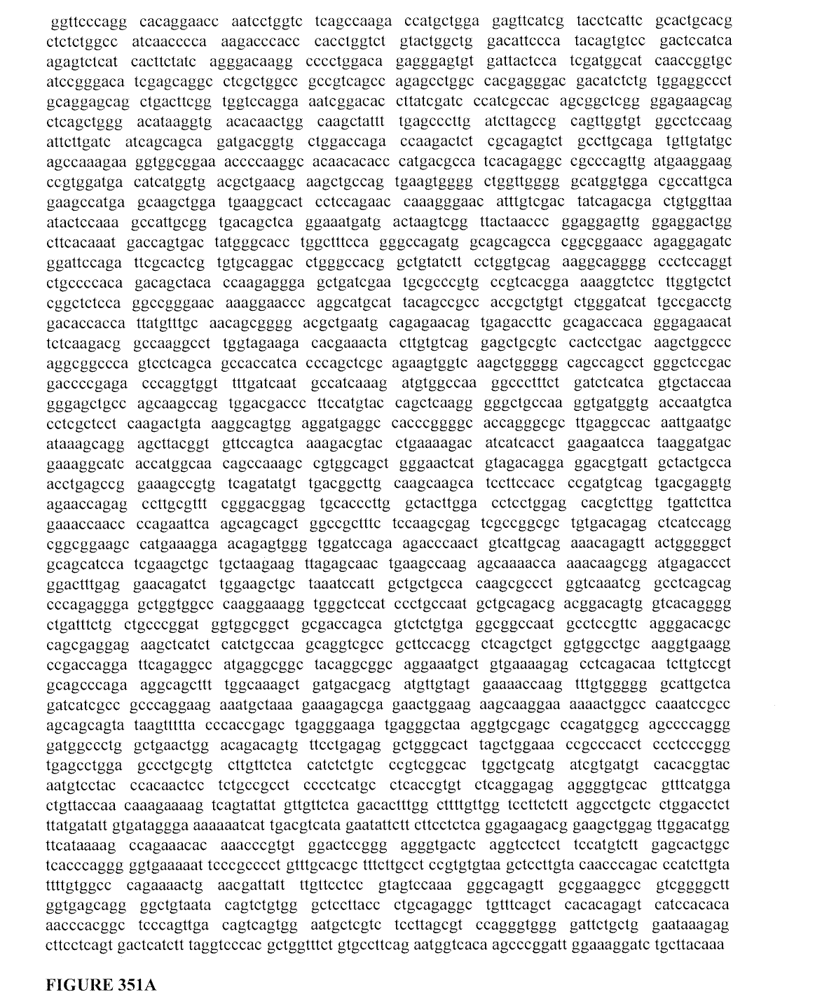

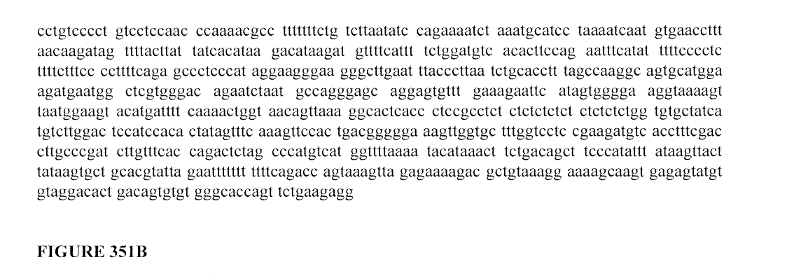

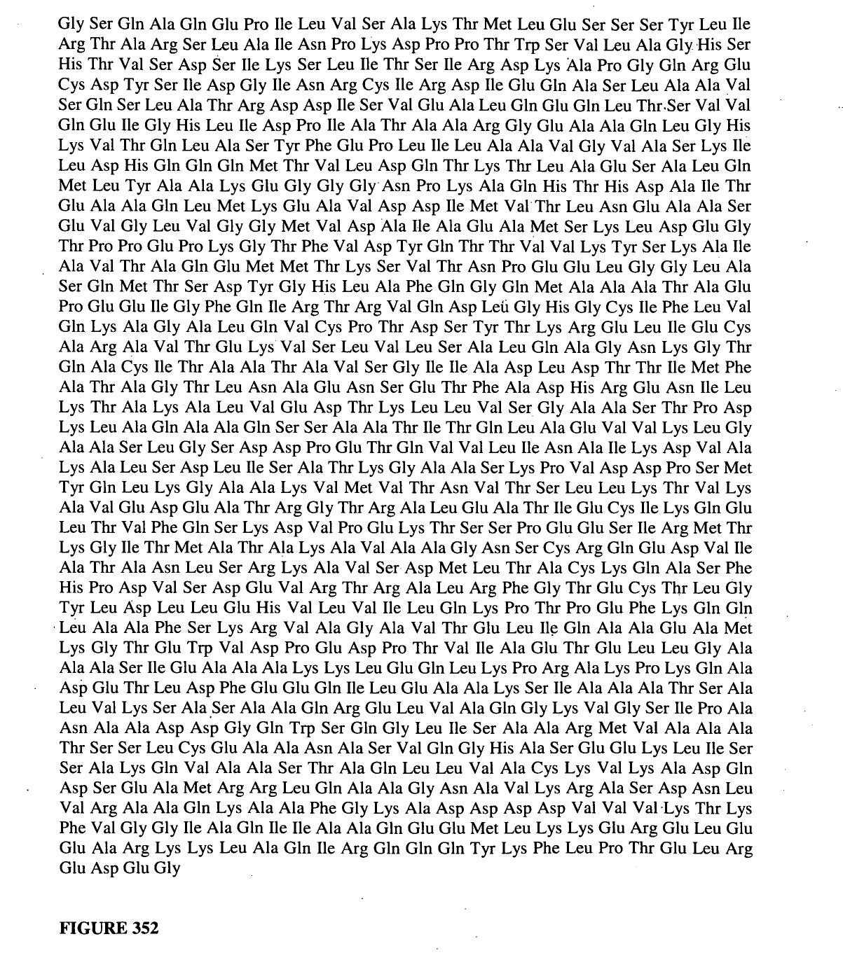

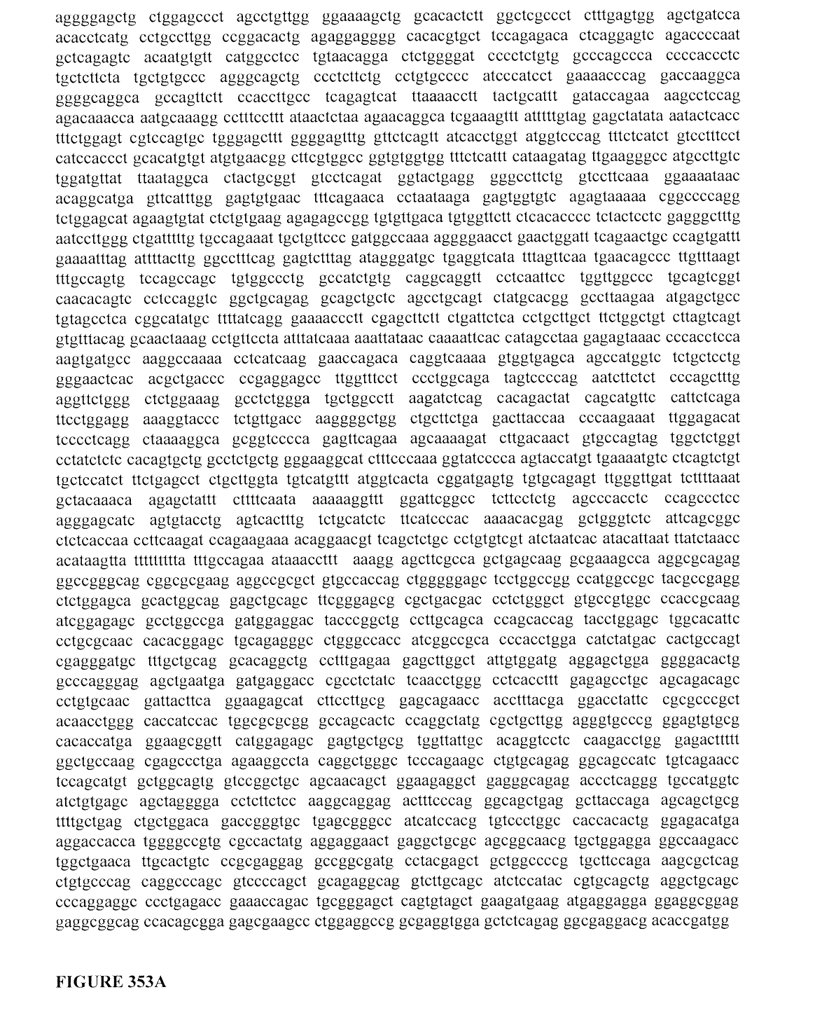

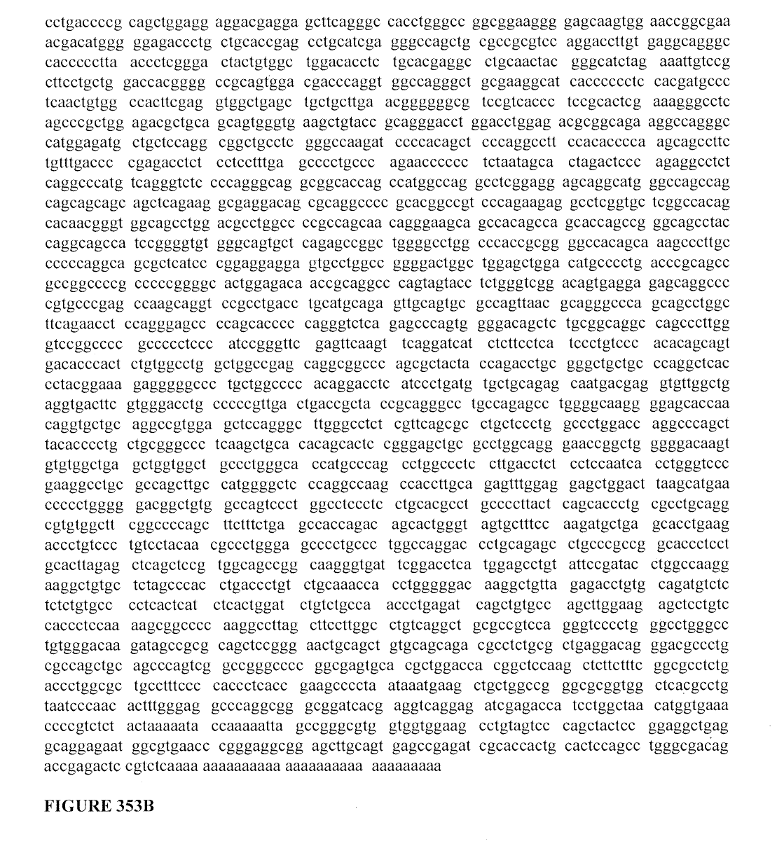

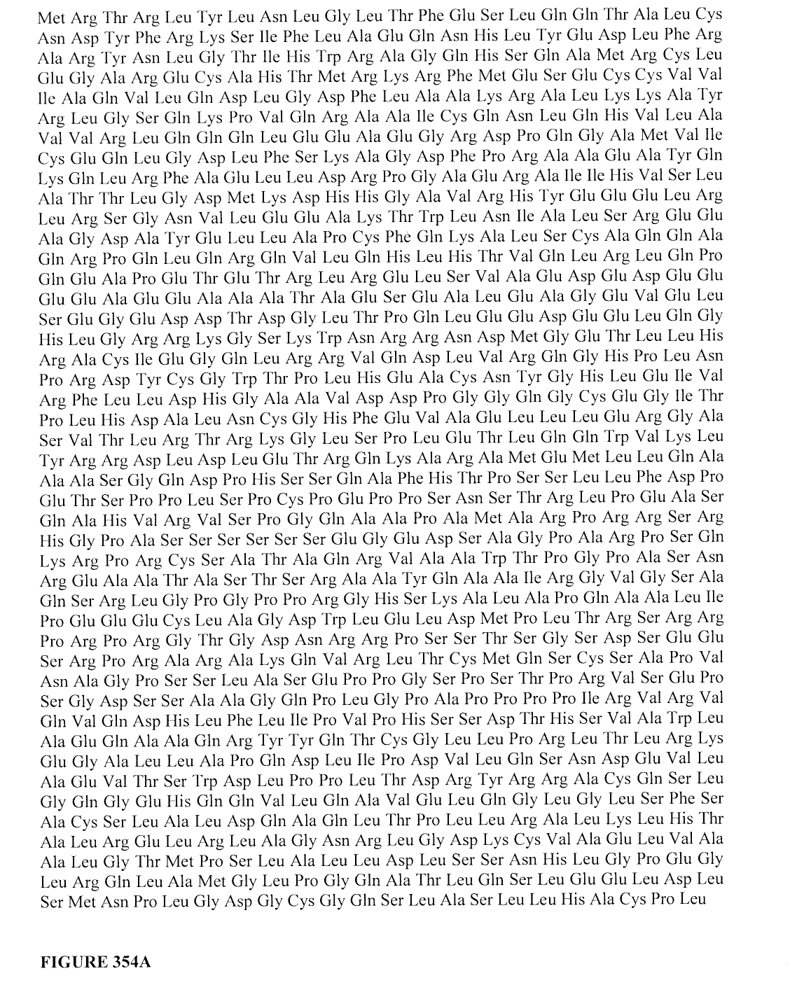

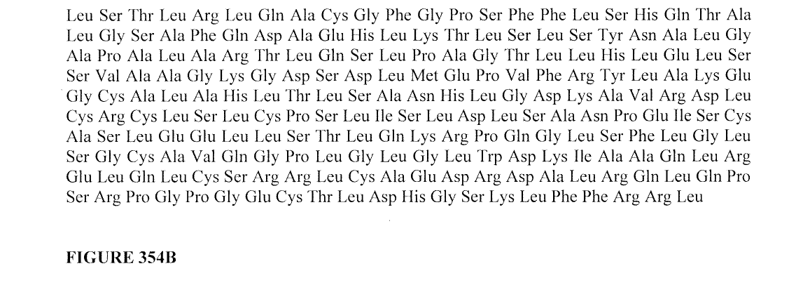

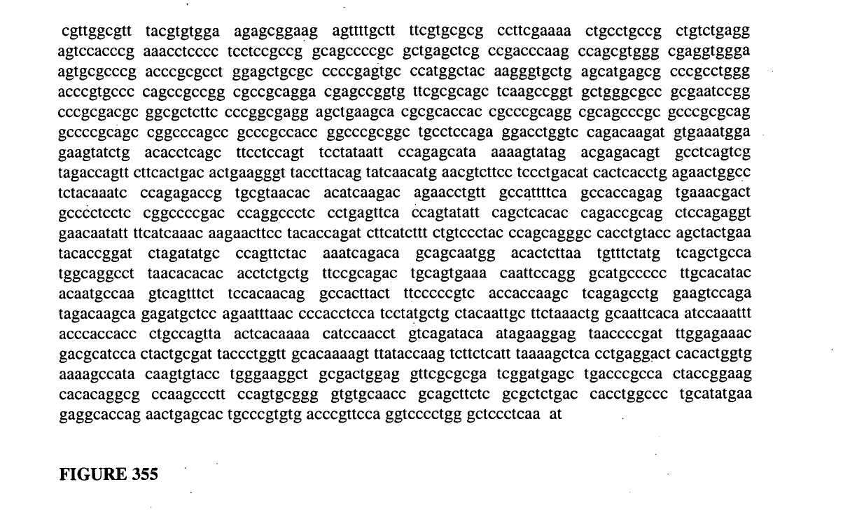

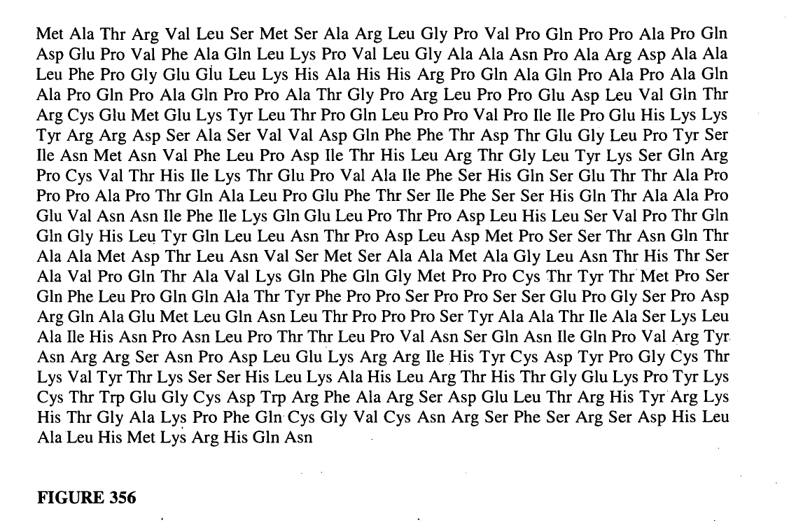

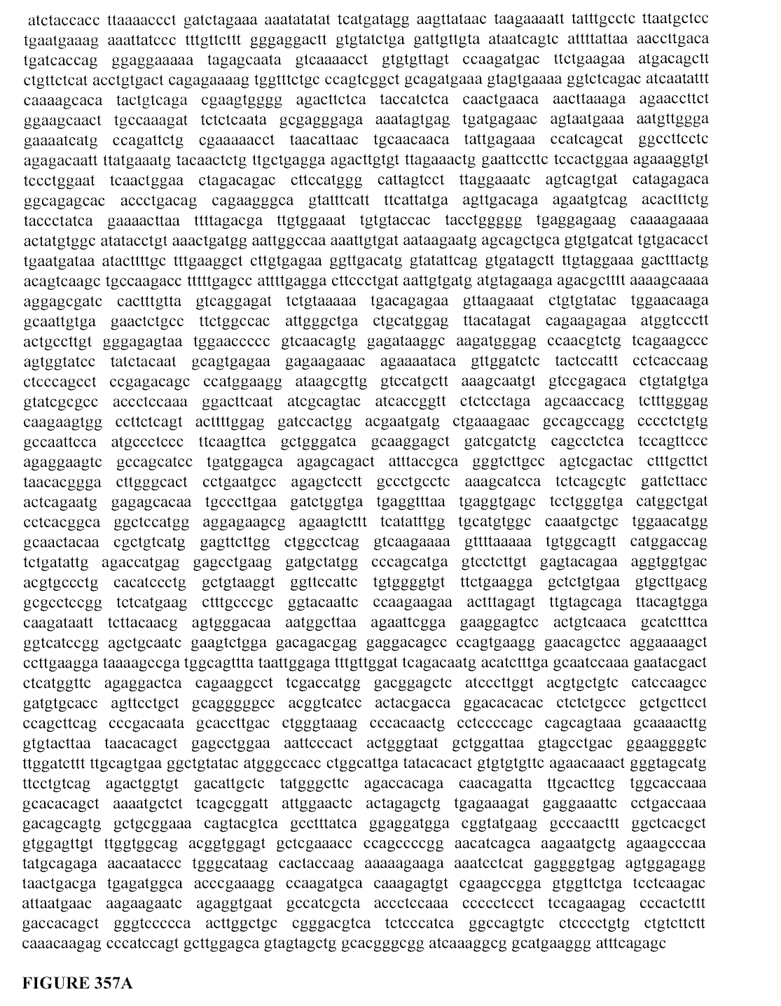

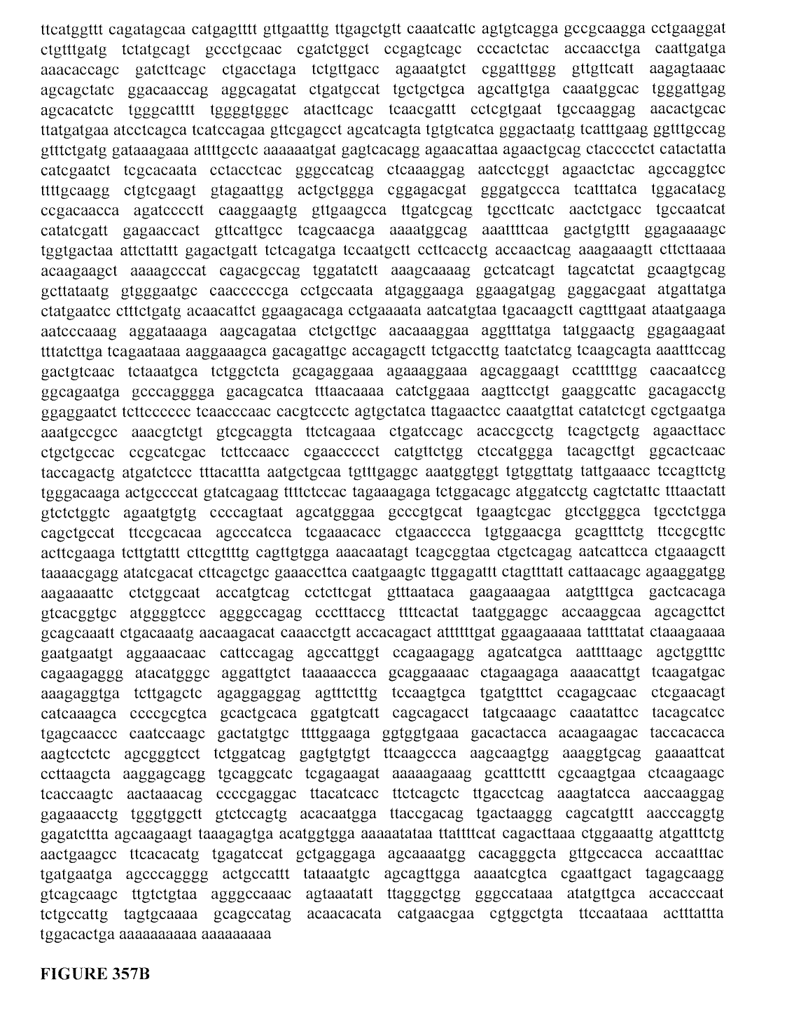

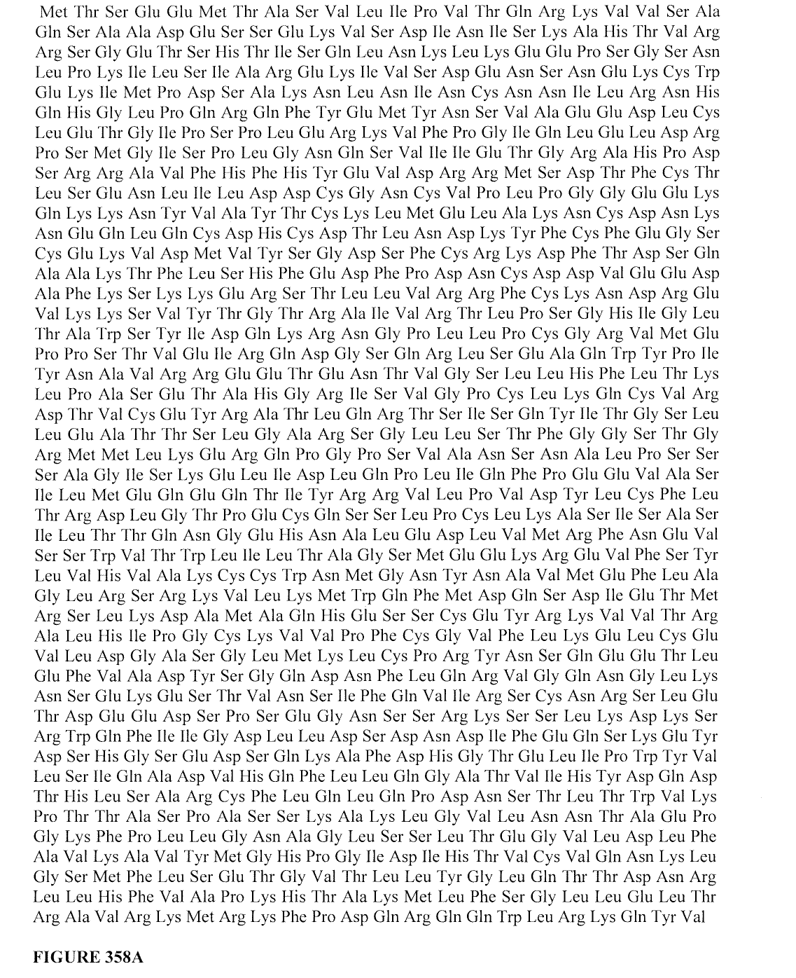

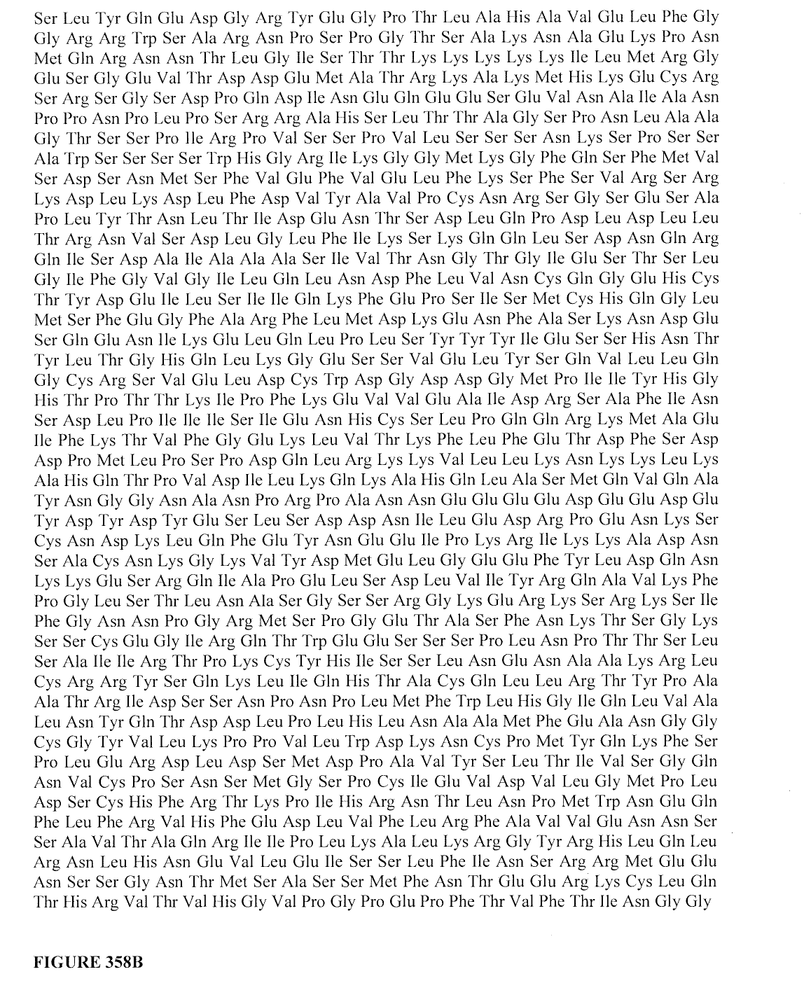

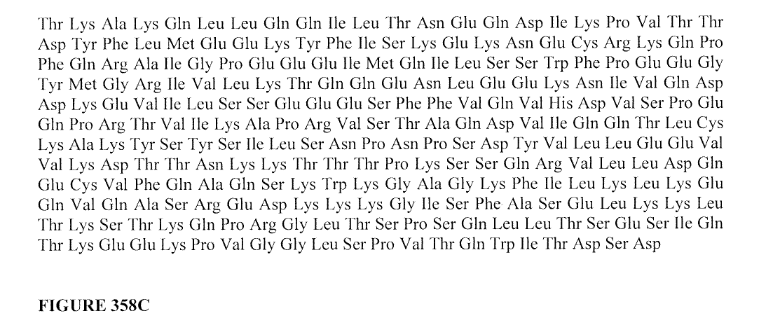

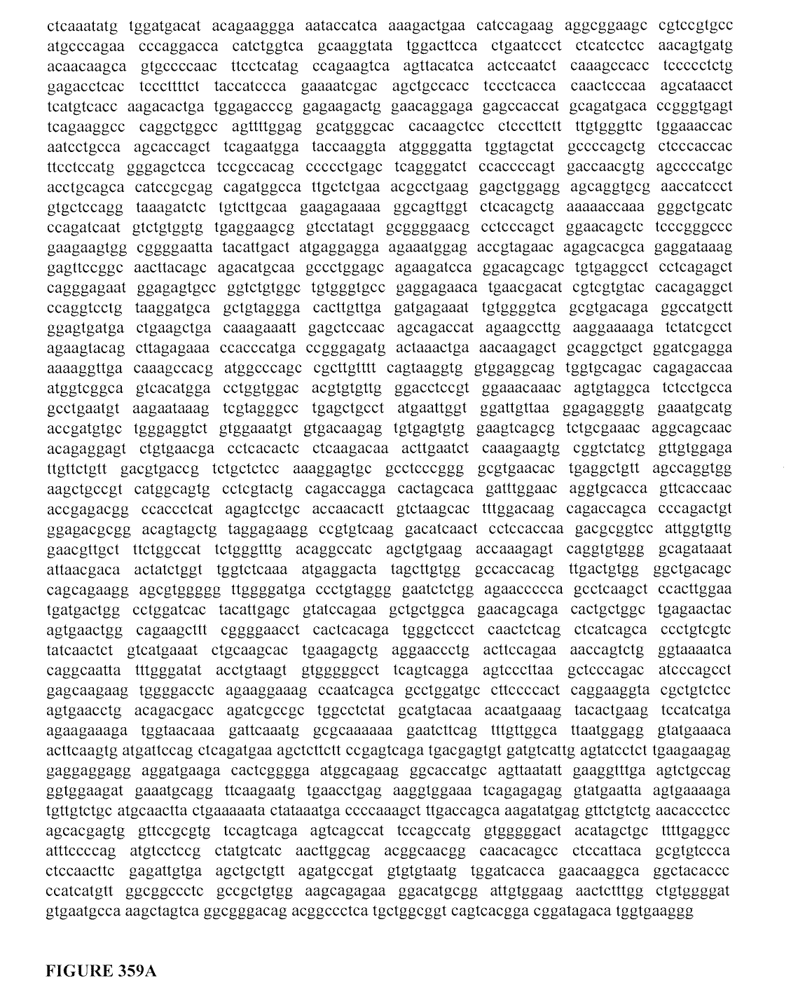

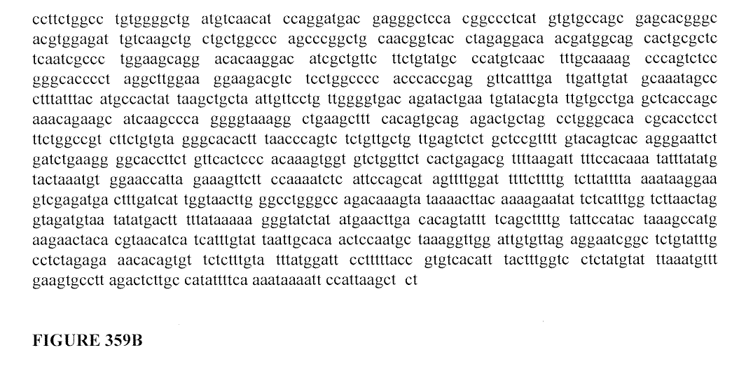

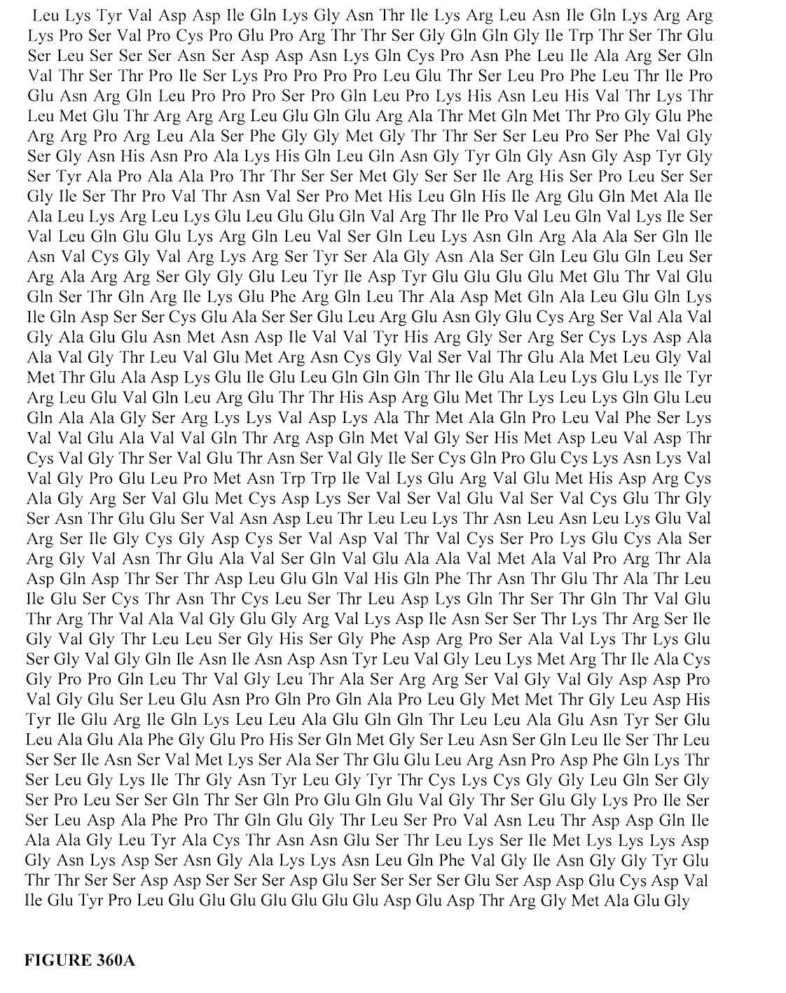

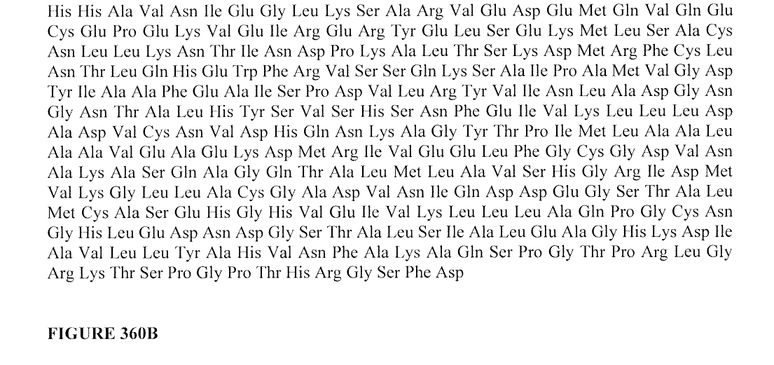

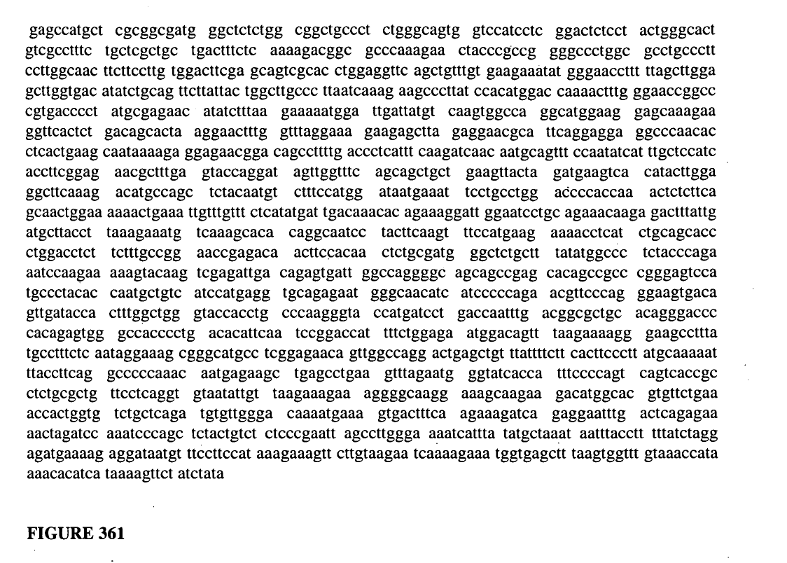

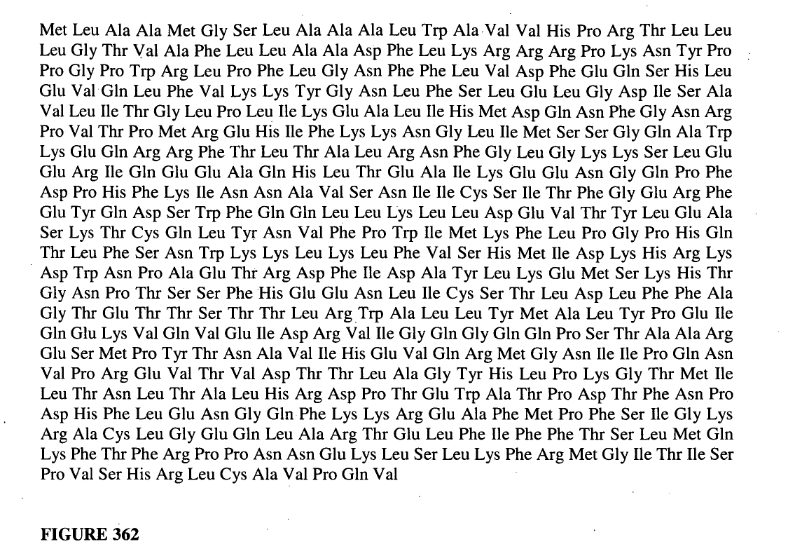

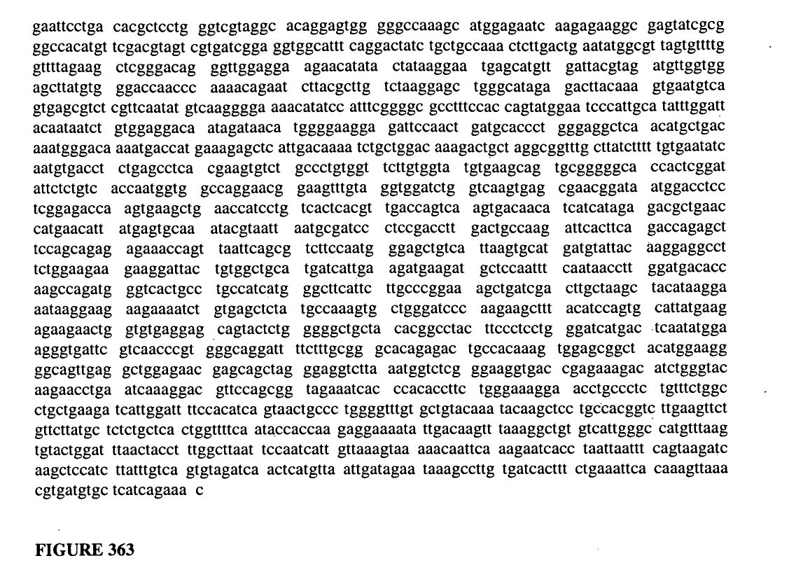

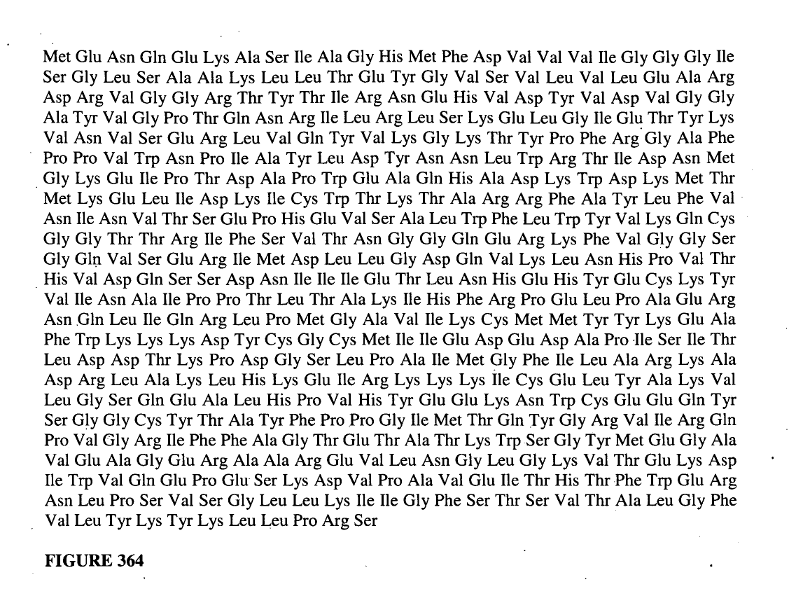

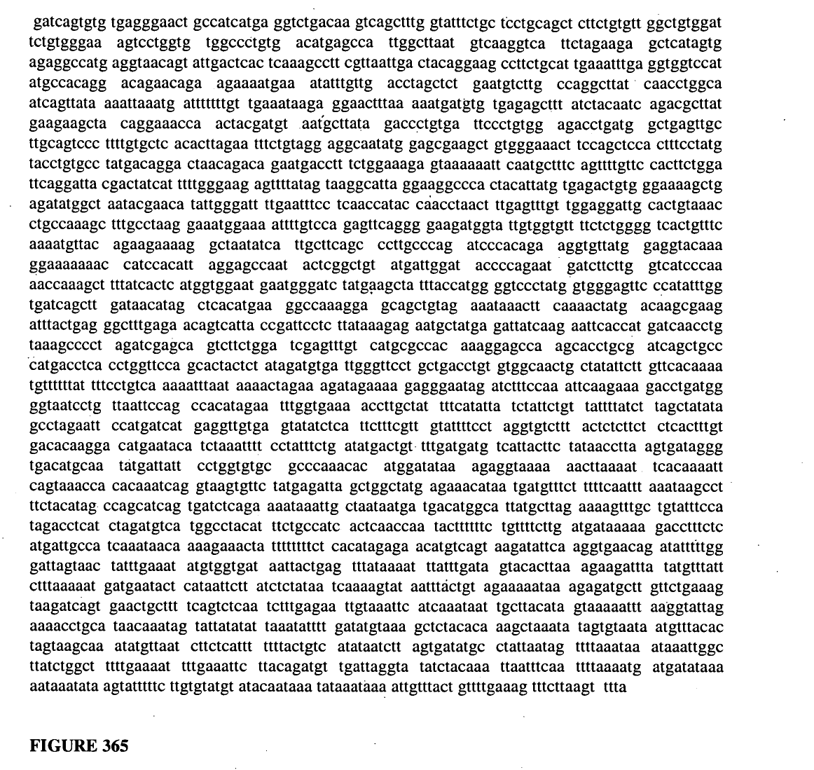

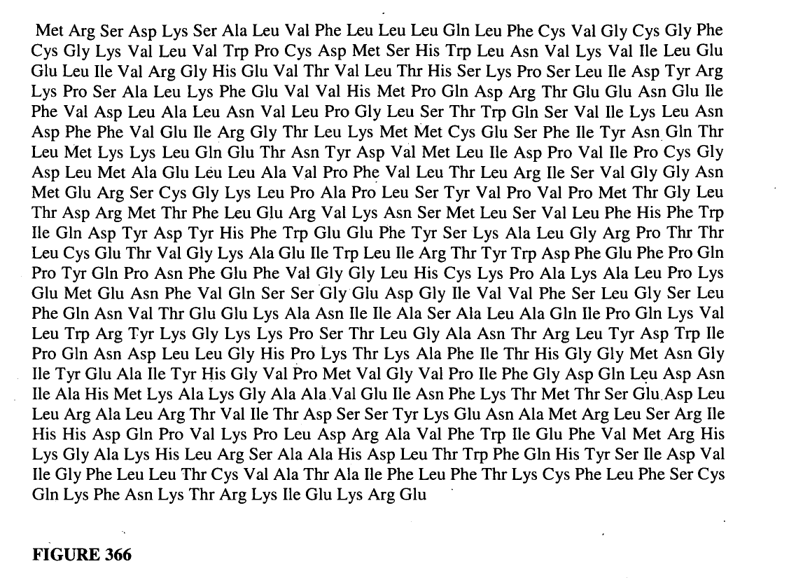

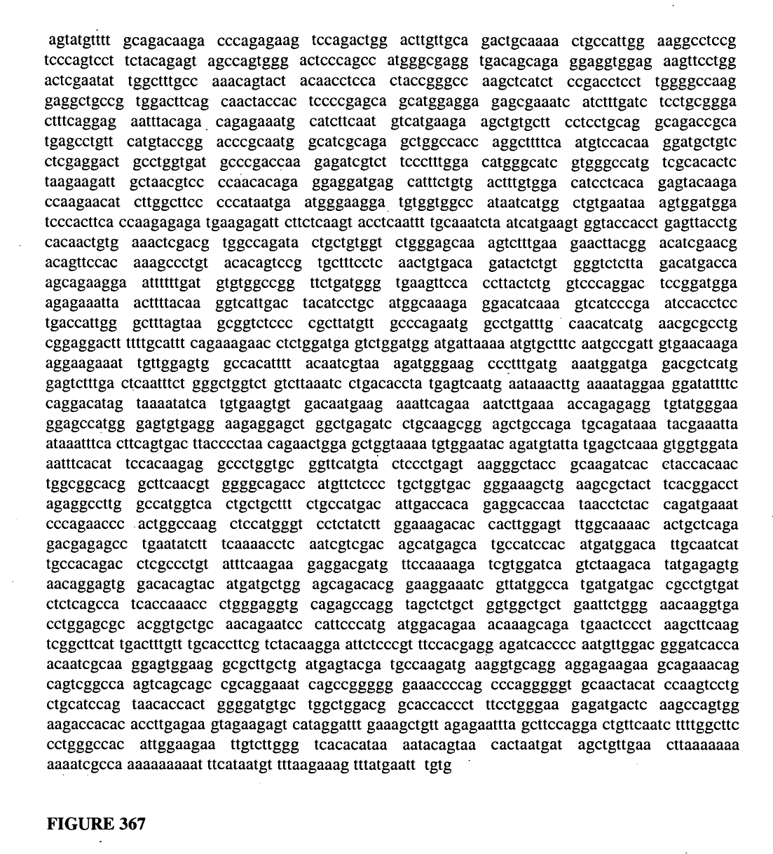

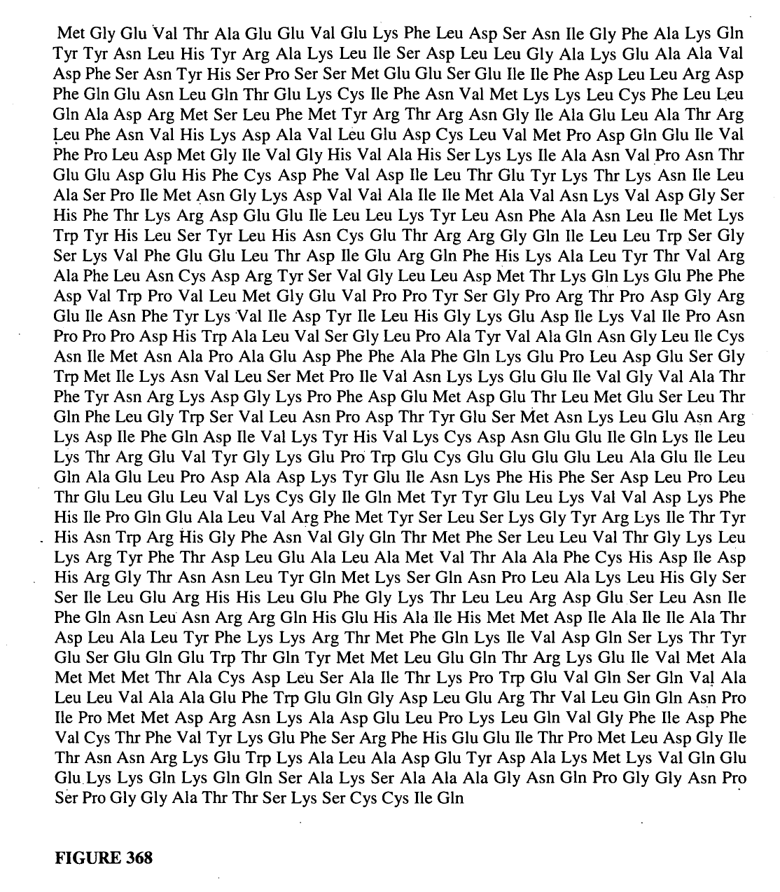

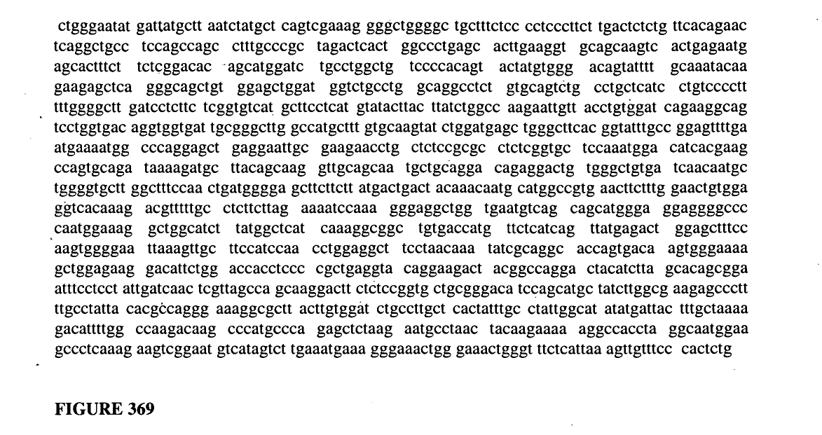

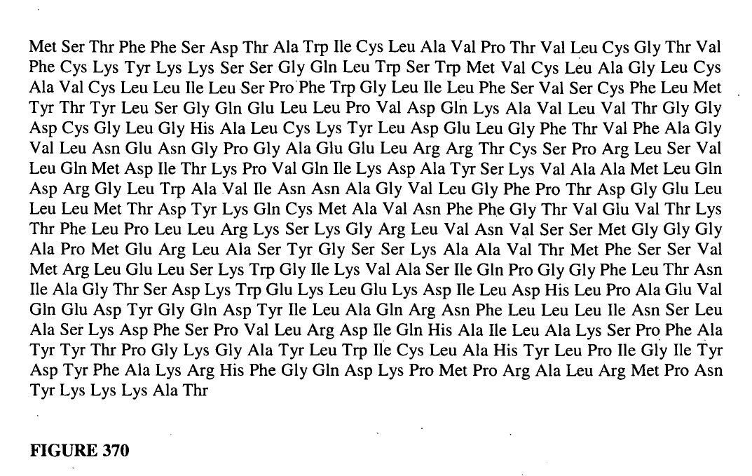

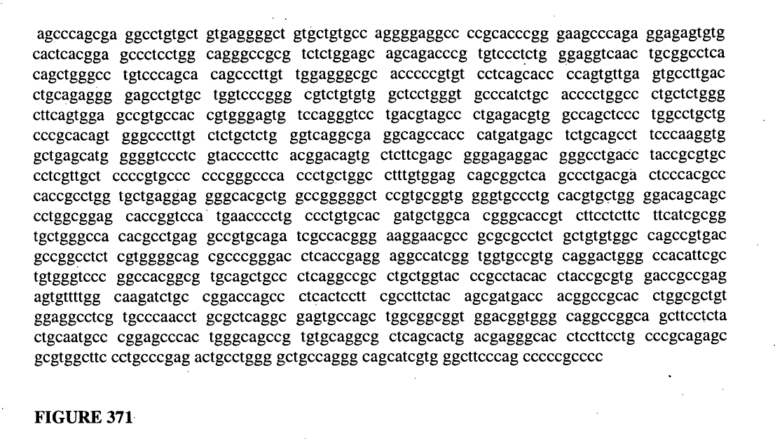

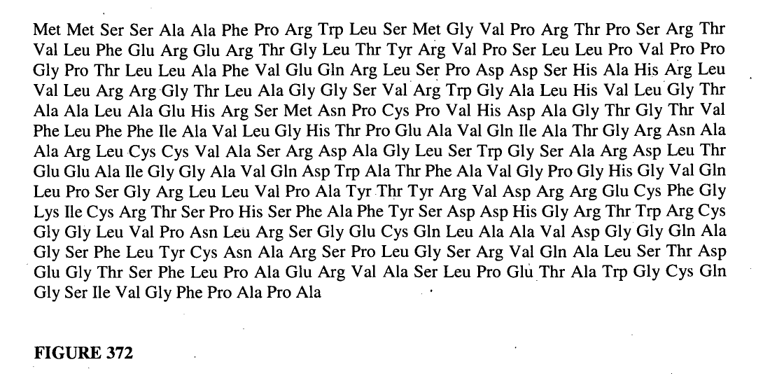

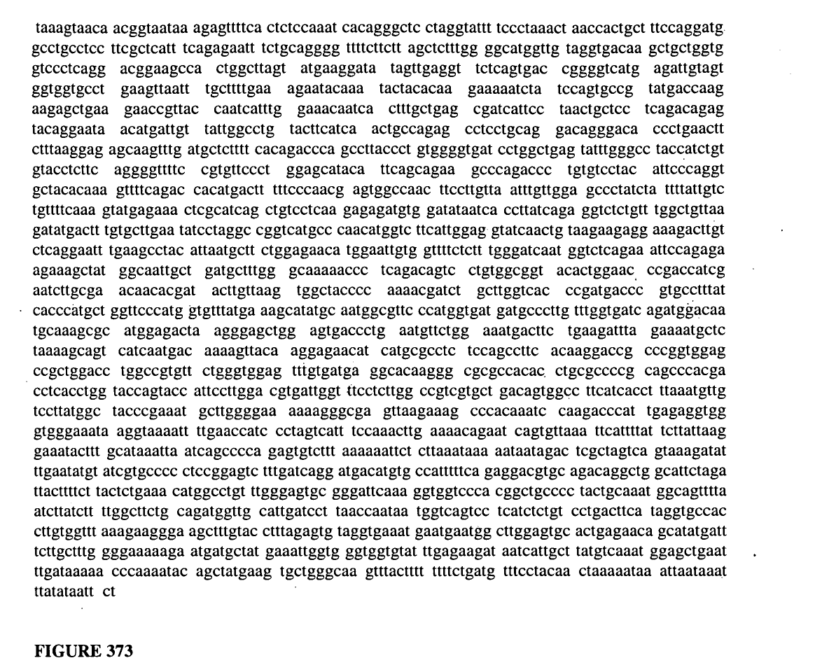

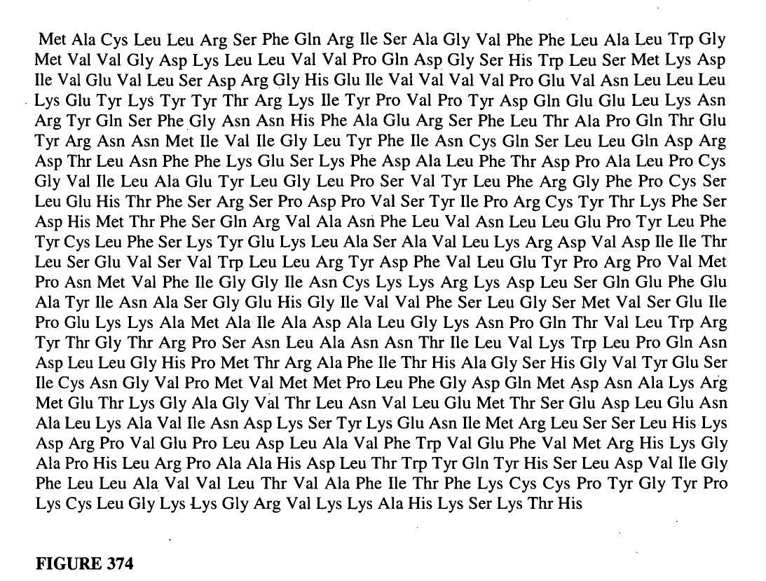

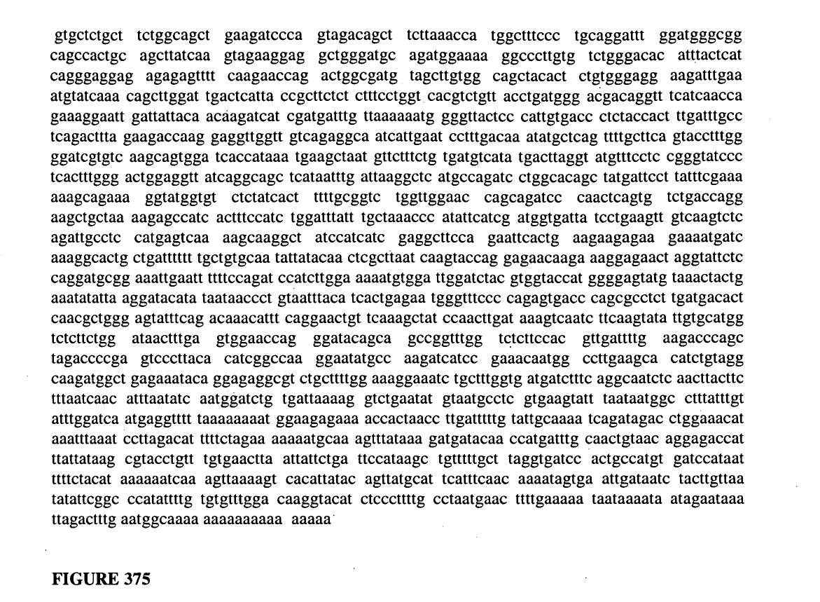

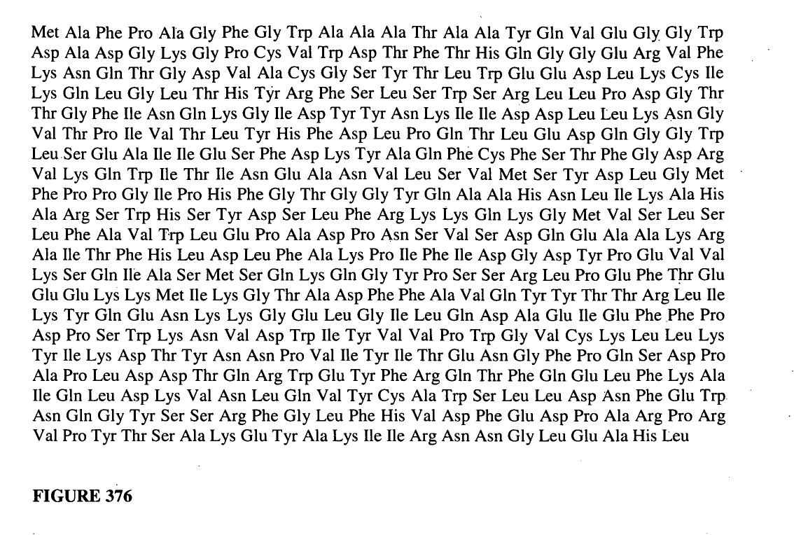

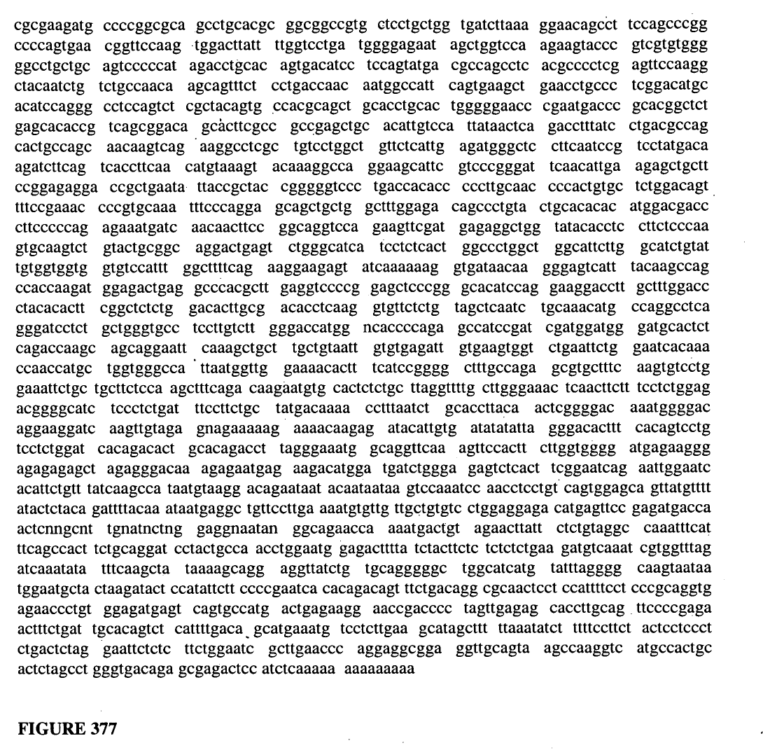

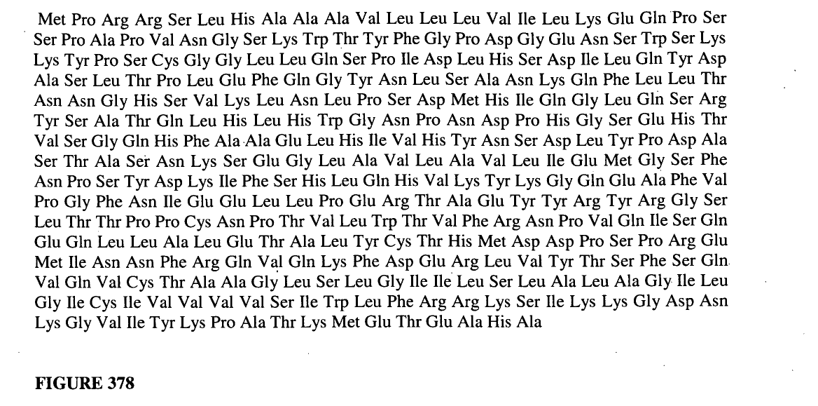

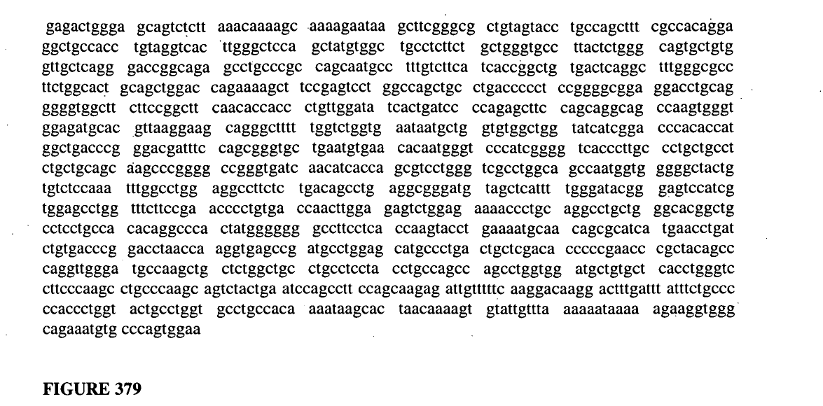

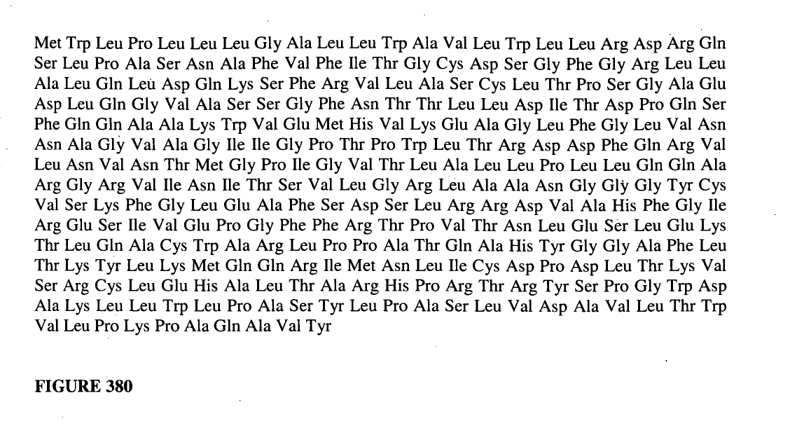

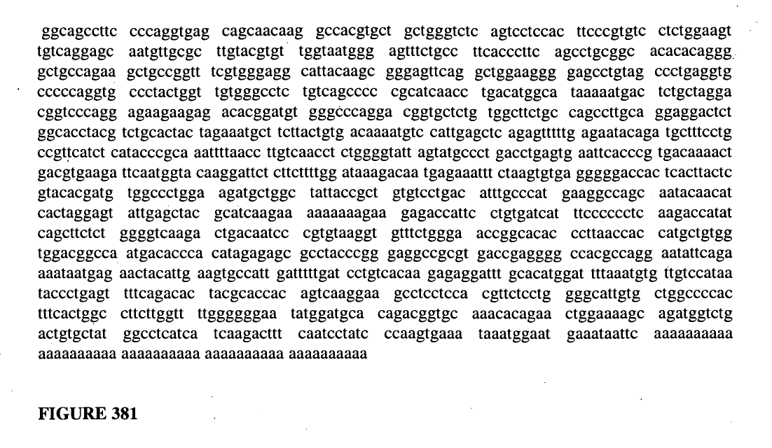

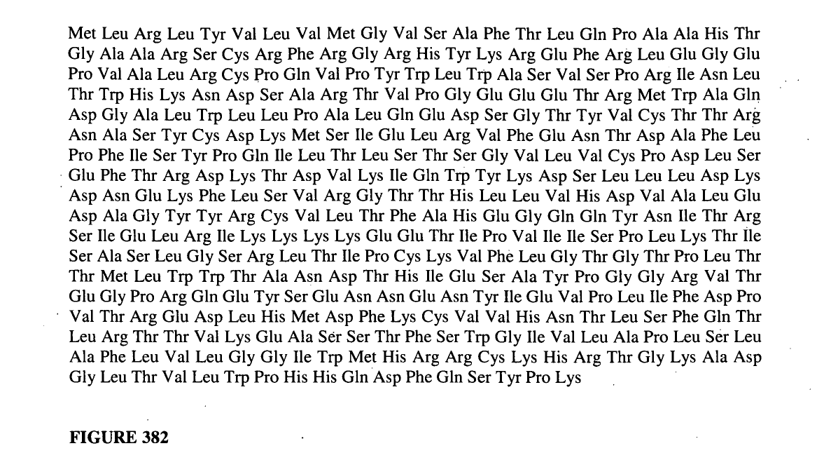

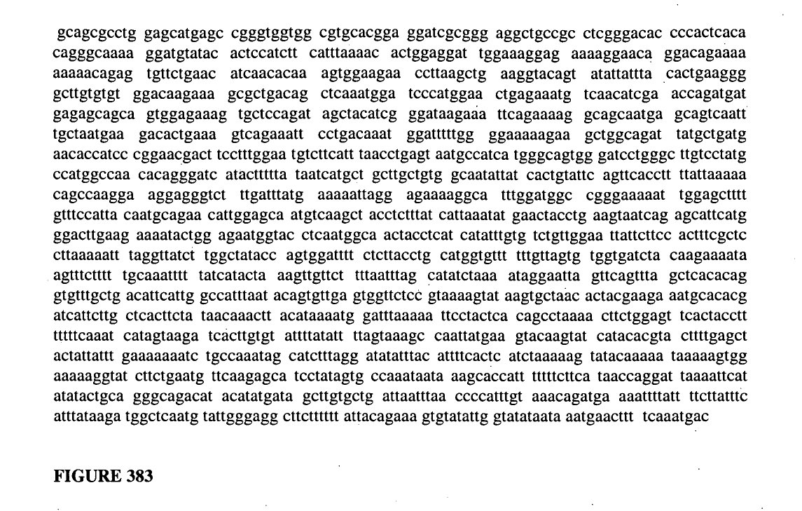

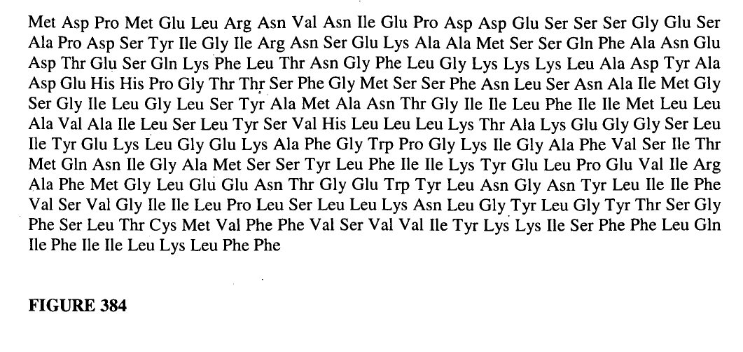

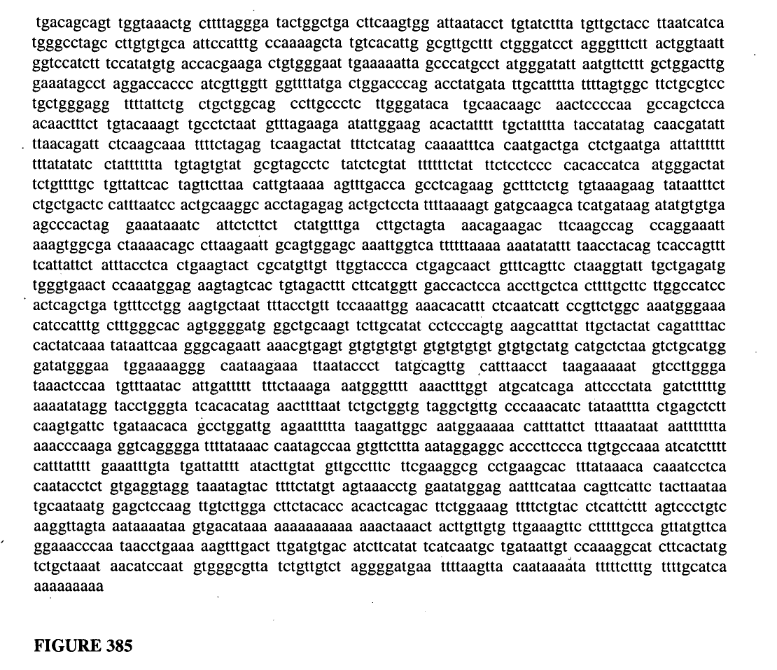

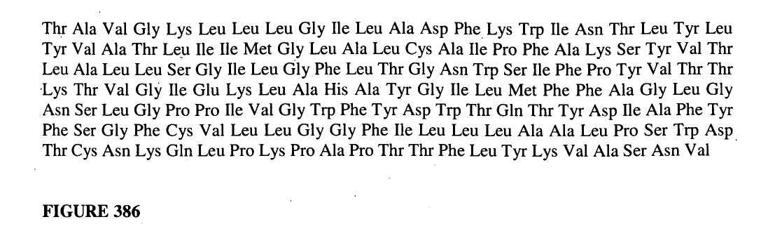

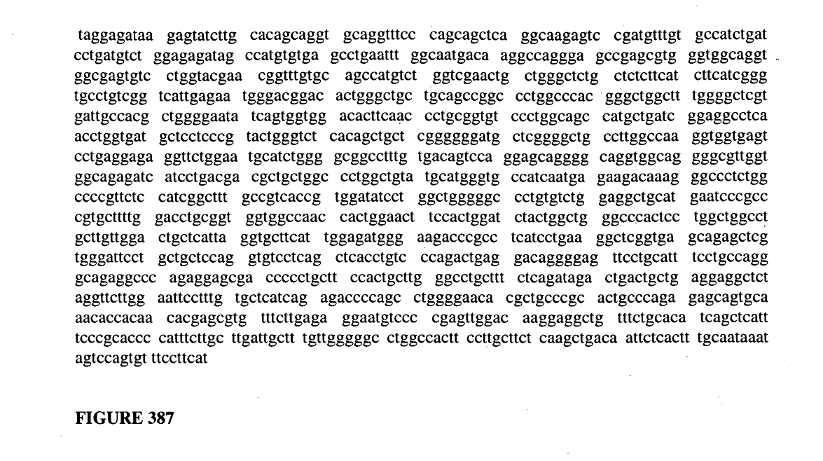

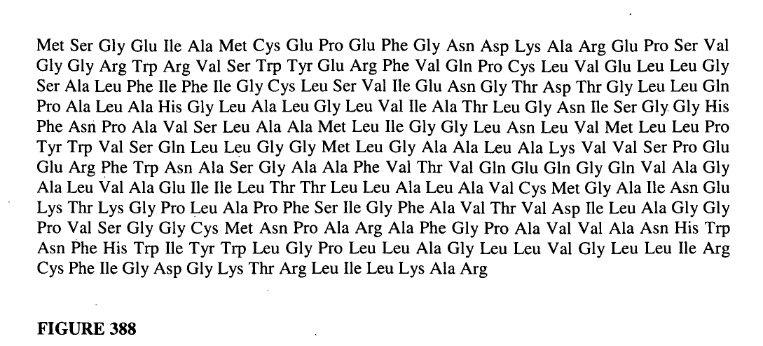

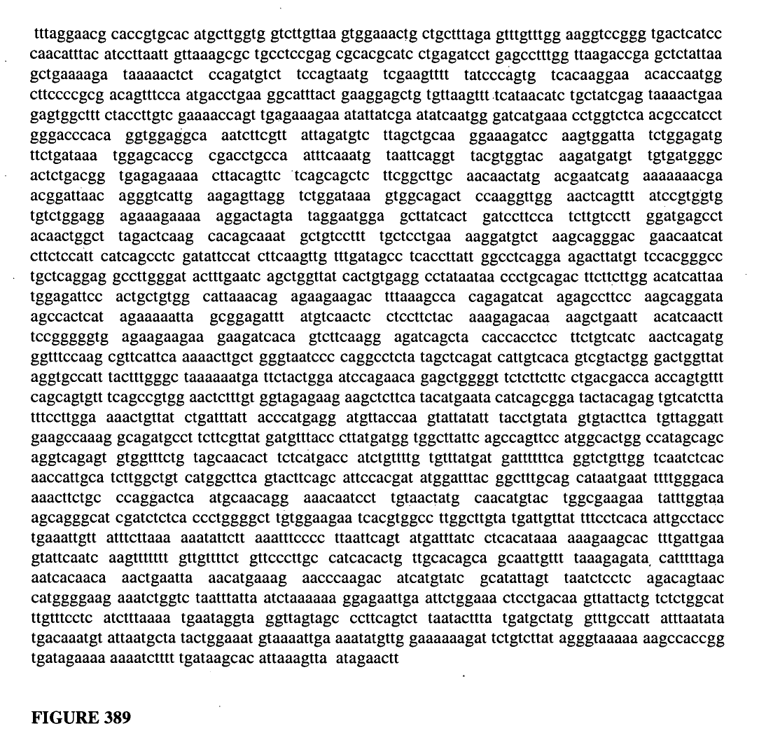

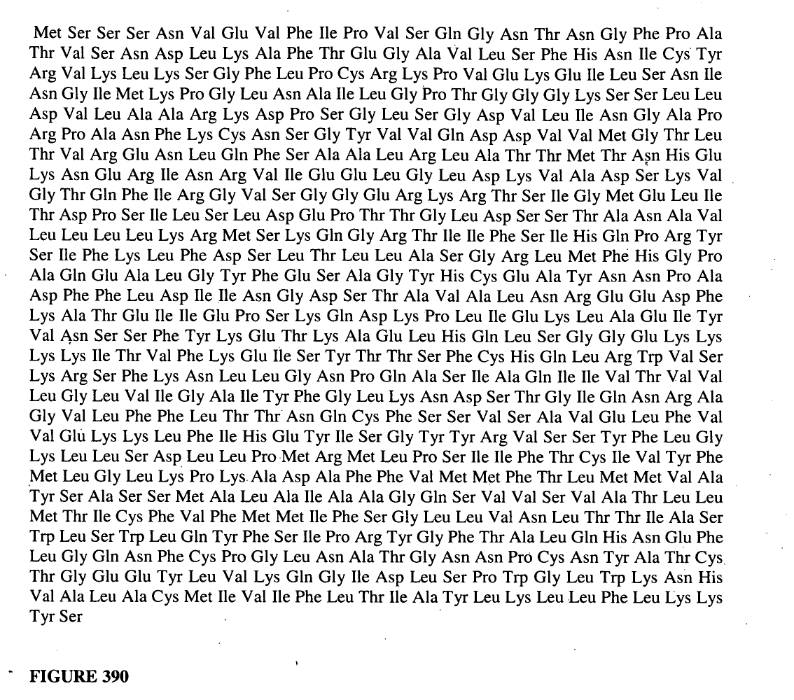

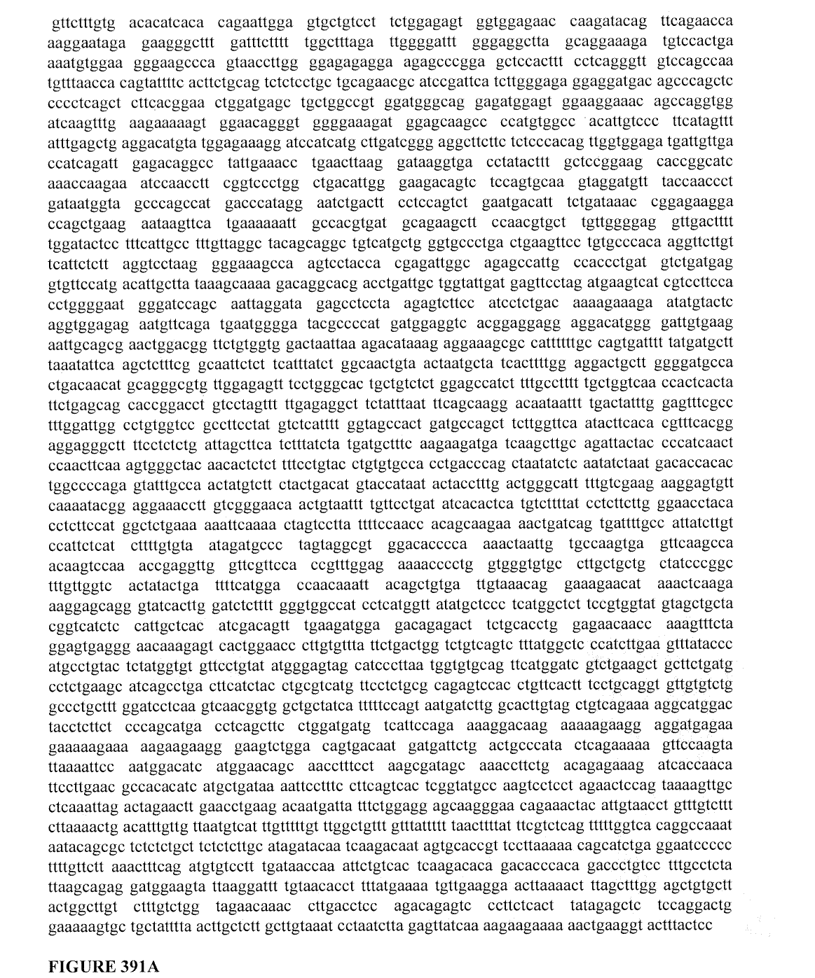

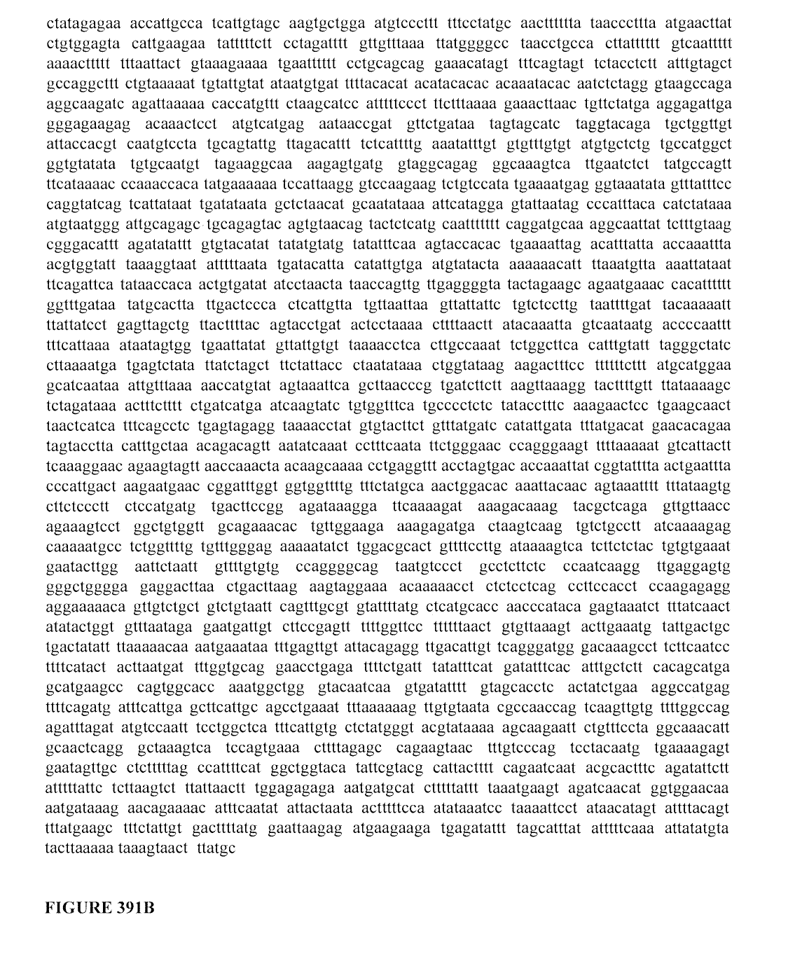

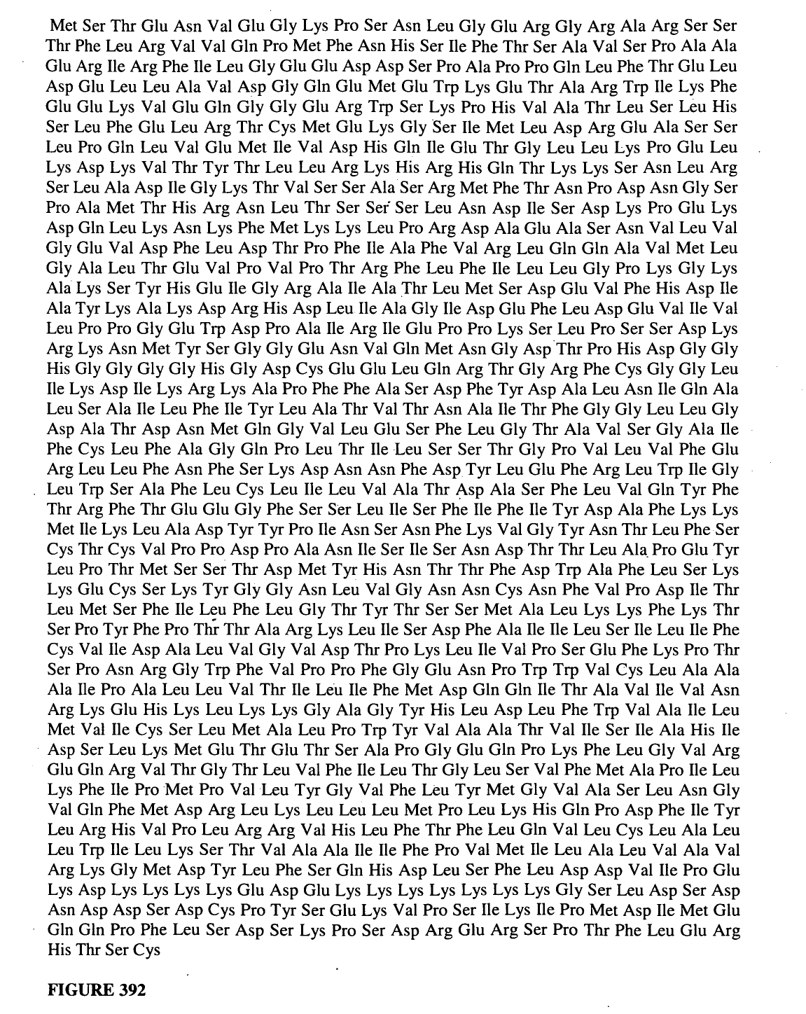

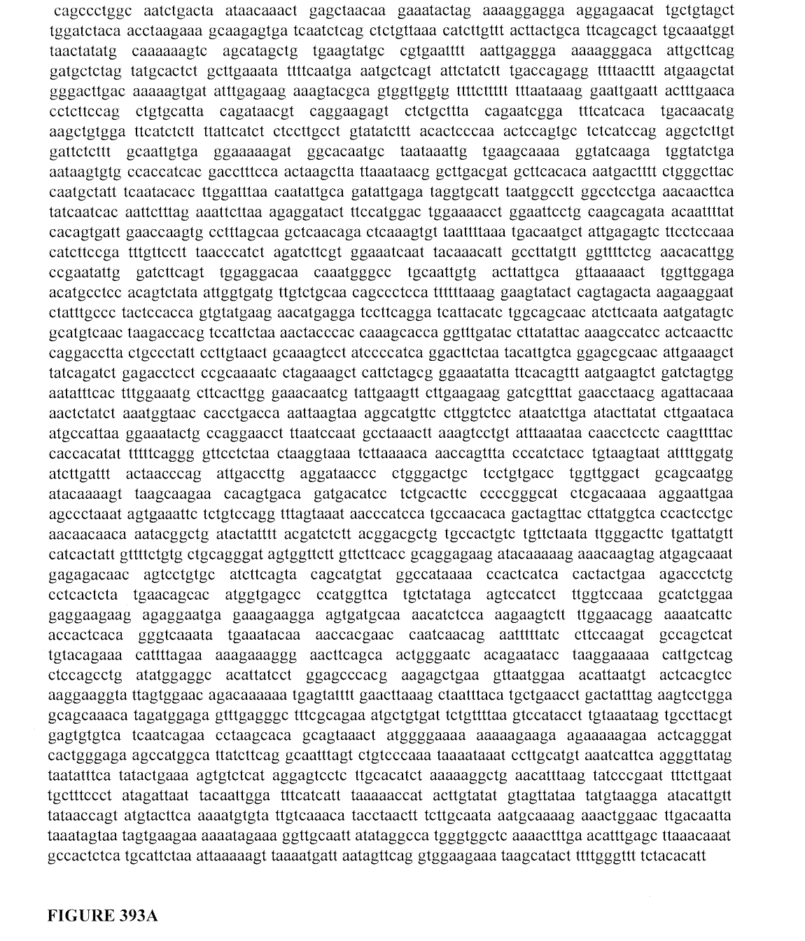

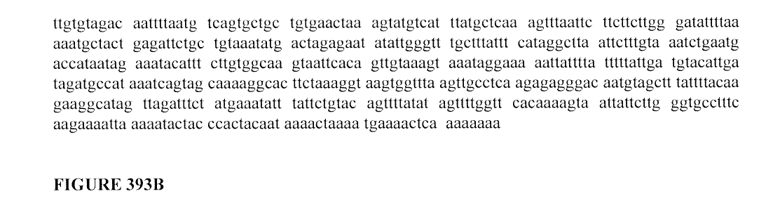

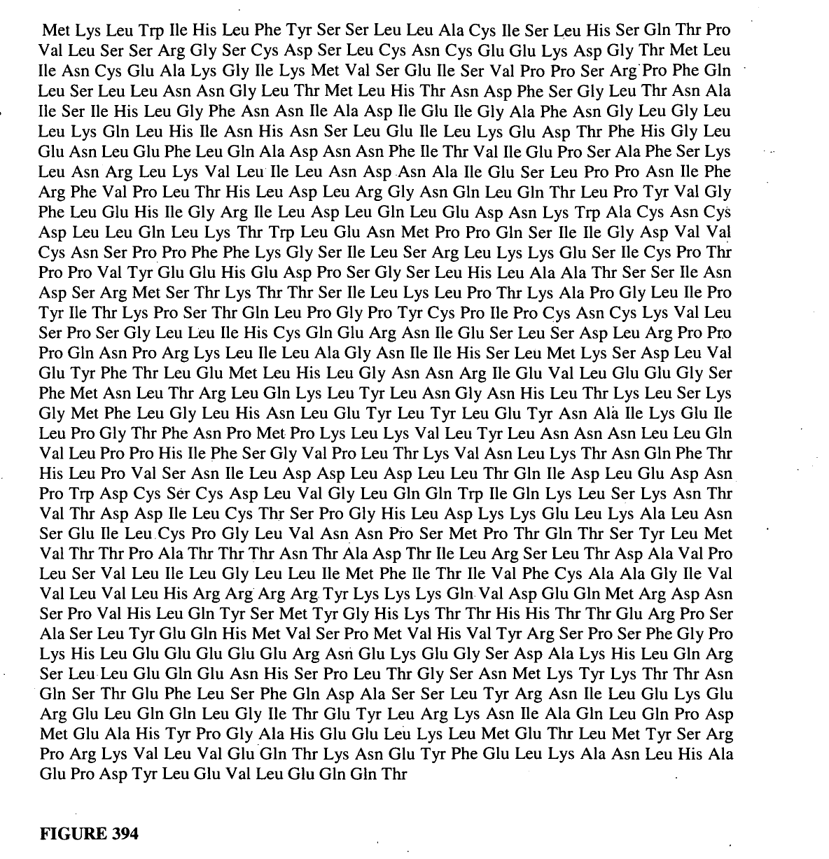

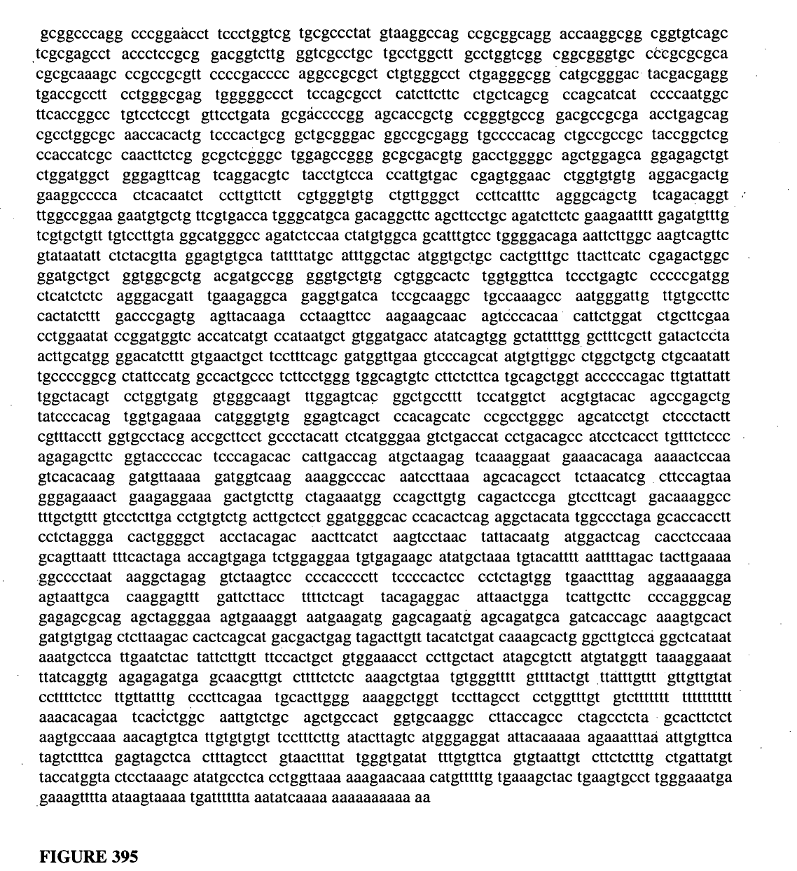

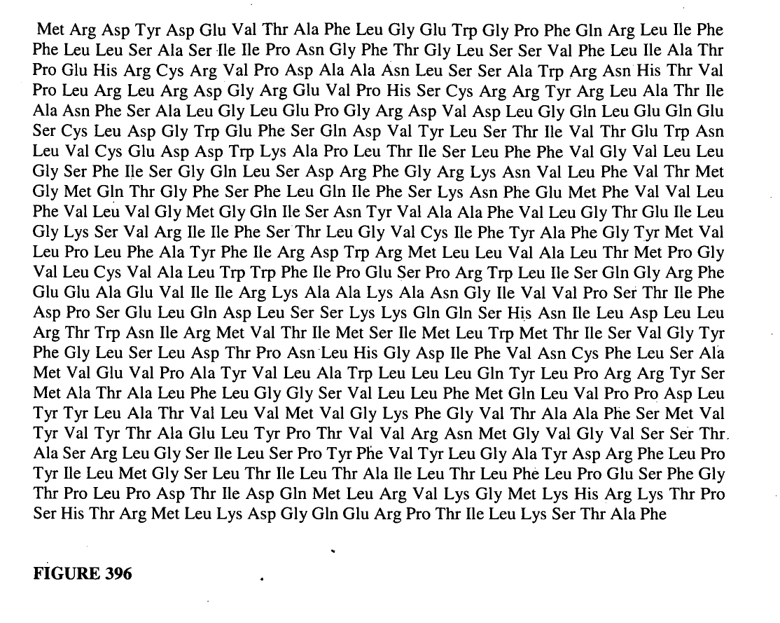

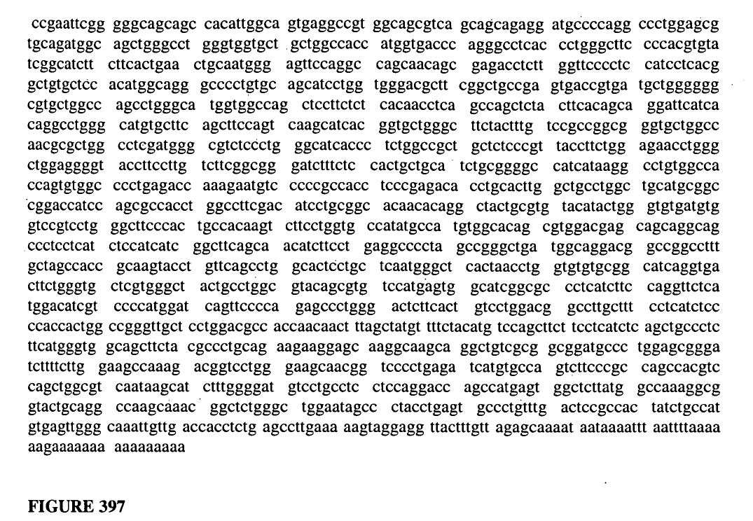

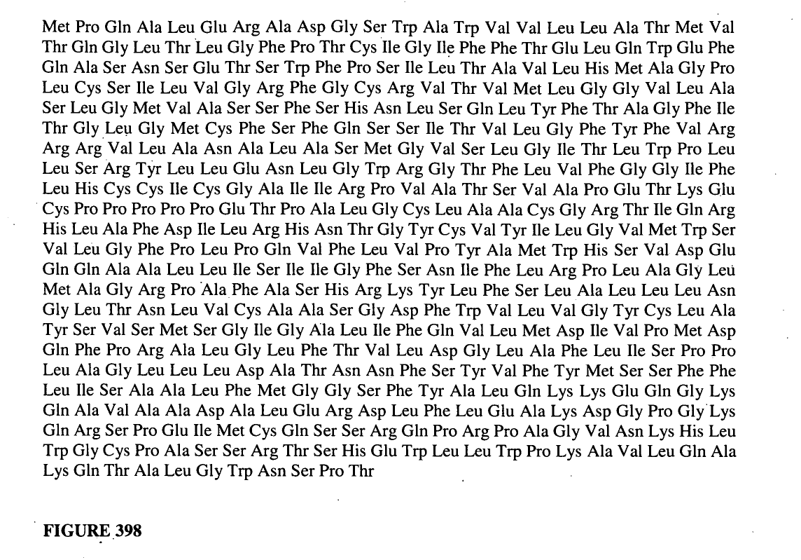

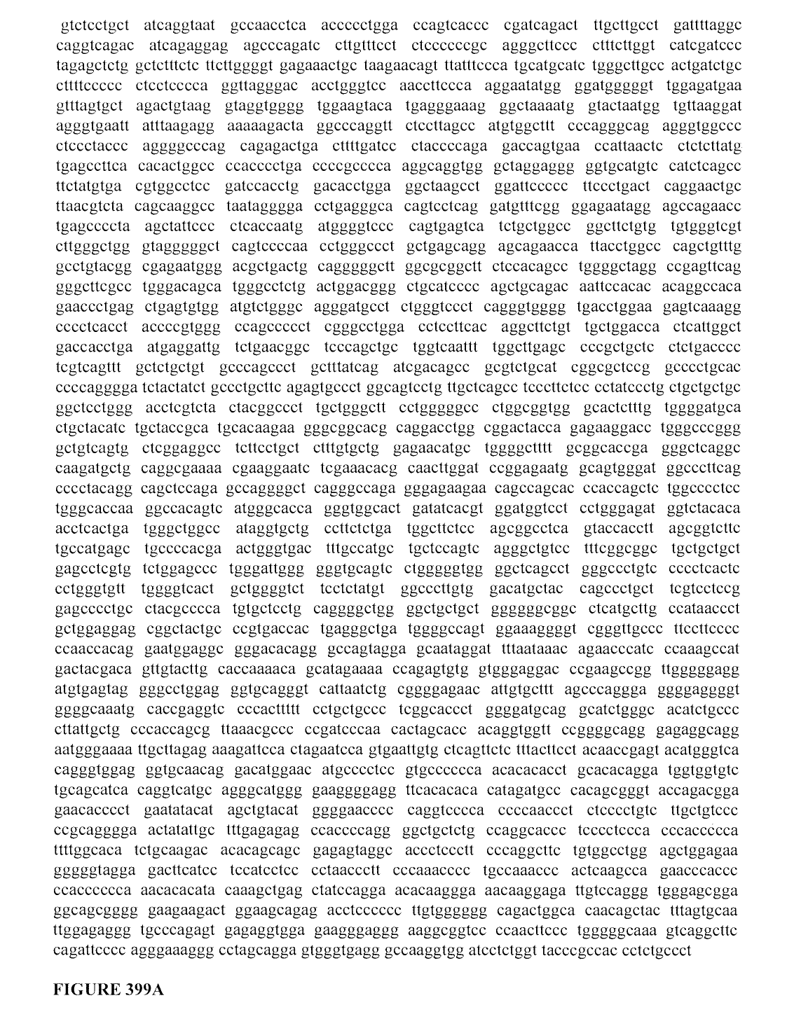

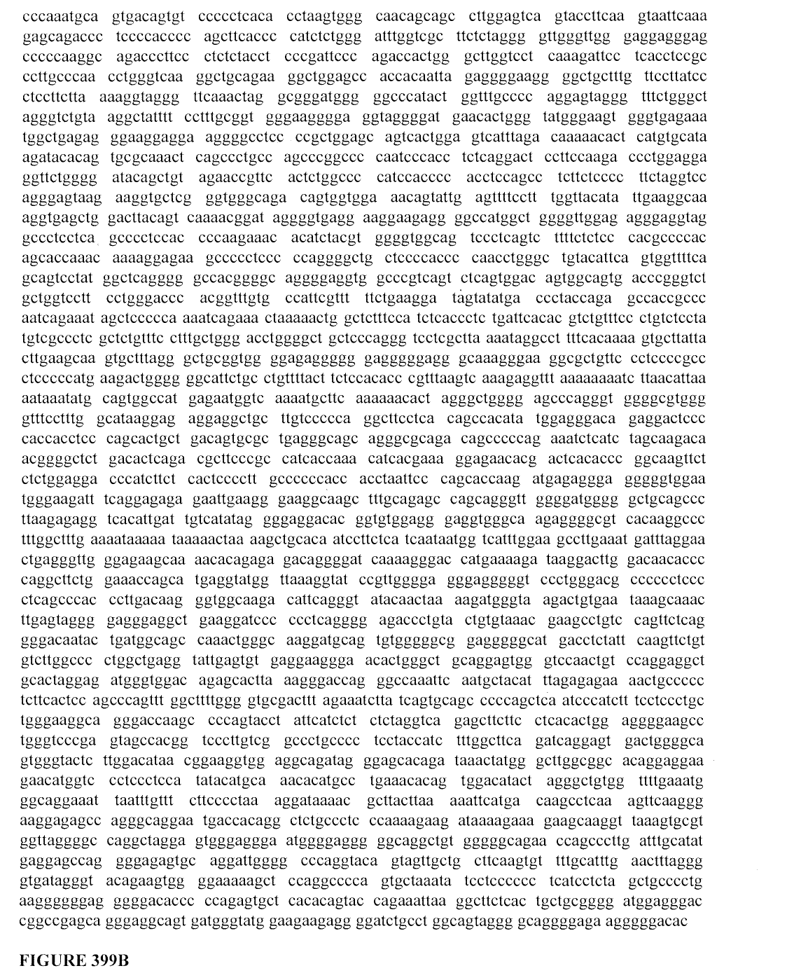

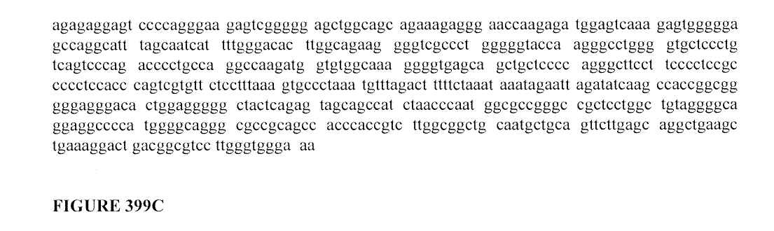

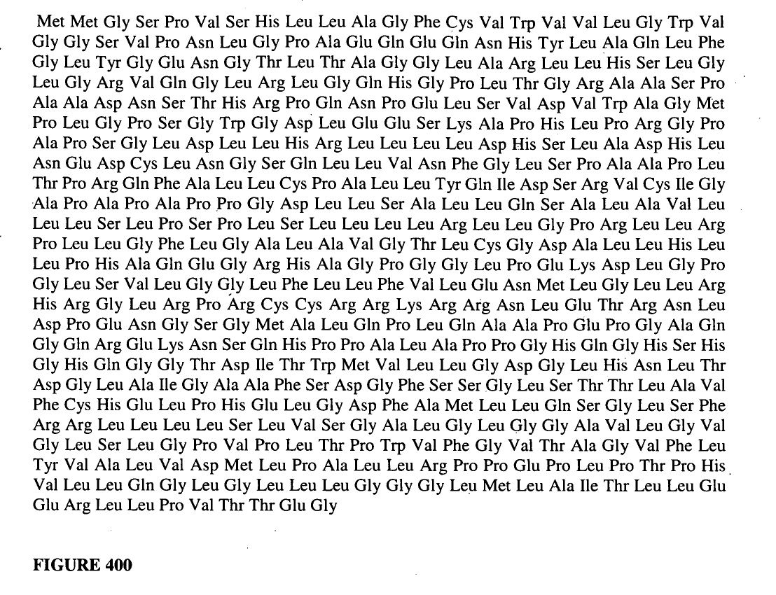

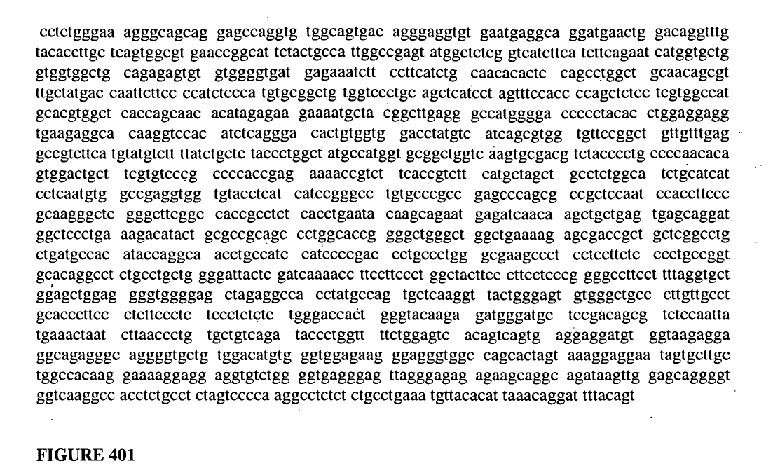

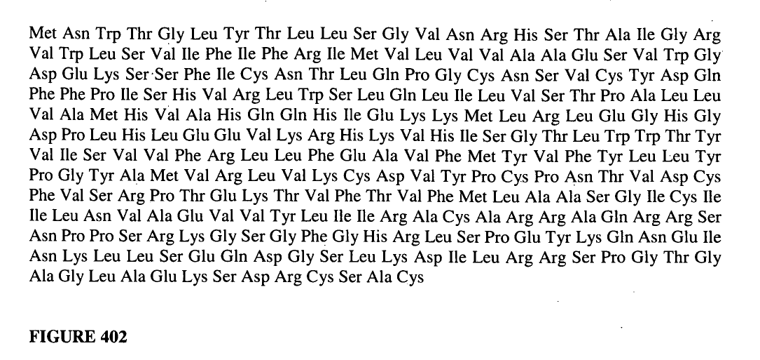

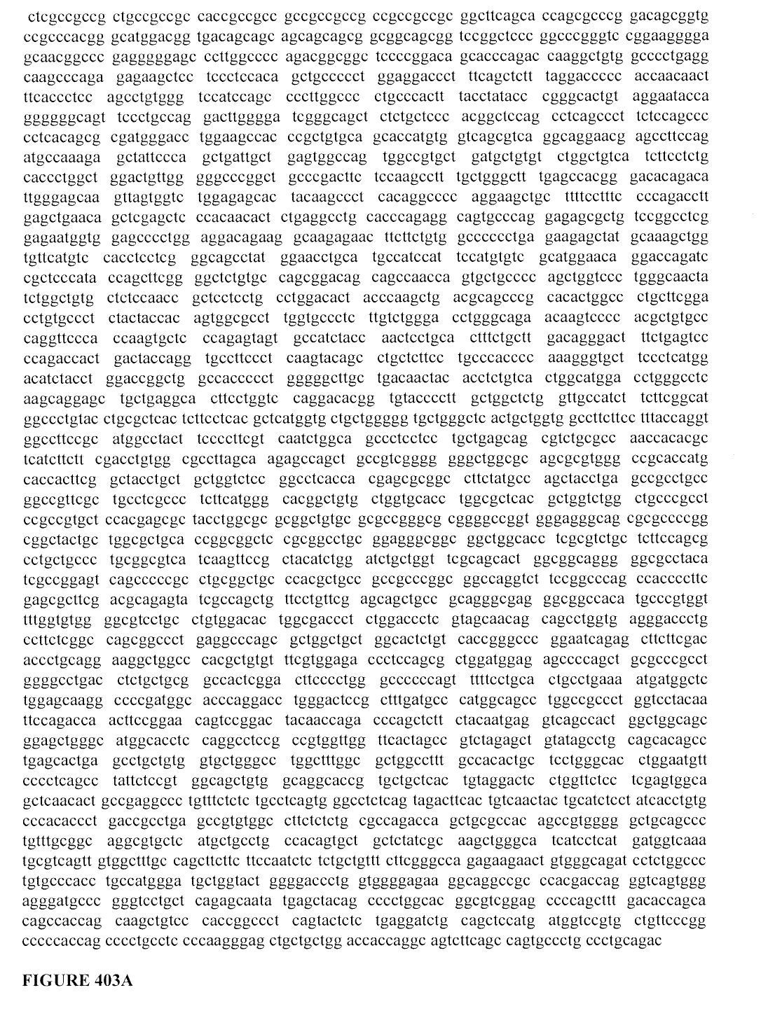

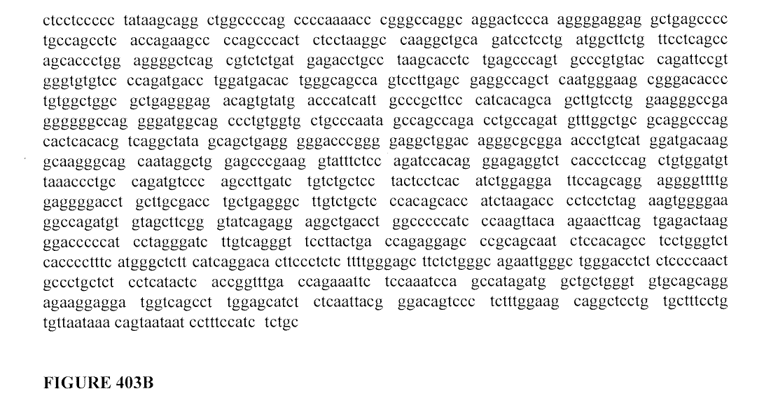

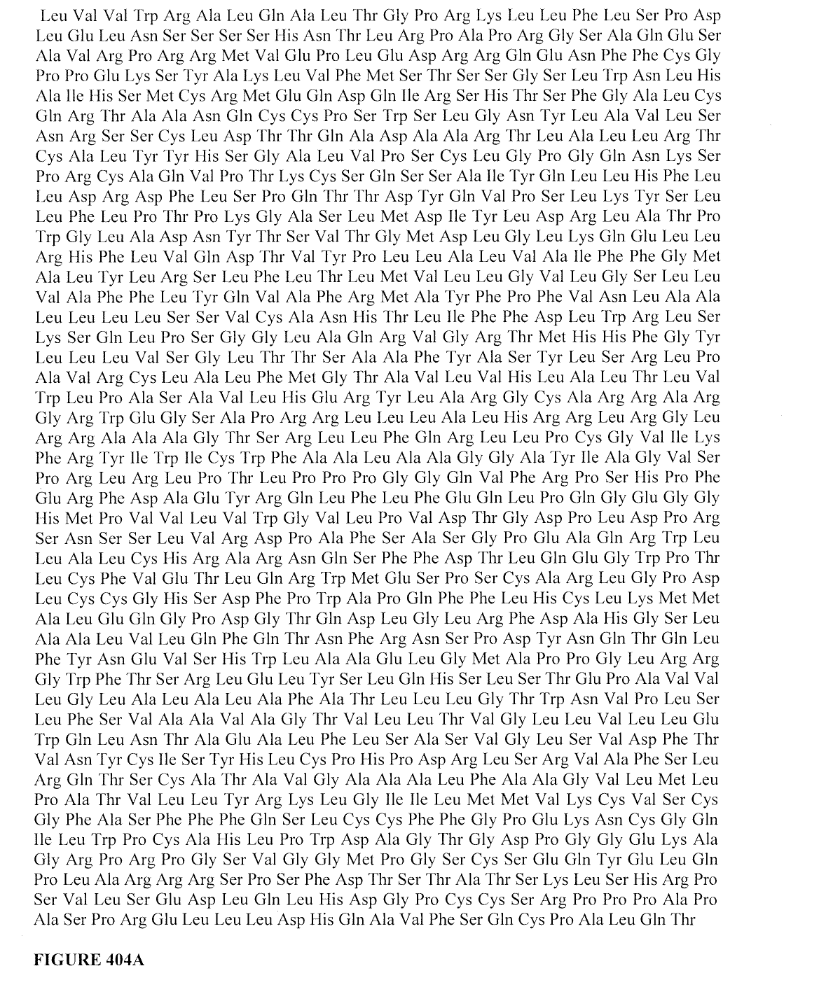

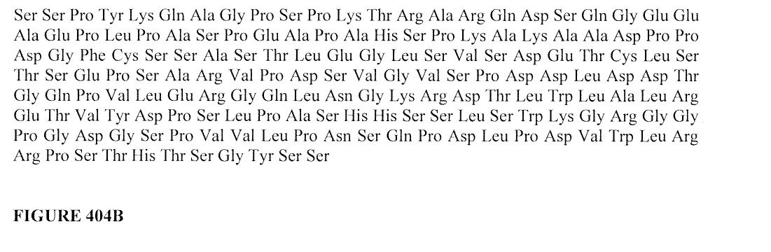

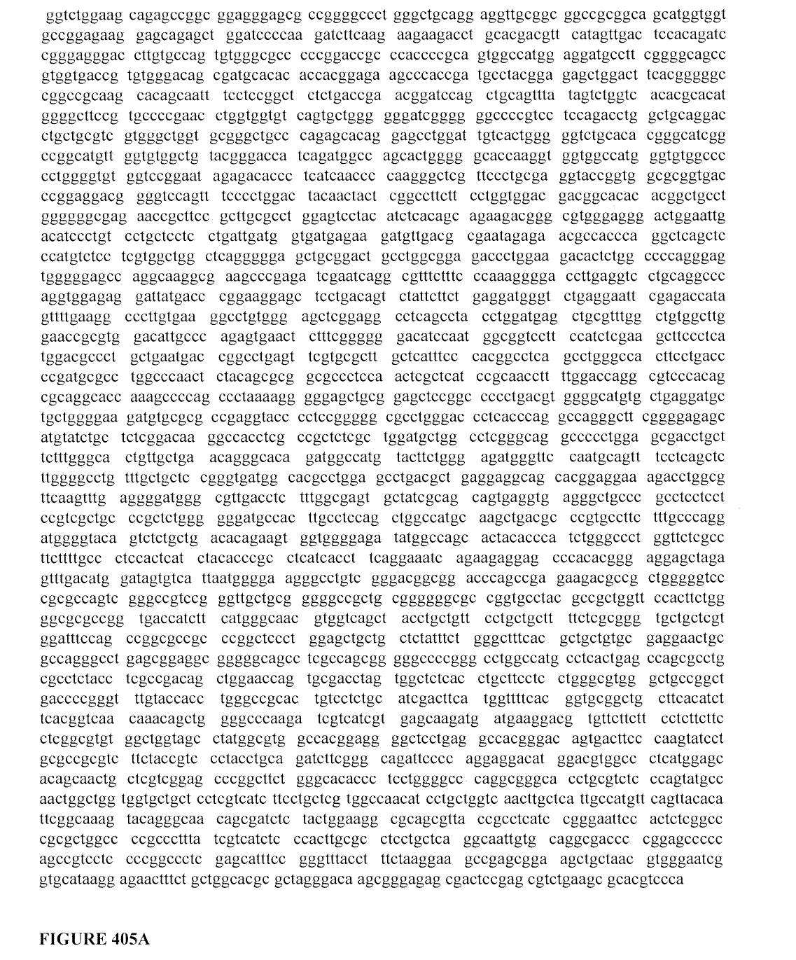

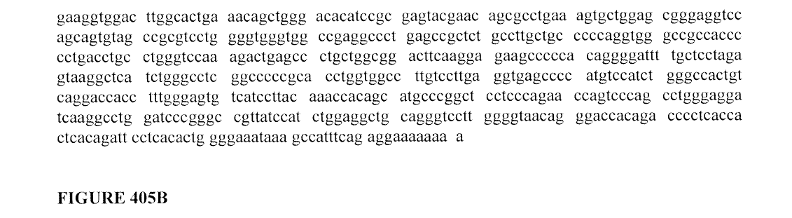

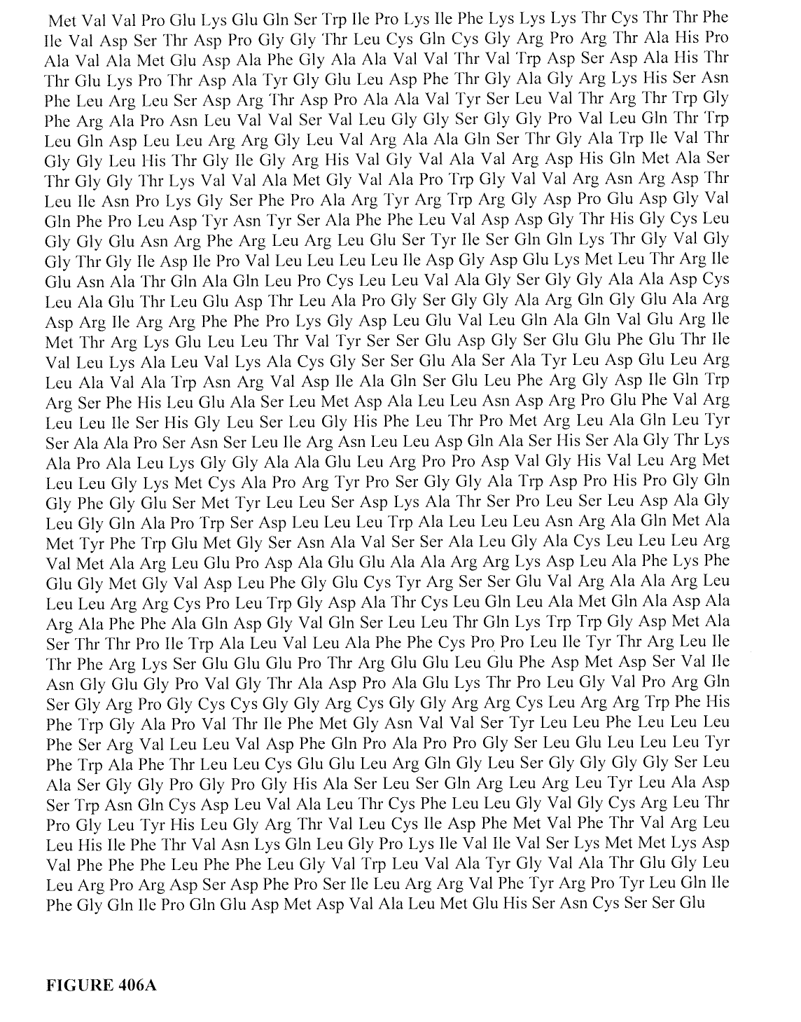

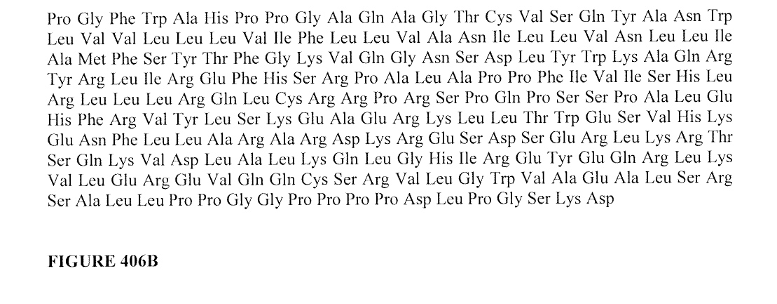

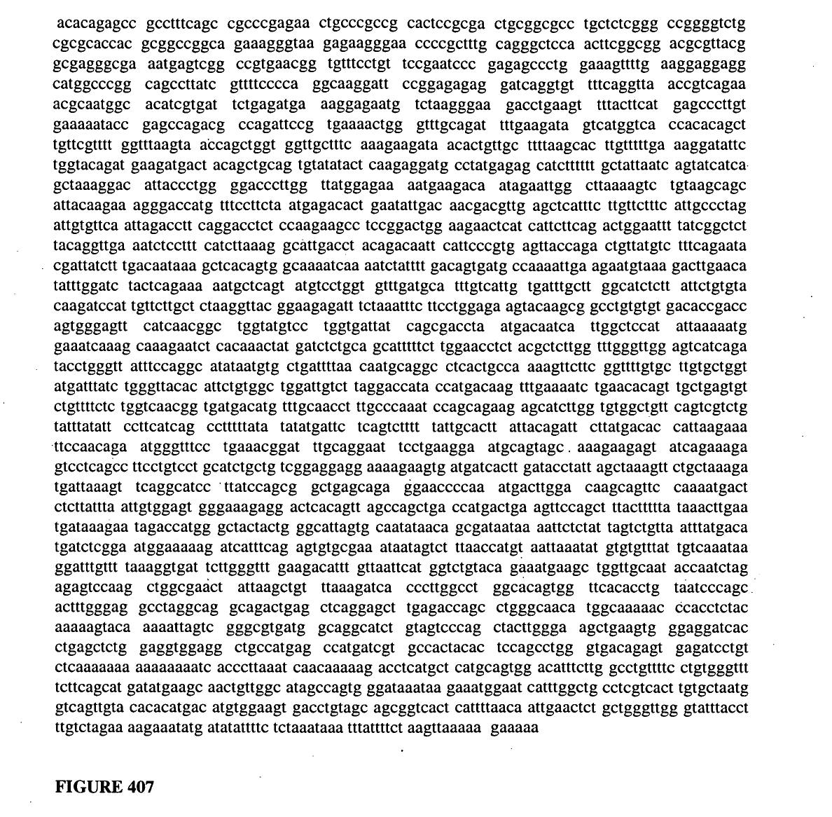

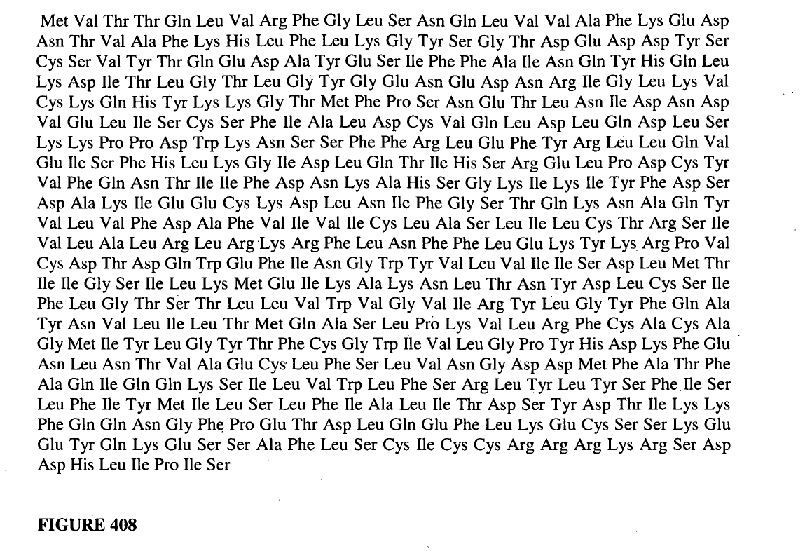

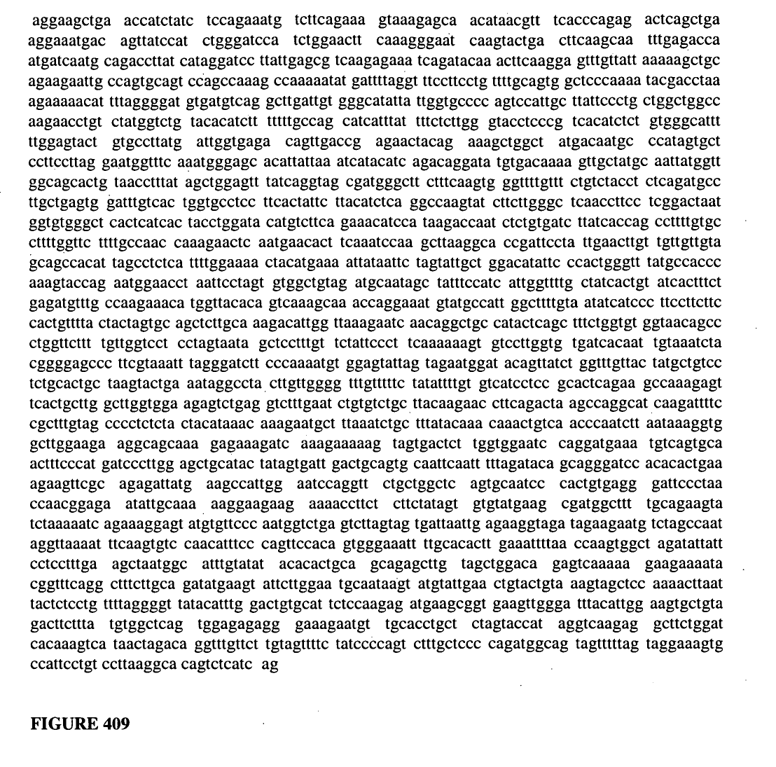

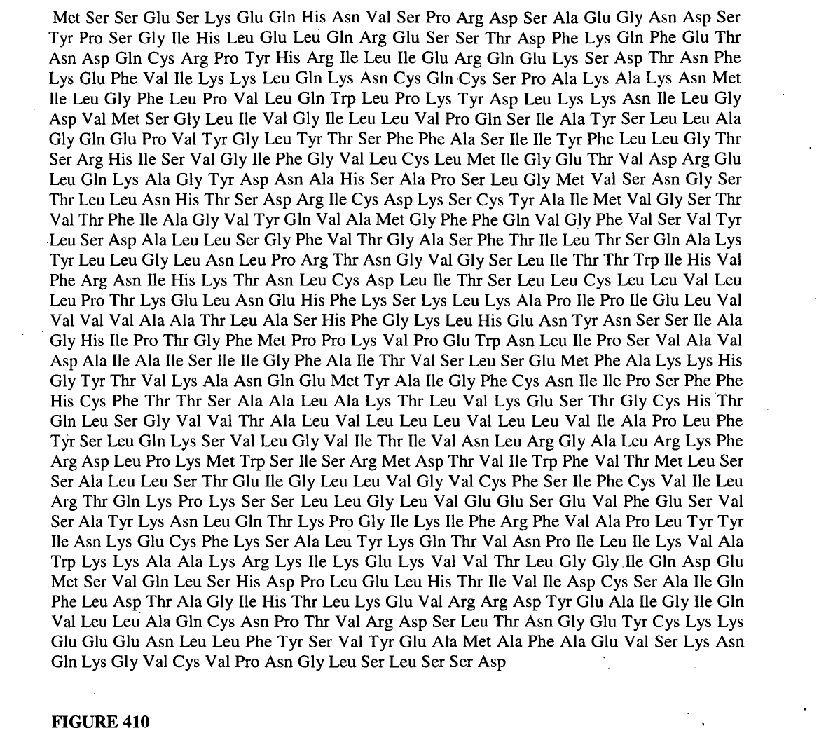

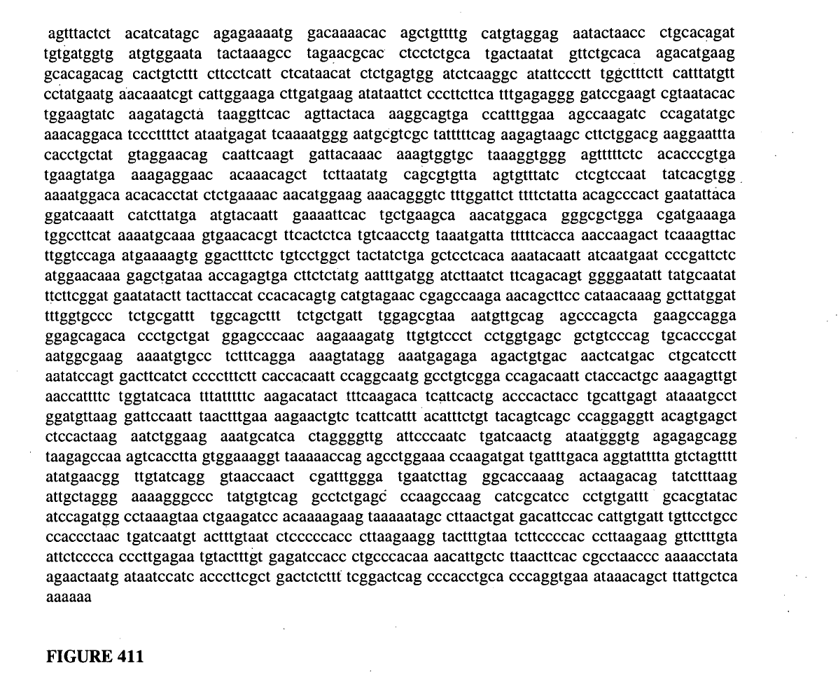

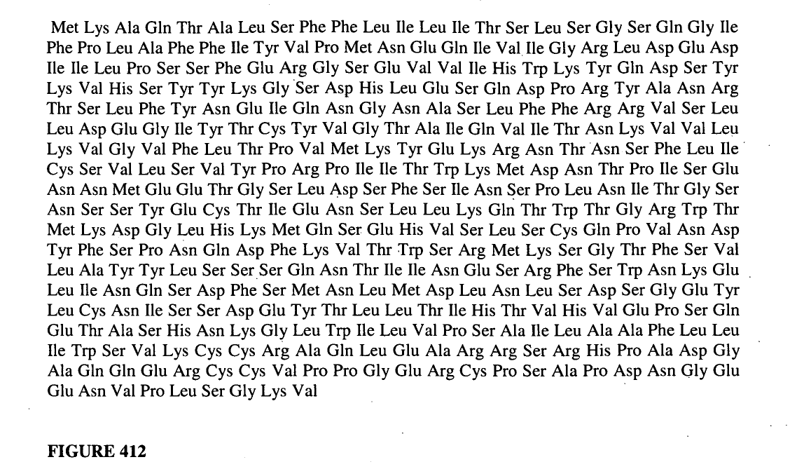

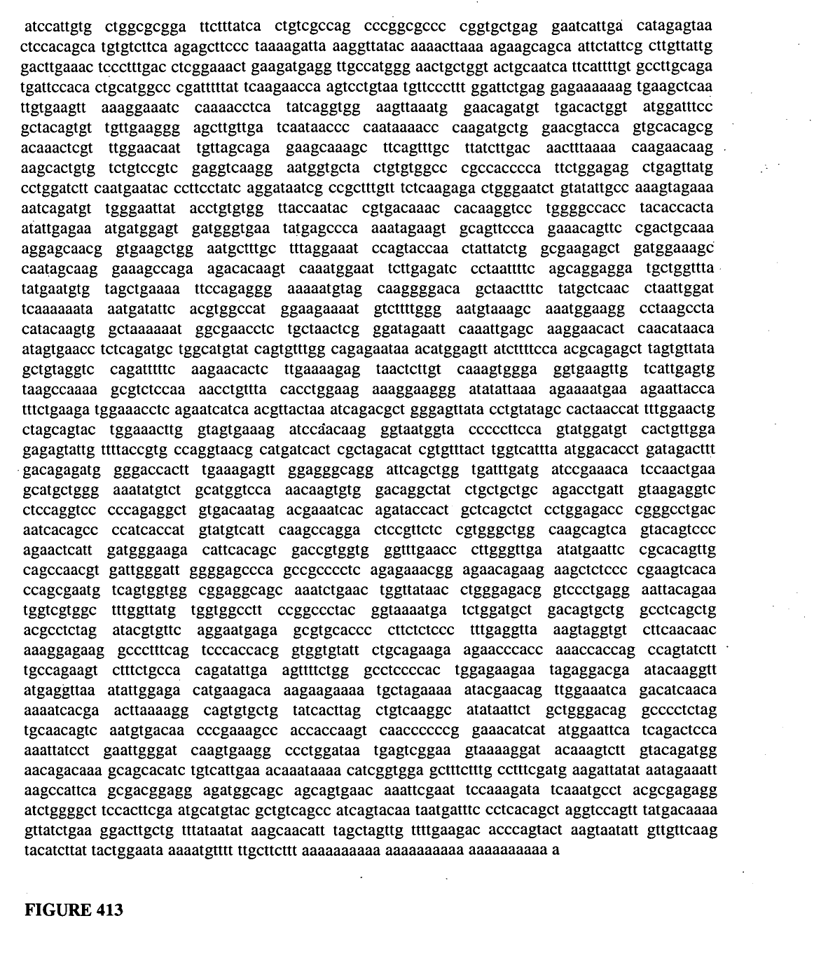

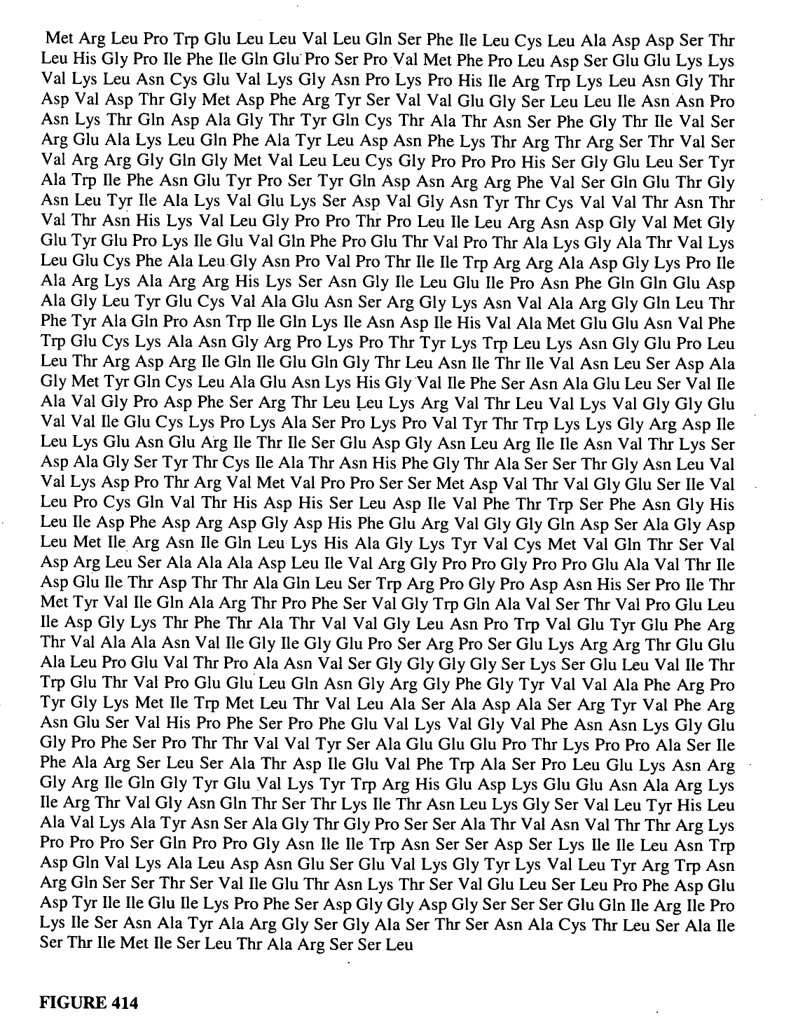

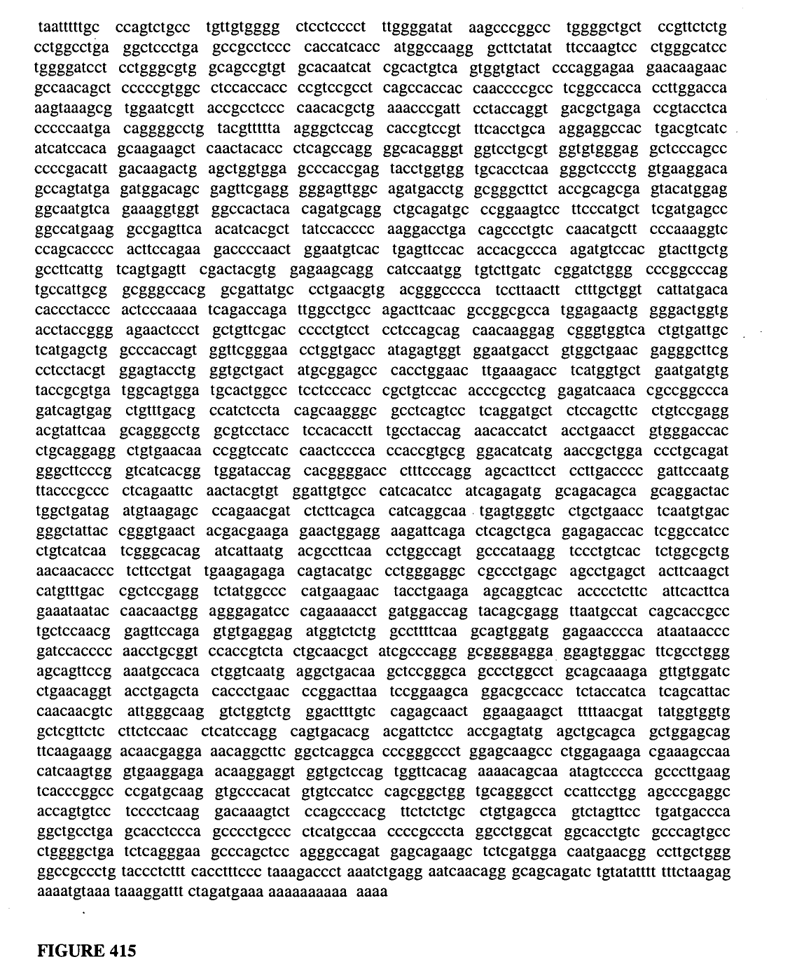

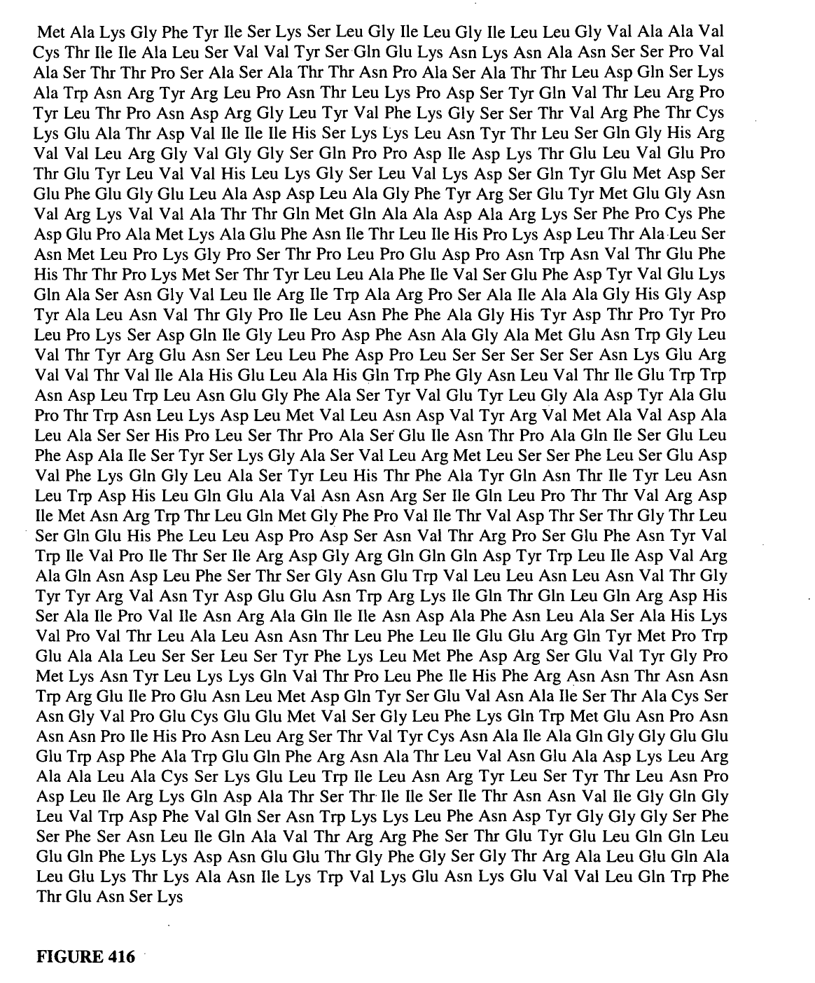

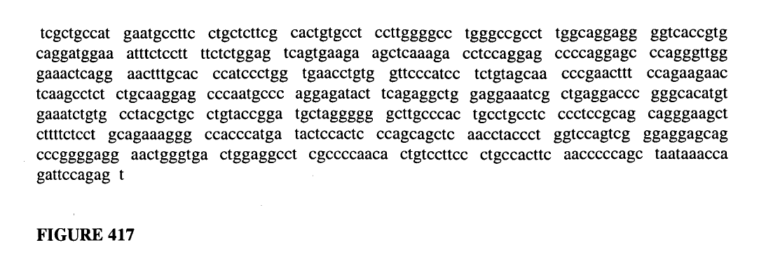

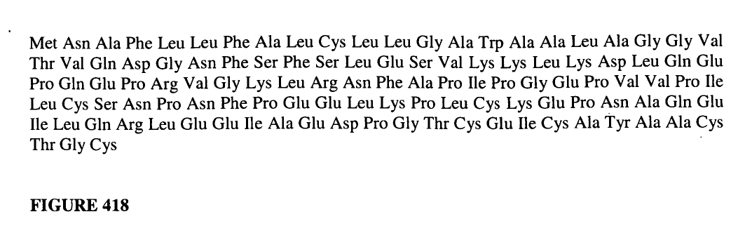

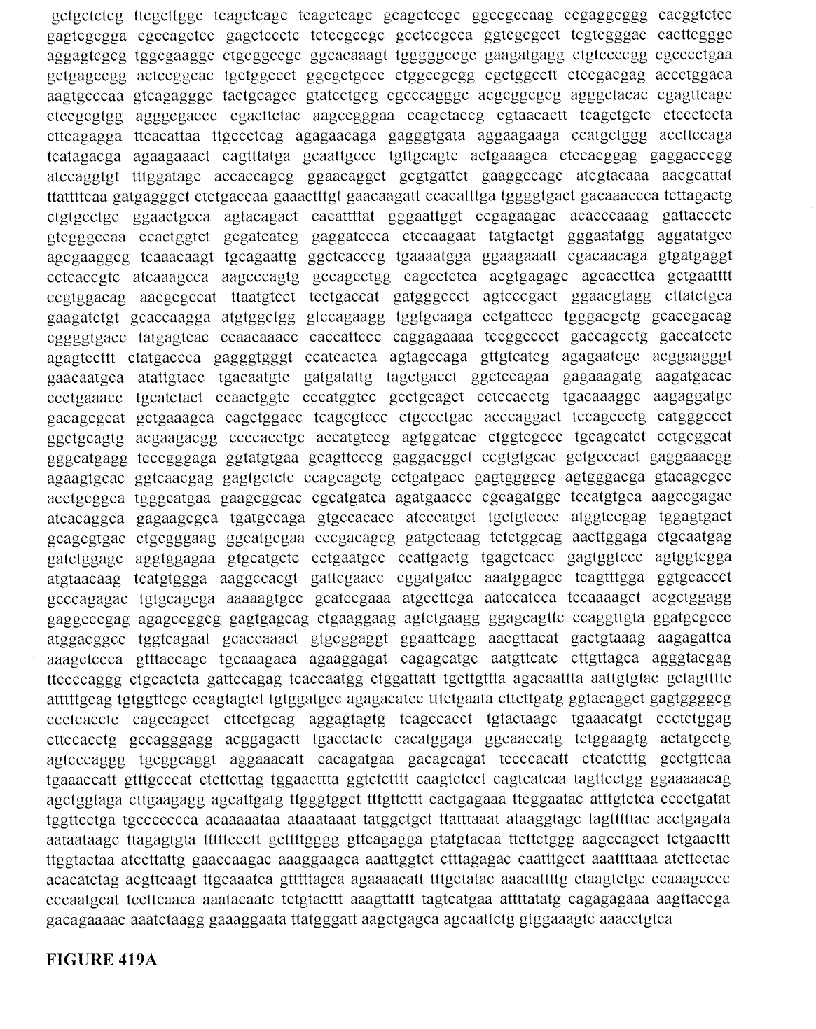

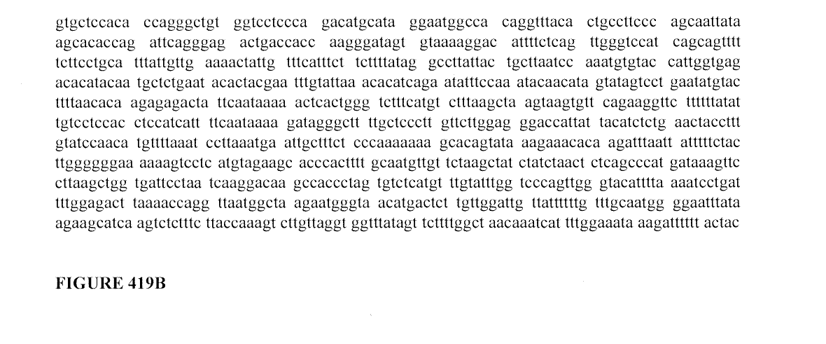

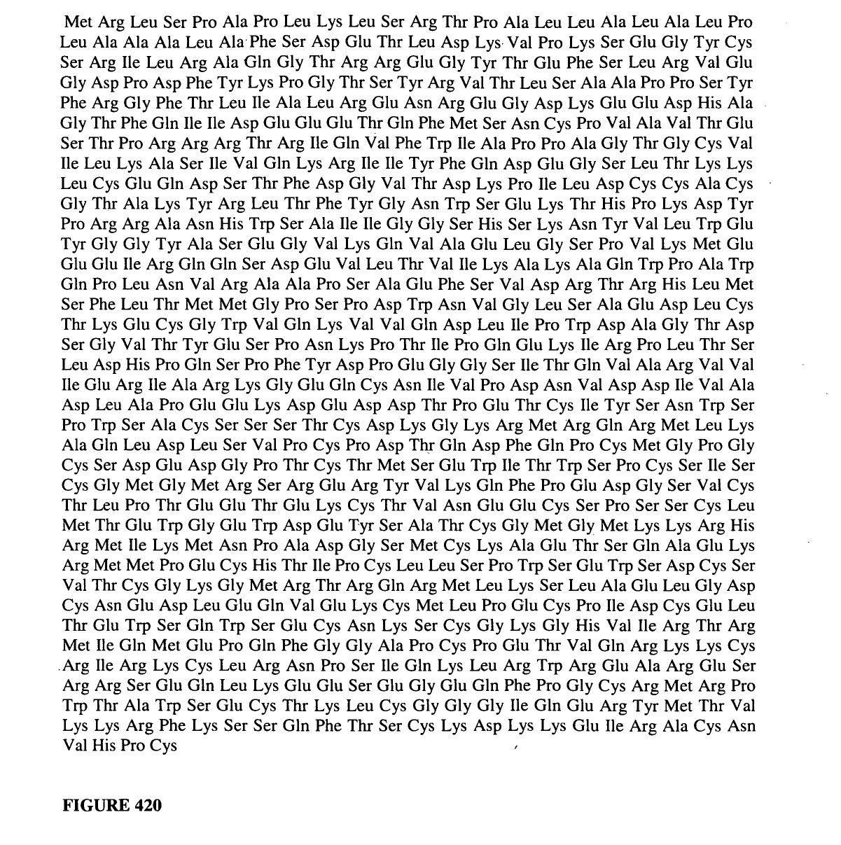

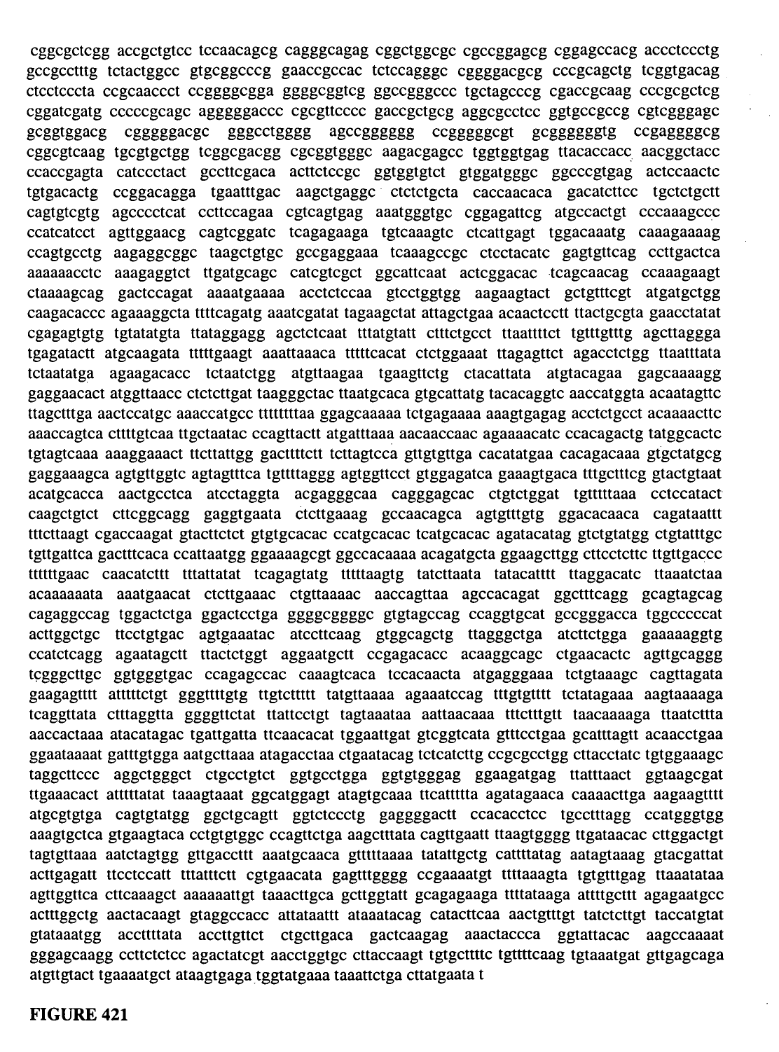

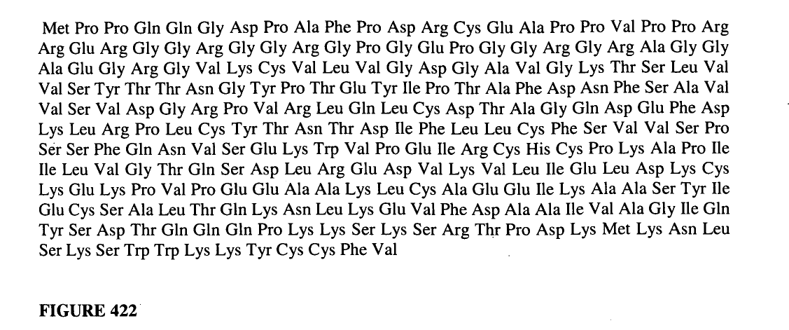

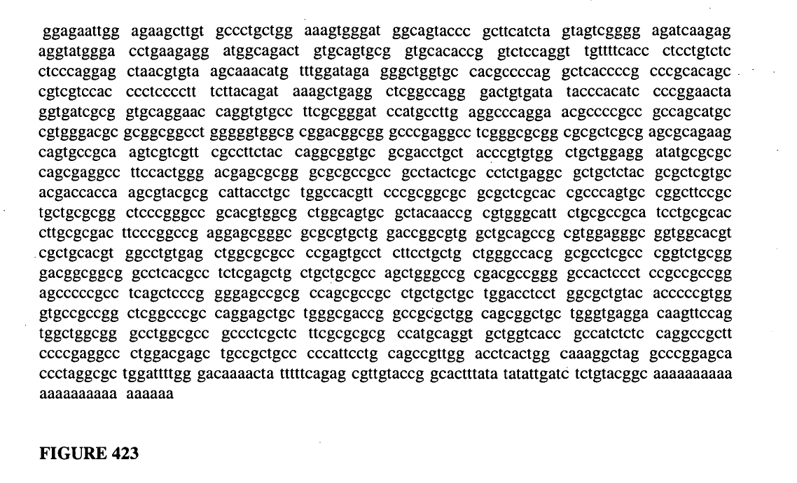

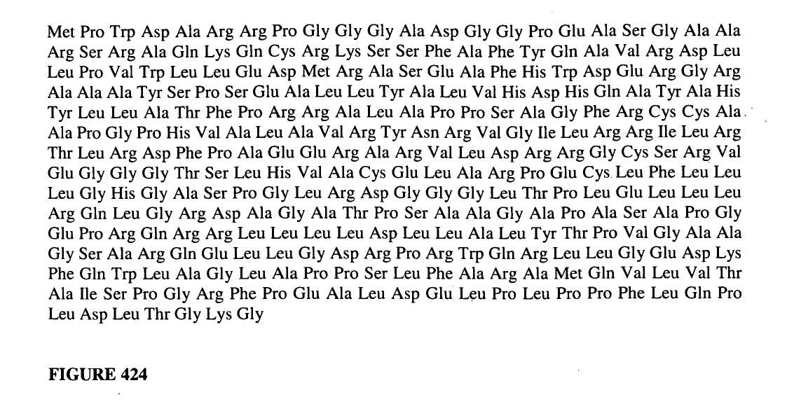

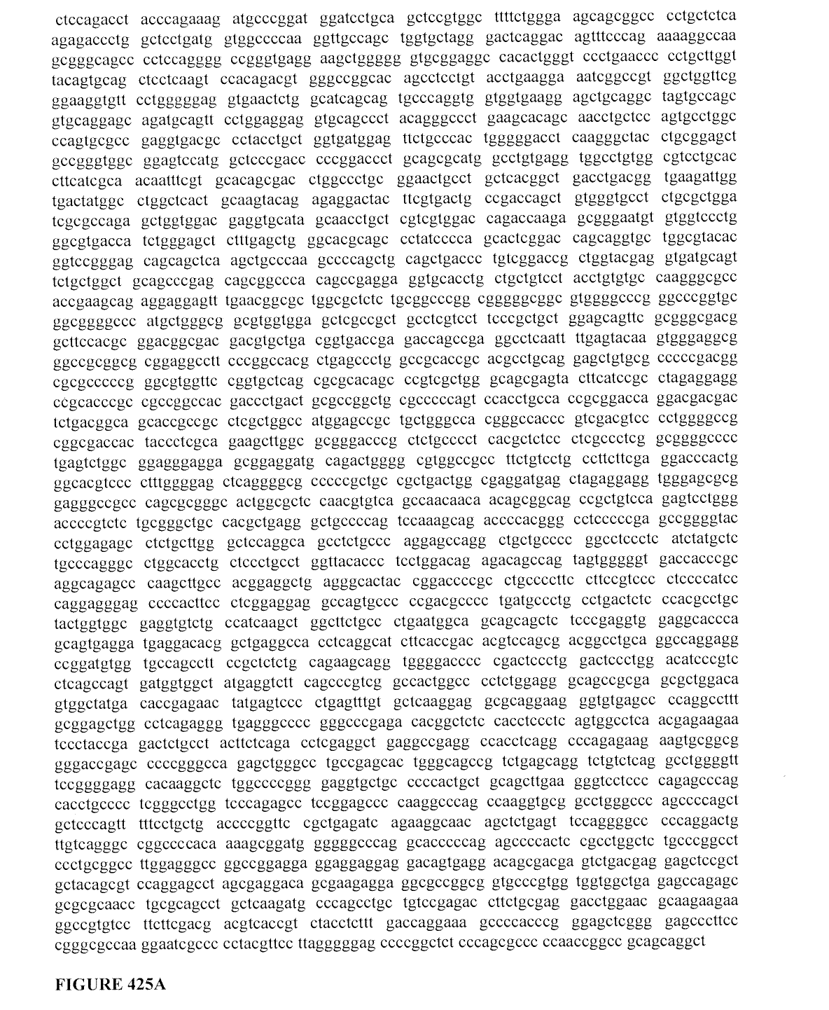

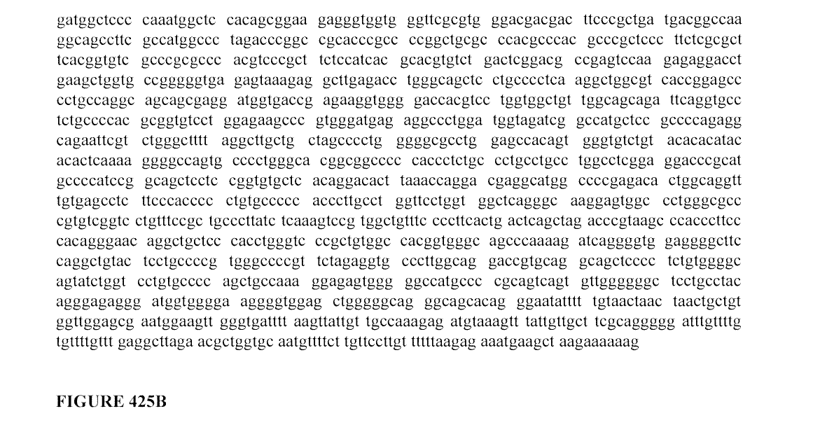

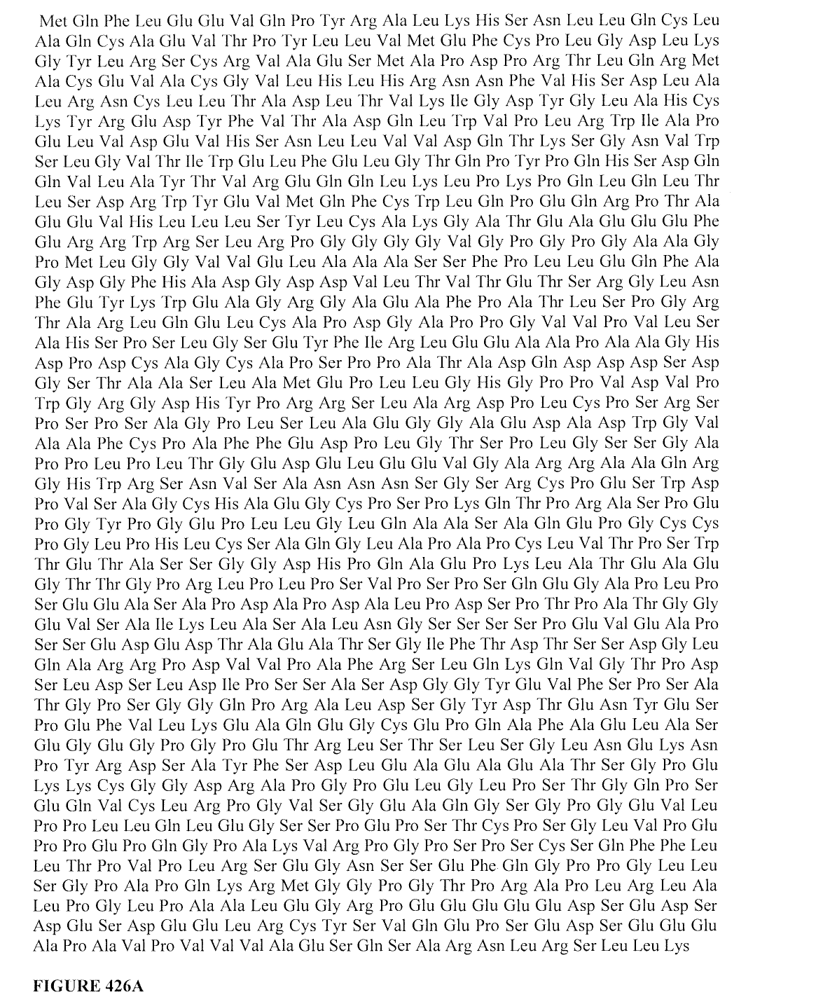

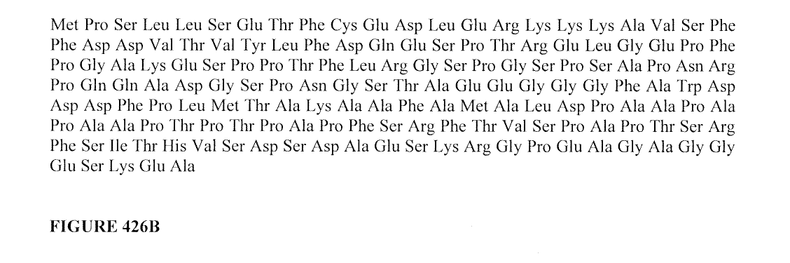

In one embodiment, an expression cluster identified herein includes genes upregulated in the left colon (FIG. 1).

In another embodiment, an expression cluster identified herein includes genes upregulated in the right colon (FIG. 1).

In one other embodiment, an expression cluster identified herein includes genes upregulated in the terminal ileum (FIG. 1).

In other embodiments, the expression cluster identified herein includes genes in the IBD2 locus (Table 7); or in the IBD5 locus (Table 8).

In some embodiments, the expression cluster identified herein includes genes classified under an immune response.

In other embodiments, the expression cluster identified herein includes genes classified under a response to wounding.

k. Design of Intron-Based PCR Primers and Probes

According to one aspect of the present invention, PCR primers and probes are designed based upon intron sequences present in the gene to be amplified. Accordingly, the first step in the primer/probe design is the delineation of intron sequences within the genes. This can be done by publicly available software, such as the DNA BLAT software developed by Kent, W. J., Genome Res. 12(4):656-64 (2002), or by the BLAST software including its variations. Subsequent steps follow well established methods of PCR primer and probe design.

In order to avoid non-specific signals, it is important to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. In: Krawetz S, Misener S (eds) Bioinformatics Methods and Protocols: Methods in Molecular Biology. Humana Press, Totowa, N. J., pp 365-386).

The most important factors considered in PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases. Tm's between 50 and 80° C., e.g. about 50 to 70° C. are typically preferred.

For further guidelines for PCR primer and probe design see, e.g. Dieffenbach, C. W. et al., “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods Mol. Biol. 70:520-527 (1997), the entire disclosures of which are hereby expressly incorporated by reference.

1. IBD Gene Set, Assayed Gene Subsequences, and Clinical Application of Gene Expression Data

An important aspect of the present invention is to use the measured expression of certain genes by colonic issue to provide diagnostic information. For this purpose it is necessary to correct for (normalize away) both differences in the amount of RNA assayed and variability in the quality of the RNA used. Therefore, the assay typically measures and incorporates the expression of certain normalizing genes, including well known housekeeping genes, such as GAPDH and Cyp1. Alternatively, normalization can be based on the mean or median signal (Ct) of all of the assayed genes or a large subset thereof (global normalization approach). On a gene-by-gene basis, measured normalized amount of a patient colonic tissue mRNA is compared to the amount found in an appropriate tissue reference set. The number (N) of tissues in this reference set should be sufficiently high to ensure that different reference sets (as a whole) behave essentially the same way. If this condition is met, the identity of the individual colonic tissues present in a particular set will have no significant impact on the relative amounts of the genes assayed. Usually, the tissue reference set consists of at least about 30, preferably at least about 40 different IBD tissue specimens. Unless noted otherwise, normalized expression levels for each mRNA/tested tissue/patient will be expressed as a percentage of the expression level measured in the reference set. More specifically, the reference set of a sufficiently high number (e.g. 40) of IBD samples yields a distribution of normalized levels of each mRNA species. The level measured in a particular sample to be analyzed falls at some percentile within this range, which can be determined by methods well known in the art. Below, unless noted otherwise, reference to expression levels of a gene assume normalized expression relative to the reference set although this is not always explicitly stated.

m. Production of Antibodies

The present invention further provides anti-IBD marker antibodies. Exemplary antibodies include polyclonal, monoclonal, humanized, bispecific, and heteroconjugate antibodies. As discussed herein, the antibodies may be used in the diagnostic methods for IBD, and in some cases in methods of treatment of IBD.

(1) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(2) Monoclonal Antibodies

Various methods for making monoclonal antibodies herein are available in the art. For example, the monoclonal antibodies may be made using the hybridoma method first described by Kohler et al., Nature, 256:495 (1975), by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional antibody purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy chain and light chain constant domains in place of the homologous murine sequences (U.S. Pat. No. 4,816,567; and Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(4) Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies. An example of a humanized antibody used to treat IBD is infliximab (Remicade®), an engineered murine-human chimeric monoclonal antibody. The antibody binds the cytokine TNF-alpha and prevents it from binding its receptors to trigger and sustain an inflammatory response. Infliximab is used to treat both CD and UC.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of the humanized antibody are contemplated. For example, the humanized antibody may be an antibody fragment, such as a Fab, which is optionally conjugated with one or more cytotoxic agent(s) in order to generate an immunoconjugate. Alternatively, the humanized antibody may be an intact antibody, such as an intact IgG1 antibody.

(5) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807. Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

(6) Antibody Fragments

Various techniques have been developed for the production of antibody fragments comprising one or more antigen binding regions. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a Alinear antibody@, e.g., as described in U.S. Pat. No. 5,641,870 for example. Such linear antibody fragments may be monospecific or bispecific.

(6) Bispecific Antibodies

Bispecific antibodies are antibodies that have binding specificities for at least two different epitopes. Exemplary bispecific antibodies may bind to two different epitopes of an IBD marker protein. Bispecific antibodies may also be used to localize agents to cells which express an IBD marker protein.

These antibodies possess an IBD marker-binding arm and an arm which binds an agent (e.g. an aminosalicylate). Bispecific antibodies can be prepared as full length antibodies or antibody fragments (e.g. F(ab′)2 bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

(7) Other Amino Acid Sequence Modifications

Amino acid sequence modification(s) of the antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the antibody that are preferred locations for mutagenesis is called “alanine scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in Table 1 under the heading of “preferred substitutions”. If such substitutions result in a change in biological activity, then more substantial changes, denominated “exemplary substitutions” in the following table, or as further described below in reference to amino acid classes, may be introduced and the products screened.

| Original Residue | Exemplary Substitutions | Preferred Substitutions |
|---|---|---|
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; met; ala; | ile |
| | phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |
| | norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)): non-polar: Ala (A), Val (V), Leu (L), Ile (I), Pro (P), Phe (F), Trp (W), Met (M); uncharged polar: Gly (G), Ser (S), Thr (T), Cys (C), Tyr (Y), Asn (N), Gln (O); acidic: Asp (D), Glu (E); and basic: Lys (K), Arg (R), His (H).

Alternatively, naturally occurring residues may be divided into groups based on common side-chain properties: hydrophobic: Norleucine, Met, Ala, Val, Leu, Ile; neutral hydrophilic: Cys, Ser, Thr, Asn, Gln; acidic: Asp, Glu; basic: His, Lys, Arg; residues that influence chain orientation: Gly, Pro; and aromatic: Trp, Tyr, Phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and an IBD marker protein. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Engineered antibodies with three or more (preferably four) functional antigen binding sites are also contemplated (US Appln. No. US2002/0004587 A1, Miller et al.).

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

### B.3 Kits of the Invention

The materials for use in the methods of the present invention are suited for preparation of kits produced in accordance with well known procedures. The invention thus provides kits comprising agents, which may include gene-specific or gene-selective probes and/or primers, for quantitating the expression of the disclosed genes for IBD. Such kits may optionally contain reagents for the extraction of RNA from samples, in particular fixed paraffin-embedded tissue samples and/or reagents for RNA amplification. In addition, the kits may optionally comprise the reagent(s) with an identifying description or label or instructions relating to their use in the methods of the present invention. The kits may comprise containers (including microtiter plates suitable for use in an automated implementation of the method), each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more probes and primers of the present invention (e.g., appropriate length poly(T) or random primers linked to a promoter reactive with the RNA polymerase).

### B.4 Reports of the Invention

The methods of this invention, when practiced for commercial diagnostic purposes generally produce a report or summary of the normalized expression levels of one or more of the selected genes. The methods of this invention will produce a report comprising a prediction of the clinical outcome of a subject diagnosed with an IBD before and after any surgical procedure to treat the IBD. The methods and reports of this invention can further include storing the report in a database. Alternatively, the method can further create a record in a database for the subject and populate the record with data. In one embodiment the report is a paper report, in another embodiment the report is an auditory report, in another embodiment the report is an electronic record. It is contemplated that the report is provided to a physician and/or the patient. The receiving of the report can further include establishing a network connection to a server computer that includes the data and report and requesting the data and report from the server computer.

The methods provided by the present invention may also be automated in whole or in part.

All aspects of the present invention may also be practiced such that a limited number of additional genes that are co-expressed with the disclosed genes, for example as evidenced by high Pearson correlation coefficients, are included in a prognostic or predictive test in addition to and/or in place of disclosed genes.

Having described the invention, the same will be more readily understood through reference to the following Example, which is provided by way of illustration, and is not intended to limit the invention in any way.

### EXAMPLES

### Example 1

### Microarray Analysis

Clinically, IBD is characterized by diverse manifestations often resulting in a chronic, unpredictable course. Bloody diarrhea and abdominal pain are often accompanied by fever and weight loss. Anemia is common, as is severe fatigue. Joint manifestations ranging from arthralgia to acute arthritis as well as abnormalities in liver function are commonly associated with IBD. Patients with IBD also have an increased risk of colon carcinomas compared to the general population. During acute “attacks” of IBD, work and other normal activity are usually impossible, and often a patient is hospitalized.

Although the cause of IBD remains unknown, several factors such as genetic, infectious and immunologic susceptibility have been implicated. IBD is much more common in Caucasians, especially those of Jewish descent. The chronic inflammatory nature of the condition has prompted an intense search for a possible infectious cause. Although agents have been found which stimulate acute inflammation, none has been found to cause the chronic inflammation associated with IBD. The hypothesis that IBD is an autoimmune disease is supported by the previously mentioned extraintestinal manifestation of IBD as joint arthritis, and the known positive response to IBD by treatment with therapeutic agents such as adrenal glucocorticoids, cyclosporine and azathioprine, which are known to suppress immune response. In addition, the GI tract, more than any other organ of the body, is continuously exposed to potential antigenic substances such as proteins from food, bacterial byproducts (LPS), etc. The subtypes of IBD are UC and CD.

CD differs from UC in that the inflammation extends through all layers of the intestinal wall and involves mesentery as well as lymph nodes. CD may affect any part of the alimentary canal from mouth to anus. The disease is often discontinuous, i.e., severely diseased segments of bowel are separated from apparently disease-free areas. In CD, the bowel wall also thickens which can lead to obstructions. In addition, fistulas and fissures are not uncommon.

Both UC and CD are typically diagnosed by endoscopy, which shows the afflicted areas. However, the use of microarray technology has shed light on the molecular pathology of UC. A study published in 2000 used microarray technology to analyze eight UC patients. (Dieckgraefe et al., Physiol. Genomics 2000; 4:1-11). 6500 genes were analyzed in this study and the results confirmed increased expression of genes previously implicated in UC pathogenesis, namely IL-1, IL-1RA and IL-8. Endoscopic diagnosis coupled with the ability to take pinch mucosal biopsies have allowed investigators to further diagnose UC using microarray analysis, and allowed investigators to analyze afflicted tissue against normal tissue and to analyze tissue from a larger range of patients with varying degrees of severity. Biopsies of macroscopically unaffected areas of the colon and terminal ileum were microarrayed. (Langman et al., Gastroent. 2004; 127:26-40). Langman analyzed 22,283 genes and found that genes involved in cellular detoxification, such as pregnane X receptor and MDR1 were significantly downregulated in the colon of patients with UC, but there was no change in expression of these genes in CD patients.

Nucleic acid microarrays, often containing thousands of gene sequences, are useful for identifying differentially expressed genes in diseased tissues as compared to their normal counterparts. Using nucleic acid microarrays, test and control mRNA samples from test and control tissue samples are reverse transcribed and labeled to generate cDNA probes. The cDNA probes are then hybridized to an array of nucleic acids immobilized on a solid support. The array is configured such that the sequence and position of each member of the array is known. For example, a selection of genes known to be expressed in certain disease states may be arrayed on a solid support. Hybridization of a labeled probe with a particular array member indicates that the sample from which the probe was derived expresses that gene. If the hybridization signal of a probe from a test (for example, disease tissue) sample is greater than hybridization signal of a probe from a control, normal tissue sample, the gene or genes overexpressed in the disease tissue are identified. The implication of this result is that an overexpressed protein in a disease tissue is useful not only as a diagnostic marker for the presence of the disease condition, but also as a therapeutic target for treatment of the disease condition.

The methodology of hybridization of nucleic acids and microarray technology is well known in the art. In one example, the specific preparation of nucleic acids for hybridization and probes, slides, and hybridization conditions are all detailed in PCT Patent Application Serial No. PCT/US01/10482, filed on Mar. 30, 2001 and which is herein incorporated by reference.

Microarray analysis was used to find genes that are overexpressed in CD as compared to normal bowel tissue. For this study, sixty seven patients with CD and thirty-one control patients undergoing colonoscopy were recruited. Patient symptoms were evaluated at the time of colonoscopy using the simple clinical colitis activity index (SCCAI). (Walmsley et al., Gut. 1998; 43:29-32). Quiescent disease showing no histological inflammation was defined as a SCCAI of 2 or less. Active disease with histologically acute or chronic inflammation was defined as a SCCAI of greater than 2. The severity of the CD itself was determined by the criteria of Leonard-Jones. (Lennard-Jones Scand. J. Gastroent. 1989; 170:2-6). The CD patients provided well phenotyped biopsies for analysis of inflammatory pathways of CD at the molecular level, thus identifying novel candidate genes and potential pathways for therapeutic intervention. Paired biopsies were taken from each anatomical location.

All biopsies were stored at −70° C. until ready for RNA isolation. The biopsies were homogenized in 600 μl of RLT buffer (+BME) and RNA was isolated using Qiagen™ Rneasy Mini columns (Qiagen™) with on-column DNase treatment following the manufacturer's guidelines. Following RNA isolation, RNA was quantitated using RiboGreen™ (Molecular Probes) following the manufacturer's guidelines and checked on agarose gels for integrity. Appropriate amounts of RNA were labeled for microarray analysis and samples were run on proprietary Genentech microarray and Affymetrics™ microarrays. Genes were compared whose expression was upregulated in UC tissue vs normal bowel, matching biopsies from normal bowel and CD tissue from the same patient. The results of this experiment showed that the nucleic acids as shown in SEQ ID NOS:1-426 are differentially expressed in CD tissue in comparison to normal tissue. More specifically, SEQ ID NOS:1-316 represent polynucleotides and their encoded polypeptide which are significantly up-regulated/overexpressed in CD. SEQ ID NO:317-426 represent polynucleotides and their encoded polypeptide which are significantly down-regulated/under-expressed in CD.

What is claimed is:

1. A method of diagnosing the presence of an inflammatory bowel disease (IBD) in a mammalian subject, comprising determining that the level of expression of a nucleic acid encoding a polypeptide shown as any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, and 316 in a test sample obtained from said subject is higher relative to the level of expression in a control, wherein said higher level of expression is indicative of the presence of an IBD in the subject from which the test sample was obtained.

2. A method of diagnosing the presence of an inflammatory bowel disease (IBD) in a mammalian subject, comprising determining that the level of expression of a nucleic acid encoding a polypeptide shown as any one of SEQ ID NOS: 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, and 426 in a test sample obtained from said subject is lower relative to the level of expression in a control, wherein said lower level of expression is indicative of the presence of an IBD in the subject from which the test sample was obtained.

3. The method of claim 1 or 2 wherein said mammalian subject is a human patient.

4. The method of claim 3 wherein evidence of said expression level is obtained by a method of gene expression profiling.

5. The method of claim 3 wherein said method is a PCR-based method.

6. The method of claim 4 wherein said expression levels are normalized relative to the expression levels of one or more reference genes, or their expression products.

7. The method of claim 1 or 2 comprising determining evidence of the expression levels of at least two of said nucleic acids.

8. The method of claim 1 or 2 comprising determining evidence of the expression levels of at least three of said nucleic acids.

9. The method of claim 1 or 2 comprising determining evidence of the expression levels of at least four of said nucleic acids.

10. The method of claim 1 or 2 comprising determining evidence of the expression levels of at least five of said nucleic acids.

11. The method of claim 1 or 2 further comprising the step of creating a report summarizing said IBD detection.

12. The method of claim 1 or 2, wherein said IBD is Crohn's disease.

13. The method of claim 1 or 2, wherein said test sample is from a colonic tissue biopsy.

14. The method of claim 13, wherein said biopsy is from a tissue selected from the group consisting of the ascending colon, the descending colon, and the sigmoid colon.

15. The method of claim 13, wherein said biopsy is from an inflamed colonic area.

16. The method of claim 13, wherein said biopsy is from a non-inflamed colonic area.

17. The method of claim 1 or 2, wherein said determining step is indicative of a recurrence of an IBD in said mammalian subject, and wherein said mammalian subject was previously diagnosed with an IBD and treated for said previously diagnosed IBD.

18. The method of claim 17, wherein said treatment comprised surgery.

19. The method of claim 17, wherein said recurrence is a flare-up of an inactive IBD in said mammalian subject.

20. A method of treating an inflammatory bowel disorder (IBD) in a mammalian subject in need thereof, the method comprising the steps of
(a) determining that the level of expression of a nucleic acid encoding the polypeptide shown as any one of SEQ ID NOS:SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, and 316 in a test sample obtained from said subject is higher relative to the level of expression in a control, wherein said higher level of expression is indicative of the presence of an IBD in the subject from which the test sample was obtained; and
(b) administering to said subject an effective amount of an IBD therapeutic agent.

21. A method of treating an inflammatory bowel disorder (IBD) in a mammalian subject in need thereof, the method comprising the steps of
(a) determining that the level of expression of a nucleic acid encoding the polypeptide shown as any one of SEQ ID NOS:SEQ ID NOS: 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, and 426 in a test sample obtained from said subject is lower relative to the level of expression in a control, wherein said lower level of expression is indicative of the presence of an IBD in the subject from which the test sample was obtained; and
(b) administering to said subject an effective amount of an IBD therapeutic agent.

22. The method of claim 20 or 21 wherein said mammalian subject is a human patient.

23. The method of claim 22 wherein evidence of said expression level is obtained by a method of gene expression profiling.

24. The method of claim 22 wherein said method is a PCR-based method.

25. The method of claim 23 wherein said expression levels are normalized relative to the expression levels of one or more reference genes, or their expression products.

26. The method of claim 20 or 21 comprising determining evidence of the expression levels of at least two of said nucleic acids.

27. The method of claim 20 or 21 comprising determining evidence of the expression levels of at least three of said nucleic acids.

28. The method of claim 20 or 21 comprising determining evidence of the expression levels of at least four of said nucleic acids.

29. The method of claim 20 or 21 comprising determining evidence of the expression levels of at least five of said nucleic acids.

30. The method of claim 20 or 21 further comprising the step of creating a report summarizing said IBD detection.

31. The method of claim 20 or 21, wherein said IBD is Crohn's disease.

32. The method of claim 20 or 21, wherein said test sample is from a colonic tissue biopsy.

33. The method of claim 32, wherein said biopsy is from a tissue selected from the group consisting of the terminal ileum, the ascending colon, the descending colon, and the sigmoid colon.

34. The method of claim 32, wherein said biopsy is from an inflamed colonic area.

35. The method of claim 32, wherein said biopsy is from a non-inflamed colonic area.

36. The method of claim 20 or 21, wherein said determining step is indicative of a recurrence of an IBD in said mammalian subject, and wherein said mammalian subject was previously diagnosed with an IBD and treated for said previously diagnosed IBD.

37. The method of claim 36, wherein said treatment comprised surgery.

38. The method of claim 20 or 21, wherein said determining step is indicative of a flare-up of said IBD in said mammalian subject.

39. The method of claim 20 or 21, wherein said IBD therapeutic agent is an aminosalicylate.

40. The method of claim 20 or 21, wherein said IBD therapeutic agent is a corticosteroid.

41. The method of claim 20 or 21, wherein said IBD therapeutic agent is an immunosuppressive agent.
